(19) United States
(12) Patent Application Publication
(10) Pub. No.: US 2023/0106583 A1
(43) Pub. Date: Apr. 6, 2023

(54) HETEROARYL COMPOUNDS

(71) Applicant: Vivace Therapeutics, Inc., San Mateo, CA (US)

(72) Inventors: Andrei W. KONRADI, Burlingame, CA (US); Tracy Tzu-Ling Tang LIN, Redwood City, CA (US)

(21) Appl. No.: 17/777,716

(22) PCT Filed: Nov. 19, 2020
(86) PCT No.: PCT/US2020/061387

§ 371 Date: May 18, 2022

Related U.S. Application Data
(60) Provisional application No. 62/938,097, filed on Nov. 20, 2019.

(51) Int. Cl. C07D 413/04; C07D 413/14; C07D 401/14; A61P 35/00

(52) U.S. Cl. CPC C07D 413/04; C07D 413/14; C07D 401/14; A61P 35/00

(57) ABSTRACT

Provided herein are compounds and pharmaceutical compositions comprising said compounds that are useful for treating cancers. Specific cancers include those that are mediated by YAP/TAZ or those that are modulated by the interaction between YAP/TAZ and TEAD.

### CROSS-REFERENCE

This application claims benefit of U.S. Provisional Patent Application No. 62/938,097 filed on Nov. 20, 2019, which is incorporated herein by reference in its entirety.

### BACKGROUND OF THE DISCLOSURE

YAP and TAZ are transcriptional co-activators of the Hippo pathway network and regulate cell proliferation, migration, and apoptosis. Inhibition of the Hippo pathway promotes YAP/TAZ translocation to the nucleus, wherein YAP/TAZ interact with transcriptional enhancer associate domain (TEAD) transcription factors and coactivate the expression of target genes and promote cell proliferation. Hyperactivation of YAP and TAZ and/or mutations in one or more members of the Hippo pathway network have been implicated in numerous cancers. Described herein are inhibitors associated with one or more members of the Hippo pathway network, such as inhibitors of YAP/TAZ or inhibitors that modulate the interaction between YAP/TAZ and TEAD.

### SUMMARY OF THE DISCLOSURE

Provided herein are heteroaryl compounds and pharmaceutical compositions comprising said compounds. In some embodiments, the subject compounds are useful for the treatment of cancer.

In one aspect, the present disclosure provides a compound of Formula (A) or a pharmaceutically acceptable salt or solvate thereof:

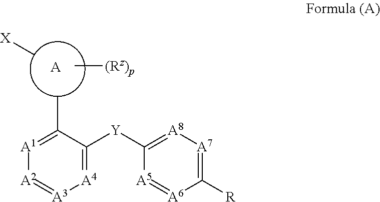

wherein
ring A is a 5-membered heteroaryl;
each A¹, A², A³, and A⁴ is independently N or CR¹;
each A⁵, A⁶, A⁷, and A⁸ is independently N or CR²; wherein at least one of A¹, A², A³, A⁴, A⁵, A⁶, A⁷, and A⁸ is N;
R is halogen, nitro, —CN, —O(C₁-C₆fluoroalkyl), —S(C₁-C₆fluoroalkyl), —S(halogen)₅, or substituted or unsubstituted C₁-C₆fluoroalkyl;
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
Y is O, S, or NR³;
X is H, —CN, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, -L²-Y², -L²-L³-Y², or -L²-L³-L⁴-Y².
each L² and L⁴ is independently absent, substituted or unsubstituted C₁-C₆alkylene, substituted or unsubstituted C₃-C₁₀cycloalkylene, or substituted or unsubstituted C₂-C₁₀heterocycloalkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —O—NR³(C═O)—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —(C═O)—NR³S(═O)₂—, —S(═O)₂NR³—(C═O)O—, —O(C═O)—NR³S(═O)₂—, —NR³S(═O)₂NR³—(C═O)—, —(C═O)—NR³S(═O)₂NR³—, —O(C═O)—NR³S(═O)₂—NR³—, —NR³S(═O)₂NR³—(C═O)O—, —OS(═O)₂—, or —S(═O)₂O—;
Y² is H, —CN, —N₃, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b};
each R³ is independently H, —CN, —S(═O)₂(C₁-C₄alkyl), or substituted or unsubstituted C₁-C₆alkyl;
or R³ and Y² on the same N atom are taken together with the N atom to which they are attached to form a substituted or unsubstituted N-containing heterocycle;
each R⁴, R^{4a}, and R^{4b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{4a} and R^{4b} on the same N atom are taken together with the N atom to which they are attached to form a substituted or unsubstituted N-containing heterocycle;
each R⁵, R^{5a}, and R^{5b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{5a} and R^{5b} on the same N atom are taken together with the N atom to which they are attached to form a substituted or unsubstituted N-containing heterocycle; and
each R⁶, R^{6a}, and R^{6b} is independently H or substituted or unsubstituted C₁-C₆alkyl; and
or R^{6a} and R^{6b} on the same N atom are taken together with the N atom to which they are attached to form a substituted or unsubstituted N-containing heterocycle;
each R^{z} is independently H, halogen, or substituted or unsubstituted C₁-C₆alkyl; and
p is 1, 2, or 3.

In another aspect, the present disclosure provides a compound of Formula (I) or a pharmaceutically acceptable salt or solvate thereof:

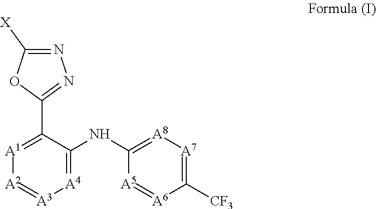

wherein
each A¹, A², A³, and A⁴ is independently N or CR¹;
each A⁵, A⁶, A⁷, and A⁸ is independently N or CR²; wherein at least one of A¹, A², A³, A⁴, A⁵, A⁶, A⁷, and A⁸ is N;
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, or -L⁷-Y³;
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, or

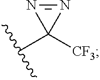 [SMILES: *C1(C(F)(F)F)N=N1]

X is H, —CN, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, -L¹-Y¹, -L²-L³-Y², -L²-L³-L⁴-Y², -L⁵-L⁶-L³-Y², or -L⁶-L⁵-L³-Y²;
L¹ is substituted or unsubstituted C₁-C₆alkylene;
L² is absent, substituted or unsubstituted C₁-C₆alkylene, substituted or unsubstituted C₃-C₁₀cycloalkylene, or substituted or unsubstituted C₂-C₁₀heterocycloalkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —O—NR³(C═O)—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —(C═O)—NR³S(═O)₂—, —S(═O)₂NR³—(C═O)O—, —O(C═O)—NR³S(═O)₂—, —NR³S(═O)₂NR³—(C═O)—, —(C═O)—NR³S(═O)₂NR³—, —O(C═O)—NR³S(═O)₂—NR³—, —NR³S(═O)₂NR³—(C═O)O—, —OS(═O)₂—, or —S(═O)₂O—;
L⁴ is substituted or unsubstituted C₁-C₆alkylene;
L⁵ is substituted or unsubstituted C₁-C₆alkylene;
L⁶ is substituted or unsubstituted C₃-C₁₀cycloalkylene;
L⁷ is substituted or unsubstituted C₁-C₆alkylene;
Y¹ is —N₃, substituted or unsubstituted C₃-C₁₀cycloalkyl, substituted or unsubstituted C₂-C₁₀ heterocycloalkyl, substituted or unsubstituted aryl, or substituted or unsubstituted heteroaryl;
Y² is H, —CN, —N₃, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b};
Y³ is —Si(R⁷)₃;
each R³ is independently H, —CN, —S(═O)₂(C₁-C₄alkyl), or substituted or unsubstituted C₁-C₆alkyl;
or R³ and Y² on the same N atom are taken together with the N atom to which they are attached to form a substituted or unsubstituted N-containing heterocycle;
each R⁴, R^{4a}, and R^{4b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{4a} and R^{4b} on the same N atom are taken together with the N atom to which they are attached to form a substituted or unsubstituted N-containing heterocycle;
each R⁵, R^{5a}, and R^{5b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{5a} and R^{5b} on the same N atom are taken together with the N atom to which they are attached to form a substituted or unsubstituted N-containing heterocycle; and
each R⁶, R^{6a}, and R^{6b} is independently H or substituted or unsubstituted C₁-C₆alkyl;
or R^{6a} and R^{6b} on the same N atom are taken together with the N atom to which they are attached to form a substituted or unsubstituted N-containing heterocycle; and
each R⁷ is independently substituted or unsubstituted C₁-C₆alkyl.

Any combination of the groups described above for the various variables is contemplated herein. Throughout the specification, groups and substituents thereof are chosen by one skilled in the relevant field to provide stable moieties and compounds.

In another aspect, the present disclosure provides a compound or pharmaceutically acceptable salt or solvate thereof, wherein the compound is a compound from Table 1, or a pharmaceutically acceptable salt or solvate thereof.

In another aspect, the present disclosure provides a pharmaceutical composition comprising a pharmaceutically acceptable excipient and a compound disclosed herein or a pharmaceutically acceptable salt or solvate thereof.

In another aspect, the present disclosure provides a method of inhibiting one or more of proteins encompassed by, or related to, the Hippo pathway in a subject, comprising administering to a subject a compound disclosed herein, or a pharmaceutically acceptable salt or solvate thereof.

In another aspect, the present disclosure provides a method of inhibiting transcriptional coactivator with PDZ binding motif/Yes-associated protein transcriptional coactivator (TAZ/YAP) in a subject comprising administering to a subject a compound disclosed herein, or a pharmaceutically acceptable salt or solvate thereof. In some embodiments, the subject has cancer, polycystic kidney disease, or liver fibrosis. In some embodiments, the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

In another aspect, the present disclosure provides a method of treating cancer in a subject in need thereof comprising administering to the subject in need thereof a therapeutically effective amount of a compound disclosed herein, or a pharmaceutically acceptable salt or solvate thereof. In some embodiments, the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

In another aspect, the present disclosure provides a method of treating polycystic kidney disease or liver fibrosis in a subject in need thereof comprising administering to the subject in need thereof a therapeutically effective amount of a compound disclosed herein, or a pharmaceutically acceptable salt or solvate thereof.

### DETAILED DESCRIPTION OF THE DISCLOSURE

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, in some embodiments, ranges and amounts are expressed as “about” particular value or range. About also includes the exact amount. Hence “about 5 μL” means “about 5 μL” and also “5 μL.” Generally, the term “about” includes an amount that is expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly, or a hospice worker).

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —

SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each R^{a} is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —

SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each R^{a} is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —

SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each R^{a} is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. In some embodiments, the points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —

SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each R^{a} is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each R^{f} is independently alkyl, fluoroalkyl, carbocyclyl, carbocyclylalkyl, aryl, aralkyl, heterocyclyl, heterocyclylalkyl, heteroaryl, or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin, and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted

heteroarylalkyl, —R^{b}—CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, and in some embodiments, include fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. In some embodiments, the carbocyclyl is saturated, (i.e., containing single C—C bonds only) or unsaturated (i.e., containing one or more double bonds or triple bonds.) A carbocyclyl radical is also referred to as “cycloalkyl.” In some embodiments, cycloalkyl radical is fused with an aromatic ring (in which case the cycloalkyl is bonded through a non-aromatic ring carbon atom). Examples of monocyclic cycloalkyls include, e.g., cyclopropyl, cyclobutyl, cyclopentyl, cyclohexyl, cycloheptyl, cyclooctyl, cyclopentenyl, cyclohexenyl, cycloheptenyl, and cyclooctenyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted

heteroarylalkyl, —CN, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Halo” or “halogen” refers to bromo, chloro, fluoro, or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycle” refers to a stable 3- to 18-membered non-aromatic ring radical that comprises two to fifteen carbon atoms and from one to six heteroatoms each independently selected from nitrogen, oxygen, and sulfur. In some embodiments, heterocyclyl radical comprises two to twelve carbon atoms. Unless stated otherwise in the specification, the heterocyclyl radical is a monocyclic, bicyclic, tricyclic, or tetracyclic ring system, which include fused, bridged, or spiro ring systems in some embodiments. In some embodiments, when fused with an aryl or a heteroaryl ring, the heterocyclyl is bonded through a non-aromatic ring atom. The heteroatoms in the heterocyclyl radical are optionally oxidized. One or more nitrogen atoms, if present, are optionally quaternized. In some embodiments, heterocyclyl radical comprises 2-12 C atoms, 0-6 N atoms, 0-4 O atoms, and 0-4 S atoms. In some embodiments, heterocyclyl radical comprises 2-10 C atoms, 0-4 N atoms, 0-2 O atoms, and 0-2 S atoms. In some embodiments, heterocyclyl radical comprises 2-8 C atoms, 0-3 N atoms, 0-1 O atoms, and 0-1 S atoms. In some embodiments, heterocyclyl radical is a saturated or partially unsaturated 3-7 membered monocyclic, 6-10 membered bicyclic, or 13-16 membered polycyclic (e.g., tricyclic or tetracyclic) ring system having 1, 2, 3, or 4 heteroatom ring members each independently selected from N, O, and S. In some embodiments, heterocyclyl radical comprises 1 or 2 heteroatom ring members each independently selected from N, O, and S. In some embodiments, the heterocyclyl is attached to the rest of the molecule through any atom of the ring(s). In some embodiments, the heterocyclyl is saturated, (i.e., containing single bonds only) or partially unsaturated (i.e., containing one or more double bonds or triple bonds.) A heterocyclyl radical is also referred to as “heterocycloalkyl.” Examples of such heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, dihydroindolyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —CN, —R^{b}—CN,

—R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl. In some embodiments, the alkyl part of the heteroalkyl radical is optionally substituted as defined for an alkyl group.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, in some embodiments, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C (O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

In some embodiments, the compounds disclosed herein contain one or more asymmetric centers and thus give rise to enantiomers, diastereomers, and other stereoisomeric forms that are defined, in terms of absolute stereochemistry, as (R)— or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

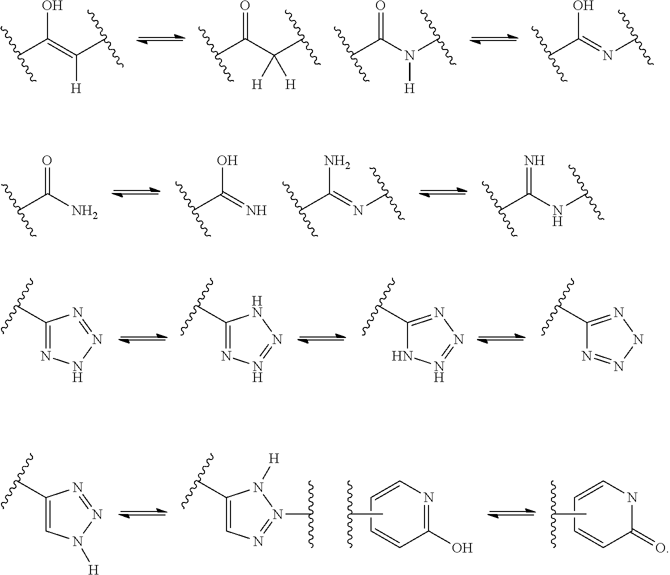

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the compounds described herein is intended to encompass any and all pharmaceutically suitable salt forms. Pharmaceutically acceptable salts of the compounds described herein are optionally pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). In some embodiments, acid addition salts of basic compounds are prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines, and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins, and the like. See Berge et al., supra.

As used herein, “treatment” or “treating” or “palliating” or “ameliorating” are used interchangeably herein. These terms refer to an approach for obtaining beneficial or desired results including, but not limited to, therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is afflicted with the underlying disorder in some embodiments. For prophylactic benefit, in some embodiments, the compositions are administered to a patient at risk of developing a particular disease, or to a patient reporting one or more of the physiological symptoms of a disease, even though a diagnosis of this disease has not been made.

“Prodrug” is meant to indicate a compound that is converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. In some embodiments, prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of prodrugs include, but are not limited to, acetate, formate, and benzoate derivatives of alcohol or amine functional groups in the active compounds and the like.

### Compounds

In some embodiments, the compounds disclosed herein are heteroaryl compounds.

In one aspect, the present disclosure provides a compound of Formula (A) or a pharmaceutically acceptable salt or solvate thereof:

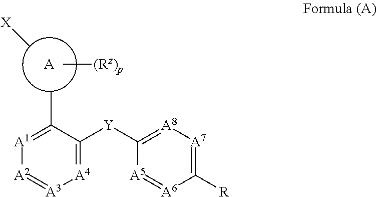

wherein
ring A is a 5-membered heteroaryl;
each A¹, A², A³, and A⁴ is independently N or CR¹;
each A⁵, A⁶, A⁷, and A⁸ is independently N or CR²; wherein at least one of A¹, A², A³, A⁴, A⁵, A⁶, A⁷, and A⁸ is N;
R is halogen, nitro, —CN, —O(C₁-C₆fluoroalkyl), —S(C₁-C₆fluoroalkyl), —S(halogen)₅, or substituted or unsubstituted C₁-C₆fluoroalkyl;
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
Y is O, S, or NR³;
X is H, —CN, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, -L²-Y², -L²-L³-Y², or -L²-L³-L⁴-Y²;
each L² and L⁴ is independently absent, substituted or unsubstituted C₁-C₆alkylene, substituted or unsubstituted C₃-C₁₀cycloalkylene, or substituted or unsubstituted C₂-C₁₀heterocycloalkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —O—NR³(C═O)—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —(C═O)—NR³S(═O)₂—, —S(═O)₂NR³—(C═O)O—, —O(C═O)—NR³S(═O)₂—, —NR³S(═O)₂NR³—(C═O)—, —(C═O)—NR³S(═O)₂NR³—, —O(C═O)—NR³S(═O)₂—NR³—, —NR³S(═O)₂NR³—(C═O)O—, —OS(═O)₂—, or —S(═O)₂O—;
Y² is H, —CN, —N₃, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b};
each R³ is independently H, —CN, —S(═O)₂(C₁-C₄alkyl), or substituted or unsubstituted C₁-C₆alkyl;
or R³ and Y² on the same N atom are taken together with the N atom to which they are attached to form a substituted or unsubstituted N-containing heterocycle;
each R⁴, R^{4a}, and R^{4b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{4a} and R^{4b} on the same N atom are taken together with the N atom to which they are attached to form a substituted or unsubstituted N-containing heterocycle;
each R⁵, R^{5a}, and R^{5b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{5a} and R^{5b} on the same N atom are taken together with the N atom to which they are attached to form a substituted or unsubstituted N-containing heterocycle; and
each R⁶, R^{6a}, and R^{6b} is independently H or substituted or unsubstituted C₁-C₆alkyl; and
or R^{6a} and R^{6b} on the same N atom are taken together with the N atom to which they are attached to form a substituted or unsubstituted N-containing heterocycle;
each R^{z} is independently H, halogen, or substituted or unsubstituted C₁-C₆alkyl; and
p is 1, 2, or 3.

In another aspect, the present disclosure provides a compound of Formula (I), or a pharmaceutically acceptable salt or solvate thereof:

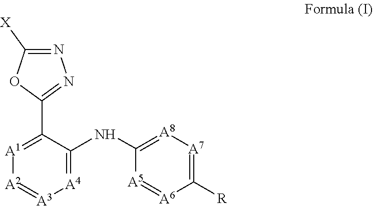

wherein
each A¹, A², A³, and A⁴ is independently N or CR¹;
each A⁵, A⁶, A⁷, and A⁸ is independently N or CR²; wherein at least one of A¹, A², A³, A⁴, A⁵, A⁶, A⁷, and A⁸ is N;
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, or -L⁷-Y³;
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, or

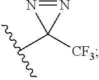 [SMILES: *C1(C(F)(F)F)N=N1]

X is H, —CN, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, -L¹-Y¹, -L²-L³-Y², -L²-L³-L⁴-Y², -L⁵-L⁶-L³-Y², or -L⁶-L⁵-L³-Y²;
L¹ is substituted or unsubstituted C₁-C₆alkylene;
L² is absent, substituted or unsubstituted C₁-C₆alkylene, substituted or unsubstituted C₃-C₁₀cycloalkylene, or substituted or unsubstituted C₂-C₁₀heterocycloalkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —O—NR³(C═O)—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —(C═O)—NR³S(═O)₂—, —S(═O)₂NR³—(C═O)O—, —O(C═O)—NR³S(═O)₂—, —NR³S(═O)₂NR³—(C═O)—, —(C═O)—NR³S(═O)₂NR³—, —O(C═O)—NR³S(═O)₂—NR³—, —NR³S(═O)₂NR³—(C═O)O—, —OS(═O)₂—, or —S(═O)₂O—;
L⁴ is substituted or unsubstituted C₁-C₆alkylene;
L⁵ is substituted or unsubstituted C₁-C₆alkylene;
L⁶ is substituted or unsubstituted C₃-C₁₀cycloalkylene;
L⁷ is substituted or unsubstituted C₁-C₆alkylene;
Y¹ is —N₃, substituted or unsubstituted C₃-C₁₀cycloalkyl, substituted or unsubstituted C₂-C₁₀ heterocycloalkyl, substituted or unsubstituted aryl, or substituted or unsubstituted heteroaryl;
Y² is H, —CN, —N₃, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b};
Y³ is —Si(R⁷)₃;
each R³ is independently H, —CN, —S(═O)₂(C₁-C₄alkyl), or substituted or unsubstituted C₁-C₆alkyl;
or R³ and Y² on the same N atom are taken together with the N atom to which they are attached to form a substituted or unsubstituted N-containing heterocycle;
each R⁴, R^{4a}, and R^{4b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{4a} and R^{4b} on the same N atom are taken together with the N atom to which they are attached to form a substituted or unsubstituted N-containing heterocycle;
each R⁵, R^{5a}, and R^{5b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{5a} and R^{5b} on the same N atom are taken together with the N atom to which they are attached to form a substituted or unsubstituted N-containing heterocycle; and
each R⁶, R^{6a}, and R^{6b} is independently H or substituted or unsubstituted C₁-C₆alkyl;
or R^{6a} and R^{6b} on the same N atom are taken together with the N atom to which they are attached to form a substituted or unsubstituted N-containing heterocycle; and
each R⁷ is independently substituted or unsubstituted C₁-C₆alkyl.

In some embodiments, A⁵ is N. In some embodiments, A⁵ is CR². In some embodiments, A⁶ is N. In some embodiments, A⁶ is CR². In some embodiments, A⁶ is N. In some embodiments, A⁷ is CR². In some embodiments, A⁸ is N. In some embodiments, A⁸ is CR².

In some embodiments, A⁵ is CR²; A⁶ is CR²; A⁷ is CR²; and A⁸ is CR². In some embodiments, A⁵ is N; A⁶ is CR²; A⁷ is CR²; and A⁸ is CR². In some embodiments, A⁵ is CR²; A⁶ is N; A⁷ is CR²; and A⁸ is CR². In some embodiments, A⁵ is CR²; A⁶ is CR²; A⁷ is N; and A⁸ is CR². In some embodiments, A⁵ is CR²; A⁶ is CR²; A⁷ is CR²; and A⁸ is N.

In some embodiments, a compound of Formula (A) has a structure of Formula (A-1):

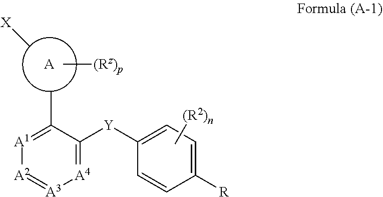

wherein, Y is O, S, or NH; and n is 1, 2, 3, or 4.

In some embodiments, Y is O. In some embodiments, Y is S. In some embodiments, Y is NH.

In some embodiments, a compound of Formula (A) has a structure of Formula (Ia):

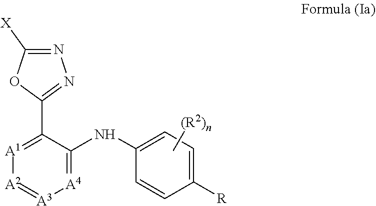

wherein, n is 1, 2, 3, or 4.

In some embodiments, a compound of Formula (A) has a structure of Formula (Ia-1):

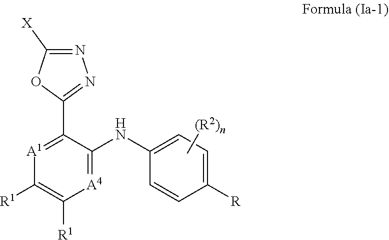

wherein, n is 1, 2, 3, or 4.

In some embodiments, a compound of Formula (A) has a structure of Formula (Ib):

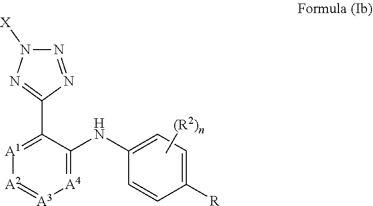

wherein, n is 1, 2, 3, or 4.

In some embodiments, a compound of Formula (A) has a structure of Formula (Ib-1):

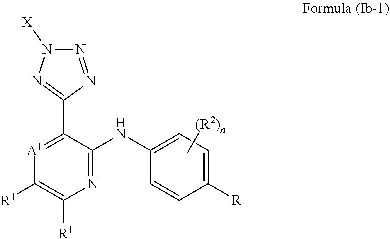

wherein, n is 1, 2, 3, or 4.

In some embodiments, A¹ is N. In some embodiments, A¹ is CR². In some embodiments, A² is N. In some embodiments, A² is CR². In some embodiments, A³ is N. In some embodiments, A³ is CR². In some embodiments, A⁴ is N. In some embodiments, A⁴ is CR².

In some embodiments, a ring comprising A¹, A², A³, and A⁴ variables is a 6-membered heteroaryl ring. In some embodiments, a ring comprising A¹, A², A³, and A⁴ variables is a 6-membered heteroaryl ring comprising 1-4 N ring atoms. In some embodiments, a ring comprising A¹, A², A³, and A⁴ variables is a 6-membered heteroaryl ring comprising 1-2 N ring atoms. In some embodiments, a ring comprising A¹, A², A³, and A⁴ variables is pyridine, pyridazine, pyrimidine, pyrazine, or triazine. In some embodiments, a ring comprising A¹, A², A³, and A⁴ variables is pyridine. In some embodiments, a ring comprising A¹, A², A³, and A⁴ variables is pyrazine. In some embodiments,

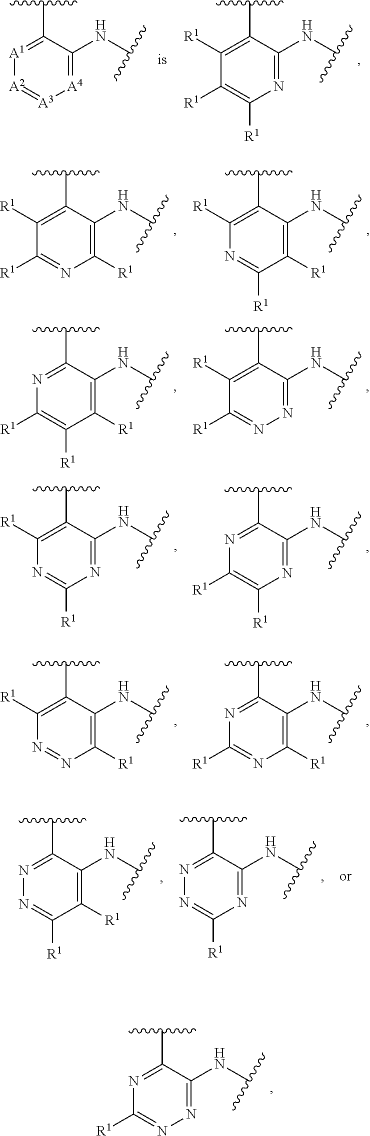

and each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl. In some embodiments,

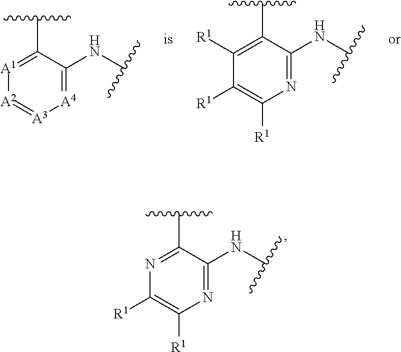

and each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl. In some embodiments,

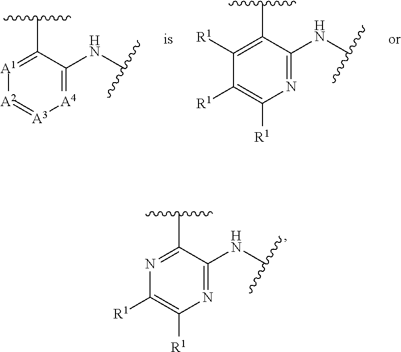

and each R¹ is independently H, halogen, —CN, —OCH₃, or —CH₃.

In some embodiments, a compound of Formula (A) or Formula (A-1) has a structure of Formula (A-2):

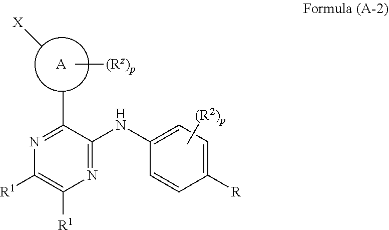

wherein, n is 1, 2, 3, or 4.

In some embodiments, a compound of Formula (A) or Formula (A-1) has a structure of Formula (A-3):

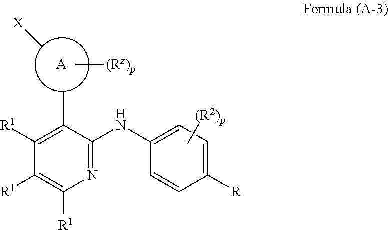

wherein, n is 1, 2, 3, or 4.

In some embodiments, a compound of Formula (A) or Formula (A-1) has a structure of Formula (A-4):

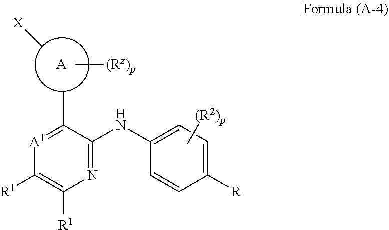

wherein, n is 1, 2, 3, or 4.

In some embodiments, a compound of Formula (A) or Formula (I) has a structure of Formula (Ic):

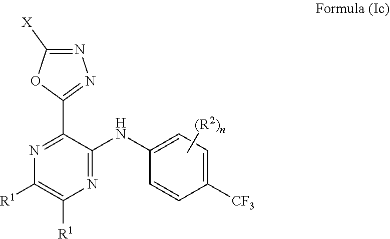

wherein, n is 1, 2, 3, or 4.

In some embodiments, a compound of Formula (A) or Formula (I) has a structure of Formula (Id):

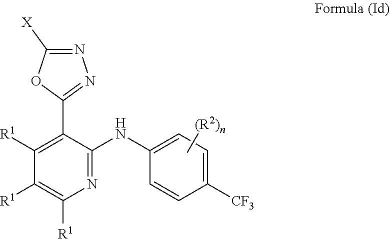

wherein, n is 1, 2, 3, or 4.

In some embodiments, each R¹ is independently H, halogen, —CN, —OR⁴, —SR⁴, —NR^{4a}R^{4b}, substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, or substituted or unsubstituted monocyclic heteroaryl. In some embodiments, each R¹ is independently H, halogen, substituted or unsubstituted C₁-C₄alkyl, or substituted or unsubstituted C₁-C₄haloalkyl. In some embodiments, each R¹ is independently H, F, Cl, Br, I, —CH₃, —CH₂CH₃, —CH₂CH₂CH₃, —CH(CH₃)₂, —CH₂CH₂CH₂CH₃, —CH₂CH(CH₃)₂, —C(CH₃)₃, —CH₂F, —CHF₂, —CF₃, or —CH₂CF₃. In some embodiments, each R¹ is H.

In some embodiments, ring A is a 5-membered heteroaryl comprising 1-4 N, 0-1 O, and 0-1 S ring atoms. In some embodiments, ring A is a 5-membered heteroaryl comprising 2-4 N ring atoms. In some embodiments, ring A is a 5-membered heteroaryl comprising 2 N ring atoms. In some embodiments, ring A is a 5-membered heteroaryl comprising 3 N ring atoms. In some embodiments, ring A is a 5-membered heteroaryl comprising 4 N ring atoms. In some embodiments, ring A is a 5-membered heteroaryl comprising 1 N and 1 O ring atoms. In some embodiments, ring A is a 5-membered heteroaryl comprising 1 N and 1 S ring atoms. In some embodiments, ring A is a 5-membered heteroaryl comprising 2 N and 1 O ring atoms. In some embodiments, ring A is a 5-membered heteroaryl comprising 2 N and 1 S ring atoms. In some embodiments,

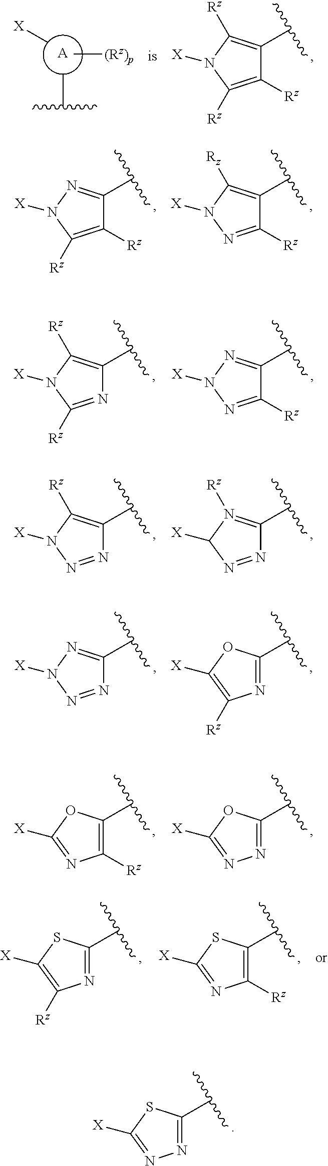

In some embodiments,

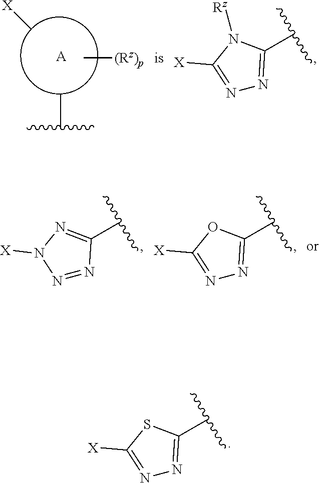

In some embodiments,

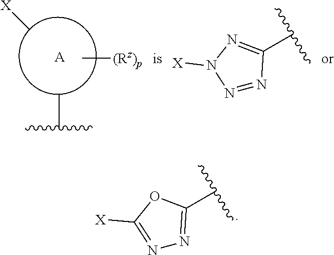

In some embodiments,

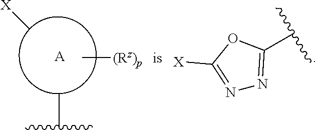

In some embodiments,

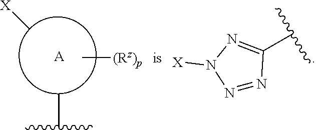

In some embodiments, R² is H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₁-C₆heteroalkyl, substituted or unsubstituted aryl, or substituted or unsubstituted heteroaryl. In some embodiments, R² is H, halogen, —N₃, —CN, —OR⁵, —SR⁵, —S(═O)₂R⁵, —NR^{5a}R^{5b}, —C(═O)OR⁵, substituted or unsubstituted C₁-C₄alkyl, substituted or unsubstituted C₁-C₄haloalkyl, substituted or unsubstituted C₁-C₄heteroalkyl, substituted or unsubstituted benzyl, substituted or unsubstituted phenyl, or substituted or unsubstituted monocyclic heteroaryl; and each R⁵, R^{5a}, and R^{5b} is independently H, or substituted or unsubstituted C₁-C₄alkyl. In some embodiments, R² is H, halogen, —N₃, —OR⁵, —S(═O)₂R⁵, —C(═O)OR⁵, substituted or unsubstituted C₁-C₄alkyl, or substituted or unsubstituted C₁-C₄haloalkyl. In some embodiments, each R² is independently H, halogen, —N₃, —OR⁵, —S(═O)₂R⁵, —C(═O)OR⁵, substituted or unsubstituted C₁-C₄alkyl, or substituted or unsubstituted C₁-C₄haloalkyl. In some embodiments, each R² is independently H, halogen, —OR⁵, substituted or unsubstituted C₁-C₄alkyl, or substituted or unsubstituted C₁-C₄haloalkyl. In some embodiments, each R² is independently H, F, Cl, —OCF₃, or —CF₃. In some embodiments, each R² is independently H, F, or Cl. In some embodiments, each R² is H.

In some embodiments, a compound of Formula (A) has a structure of Formula (A-4):

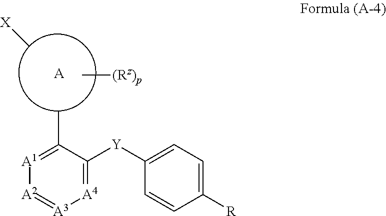

wherein, Y is O, S, or NH.

In some embodiments, a compound of Formula (A) or Formula (I) has a structure of Formula (Ie):

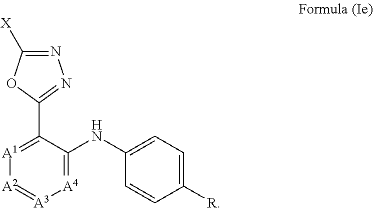

In some embodiments, a compound of Formula (A) has a structure of Formula (If):

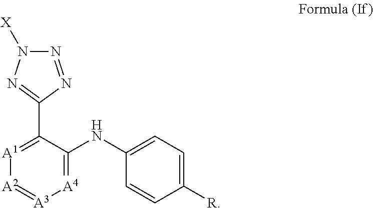

In some embodiments, X is substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl. In some embodiments, X is substituted or unsubstituted C₁-C₆alkyl, substituted or unsubstituted C₁-C₆haloalkyl, substituted or unsubstituted C₂-C₁₀heterocycloalkyl, substituted or unsubstituted aryl, or substituted or unsubstituted heteroaryl. In some embodiments, X is substituted or unsubstituted C₁-C₆alkyl, substituted or unsubstituted C₁-C₆haloalkyl, substituted or unsubstituted C₃-C₁₀cycloalkyl, substituted or unsubstituted C₂-C₁₀heterocycloalkyl, or substituted or unsubstituted heteroaryl. In some embodiments, X is substituted or unsubstituted C₁-C₆alkyl, substituted or unsubstituted C₃-C₁₀cycloalkyl, or substituted or unsubstituted C₂-C₁₀heterocycloalkyl. In some embodiments, X is substituted or unsubstituted C₁-C₆alkyl. In some embodiments, X is —CH₃, —CH₂CH₃, —CH₂CH₂CH₃, —CH(CH₃)₂, —CH₂CH₂CH₂CH₃, —CH₂CH(CH₃)₂, or —C(CH₃)₃. In some embodiments, X is substituted C₁-C₆alkyl.

In some embodiments,
X is C₁-C₆alkyl substituted with 1, 2, or 3 substituents each independently selected from —OR¹¹, —(C═O)R¹¹, NR¹¹(C═O)R¹¹, —(C═O)OR¹¹, —NR¹¹(C═O)OR¹¹, —O(C═O)OR¹¹, —NR^{11a}R^{11b}, —(C═O)NR^{11a}R^{11b}, or —O(C═O)NR^{11a}R^{11b}; wherein
each R¹¹ is independently H or substituted or unsubstituted C₁-C₆alkyl;
each R^{11a} and R^{11b} is independently H, —CN, —OR¹², —SR¹², substituted or unsubstituted C₁-C₆alkyl, or substituted or unsubstituted C₁-C₆haloalkyl; or
R^{11a} and R^{11b} are taken together with the N atom to which they are attached to form a substituted or unsubstituted N-containing heterocycle; and
each R¹² is independently H or substituted or unsubstituted C₁-C₆alkyl.

In some embodiments,
X is C₁-C₆alkyl substituted with 1, 2, or 3 substituents each independently selected from —OR¹¹, NR¹¹(C═O)R¹¹, —NR¹¹(C═O)OR¹¹, —O(C═O)OR¹¹, —NR^{11a}R^{11b}, or —(C═O)NR^{11a}R^{11b}; wherein
each R¹¹ is independently H or substituted or unsubstituted C₁-C₆alkyl;
each R^{11a} and R^{11b} is independently H, —CN, —OR¹², —SR¹², substituted or unsubstituted C₁-C₆alkyl, or substituted or unsubstituted C₁-C₆haloalkyl; or
R^{11a} and R^{11b} are taken together with the N atom to which they are attached to form a substituted or unsubstituted N-containing heterocycle; and
each R¹² is independently H or substituted or unsubstituted C₁-C₆alkyl.

In some embodiments,
X is C₁-C₆alkyl substituted with —NR^{11a}R^{11b}; wherein
each R^{11a} and R^{11b} is independently H, —CN, or substituted or unsubstituted C₁-C₆alkyl; or
R^{11a} and R^{11b} are taken together with the N atom to which they are attached to form a substituted or unsubstituted N-containing heterocycle.

In some embodiments, X is C₁-C₆alkyl substituted with —NH₂, —N(H)CH₃, —N(H)CH₂CH₃, —N(H)CH₂CH₂CH₃, —N(H)CH(CH₃)₂, —N(H)cyclopropyl, —N(CN)CH₃, —N(CN)CH₂CH₃, —N(CN)CH₂CH₂CH₃, —N(CN)CH(CH₃)₂, or —N(CN)cyclopropyl.

In some embodiments, X is substituted or unsubstituted C₁-C₄haloalkyl. In some embodiments, X is —CH₂F, —CHF₂, —CF₃, —CF₂CH₃, or —CH₂CF₃.

In some embodiments, X is substituted or unsubstituted C₃-C₆cycloalkyl. In some embodiments, X is substituted or unsubstituted cyclopropyl, substituted or unsubstituted cyclobutyl, substituted or unsubstituted cyclopentyl, or substituted or unsubstituted cyclohexyl.

In some embodiments, X is substituted or unsubstituted C₂-C₆heterocycloalkyl. In some embodiments, X is substituted or unsubstituted C₂-C₆heterocycloalkyl comprising 0-2 N, 0-2 O, and 0-2 S ring atoms. In some embodiments, X is substituted or unsubstituted C₂-C₆heterocycloalkyl comprising 1-2 N ring atoms. In some embodiments, X is substituted or unsubstituted C₂-C₆heterocycloalkyl comprising 1 N ring atom. In some embodiments, X is substituted or unsubstituted C₂-C₆heterocycloalkyl comprising 2 N ring atoms. In some embodiments, X is substituted or unsubstituted aziridinyl, substituted or unsubstituted azetidinyl, substituted or unsubstituted pyrrolidinyl, substituted or unsubstituted pyrrolidinonyl, substituted or unsubstituted piperidinyl, substituted or unsubstituted oxetanyl, substituted or unsubstituted tetrahydrofuranyl, substituted or unsubstituted tetrahydropyranyl, substituted or unsubstituted thietanyl, substituted or unsubstituted tetrahydrothienyl, substituted or unsubstituted tetrahydrothiopyranyl, substituted or unsubstituted morpholinyl, or substituted or unsubstituted piperazinyl, substituted or unsubstituted 1,3-dioxolanyl, substituted or unsubstituted 1,3-dioxolan-2-onyl, substituted or unsubstituted oxazolidinonyl, substituted or unsubstituted imidazolidin-2-onyl, or substituted or unsubstituted oxadiazolonyl. In some embodiments, X is substituted or unsubstituted pyrrolidinyl, substituted or unsubstituted pyrrolidinonyl, substituted or unsubstituted piperidinyl, substituted or unsubstituted 1,3-dioxolanyl, substituted or unsubstituted 1,3-dioxolan-2-onyl, substituted or unsubstituted oxazolidinonyl, substituted or unsubstituted oxadiazolidinonyl, substituted or unsubstituted isoxazolidinonyl, substituted or unsubstituted imidazolidin-2-onyl, or substituted or unsubstituted oxadiazolonyl. In some embodiments, X is pyrrolidinyl, pyrrolidinonyl, oxazolidinonyl, isoxazolidinonyl, or imidazolidin-2-onyl, each substituted with 1, 2, 3, or 4 substituents each independently selected from F, —OH, —CN, substituted or unsubstituted C₁-C₆alkyl, substituted or unsubstituted C₁-C₆alkoxy, substituted or unsubstituted C₁-C₆hydroxyalkyl, substituted or unsubstituted C₁-C₆alkylamino, and substituted or unsubstituted C₁-C₆aminoalkyl. In some embodiments, X is pyrrolidinyl, pyrrolidinonyl, oxazolidinonyl, isoxazolidinonyl, or imidazolidin-2-onyl, each substituted with 1, 2, 3, or 4 substituents each independently selected from F, —OH, —CN, C₁-C₆alkyl, C₁-C₆alkoxy, C₁-C₆hydroxyalkyl, C₁-C₆alkylamino, and C₁-C₆aminoalkyl. In some embodiments, X is pyrrolidinyl, pyrrolidinonyl, oxazolidinonyl, isoxazolidinonyl, or imidazolidin-2-onyl, each substituted with 1 or 2 substituents each independently selected from F, —OH, —CN, —CH₃, —CH₂CH₃, —OCH₃, and —OCH₂CH₃. In some embodiments, X comprises a stereocenter. In some embodiments, the stereocenter is in the R-configuration. In some embodiments, the stereocenter is in the S-configuration. In some embodiments, the R-isomer of X is provided in at least 20%, 30%, 40%, 50%, 55%, 60%, 65%, 70%, 75%, 80%, 85%, 88%, 90%, 91%, 92%, 93%, 94%, 95%, 96%, 97%, 98%, 99%, 99.5%, or 99.9% excess over the S-isomer. In some embodiments, the S-isomer of X is provided in at least 20%, 30%, 40%, 50%, 55%, 60%, 65%, 70%, 75%, 80%, 85%, 88%, 90%, 91%, 92%, 93%, 94%, 95%, 96%, 97%, 98%, 99%, 99.5%, or 99.9% excess over the R-isomer. In some embodiments, X is

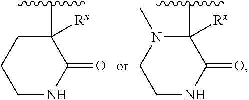

wherein R^{X} is C₁-C₃ alkyl. In some embodiments, a carbon atom attached X is

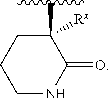

In some embodiments, X is

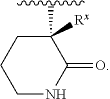

In some embodiments, X is

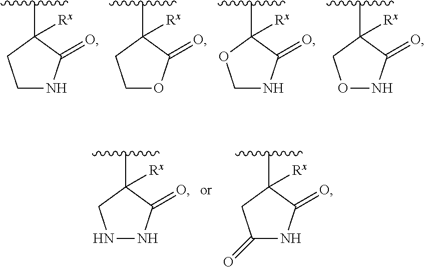

wherein R^{X} is C₁-C₃ alkyl. In some embodiments, X is

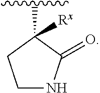

In some embodiments, X is

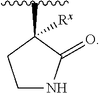

In some embodiments, R^{X} is —CH₃, —CH₂CH₃, or —CH₂CH₂CH₃. In some embodiments, R^{X} is —CH₃. In some embodiments, R^{X} is —CH₂CH₃. In some embodiments, X is

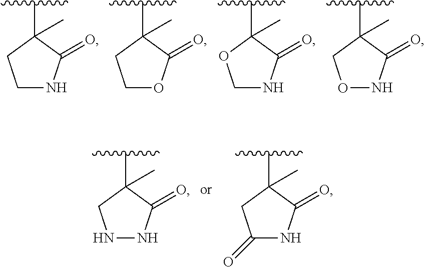

In some embodiments, X is

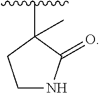 [SMILES: *C1(C)CCNC1=O]

In some embodiments, X is substituted or unsubstituted heteroaryl. In some embodiments, X is substituted or unsubstituted monocyclic heteroaryl. In some embodiments, X is substituted or unsubstituted pyridinyl, substituted or unsubstituted imidazolyl, substituted or unsubstituted pyrimidinyl, substituted or unsubstituted pyrazolyl, substituted or unsubstituted triazolyl, substituted or unsubstituted pyrazinyl, substituted or unsubstituted tetrazolyl, substituted or unsubstituted furyl, substituted or unsubstituted thienyl, substituted or unsubstituted isoxazolyl, substituted or unsubstituted thiazolyl, substituted or unsubstituted oxazolyl, substituted or unsubstituted isothiazolyl, substituted or unsubstituted pyrrolyl, substituted or unsubstituted pyridazinyl, substituted or unsubstituted triazinyl, substituted or unsubstituted oxadiazolyl, substituted or unsubstituted thiadiazolyl, or substituted or unsubstituted furazanyl.

In some embodiments, X is -L¹-Y¹.

In some embodiments,
L¹ is substituted or unsubstituted C₁-C₄alkylene; and
Y¹ is substituted or unsubstituted C₃-C₆cycloalkyl.

In some embodiments,

Y¹ is substituted or unsubstituted cyclopropyl, substituted or unsubstituted cyclobutyl, substituted or unsubstituted cyclopentyl, substituted or unsubstituted cyclohexyl.

In some embodiments,
L¹ is substituted or unsubstituted C₁-C₄alkylene; and
Y¹ is substituted or unsubstituted C₂-C₆ heterocycloalkyl.

In some embodiments, Y¹ is substituted or unsubstituted aziridinyl, substituted or unsubstituted azetidinyl, substituted or unsubstituted pyrrolidinyl, substituted or unsubstituted piperidinyl, substituted or unsubstituted oxetanyl, substituted or unsubstituted tetrahydrofuranyl, substituted or unsubstituted tetrahydropyranyl, substituted or unsubstituted thietanyl, substituted or unsubstituted tetrahydrothienyl, substituted or unsubstituted tetrahydrothiopyranyl, substituted or unsubstituted morpholinyl, or substituted or unsubstituted piperazinyl, substituted or unsubstituted 1,3-dioxolanyl, substituted or unsubstituted 1,3-dioxolan-2-onyl, substituted or unsubstituted oxazolidinonyl, substituted or unsubstituted imidazolidin-2-onyl, or substituted or unsubstituted oxadiazolonyl.

In some embodiments,
L¹ is substituted or unsubstituted C₁-C₄alkylene; and
Y¹ is substituted or unsubstituted phenyl.

In some embodiments,
L¹ is substituted or unsubstituted C₁-C₄alkylene; and
Y¹ is substituted or unsubstituted heteroaryl.

In some embodiments, Y¹ is substituted or unsubstituted monocyclic heteroaryl. In some embodiments, Y¹ is substituted or unsubstituted pyridinyl, substituted or unsubstituted imidazolyl, substituted or unsubstituted pyrimidinyl, substituted or unsubstituted pyrazolyl, substituted or unsubstituted triazolyl, substituted or unsubstituted pyrazinyl, substituted or unsubstituted tetrazolyl, substituted or unsubstituted furyl, substituted or unsubstituted thienyl, substituted or unsubstituted isoxazolyl, substituted or unsubstituted thiazolyl, substituted or unsubstituted oxazolyl, substituted or unsubstituted isothiazolyl, substituted or unsubstituted pyrrolyl, substituted or unsubstituted pyridazinyl, substituted or unsubstituted triazinyl, substituted or unsubstituted oxadiazolyl, substituted or unsubstituted thiadiazolyl, or substituted or unsubstituted furazanyl.

In some embodiments,
X is -L²-L³-Y².

In some embodiments,
L² is substituted or unsubstituted C₁-C₄alkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —S(═O)₂NR³—(C═O)O—, —NR³S(═O)₂NR³—(C═O)—, —NR³S(═O)₂NR³—(C═O)O—, or —O—S(═O)₂—;
Y² is independently H, —CN, —N₃, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b}.

In some embodiments,
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —NR³(C═O)O—, —NR³S(═O)₂—, —S(═O)₂NR³—, or —OS(═O)₂—;
Y² is independently H, —CN, —N₃, substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, substituted or unsubstituted monocyclic heteroaryl, —OR⁶, —(C═O)OR⁶, —NR^{6a}R^{6b}, or —(C═O)NR^{6a}R^{6b}; and
each R⁶, R^{6a}, and R^{6b} is independently H or substituted or unsubstituted C₁-C₄alkyl.

In some embodiments,
L² is absent;
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —S(═O)₂NR³—(C═O)O—, —NR³S(═O)₂NR³—(C═O)—, —NR³S(═O)₂NR³—(C═O)O—, or —OS(═O)₂—;
Y² is independently H, —CN, —N₃, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b}.

In some embodiments,
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —NR³(C═O)O—, —NR³S(═O)₂—, —S(═O)₂NR³—, or —OS(═O)₂—;
Y² is independently H, —CN, —N₃, substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, substituted or unsubstituted monocyclic heteroaryl, —OR⁶, —(C═O)OR⁶, —NR^{6a}R^{6b}, or —(C═O)NR^{6a}R^{6b}; and
each R⁶, R^{6a}, and R^{6b} is independently H or substituted or unsubstituted C₁-C₄alkyl.

In some embodiments,
X is -L²-L³-L⁴-Y².

In some embodiments,
L² is substituted or unsubstituted C₁-C₄alkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —S(═O)₂NR³—(C═O)O—, —NR³S(═O)₂NR³—(C═O)—, —NR³S(═O)₂NR³—(C═O)O—, or —OS(═O)₂—;
L⁴ is substituted or unsubstituted C₁-C₄alkylene;
Y² is independently H, —CN, —N₃, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b}.

In some embodiments,
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —NR³(C═O)O—, —NR³S(═O)₂—, —S(═O)₂NR³—, or —OS(═O)₂—;
Y² is independently H, —CN, —N₃, substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, substituted or unsubstituted monocyclic heteroaryl, —OR⁶, —(C═O)OR⁶, —NR^{6a}R^{6b}, or —(C═O)NR^{6a}R^{6b}; and each R⁶, R^{6a}, and R^{6b} is independently H or substituted or unsubstituted C₁-C₄alkyl.

In some embodiments, R is F, Cl, —SF₅, —CN, —OCF₃, —CHF₂, or —CF₃. In some embodiments, R is F, Cl, —OCF₃, —CHF₂, or —CF₃. In some embodiments, R is —CF₃ or —SF₅. In some embodiments, R is —CF₃. In some embodiments, R is —SF₅.

In another aspect, the present disclosure provides a compound or a pharmaceutically acceptable salt or solvate thereof, wherein the compound is a compound from Table 1, or a pharmaceutically acceptable salt or solvate thereof.

TABLE 1

| Compound | | |
|---|---|---|
| No. | Structure | Name |
| 1 | 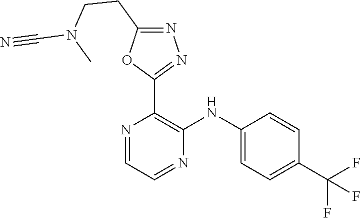 [SMILES: CN(C#N)CCc1nnc(-c2nccnc2Nc2ccc(C(F)(F)F)cc2)o1] | methyl-[2-[5-[3-[4- (trifluoromethyl)anilino]pyrazin-2-yl]- 1,3,4-oxadiazol-2-yl]ethyl]cyanamide |
| 2 | 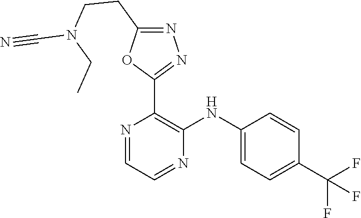 [SMILES: CCN(C#N)CCc1nnc(-c2nccnc2Nc2ccc(C(F)(F)F)cc2)o1] | ethyl-[2-[5-[3-[4- (trifluoromethyl)anilino]pyrazin-2-yl]- 1,3,4-oxadiazol-2-yl]ethyl]cyanamide |
| 3 | 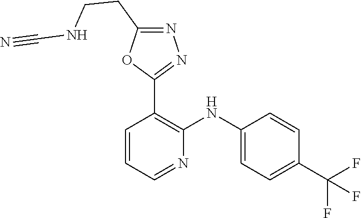 [SMILES: N#CNCCc1nnc(-c2cccnc2Nc2ccc(C(F)(F)F)cc2)o1] | N-(2-(5-(2-((4- (trifluoromethyl)phenyl)amino)pyridin- 3-yl)-1,3,4-oxadiazol-2- yl)ethyl)cyanamide |
| 4 | 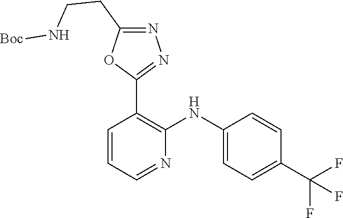 [SMILES: CC(C)(C)OC(=O)NCCc1nnc(-c2cccnc2Nc2ccc(C(F)(F)F)cc2)o1] | tert-butyl (2-(5-(2-((4- (trifluoromethyl)phenyl)amino)pyridin- 3-yl)-1,3,4-oxadiazol-2- yl)ethyl)carbamate |
| 5 | 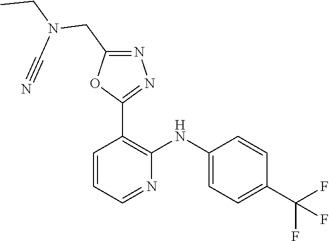 [SMILES: CCN(C#N)Cc1nnc(-c2cccnc2Nc2ccc(C(F)(F)F)cc2)o1] | ethyl-[[5-[2-[4-(trifluoromethyl)anilino]- 3-pyridyl]-1,3,4-oxadiazol-2- yl]methyl]cyanamide |
| 6 | 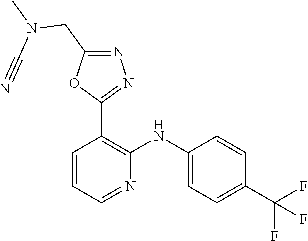 [SMILES: CN(C#N)Cc1nnc(-c2cccnc2Nc2ccc(C(F)(F)F)cc2)o1] | methyl-[[5-[2-[4- (trifluoromethyl)anilino]-3-pyridyl]- 1,3,4-oxadiazol-2-yl]methyl]cyanamide |
| 7 | 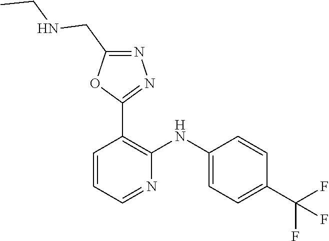 [SMILES: CCNCc1nnc(-c2cccnc2Nc2ccc(C(F)(F)F)cc2)o1] | 3-[5-(ethylaminomethyl)-1,3,4- oxadiazol-2-yl]-N-[4- (trifluoromethyl)phenyl]pyridin-2-amine |
| 8 | 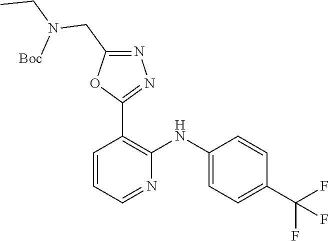 [SMILES: CCN(Cc1nnc(-c2cccnc2Nc2ccc(C(F)(F)F)cc2)o1)C(=O)OC(C)(C)C] | tert-butyl N-ethyl-N-[[5-[2-[4- (trifluoromethyl)anilino]-3-pyridyl]- 1,3,4-oxadiazol-2-yl]methyl]carbamate |
| 9 | 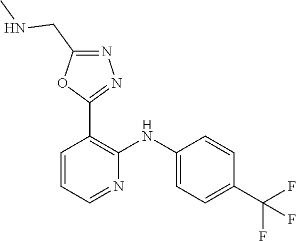 [SMILES: CNCc1nnc(-c2cccnc2Nc2ccc(C(F)(F)F)cc2)o1] | 3-[5-(methylaminomethyl)-1,3,4- oxadiazol-2-yl]-N-[4- (trifluoromethyl)phenyl]pyridin-2-amine |
| 10 | 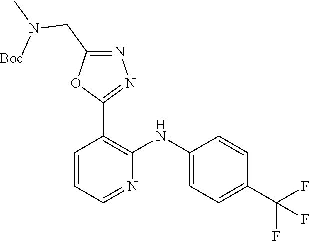 [SMILES: CN(Cc1nnc(-c2cccnc2Nc2ccc(C(F)(F)F)cc2)o1)C(=O)OC(C)(C)C] | tert-butyl N-methyl-N-[[5-[2-[4- (trifluoromethyl)anilino]-3-pyridyl]- 1,3,4-oxadiazol-2-yl]methyl]carbamate |
| 11 | 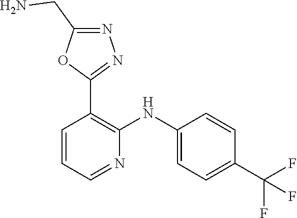 [SMILES: NCc1nnc(-c2cccnc2Nc2ccc(C(F)(F)F)cc2)o1] | 3-[5-(aminomethyl)-1,3,4-oxadiazol-2- yl]-N-[4- (trifluoromethyl)phenyl]pyridin-2-amine |
| 12 | 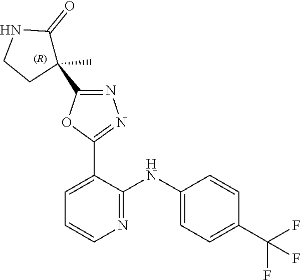 | (R)-3-methyl-3-(5-(2-((4- (trifluoromethyl)phenyl)amino)pyridin- 3-yl)-1,3,4-oxadiazol-2-yl)pyrrolidin-2- one |
| 13 | 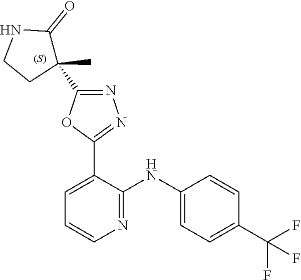 | (S)-3-methyl-3-(5-(2-((4- (trifluoromethyl)phenyl)amino)pyridin- 3-yl)-1,3,4-oxadiazol-2-yl)pyrrolidin-2- one |
| 14 | 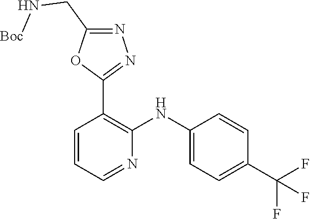 [SMILES: CC(C)(C)OC(=O)NCc1nnc(-c2cccnc2Nc2ccc(C(F)(F)F)cc2)o1] | tert-butyl N-[[5-[2-[4- (trifluoromethyl)anilino]-3-pyridyl]- 1,3,4-oxadiazol-2-yl]methyl]carbamate |
| 15 | 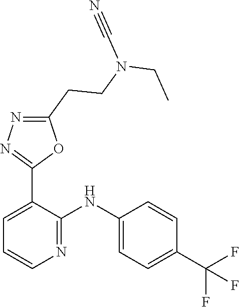 [SMILES: CCN(C#N)CCc1nnc(-c2cccnc2Nc2ccc(C(F)(F)F)cc2)o1] | ethyl-[2-[5-[2-[4- (trifluoromethyl)anilino]-3-pyridyl]- 1,3,4-oxadiazol-2-yl]ethyl]cyanamide |
| 16 | 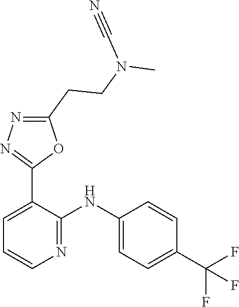 [SMILES: CN(C#N)CCc1nnc(-c2cccnc2Nc2ccc(C(F)(F)F)cc2)o1] | methyl-[2-[5-[2-[4- (trifluoromethyl)anilino]-3-pyridyl]- 1,3,4-oxadiazol-2-yl]ethyl]cyanamide |
| 17 | 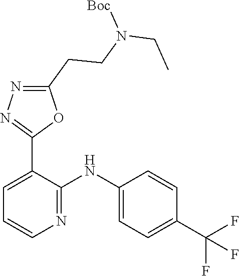 [SMILES: CCN(CCc1nnc(-c2cccnc2Nc2ccc(C(F)(F)F)cc2)o1)C(=O)OC(C)(C)C] | tert-butyl N-ethyl-N-[2-[5-[2-[4- (trifluoromethyl)anilino]-3-pyridyl]- 1,3,4-oxadiazol-2-yl]ethyl]carbamate |
| 18 | 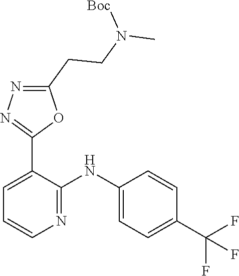 [SMILES: CN(CCc1nnc(-c2cccnc2Nc2ccc(C(F)(F)F)cc2)o1)C(=O)OC(C)(C)C] | tert-butyl N-methyl-N-[2-[5-[2-[4- (trifluoromethyl)anilino]-3-pyridyl]- 1,3,4-oxadiazol-2-yl]ethyl]carbamate |
| 19 | 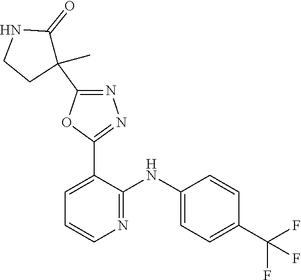 [SMILES: CC1(c2nnc(-c3cccnc3Nc3ccc(C(F)(F)F)cc3)o2)CCNC1=O] | 3-methyl-3-(5-(2-((4- (trifluoromethyl)phenyl)amino)pyridin- 3-yl)-1,3,4-oxadiazol-2-yl)pyrrolidin-2- one |
| 20 | 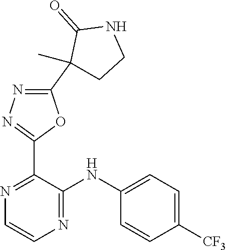 [SMILES: CC1(c2nnc(-c3nccnc3Nc3ccc(C(F)(F)F)cc3)o2)CCNC1=O] | 3-methyl-3-{5-[3-(4-trifluoromethyl- phenylamino)-pyrazin-2-yl]- [1,3,4]oxadiazol-2-yl}-pyrrolidin-2-one |
| 21 | 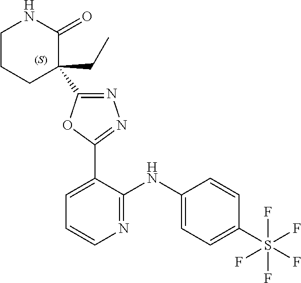 | (3S)-3-ethyl-3-[5-[2-[4-(pentafluoro- λ6-sulfanyl)anilino]-3-pyridyl]-1,3,4- oxadiazol-2-yl]piperidin-2-one |
| 22 | 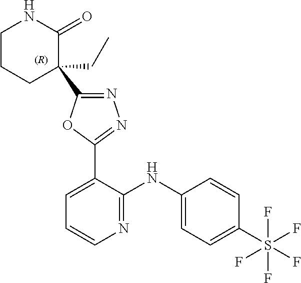 | (3R)-3-ethyl-3-[5-[2-[4-(pentafluoro- λ6-sulfanyl)anilino]-3-pyridyl]-1,3,4- oxadiazol-2-yl]piperidin-2-one |
| 23 | 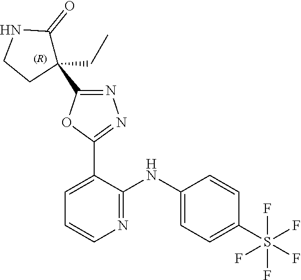 | (3R)-3-ethyl-3-[5-[2-[4-(pentafluoro- λ⁶-sulfanyl)anilino]-3-pyridyl]-1,3,4- oxadiazol-2-yl]pyrrolidin-2-one |
| 24 | 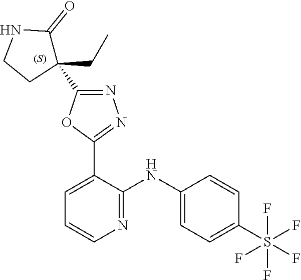 | (3S)-3-ethyl-3-[5-[2-[4-(pentafluoro- λ⁶-sulfanyl)anilino]-3-pyridyl]-1,3,4- oxadiazol-2-yl]pyrrolidin-2-one |
| 25 | 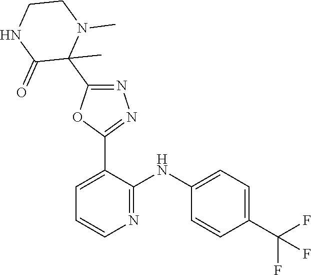 [SMILES: CN1CCNC(=O)C1(C)c1nnc(-c2cccnc2Nc2ccc(C(F)(F)F)cc2)o1] | 3,4-dimethyl-3-[5-[2-[4- (trifluoromethyl)anilino]-3-pyridyl]- 1,3,4-oxadiazol-2-yl]piperazin-2-one |
| 26 | 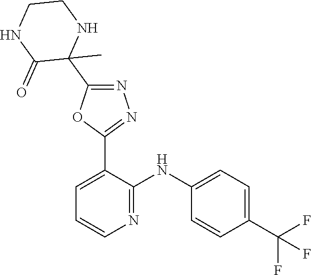 [SMILES: CC1(c2nnc(-c3cccnc3Nc3ccc(C(F)(F)F)cc3)o2)NCCNC1=O] | 3-methyl-3-[5-[2-[4- (trifluoromethyl)anilino]-3-pyridyl]- 1,3,4-oxadiazol-2-yl]piperazin-2-one |
| 27 | 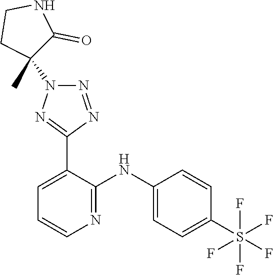 [SMILES: C[C@@]1(n2nnc(-c3cccnc3Nc3ccc(S(F)(F)(F)(F)F)cc3)n2)CCNC1=O] | (3R)-3-methyl-3-[5-[2-[4- (pentafluoro-λ⁶-sulfanyl)anilino]-3- pyridyl]tetrazol-2-yl]pyrrolidin-2-one |
| 28 | 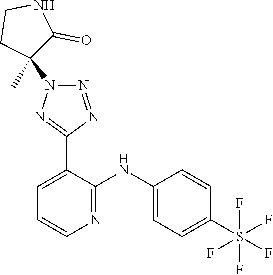 [SMILES: C[C@]1(n2nnc(-c3cccnc3Nc3ccc(S(F)(F)(F)(F)F)cc3)n2)CCNC1=O] | (3S)-3-methyl-3-[5-[2-[4- (pentafluoro-λ⁶-sulfanyl)anilino]-3- pyridyl]tetrazol-2-yl]pyrrolidin-2-one |
| 29 | 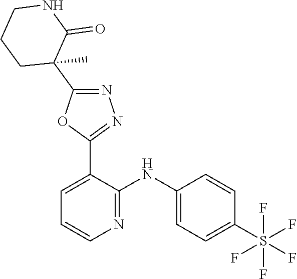 [SMILES: C[C@]1(c2nnc(-c3cccnc3Nc3ccc(S(F)(F)(F)(F)F)cc3)o2)CCCNC1=O] | (3R)-3-methyl-3-[5-[2-[4- (pentafluoro-λ⁶-sulfanyl)anilino]-3- pyridyl]-1,3,4-oxadiazol-2- yl]piperidin-2-one |
| 30 | 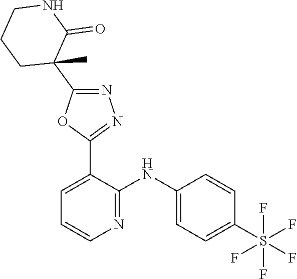 [SMILES: C[C@@]1(c2nnc(-c3cccnc3Nc3ccc(S(F)(F)(F)(F)F)cc3)o2)CCCNC1=O] | (3S)-3-methyl-3-[5-[2-[4- (pentafluoro-λ⁶-sulfanyl)anilino]-3- pyridyl]-1,3,4-oxadiazol-2- yl]piperidin-2-one |
| 31 | 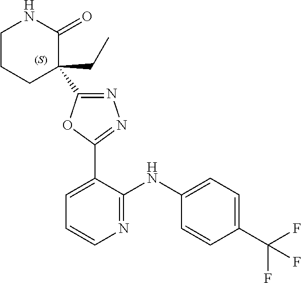 | (3S)-3-ethyl-3-[5-[2-[4- (trifluoromethyl)anilino]-3-pyridyl]- 1,3,4-oxadiazol-2-yl]piperidin-2-one |
| 32 | 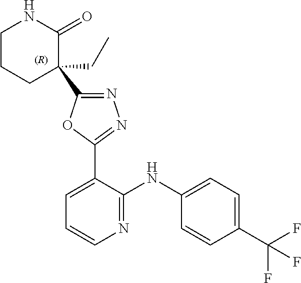 | (3R)-3-ethyl-3-[5-[2-[4- (trifluoromethyl)anilino]-3-pyridyl]- 1,3,4-oxadiazol-2-yl]piperidin-2-one |
| 33 | 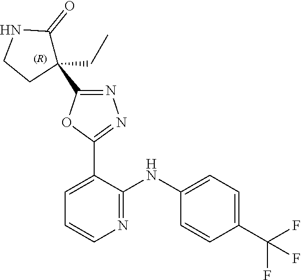 | (3R)-3-ethyl-3-[5-[2-[4- (trifluoromethyl)anilino]-3-pyridyl]- 1,3,4-oxadiazol-2-yl]pyrrolidin-2-one |
| 34 | 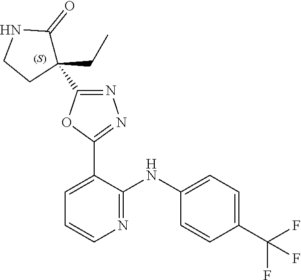 | (3S)-3-Ethyl-3-[5-[2-[4- (trifluoromethyl)anilino]-3-pyridyl]- 1,3,4-oxadiazol-2-yl]pyrrolidin-2-one |
| 35 | 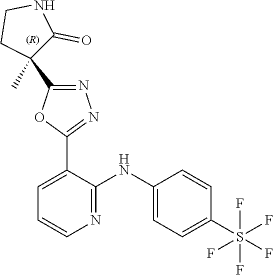 | (3R)-3-methyl-3-[5-[2-[4- (pentafluoro-λ⁶-sulfanyl)anilino]-3- pyridyl]-1,3,4-oxadiazol-2- yl]pyrrolidin-2-one |
| 36 | 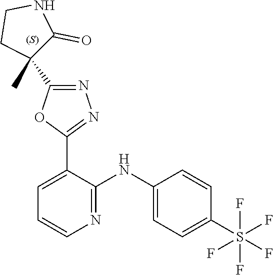 | (3S)-3-methyl-3-[5-[2-[4- (pentafluoro-λ⁶-sulfanyl)anilino]-3- pyridyl]-1,3,4-oxadiazol-2- yl]pyrrolidin-2-one |
| 37 | 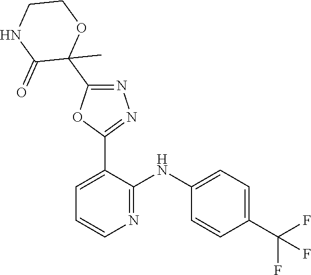 [SMILES: CC1(c2nnc(-c3cccnc3Nc3ccc(C(F)(F)F)cc3)o2)OCCNC1=O] | 2-methyl-2-(5-(2-((4- (trifluoromethyl)phenyl)amino)pyridin- 3-yl)-1,3,4-oxadiazol-2- yl)morpholin-3-one |
| 38 | 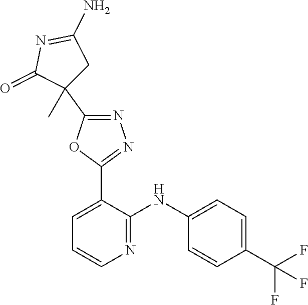 [SMILES: CC1(c2nnc(-c3cccnc3Nc3ccc(C(F)(F)F)cc3)o2)CC(N)=NC1=O] | 2-amino-4-methyl-4-[5-[2-[4- (trifluoromethyl)anilino]-3-pyridyl]- 1,3,4-oxadiazol-2-yl]-3H-pyrrol-5- one |
| 39 | 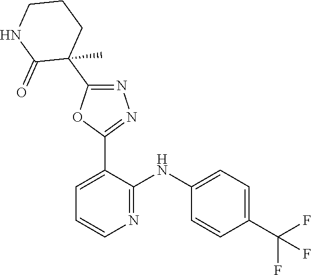 [SMILES: C[C@@]1(c2nnc(-c3cccnc3Nc3ccc(C(F)(F)F)cc3)o2)CCCNC1=O] | (S)-3-methyl-3-(5-(2-((4- (trifluoromethyl)phenyl)amino) pyridin-3-yl)-1,3,4-oxadiazol-2- yl)piperidin-2-one |
| 40 | 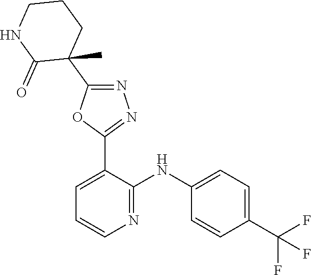 [SMILES: C[C@]1(c2nnc(-c3cccnc3Nc3ccc(C(F)(F)F)cc3)o2)CCCNC1=O] | (R)-3-methyl-3-(5-(2-((4- (trifluoromethyl)phenyl)amino)pyridin- 3-yl)-1,3,4-oxadiazol-2- yl)piperidin-2-one |
| 41 | 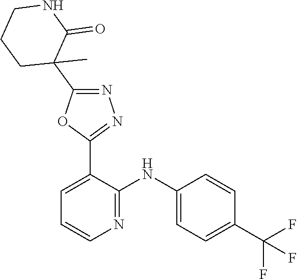 [SMILES: CC1(c2nnc(-c3cccnc3Nc3ccc(C(F)(F)F)cc3)o2)CCCNC1=O] | 3-methyl-3-(5-(2-((4- (trifluoromethyl)phenyl)amino)phenyl)- 1,3,4-oxadiazol-2-yl)piperidin-2- one |
| 42 | 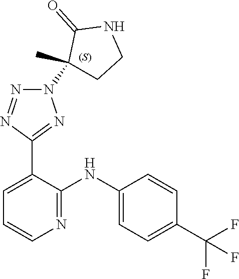 | (3S)-3-methyl-3-[5-[2-[4- (trifluoromethyl)anilino]-3- pyridyl]tetrazol-2-yl]pyrrolidin-2-one |
| 43 | 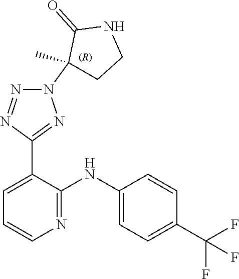 | (3R)-3-methyl-3-[5-[2-[4- (trifluoromethyl)anilino]-3- pyridyl]tetrazol-2-yl]pyrrolidin-2-one |
| 44 | 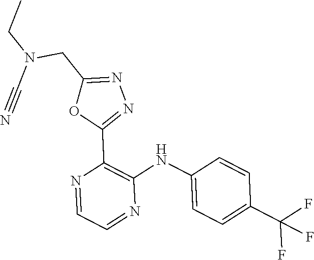 [SMILES: CCN(C#N)Cc1nnc(-c2nccnc2Nc2ccc(C(F)(F)F)cc2)o1] | ethyl-[[5-[3-[4- (trifluoromethyl)anilino]pyrazin-2- yl]-1,3,4-oxadiazol-2- yl]methyl]cyanamide |
| 45 | 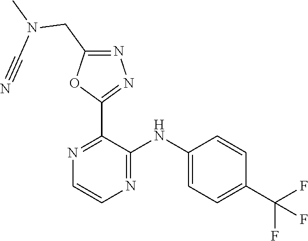 [SMILES: CN(C#N)Cc1nnc(-c2nccnc2Nc2ccc(C(F)(F)F)cc2)o1] | methyl-[[5-[3-[4- (trifluoromethyl)anilino]pyrazin-2- yl]-1,3,4-oxadiazol-2- yl]methyl]cyanamide |
| 46 | 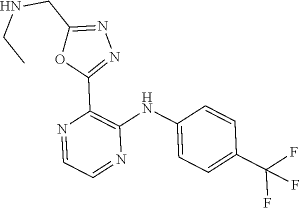 [SMILES: CCNCc1nnc(-c2nccnc2Nc2ccc(C(F)(F)F)cc2)o1] | 3-[5-(ethylaminomethyl)-1,3,4- oxadiazol-2-yl]-N-[4- (trifluoromethyl)phenyl]pyrazin-2- amine |
| 47 | 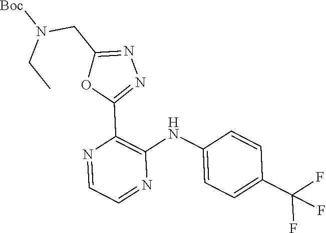 [SMILES: CCN(Cc1nnc(-c2nccnc2Nc2ccc(C(F)(F)F)cc2)o1)C(=O)OC(C)(C)C] | tert-butyl N-ethyl-N-[[5-[3-[4- (trifluoromethyl)anilino]pyrazin-2- yl]-1,3,4-oxadiazol-2- yl]methyl]carbamate |
| 48 | 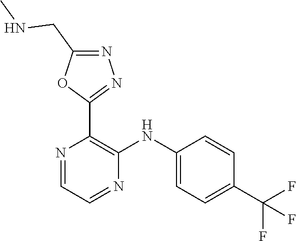 [SMILES: CNCc1nnc(-c2nccnc2Nc2ccc(C(F)(F)F)cc2)o1] | 3-[5-(methylaminomethyl)-1,3,4- oxadiazol-2-yl]-N-[4- (trifluoromethyl)phenyl]pyrazin-2- amine |
| 49 | 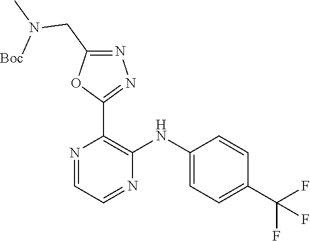 [SMILES: CN(Cc1nnc(-c2nccnc2Nc2ccc(C(F)(F)F)cc2)o1)C(=O)OC(C)(C)C] | tert-butyl N-methyl-N-[[5-[3-[4- (trifluoromethyl)anilino]pyrazin-2- yl]-1,3,4-oxadiazol-2- yl]methyl]carbamate |

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Methods known to one of ordinary skill in the art are identified through various reference books and databases. Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In some instances, specific and analogous reactants are identified through the indices of known chemicals prepared by the Chemical Abstract Service of the American Chemical Society, which are available in most public and university libraries, as well as through on-line databases (the American Chemical Society, Washington, D.C., is contacted for more details). Chemicals that are known but not commercially available in catalogs are prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, the compounds disclosed herein are prepared as described in the Examples section.

### Further Forms of Compounds Disclosed Herein

### Isomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, disclosed herein are dissociable complexes (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. In some embodiments, examples of isotopes that are incorporated into compounds of the disclosure include isotopes of hydrogen, carbon, nitrogen, oxygen, phosphorous, sulfur, fluorine, and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvates, hydrates, or derivatives thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the disclosure, or by separately reacting a purified compound in its free form with a suitable acid or base, and isolating the salt thus formed.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. In some embodiments, solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or methanol. In some embodiments, the compounds provided herein exist in unsolvated as well as solvated forms. In general, the solvated forms are considered equivalent to the unsolvated forms for the purposes of the compounds and methods provided herein.

### Prodrugs

In some embodiments, the compounds described herein exist in prodrug form. The disclosure provides for methods of treating diseases by administering such prodrugs. The disclosure further provides for methods of treating diseases by administering such prodrugs as pharmaceutical compositions.

In some embodiments, prodrugs include compounds wherein an amino acid residue, or a polypeptide chain of two or more (e. g., two, three, or four) amino acid residues is covalently joined through an amide or ester bond to a free amino, hydroxy, or carboxylic acid group of compounds of the present disclosure. The amino acid residues include, but are not limited to, the 20 naturally occurring amino acids and also includes 4-hydroxyproline, hydroxylysine, demosine, isodemosine, 3-methylhistidine, norvaline, beta-alanine, gamma-aminobutyric acid, citrulline, homocysteine, homoserine, ornithine, and methionine sulfone. In other embodiments, prodrugs include compounds wherein a nucleic acid residue, or an oligonucleotide of two or more (e. g., two, three or four) nucleic acid residues is covalently joined to a compound of the present disclosure.

Pharmaceutically acceptable prodrugs of the compounds described herein also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, metal salts, and sulfonate esters. In some embodiments, compounds having free amino, amido, hydroxy, or carboxylic groups are converted into prodrugs. For instance, free carboxyl groups are derivatized as amides or alkyl esters. In certain instances, all of these prodrug moieties incorporate groups including, but not limited to, ether, amine, and carboxylic acid functionalities.

Hydroxy prodrugs include esters such as, though not limited to, acyloxyalkyl (e.g. acyloxymethyl, acyloxyethyl) esters, alkoxycarbonyloxyalkyl esters, alkyl esters, aryl esters, sulfonate esters, sulfate esters and disulfide containing esters, ethers, amides, carbamates, hemisuccinates, dimethylaminoacetates, and phosphoryloxymethyloxycarbonyls, as outlined in Advanced Drug Delivery Reviews 1996, 19, 115.

Amine derived prodrugs include, but are not limited to, the following groups and combinations of groups:

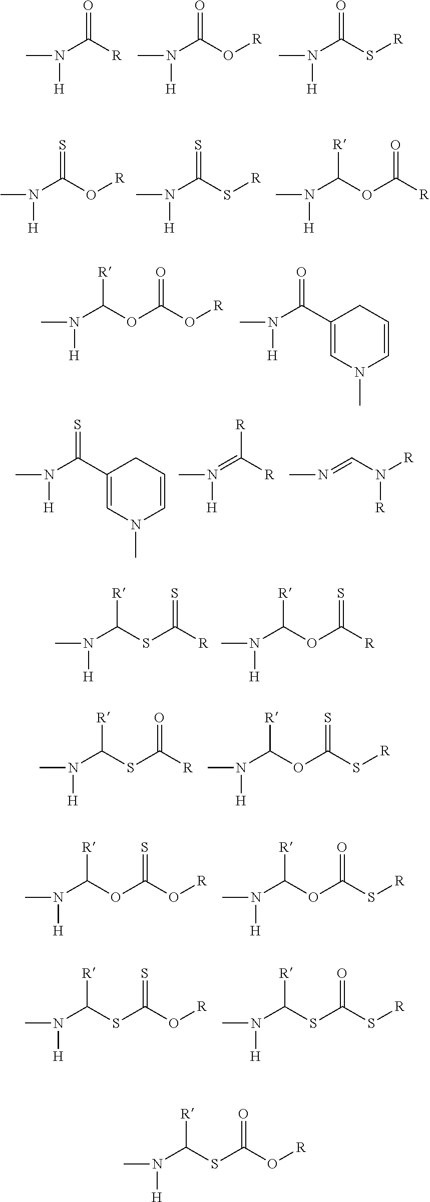

as well as sulfonamides and phosphonamides.

In certain instances, sites on any aromatic ring portions are susceptible to various metabolic reactions, therefore incorporation of appropriate substituents on the aromatic ring structures reduce, minimize, or eliminate this metabolic pathway.

### Metabolites

In some embodiments, compounds described herein are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen or an alkyl group.

In additional or further embodiments, the compounds described herein are metabolized upon administration to an organism in need to produce a metabolite that is then used to produce a desired effect, including a desired therapeutic effect.

### Pharmaceutical Compositions

In certain embodiments, the compound as described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)), the disclosure of which is hereby incorporated herein by reference in its entirety.

Accordingly, provided herein is a pharmaceutical composition comprising at least one compound described herein, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (A), Formula (A-1), Formula (A-2), Formula (A-3), Formula (A-4), Formula (I), Formula (Ia), Formula (Ib), Formula (Ic), Formula (Id), Formula (Ie), or Formula (If), or a pharmaceutically acceptable salt or solvate thereof.

Another embodiment provides a pharmaceutical composition consisting essentially of a pharmaceutically acceptable carrier and a compound of Formula (A), Formula (A-1), Formula (A-2), Formula (A-3), Formula (A-4), Formula (I), Formula (Ia), Formula (Ib), Formula (Ic), Formula (Id), Formula (Ie), or Formula (If), or a pharmaceutically acceptable salt or solvate thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions are formulated as a unit dose, and/or are formulated for oral or subcutaneous administration.

In some instances, exemplary pharmaceutical compositions are used in the form of a pharmaceutical preparation, for example, in solid, semisolid, or liquid form, which includes one or more of a disclosed compound, as an active ingredient, in admixture with an organic or inorganic carrier or excipient suitable for external, enteral, or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets in some instances, the principal active ingredient is mixed with a pharmaceutical carrier, e.g., conventional tableting ingredients such as corn starch, lactose, sucrose, sorbitol, talc, stearic acid, magnesium stearate, dicalcium phosphate, or gums, and other pharmaceutical diluents, e.g., water, to form a solid preformulation composition containing a homogeneous mixture of a disclosed compound or a non-toxic pharmaceutically acceptable salt or a non-toxic pharmaceutically acceptable solvate thereof. When referring to these preformulation compositions as homogeneous, it is meant that the active ingredient is dispersed evenly throughout the composition so that the composition is readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions also comprise buffering agents in some embodiments. Solid compositions of a similar type are also employed as fillers in soft and hard-filled gelatin capsules using such excipients as lactose or milk sugars, as well as high molecular weight polyethylene glycols and the like.

In some instances, a tablet is made by compression or molding, optionally with one or more accessory ingredients. Compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets are made by molding in a suitable machine a mixture of the subject composition moistened with an inert liquid diluent. Tablets, and other solid dosage forms, such as dragees, capsules, pills and granules, are optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms contain optionally inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, optionally contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, formulations for rectal or vaginal administration are presented as a suppository, which are prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component is optionally mixed under sterile conditions with a pharmaceutically acceptable carrier, and with any preservatives, buffers, or propellants which are required in some embodiments.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, excipients such as lactose, talc, silicic acid, aluminum hydroxide, calcium silicates and polyamide powder, or mixtures of these substances. Sprays additionally contain customary propellants, such as chlorofluorohydrocarbons and volatile unsubstituted hydrocarbons, such as butane and propane.

Compositions and compounds disclosed herein are alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are used because they minimize exposing the agent to shear, which result in degradation of the compounds contained in the subject compositions in some embodiments. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which optionally contain antioxidants, buffers, bacteriostats, solutes which render the formulation isotonic with the blood of the intended recipient or suspending or thickening agents.

Examples of suitable aqueous and non-aqueous carriers employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. In some embodiments, proper fluidity is maintained, for example, by the use of coating materials, such as lecithin, by the maintenance of the required particle size in the case of dispersions, and by the use of surfactants

Also contemplated are enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure will recognize that it is not comprehensive and that there are other enteric materials that meet the objectives of the present disclosure.

In some embodiments, the doses of the composition comprising at least one compound as described herein differ, depending upon the patient's (e.g., human) condition, that is, stage of the disease, general health status, age, and other factors that a person skilled in the medical art will use to determine dose.

In some instances, pharmaceutical compositions are administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

In some embodiments, oral doses typically range from about 1.0 mg to about 1000 mg, one to four times, or more, per day.

### The Hippo Signaling Network

The Hippo signaling network (also known as the Salvador/Warts/Hippo (SWH) pathway) is a master regulator of cell proliferation, death, and differentiation. In some embodiments, the main function of the Hippo signaling pathway is to regulate negatively the transcriptional co-activators Yes-associated protein (YAP) and its paralogue, the transcriptional co-activator with PDZ-binding motif (TAZ; also known as WWTR1). The Hippo kinase cascade phosphorylates and inhibits YAP/TAZ by promoting its cytoplasmic retention and degradation, thereby inhibiting the growth promoting function regulated under the YAP/TAZ control. In an un-phosphorylated/de-phosphorylated state, YAP, also known as YAP1 or YAP65, together with TAZ, are transported into the nucleus where they interact with TEAD family of transcription factors to upregulate genes that promote proliferation and migration, and inhibit apoptosis. In some instances, unregulated upregulation of these genes involved in proliferation, migration, and anti-apoptosis leads to development of cancer. In some instances, overexpression of YAP/TAZ is associated with cancer.

Additional core members of the Hippo signaling pathway comprise the serine/threonine kinases MST1/2 (homologues of Hippo/Hpo in Drosophila), Lats1/2 (homologues of Warts/Wts), and their adaptor proteins Sav1 (homologue of Salvador/Sav) and Mob (MOBKL1A and MOBKL1B; homologues of Mats), respectively. In general, MST1/2 kinase complexes with the scaffold protein Sav1, which in turn phosphorylates and activates Lats1/2 kinase. Lats1/2 is also activated by the scaffold protein Mob. The activated Lats1/2 then phosphorylates and inactivates YAP or its paralog TAZ. The phosphorylation of YAP/TAZ leads to their nuclear export, retention within the cytoplasm, and degradation by the ubiquitin proteasome system.

In some instances, Lats1/2 phosphorylates YAP at the [HXRXXS] consensus motifs. YAP comprises five [HXRXXS] consensus motifs, wherein X denotes any amino acid residue. In some instances, Lats1/2 phosphorylates YAP at one or more of the consensus motifs. In some instances, Lats1/2 phosphorylates YAP at all five of the consensus motifs. In some instances, Lats1/2 phosphorylate at the S127 amino acid position. The phosphorylation of YAP S127 promotes 14-3-3 protein binding and results in cytoplasmic sequestration of YAP. Mutation of YAP at the S127 position thereby disrupts its interaction with 14-3-3 and subsequently promotes nuclear translocation.

Additional phosphorylation occurs at the S381 amino acid position in YAP. Phosphorylation of YAP at the S381 position and on the corresponding site in TAZ primes both proteins for further phosphorylation events by CK1δ/ε in the degradation motif, which then signals for interaction with the β-TRCP E3 ubiquitin ligase, leading to polyubiquitination and degradation of YAP.

In some instances, Lats1/2 phosphorylates TAZ at the [HXRXXS] consensus motifs. TAZ comprises four [HXRXXS] consensus motifs, wherein X denotes any amino acid residues. In some instances, Lats1/2 phosphorylates TAZ at one or more of the consensus motifs. In some instances, Lats1/2 phosphorylates TAZ at all four of the consensus motifs. In some instances, Lats1/2 phosphorylate at the S89 amino acid position. The phosphorylation of TAZ S89 promotes 14-3-3 protein binding and results in cytoplasmic sequestration of TAZ. Mutation of TAZ at the S89 position thereby disrupts its interaction with 14-3-3 and subsequently promotes nuclear translocation.

In some embodiments, phosphorylated YAP/TAZ accumulates in the cytoplasm, and undergoes SCF^{β-TRCP}-mediated ubiquitination and subsequent proteasomal degradation. In some instances, the Skp, Cullin, F-box containing complex (SCF complex) is a multi-protein E3 ubiquitin ligase complex that comprises a F-box family member protein (e.g. Cdc4), Skp1, a bridging protein, and RBX1, which contains a small RING Finger domain which interacts with E2-ubiquitin conjugating enzyme. In some cases, the F-box family comprises more than 40 members, in which exemplary members include F-box/WD repeat-containing protein 1A (FBXW1A, βTrCP1, Fbxw1, hsSlimb, plkappaBalpha-E3 receptor subunit) and S-phase kinase-associated proteins 2 (SKP2). In some embodiments, the SCF complex (e.g. SCF^{βTrCP1}) interacts with an E1 ubiquitin-activating enzyme and an E2 ubiquitin-conjugating enzyme to catalyze the transfer of ubiquitin to the YAP/TAZ substrate. Exemplary E1 ubiquitin-activating enzymes include those encoded by the following genes: UBA1, UBA2, UBA3, UBA5, UBA5, UBA7, ATG7, NAE1, and SAE1. Exemplary E2 ubiquitin-conjugating enzymes include those encoded by the following genes: UBE2A, UBE2B, UBE2C, UBE2D1, UBE2D2, UBE2D3, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2M, UBE2N, UBE2O, UBE2Q1, UBE2Q2, UBE2R1, UBE2R2, UBE2S, UBE2T, UBE2U, UBE2V1, UBE2V2, UBE2Z, ATG2, BIRC5, and UFC1. In some embodiments, the ubiquitinated YAP/TAZ further undergoes the degradation process through the 26S proteasome.

In some embodiments, the Hippo pathway is regulated upstream by several different families of regulators. In some instances, the Hippo pathway is regulated by the G-protein and its coupled receptors, the Crumbs complex, regulators upstream of the MST kinases, and the adherens junction.

YAP/TAZ Interaction with TEAD

In some embodiments, un-phosphorylated and/or dephosphorylated YAP/TAZ accumulates in the nucleus. Within the nucleus, YAP/TAZ interacts with the TEAD family of transcription factors (e.g. TEAD1, TEAD2, TEAD3, or TEAD4) to activate genes involved in anti-apoptosis and proliferation, such as for example CTFG, Cyr61, and FGF1.

In some embodiments, the compounds disclosed herein modulate the interaction between YAP/TAZ and TEAD. In some embodiments, the compounds disclosed herein bind to TEAD, YAP, or TAZ and prevent the interaction between YAP/TAZ and TEAD.

### YAP/TAZ Regulation Mediated by G-Proteins GPCRs

In some embodiments, the Hippo pathway is regulated by the G protein-coupled receptor (GPCR) and G protein (also known as guanine nucleotide-binding proteins) family of proteins. G proteins are molecular switches that transmit extracellular stimuli into the cell through GPCRs. In some instances, there are two classes of G proteins: monomeric small GTPases and heterotrimeric G protein complexes. In some instances, the latter class of complexes comprise of alpha (G_{α}), beta (G_{β}), and gamma (G_{γ}) subunits. In some cases, there are several classes of G_{α} subunits: G_{q/11}α, G_{12/13}α, G_{i/o}α (G inhibitory, G other), and Gₛα (G stimulatory).

In some instances, Gᵢα (G inhibitory), Gₒα (G other), G_{q/11}α, and G_{12/13}α coupled GPCRs activate YAP/TAZ and promote nuclear translocation. In other instances, Gₛα (G stimulatory) coupled GPCRs suppress YAP/TAZ activity, leading to YAP/TAZ degradation.

In some cases, Gᵢα (G inhibitory), Gₒα (G other), G_{q/11}α, and G_{12/13}α coupled GPCRs activate YAP/TAZ through repression of Lats1/2 activities. In contrast, Gₛα, in some embodiments, induces Lats1/2 activity, thereby promoting YAP/TAZ degradation.

### G_{q} Family

G_{q}α (also known as G_{q/11} protein), participates in the inositol trisphosphate (IP₃) signal transduction pathway and calcium (Ca²⁺) release from intracellular storage through the activation of phospholipase C (PLC). The activated PLC hydrolyzes phosphatidylinositol 4,5-bisphosphate (PIP₂) to diacyl glycerol (DAG) and IP₃. In some instances, IP₃ then diffuses through the cytoplasm into the ER or the sarcoplasmic reticulum (SR) in the case of muscle cells, and then binds to inositol trisphosphate receptor (InsP3R), which is a Ca²⁺ channel. In some cases, the binding triggers the opening of the Ca²⁺ channel, and thereby increases the release of Ca²⁺ into the cytoplasm.

In some embodiments, the GPCRs that interact with G_{q}α include, but are not limited to, 5-hydroxytryptamine receptor (5-HT receptor) types 5-HT₂ and 5-HT₃; alpha-1 adrenergic receptor; vasopressin type 1 receptors 1A and 1B; angiotensin II receptor type 1; calcitonin receptor; histamine H1 receptor; metabotropic glutamate receptor, group I; muscarinic receptors M₁, M₃, and M₅; and trace amine-associated receptor 1.

In some instances, there are several types of G_{q}α: G_{q}, G_{q/11}, G_{q/14}, and G_{q/15}. The G_{q} protein is encoded by GNAQ. G_{q/11} is encoded by GNA11. G_{q/14} is encoded by GNA14. G_{q/15} is encoded by GNA15.

In some instances, mutations or modifications of the G_{q}α genes have been associated with cancer. Indeed, studies have shown that mutations in G_{q}α promote uveal melanoma (UM) tumorigenesis. In some instances, about 80% of UM cases have been detected to contain a mutation in GNAQ and/or GNA11.

In some instances, mutations or modifications of the G_{q}α genes have been associated with congenital diseases. In some instances, mutations of G_{q}α have been observed in congenital diseases such as Port-Wine Stain and/or Sturge-Weber Syndrome. In some instances, about 92% of Port-Wine stain cases harbors a mutation in GNAQ. In some instances, about 88% of Sturge-Weber Syndrome harbors a mutation in GNAQ.

### G_{12/13} Family

G_{12/13}α modulates actin cytoskeletal remodeling in cells and regulates cell processes through guanine nucleotide exchange factors (GEFs). GEFs participate in the activation of small GTPases which acts as molecular switches in a variety of intracellular signaling pathways. Examples of small GTPases include the Ras-related GTPase superfamily (e.g. Rho family such as Cdc42), which is involved in cell differentiation, proliferation, cytoskeletal organization, vesicle trafficking, and nuclear transport.

In some embodiments, the GPCRs that interact with G_{12/13}α include, but are not limited to, purinergic receptors (e.g. P2Y₁, P2Y₂, P2Y₄, P2Y₆); muscarinic acetylcholine receptors M1 and M3; receptors for thrombin [protease-activated receptor (PAR)-1, PAR-2]; thromboxane (TXA2); sphingosine 1-phosphate (e.g. S1P₂, S1P₃, S1P₄ and S1P₅); lysophosphatidic acid (e.g. LPA₁, LPA₂, LPA₃); angiotensin II (AT1); serotonin (5-HT_{2c} and 5-HT₄); somatostatin (sst₅); endothelin (ET_{A} and ET_{B}); cholecystokinin (CCK₁); V₁ₐ vasopressin receptors; D₅ dopamine receptors; fMLP formyl peptide receptors; GAL₂ galanin receptors; EP₃ prostanoid receptors; A₁ adenosine receptors; α₁ adrenergic receptors; BB₂ bombesin receptors; B₂ bradykinin receptors; calcium-sensing receptors; KSHV-ORF74 chemokine receptors; NK₁ tachykinin receptors; and thyroid-stimulating hormone (TSH) receptors.

In some instances, G_{12/13}α is further subdivided into G₁₂ and G₁₃ types which are encoded by GNA12 and GNA13, respectively.

### G_{i/o} Family

G_{i/o}α (G inhibitory, G other) (also known as Gᵢ/G₀ or Gᵢ protein) suppresses the production of 3′,5′-cyclic AMP (cAMP) from adenosine triphosphate (ATP) through an inhibition of adenylate cyclase activity, which converts ATP to cAMP.

In some embodiments, the GPCRs that interact with Gᵢα include, but are not limited to, 5-hydroxytryptamine receptor (5-HT receptor) types 5-HT₁ and 5-HT₅; muscarinic acetylcholine receptors such as M₂ and M₄; adenosine receptors such as A₁ and A₃; adrenergic receptors such as α_{2A}, α_{2B}, and α_{2C}; apelin receptors; calcium-sensing receptor; cannabinoid receptors CB1 and CB2; chemokine CXCR4 receptor; dopamines D₂, D₃, and D₄; GABA_{B} receptor; glutamate receptors such as metabotropic glutamate receptor 2 (mGluR2), metabotropic glutamate receptor 3 (mGluR3), metabotropic glutamate receptor 4 (mGluR4), metabotropic glutamate receptor 6 (mGluR6), metabotropic glutamate receptor 7 (mGluR7), and metabotropic glutamate receptor 8 (mGluR8); histamine receptors such as H₃ and H₄ receptors; melatonin receptors such as melatonin receptor type 1 (MT1), melatonin receptor type 2 (MT2), and melatonin receptor type 3 (MT3); niacin receptors such as NIACR1 and NIACR2; opioid receptors such as δ, κ, μ, and nociceptin receptors; prostaglandin receptors such as prostaglandin E receptor 1 (EP₁), prostaglandin E receptor 3 (EP₃), prostaglandin F receptor (FP), and thromboxane receptor (TP); somatostatin receptors sst1, sst2, sst3, sst4, and sst5; and trace amine-associated receptor 8.

In some instances, there are several types of Gᵢα: Gᵢα1, Gᵢα2, Gᵢα3, Gᵢα4, Gₒα, Gₜ, G_{gust}, and G_{z}. Gᵢα1 is encoded by GNAI1. Gᵢα2 is encoded by GNAI2. Gᵢα3 is encoded by GNAI3. Gₒα, the aₒ subunit, is encoded by GNAO1. Gₜ is encoded by GNAT1 and GNAT2. G_{gust} is encoded by GNAT3. G_{z} is encoded by GNAZ.

### Gₛ Family

Gₛα (also known as G stimulatory, Gₛ alpha subunit, or Gₛ protein) activates the cAMP-dependent pathway through the activation of adenylate cyclase, which convers adenosine triphosphate (ATP) to 3′,5′-cyclic AMP (cAMP) and pyrophosphate. In some embodiments, the GPCRs that interact with Gₛα include, but are not limited to, 5-hydroxytryptamine receptor (5-HT receptor) types 5-HT₄, 5-HT₆, and 5-HT₇; adrenocorticotropic hormone receptor (ACTH receptor) (also known as melanocortin receptor 2 or MC2R); adenosine receptor types A₂ₐ and A_{2b}; arginine vasopressin receptor 2 (AVPR2); β-adrenergic receptors β₁, β₂, and β₃; calcitonin receptor; calcitonin gene-related peptide receptor; corticotropin-releasing hormone receptor; dopamine receptor D₁-like family receptors such as D₁ and D₅; follicle-stimulating hormone receptor (FSH-receptor); gastric inhibitory polypeptide receptor; glucagon receptor; histamine H₂ receptor; luteinizing hormone/choriogonadotropin receptor; melanocortin receptors such as MC1R, MC2R, MC3R, MC4R, and MC5R; parathyroid hormone receptor 1; prostaglandin receptor types D₂ and I₂; secretin receptor; thyrotropin receptor; trace amine-associated receptor 1; and box jellyfish opsin.

In some instances, there are two types of Gₛα: Gₛ and G_{olf}. Gₛ is encoded by GNAS. G_{olf} is encoded by GNAL.

### Additional Regulators of the Hippo Signaling Network

In some embodiments, the additional regulator of the Hippo signaling pathway is the Crumbs (Crb) complex. The Crumbs complex is a key regulator of cell polarity and cell shape. In some instances, the Crumbs complex comprises transmembrane CRB proteins which assemble multi-protein complexes that function in cell polarity. In some instances, CRB complexes recruit members of the Angiomotin (AMOT) family of adaptor proteins that interact with the Hippo pathway components. In some instances, studies have shown that AMOT directly binds to YAP, promotes YAP phosphorylation, and inhibits its nuclear localization.

In some instances, the additional regulator of the Hippo signaling pathway comprises regulators of the MST kinase family. MST kinases monitor actin cytoskeletal integrity. In some instances, the regulators include TAO kinases and cell polarity kinase PAR-1.

In some instances, the additional regulator of the Hippo signaling pathway comprises molecules of the adherens junction. In some instances, E-Cadherin (E-cad) suppresses YAP nuclear localization and activity through regulating MST activity. In some embodiments, E-cad-associated protein α-catenin regulates YAP through sequestering YAP/14-3-3 complexes in the cytoplasm. In other instances, Ajuba protein family members interact with Lats1/2 kinase activity, thereby preventing inactivation of YAP/TAZ.

In some embodiments, additional proteins that interact with YAP/TAZ either directly or indirectly include, but are not limited to, Merlin, protocadherin Fat 1, MASK1/2, HIPK2, PTPN14, RASSF, PP2A, Salt-inducible kinases (SIKs), Scribble (SCRIB), the Scribble associated proteins Discs large (Dlg), KIBRA, PTPN14, NPHP3, LKB1, Ajuba, and ZO1/2.

In some embodiments, the compounds described herein are inhibitors of transcriptional coactivator with PDZ binding motif/Yes-associated protein transcriptional coactivator (TAZ/YAP). In some embodiments, the compounds described herein increase the phosphorylation of transcriptional coactivator with PDZ binding motif/Yes-associated protein transcriptional coactivator (TAZ/YAP) or decrease the dephosphorylation of transcriptional coactivator with PDZ binding motif/Yes-associated protein transcriptional coactivator (TAZ/YAP). In some embodiments, the compounds increase the ubiquitination of transcriptional coactivator with PDZ binding motif/Yes-associated protein transcriptional coactivator (TAZ/YAP) or decrease the deubiquitination of transcriptional coactivator with PDZ binding motif/Yes-associated protein transcriptional coactivator (TAZ/YAP).

In some embodiments, the compounds disclosed herein are inhibitors of one or more of the proteins encompassed by, or related to, the Hippo pathway. In some instances, the one or more proteins comprise a protein described herein. In some embodiments, an inhibitor of the Hippo pathway is an inhibitor of a G-protein and/or its coupled GPCR. In some embodiments, an inhibitor of the Hippo pathway is an inhibitor of a G-protein. In some embodiments, an inhibitor of the Hippo pathway is an inhibitor of the G_{q}α family proteins such as G_{q}, G_{q/11}, G_{q/14}, and G_{q/15}; the G_{12/13}α family of proteins such as G₁₂ and G₁₃; or the Gᵢα family of proteins such as Gᵢα1, Gᵢα2, Gᵢα3, Gᵢα4, Gₒα, Gₜ, G_{gust}, and G_{z}. In some embodiments, an inhibitor of the Hippo pathway is an inhibitor of G_{q}. In some embodiments, an inhibitor of the Hippo pathway is an inhibitor of G_{q/11}. In some embodiments, an inhibitor of the Hippo pathway is an inhibitor of G_{q/14}. In some embodiments, an inhibitor of the Hippo pathway is an inhibitor of G_{q/15}. In some embodiments, an inhibitor of the Hippo pathway is an inhibitor of G₁₂. In some embodiments, an inhibitor of the Hippo pathway is an inhibitor of G₁₃. In some embodiments, an inhibitor of the Hippo pathway is an inhibitor of Gᵢα1. In some embodiments, an inhibitor of the Hippo pathway is an inhibitor of Gᵢα2. In some embodiments, an inhibitor of the Hippo pathway is an inhibitor of Gᵢα3. In some embodiments, an inhibitor of the Hippo pathway is an inhibitor of Gᵢα4. In some embodiments, an inhibitor of the Hippo pathway is an inhibitor of Gₒα. In some embodiments, an inhibitor of the Hippo pathway is an inhibitor of Gₜ. In some embodiments, an inhibitor of the Hippo pathway is an inhibitor of G_{gust}. In some embodiments, an inhibitor of the Hippo pathway is an inhibitor of G_{z}.

In some embodiments, an inhibitor of the Hippo pathway is an inhibitor of a core protein of the Hippo pathway. In some embodiments, an inhibitor of the Hippo pathway is an inhibitor of Sav1. In some embodiments, an inhibitor of the Hippo pathway is an inhibitor of Mob. In some embodiments, an inhibitor of the Hippo pathway is an inhibitor of YAP. In some embodiments, an inhibitor of the Hippo pathway is an inhibitor of TAZ. In some embodiments, an inhibitor of the Hippo pathway is an inhibitor of TEAD.

In some embodiments, an inhibitor of the Hippo pathway is an inhibitor of a protein associated with the ubiquitination and proteasomal degradation pathway. In some embodiments, an inhibitor of the Hippo pathway is an inhibitor of a proteasomal degradation pathway protein (e.g. 26S proteasome).

In some embodiments, an inhibitor of the Hippo pathway is an inhibitor of a protein of the Ras superfamily of proteins. In some embodiments, an inhibitor of the Hippo pathway is an inhibitor of a protein of the Rho family of proteins. In some embodiments, an inhibitor of the Hippo pathway is an inhibitor of Cdc42.

Cdc42 is a member of the Ras superfamily of small GTPases. Specifically, Cdc42 belongs to the Rho family of GTPases, in which the family members participate in diverse and critical cellular processes such as gene transcription, cell-cell adhesion, and cell cycle progression. Cdc42 is involved in cell growth and polarity, and in some instances, Cdc42 is activated by guanine nucleotide exchange factors (GEFs). In some cases, an inhibitor of Cdc42 is a compound disclosed herein.

In some embodiments, an inhibitor of the Hippo pathway is an inhibitor of a deubiquitinating enzyme. In some embodiments, an inhibitor of the Hippo pathway is an inhibitor of a cysteine protease or a metalloprotease. In some embodiments, an inhibitor of the Hippo pathway is an inhibitor of an ubiquitin-specific protease. USP47 is a member of the ubiquitin-specific protease (USP/UBP) superfamily of cysteine proteases. In some embodiments, the compounds disclosed herein are inhibitors of USP47.

Further embodiments provided herein include combinations of one or more of the particular embodiments set forth above.

### Diseases

### Cancer

In some embodiments, the heteroaryl compounds disclosed herein are useful for treating cancer. In some embodiments, the cancer is mediated by activation of transcriptional coactivator with PDZ binding motif/Yes-associated protein transcription coactivator (TAZ/YAP). In some embodiments, the cancer is mediated by modulation of the interaction of YAP/TAZ with TEAD. In some embodiments, the cancer is characterized by a mutant Gα-protein. In some embodiments, the mutant Gα-protein is selected from G12, G13, Gq, G11, Gi, Go, and Gs. In some embodiments, the mutant Gα-protein is G12. In some embodiments, the mutant Gα-protein is G13. In some embodiments, the mutant Gα-protein is Gq. In some embodiments, the mutant Gα-protein is G11. In some embodiments, the mutant Gα-protein is Gi. In some embodiments, the mutant Gα-protein is Go. In some embodiments, the mutant Gα-protein is Gs.

In some embodiments, the cancer is a solid tumor. In some instances, the cancer is a hematologic malignancy. In some instances, the solid tumor is a sarcoma or carcinoma. In some instances, the solid tumor is a sarcoma. In some instances, the solid tumor is a carcinoma.

Exemplary sarcoma includes, but is not limited to, alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epithelioid cell differentiation, osteosarcoma, parosteal osteosarcoma, neoplasm with perivascular epithelioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, and telangiectatic osteosarcoma.

Exemplary carcinoma includes, but is not limited to, adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, and vulvar cancer. In some instances, the liver cancer is primary liver cancer.

In some instances, the cancer is selected from uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, and meningioma. In some cases, the cancer is uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, or meningioma. In some cases, the cancer is uveal melanoma, mesothelioma, esophageal cancer, or liver cancer. In some cases, the cancer is uveal melanoma. In some cases, the cancer is mesothelioma. In some cases, the cancer is esophageal cancer. In some cases, the cancer is liver cancer. In some cases, the cancer is primary liver cancer.

In some instances, the cancer is a hematologic malignancy. In some embodiments, a hematologic malignancy is a leukemia, a lymphoma, a myeloma, a non-Hodgkin's lymphoma, a Hodgkin's lymphoma, a T-cell malignancy, or a B-cell malignancy. In some instances, a hematologic malignancy is a T-cell malignancy. Exemplary T-cell malignancy includes, but is not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, and treatment-related T-cell lymphomas.

In some instances, a hematologic malignancy is a B-cell malignancy. Exemplary B-cell malignancy includes, but is not limited to, chronic lymphocytic leukemia (CLL), small lymphocytic lymphoma (SLL), high risk CLL, and a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, the cancer is a relapsed or refractory cancer. In some embodiments, the relapsed or refractory cancer is a relapsed or refractory solid tumor. In some embodiments, the relapsed or refractory solid tumor is a relapsed or refractory sarcoma or a relapsed or refractory carcinoma. In some embodiments, the relapsed or refractory carcinoma includes adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, and vulvar cancer.

In some instances, the relapsed or refractory cancer is selected from relapsed or refractory uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, and meningioma. In some cases, the relapsed or refractory cancer is relapsed or refractory uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, or meningioma. In some cases, the relapsed or refractory cancer is relapsed or refractory uveal melanoma, mesothelioma, esophageal cancer, or liver cancer. In some cases, the relapsed or refractory cancer is relapsed or refractory uveal melanoma. In some cases, the relapsed or refractory cancer is relapsed or refractory mesothelioma. In some cases, the relapsed or refractory cancer is relapsed or refractory esophageal cancer. In some cases, the relapsed or refractory cancer is relapsed or refractory liver cancer. In some cases, the relapsed or refractory cancer is relapsed or refractory primary liver cancer.

In some instances, the relapsed or refractory cancer is a relapsed or refractory hematologic malignancy. In some embodiments, a relapsed or refractory hematologic malignancy is a relapsed or refractory leukemia, a relapsed or refractory lymphoma, a relapsed or refractory myeloma, a relapsed or refractory non-Hodgkin's lymphoma, a relapsed or refractory Hodgkin's lymphoma, a relapsed or refractory T-cell malignancy, or a relapsed or refractory B-cell malignancy. In some instances, a relapsed or refractory hematologic malignancy is a relapsed or refractory T-cell malignancy. In some instances, a relapsed or refractory hematologic malignancy is a relapsed or refractory B-cell malignancy, such as for example, chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, the cancer is a metastasized cancer. In some instances, the metastasized cancer is a metastasized solid tumor. In some instances, the metastasized solid tumor is a metastasized sarcoma or a metastasized carcinoma. In some embodiments, the metastasized carcinoma includes adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, and vulvar cancer.

In some instances, the metastasized cancer is selected from metastasized uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, and meningioma. In some cases, the metastasized cancer is metastasized uveal melanoma, mesothelioma, esophageal cancer, liver cancer, breast cancer, hepatocellular carcinoma, lung adenocarcinoma, glioma, colon cancer, colorectal cancer, gastric cancer, medulloblastoma, ovarian cancer, esophageal squamous cell carcinoma, sarcoma, Ewing sarcoma, head and neck cancer, prostate cancer, or meningioma. In some cases, the metastasized cancer is metastasized uveal melanoma, mesothelioma, esophageal cancer, or liver cancer. In some cases, the metastasized cancer is metastasized uveal melanoma. In some cases, the metastasized cancer is metastasized mesothelioma. In some cases, the metastasized cancer is metastasized esophageal cancer. In some cases, the metastasized cancer is metastasized liver cancer. In some cases, the metastasized cancer is metastasized primary liver cancer.

In some instances, the metastasized cancer is a metastasized hematologic malignancy. In some embodiments, the metastasized hematologic malignancy is a metastasized leukemia, a metastasized lymphoma, a metastasized myeloma, a metastasized non-Hodgkin's lymphoma, a metastasized Hodgkin's lymphoma, a metastasized T-cell malignancy, or a metastasized B-cell malignancy. In some instances, a metastasized hematologic malignancy is a metastasized T-cell malignancy. In some instances, a metastasized hematologic malignancy is a metastasized B-cell malignancy, such as for example, chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the cancer is selected from malignant peripheral nerve sheath tumor (MPNST), schwannoma, and cutaneous neurofibromas.

### Congenital Diseases

In some embodiments, the compounds disclosed herein are useful for treating a congenital disease. In some embodiments, the congenital disease is mediated by activation of transcriptional coactivator with PDZ binding motif/Yes-associated protein transcription coactivator (TAZ/YAP). In some embodiments, the congenital disease is characterized by a mutant Gα-protein. In some embodiments, the mutant Gα-protein is selected from G12, G13, Gq, G11, Gi, Go, and Gs. In some embodiments, the mutant Gα-protein is G12. In some embodiments, the mutant Gα-protein is G13. In some embodiments, the mutant Gα-protein is Gq. In some embodiments, the mutant Gα-protein is G11. In some embodiments, the mutant Gα-protein is Gi. In some embodiments, the mutant Gα-protein is Go. In some embodiments, the mutant Gα-protein is Gs.

In some embodiments, the congenital disease is the result of a genetic abnormality, an intrauterine environment, errors related to morphogenesis, infection, epigenetic modifications on a parental germline, or a chromosomal abnormality. Exemplary congenital diseases include, but are not limited to, Sturge-Weber Syndrome, Port-Wine stain, Holt-Oram syndrome, abdominal wall defects, Becker muscular dystrophy (BMD), biotinidase deficiency, Charcot-Marie-Tooth (CMT), cleft lip, cleft palate, congenital adrenal hyperplasia, congenital heart defects, congenital hypothyroidism, congenital muscular dystrophy, cystic fibrosis, Down syndrome, Duchenne muscular dystrophy, Fragile X syndrome, Friedreich's ataxia, galactosemia, hemoglobinopathies, Krabbe disease, limb-girdle muscular dystrophy, medium chain acyl-CoA dehydrogenase deficiency, myasthenia gravis, neural tube defects, phenylketonuria, Pompe disease, severe combined immunodeficiency (SCID), Stickler syndrome (or hereditary progressive arthro-ophthalmopathy), spinal muscular atrophy, and trisomy 18. In some embodiments, the congenital disease is Sturge-Weber Syndrome or Port-Wine stain. In some embodiments, the congenital disease is Sturge-Weber Syndrome. In some embodiments, the congenital disease is Port-Wine stain.

### EXAMPLES

These examples are provided for illustrative purposes only and not to limit the scope of the claims provided herein.

### List of Abbreviations

As used above, and throughout the disclosure, the following abbreviations, unless otherwise indicated, shall be understood to have the following meanings:
ACN or MeCN acetonitrile
Ac acetyl
Bn benzyl
BOC or Boc tert-butyl carbamate
° C. degrees Celsius
DBA or dba dibenzylideneacetone
DCM dichloromethane (CH₂Cl₂)
DIPEA or DIEA diisopropylethylamine
DMF dimethylformamide
DMSO dimethylsulfoxide
Dppf or dppf 1,1′-bis(diphenylphosphino)ferrocene
EA or EtOAc ethyl acetate
eq or equiv. equivalent(s)
Et ethyl
Et₂O diethyl ether
EtOH ethanol
g gram(s)
h or hr hour(s)
HPLC high performance liquid chromatography
Hz hertz
LCMS liquid chromatography mass spectrometry
m/z mass-to-charge ratio
M molar
Me methyl
MeI methyl iodide
MeOH methanol
mg milligram(s)
MHz megahertz
umol micromole(s)
uL microliter(s)
mL milliliter(s)
mmol millimole(s)
MS mass spectroscopy
NMR nuclear magnetic resonance
PE petroleum ether
Ph phenyl
prep-HPLC preparative high pressure liquid chromatography
prep-TLC preparative thin layer chromatography
RT retention time
TEA triethylamine
TFA trifluoroacetic acid
THF tetrahydrofuran
TLC thin layer chromatography
XantPhos 4,5-bis(diphenylphosphino)-9,9-dimethylxanthene

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times were approximate and were not optimized. Column chromatography and thin layer chromatography (TLC) were performed on silica gel unless otherwise noted. Same starting materials and intermediate compounds might have different annotation.

### Example 1: methyl-[2-[5-[3-[4-(trifluoromethyl)anilino]pyrazin-2-yl]-1,3,4-oxadiazol-2-yl]ethyl]cyanamide (Compound 1)

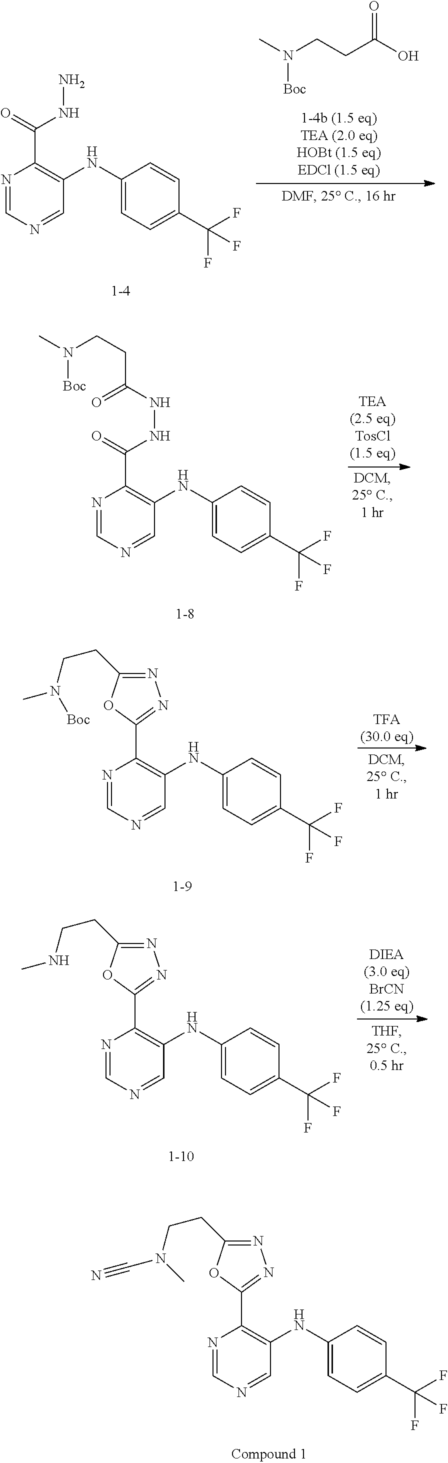 [SMILES: NNC(=O)c1ncncc1Nc1ccc(C(F)(F)F)cc1 ; CN(CCC(=O)O)C(=O)OC(C)(C)C ; CN(CCC(=O)NNC(=O)c1ncncc1Nc1ccc(C(F)(F)F)cc1)C(=O)OC(C)(C)C ; CN(CCc1nnc(-c2ncncc2Nc2ccc(C(F)(F)F)cc2)o1)C(=O)OC(C)(C)C ; CNCCc1nnc(-c2ncncc2Nc2ccc(C(F)(F)F)cc2)o1 ; CN(C#N)CCc1nnc(-c2ncncc2Nc2ccc(C(F)(F)F)cc2)o1]

### tert-butyl N-methyl-N-[3-oxo-3-[2-[3-[4-(trifluoromethyl)anilino]pyrazine-2-carbonyl]hydrazino]propyl]carbamate

To a solution of 1-4 (350 mg, 1.18 mmol, 1 eq) and 1-4b (359.0 mg, 1.77 mmol, 1.5 eq) in DMF (4 mL) were add EDCI (338.6 mg, 1.77 mmol, 1.5 eq), HOBt (238.7 mg, 1.77 mmol, 1.5 eq) and TEA (238.3 mg, 2.36 mmol, 0.33 mL, 2 eq). The mixture was stirred at 25° C. for 16 h. TLC (PE/EA=0/1, UV) showed starting material was consumed completely and new spots formed. The mixture was diluted with H₂O (10 mL), extracted with EA (20 mL*3). The combined organic layer was washed with brine (20 mL), dried over Na₂SO₄, filtered and concentrated under reduced pressure to give a residue. The residue was purified by flash silica gel chromatography to give 1-8 (260 mg, 0.41 mmol, 35.2% yield) as yellow oil. LCMS (ESI): RT=0.959 min, mass calc. for C₂₁H₂₅F₃N₆O₄ 482.19 found 483.3 [M+H]⁺

### tert-butyl N-methyl-N-[2-[5-[3-[4-(trifluoromethyl)anilino]pyrazin-2-yl]-1,3,4-oxadiazol-2-yl]ethyl]carbamate

To a solution of 1-8 (260 mg, 0.41 mmol, 1 eq) in DCM (3 mL) were added TosCl (118.7 mg, 0.62 mmol, 1.5 eq) and TEA (105.0 mg, 1.04 mmol, 0.14 mL, 2.5 eq). The mixture was stirred at 25° C. for 1 h. The mixture was diluted with H₂O (10 mL), extracted with DCM (20 mL*3). The combined organic layer was washed with brine (20 mL), dried over Na₂SO₄, filtered and concentrated under reduced pressure to give a residue. The residue was purified by flash silica gel chromatography to give 1-9 (150 mg, 0.32 mmol, 77.1% yield) as a yellow solid. LCMS (ESI): RT=0.965 min, mass calc. for C₂₁H₂₃F₃N₆O₃ 464.18, m/z found 465.1 [M+H]⁺

### 3-[5-[2-(methylamino)ethyl]-1,3,4-oxadiazol-2-yl]-N-[4-(trifluoromethyl)phenyl]pyrazin-2-amine

To a solution of 1-9 (150 mg, 0.32 mmol, 1 eq) in DCM (1 mL) was added TFA (1.10 g, 9.69 mmol, 0.72 mL, 30 eq). The mixture was stirred at 25° C. for 1 h. The mixture was concentrated under reduced pressure to give 1-10 (160 mg, crude, TFA) as yellow oil. LCMS (ESI): RT=0.719 min, mass calc. for C₁₆H₁₅F₃N₆O 364.13 found 365.0 [M+]⁺

### methyl-[2-[5-[3-[4-(trifluoromethyl)anilino]pyrazin-2-yl]-1,3,4-oxadiazol-2-yl]ethyl]cyanamide (Compound 1)

To a solution of 1-10 (150 mg, 0.41 mmol, 1 eq, TFA) and DIEA (159.6 mg, 1.24 mmol, 0.22 mL, 3 eq) in THF (1 mL) was added BrCN (65.4 mg, 0.62 mmol, 45 uL, 1.5 eq). The mixture was stirred at 25° C. for 0.5 h. The mixture was quenched with H₂O (10 mL), extracted with EA (20 mL*3). The combined organic layers were washed with brine (15 mL), dried over Na₂SO₄, filtered and concentrated under reduced pressure to give a residue. The residue was purified by prep-HPLC to give the title compound (88.4 mg, 0.23 mmol, 55.2% yield) as a yellow solid. LCMS (ESI): RT=0.849 min, mass calc. for C₁₇H₁₄F₃N₇O 389.12 found 390.0 [M+]⁺; ¹H NMR (400 MHz, CDCl₃) δ 10.37 (s, 1H), 8.39 (d, J=2.3 Hz, 1H), 8.23 (d, J=2.3 Hz, 1H), 7.92 (d, J=8.5 Hz, 2H), 7.64 (d, J=8.5 Hz, 2H), 3.68-3.58 (m, 2H), 3.45-3.35 (m, 2H), 3.00 (s, 3H).

### Example 2: ethyl-[2-[5-[3-[4-(trifluoromethyl)anilino]pyrazin-2-yl]-1,3,4-oxadiazol-2-yl]ethyl]cyanamide (Compound 2)

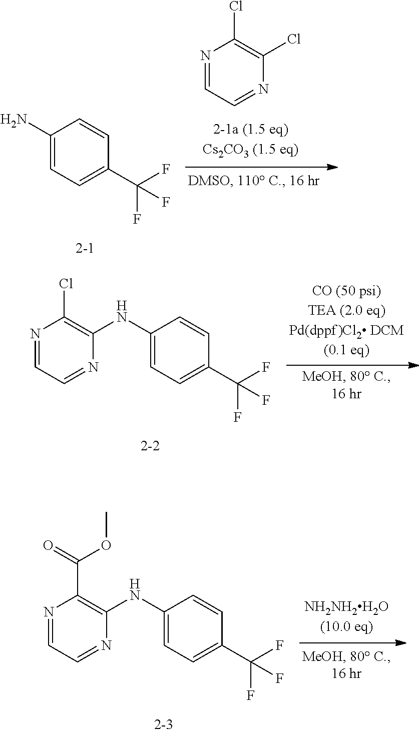 [SMILES: Clc1nccnc1Cl ; Nc1ccc(C(F)(F)F)cc1 ; FC(F)(F)c1ccc(Nc2nccnc2Cl)cc1 ; COC(=O)c1nccnc1Nc1ccc(C(F)(F)F)cc1]

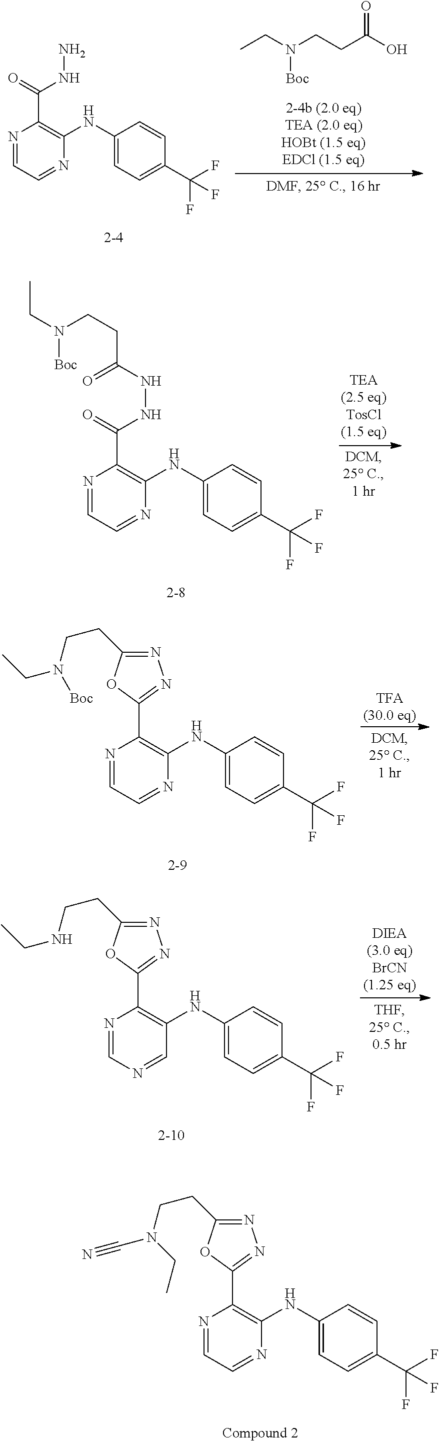 [SMILES: NNC(=O)c1nccnc1Nc1ccc(C(F)(F)F)cc1 ; CCN(CCC(=O)O)C(=O)OC(C)(C)C ; CCN(CCC(=O)NNC(=O)c1nccnc1Nc1ccc(C(F)(F)F)cc1)C(=O)OC(C)(C)C ; CCN(CCc1nnc(-c2nccnc2Nc2ccc(C(F)(F)F)cc2)o1)C(=O)OC(C)(C)C ; CCNCCc1nnc(-c2ncncc2Nc2ccc(C(F)(F)F)cc2)o1 ; CCN(C#N)CCc1nnc(-c2nccnc2Nc2ccc(C(F)(F)F)cc2)o1]

### 3-chloro-N-[4-(trifluoromethyl)phenyl]pyrazin-2-amine

To a mixture of 2-1 (5 g, 31.03 mmol, 3.85 mL, 1 eq) and 2-1a (6.93 g, 46.55 mmol, 1.5 eq) in DMSO (30 mL) was added Cs₂CO₃ (15.17 g, 46.55 mmol, 1.5 eq). The mixture was stirred at 110° C. for 16 h. The mixture was diluted with H₂O (50 mL), extracted with EA (100 mL*3). The combined organic layers were washed with brine (80 mL), dried over Na₂SO₄, filtered and concentrated under reduced pressure to give a residue. The residue was purified by flash silica gel chromatography to give 2-2 (2.7 g, 7.60 mmol, 24.5% yield) as a yellow solid. LCMS (ESI): RT=0.948 min, mass calc. for C₁₁H₇ClF₃N₃ 273.03, m/z found 274.1 [M+H]⁺

### methyl 3-[4-(trifluoromethyl)anilino]pyrazine-2-carboxylate

To a solution of 2-2 (1 g, 3.65 mmol, 1 eq) in MeOH (10 mL) was added Pd(dppf)Cl₂.CH₂Cl₂ (298.4 mg, 0.37 mmol, 0.1 eq) and TEA (739.6 mg, 7.31 mmol, 1.02 mL, 2 eq) under N₂. The suspension was degassed under vacuum and purged with CO for 3 times. The mixture was stirred under CO (50 psi) at 80° C. for 16 hours. The mixture was concentrated under reduced pressure to give a residue. The residue was purified by flash silica gel chromatography to give 2-3 (700 mg, 2.36 mmol, 64.4% yield) as a yellow solid. ¹H NMR (400 MHz, CDCl₃) δ 10.43 (br s, 1H), 8.40 (d, J=2.3 Hz, 1H), 8.17 (d, J=2.3 Hz, 1H), 7.84 (d, J=8.5 Hz, 2H), 7.62 (d, J=8.5 Hz, 2H), 4.07 (s, 3H).

### 3-[4-(trifluoromethyl)anilino]pyrazine-2-carbohydrazide

To a solution of 2-3 (700 mg, 2.36 mmol, 1 eq) in MeOH (7 mL) was added hydrazine hydrate (1.39 g, 23.55 mmol, 1.35 mL, 10 eq). The mixture was stirred at 80° C. for 16 h. The mixture was concentrated under reduced pressure to give a residue. The residue was diluted with H₂O (10 mL), extracted with EA (20 mL*3). The combined organic layers were washed with brine (10 mL), dried over Na₂SO₄, filtered and concentrated under reduced pressure to give 2-4 (700 mg, crude) as a yellow solid. LCMS (ESI): RT=0.781 min, mass calc. for C₁₂H₁₀F₃N₅O 297.08, m/z found 297.9 [M+H]⁺

### tert-butyl N-ethyl-N-[3-oxo-3-[2-[3-[4-(trifluoromethyl)anilino]pyrazine-2-carbonyl]hydrazino]propyl]carbamate

To a solution of 2-4 (300 mg, 1.01 mmol, 1 eq) and 2-4b (438.6 mg, 2.02 mmol, 2 eq) in DMF (3 mL) were add EDCI (290.2 mg, 1.51 mmol, 1.5 eq), HOBt (204.6 mg, 1.51 mmol, 1.5 eq) and TEA (204.3 mg, 2.02 mmol, 0.28 mL, 2 eq). The mixture was stirred at 25° C. for 16 h. The mixture was diluted with H₂O (10 mL), extracted with EA (20 mL*3). The combined organic layer was washed with brine (20 mL), dried over Na₂SO₄, filtered and concentrated under reduced pressure to give a residue. The residue was purified by flash silica gel chromatography 2-8 (320 mg, 0.41 mmol, 40.2% yield) as a yellow solid. LCMS (ESI): RT=0.927 min, mass calc. for C₂₂H₂₇F₃N₆O₄ 496.20 found 397.1 [M-Boc+H]⁺

### tert-butyl N-ethyl-N-[2-[5-[3-[4-(trifluoromethyl)anilino]pyrazin-2-yl]-1,3,4-oxadiazol-2-yl]ethyl]carbamate

To a solution of 2-8 (320 mg, 0.41 mmol, 1 eq) in DCM (3 mL) were added TosCl (116.1 mg, 0.61 mmol, 1.5 eq) and TEA (102.7 mg, 1.02 mmol, 0.14 mL, 2.5 eq). The mixture was stirred at 25° C. for 1 h. The mixture was diluted with H₂O (10 mL), extracted with DCM (20 mL*3). The combined organic layer was washed with brine (20 mL), dried over Na₂SO₄, filtered and concentrated under reduced pressure to give a residue. The residue was purified by flash silica gel chromatography to give 2-9 (130 mg, 0.25 mmol, 62.2% yield) as yellow oil. LCMS (ESI): RT=1.010 min, mass calc. for C₂₂H₂₅F₃N₆O₃ 478.19, m/z found 479.2 [M+H]⁺; ¹H NMR (400 MHz, CDCl₃) δ 10.46 (s, 1H), 8.37 (d, J=1.8 Hz, 1H), 8.22 (d, J=2.4 Hz, 1H), 7.92 (d, J=8.5 Hz, 2H), 7.64 (d, J=8.6 Hz, 2H), 3.73 (t, J=6.9 Hz, 2H), 3.36-3.22 (m, 4H), 1.43 (s, 9H), 1.18-1.11 (m, 3H)

### 3-[5-[2-(ethylamino)ethyl]-1,3,4-oxadiazol-2-yl]-N-[4-(trifluoromethyl)phenyl]pyrazin-2-amine

To a solution of 2-9 (130 mg, 0.27 mmol, 1 eq) in DCM (1 mL) was added TFA (929.4 mg, 8.15 mmol, 0.60 mL, 30 eq). The mixture was stirred at 25° C. for 1 h. The mixture was concentrated under reduced pressure to give 2-10 (150 mg, crude, TFA) as yellow oil. LCMS (ESI): RT=0.795 min, mass calc. for C₁₇H₁₇F₃N₆O 378.14 found 379.3 [M+H]⁺

### ethyl-[2-[5-[3-[4-(trifluoromethyl)anilino]pyrazin-2-yl]-1,3,4-oxadiazol-2-yl]ethyl]cyanamide (Compound 2)

To a solution of 2-10 (130 mg, 0.26 mmol, 1 eq, TFA) and DIEA (102.4 mg, 0.79 mmol, 0.14 mL, 3 eq) in THF (1 mL) was added BrCN (42.0 mg, 0.40 mmol, 29 uL, 1.5 eq). The mixture was stirred at 25° C. for 0.5 h. The mixture was quenched with H₂O (10 mL), extracted with EA (20 mL*3). The combined organic layers were washed with brine (15 mL), dried over Na₂SO₄, filtered and concentrated under reduced pressure to give a residue. The residue was checked by HPLC. The residue was purified by prep-HPLC (column: Waters Xbridge 150*25 mm*5 um; mobile phase: [water (0.05% NH₃H₂O+10 mM NH₄HCO₃)-ACN]; B %: 50%-80%, 7.8 min) to give the title compound (19.1 mg, 47.4 umol, 17.9% yield) as a yellow solid. LCMS (ESI): RT=0.875 min, mass calc. for C₁₈H₁₆F₃N₇O 403.14 found 404.1 [M+H]⁺; ¹H NMR (400 MHz, CDCl₃) δ 10.34 (s, 1H), 8.36 (d, J=2.3 Hz, 1H), 8.20 (d, J=2.3 Hz, 1H), 7.89 (d, J=8.5 Hz, 2H), 7.61 (d, J=8.5 Hz, 2H), 3.66-3.58 (m, 2H), 3.42-3.34 (m, 2H), 3.17 (q, J=7.3 Hz, 2H), 1.31 (t, J=7.2 Hz, 3H).

### Example 3: N-(2-(5-(2-((4-(trifluoromethyl)phenyl)amino)pyridin-3-yl)-1,3,4-oxadiazol-2-yl)ethyl)cyanamide (Compound 3)

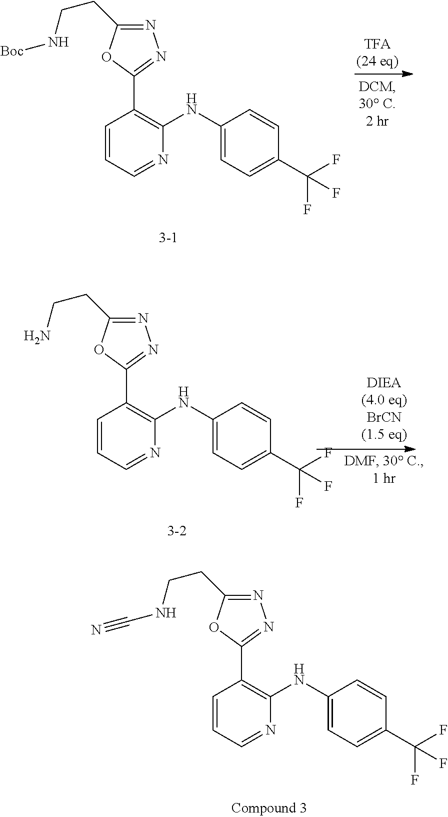 [SMILES: CC(C)(C)OC(=O)NCCc1nnc(-c2cccnc2Nc2ccc(C(F)(F)F)cc2)o1 ; NCCc1nnc(-c2cccnc2Nc2ccc(C(F)(F)F)cc2)o1 ; N#CNCCc1nnc(-c2cccnc2Nc2ccc(C(F)(F)F)cc2)o1]

### 3-(5-(2-aminoethyl)-1,3,4-oxadiazol-2-yl)-N-(4-(trifluoromethyl)phenyl)pyridin-2-amin

To a solution of 3-1 (250.0 mg, 0.56 mmol, 1 eq) in DCM (3 mL) at 30° C. was added TFA (1.5 g, 13.51 mmol, 1.0 mL, 24 eq). The mixture was stirred at 30° C. for 2 h. The residue was concentrated under reduced pressure, which was purified by prep-HPLC: (column: Welch Xtimate C18 150*25 mm*5 um; mobile phase: [water (0.05% HCl)-ACN]; B %: 20%-50%, 8.5 min) to give compound 3-2 (120.0 mg, 0.3 mmol, 61.1% yield) as a yellow solid. LCMS (ESI): RT=0.828 min, mass calc. for C₁₆H₁₄F₃N₅O 349.12, m/z found 350.2 [M+H]⁺; ¹H NMR (400 MHz, DMSO-d₆) δ 10.29 (s, 1H), 8.50 (dd, J=1.9, 4.9 Hz, 1H), 8.32 (dd, J=1.8, 7.8 Hz, 1H), 8.01 (d, J=8.5 Hz, 2H), 7.71 (d, J=8.5 Hz, 2H), 7.15 (dd, J=5.0, 7.8 Hz, 1H), 3.40-3.31 (m, 4H).

### N-(2-(5-(2-((4-(trifluoromethyl)phenyl)amino)pyridin-3-yl)-1,3,4-oxadiazol-2-yl)ethyl)cyanamide (Compound 3)

To a solution of 3-2 (70.0 mg, 0.20 mmol, 1 eq) and DIEA (103.6 mg, 0.80 mmol, 0.14 mL, 4.0 eq) in DMF (1 mL) at 30° C. was added CNBr (31.8 mg, 0.30 mmol, 22 uL, 1.5 eq), and the resulting mixture was stirred at 30° C. for 1 h. The reaction mixture was concentrated under reduced pressure to give a residue, which was purified by prep-HPLC: (column: Welch Xtimate C18 150*25 mm*5 um; mobile phase: [water (0.05% HCl)-ACN]; B %: 45%-75%, 8.5 min) to give the title compound (4.6 mg, 11 umol, 5.7% yield) as yellow oil. LCMS (ESI): RT=2.014 min, mass calc. for C₁₇H₁₃F₃N₆O 374.11, m/z found 375.1 [M+H]⁺; ¹H NMR (400 MHz, CDCl₃) δ 10.23 (s, 1H), 8.45 (dd, J=1.8, 4.8 Hz, 1H), 8.15 (dd, J=1.9, 7.8 Hz, 1H), 7.94 (d, J=8.5 Hz, 2H), 7.61 (d, J=8.6 Hz, 2H), 6.94 (dd, J=4.8, 7.8 Hz, 1H), 4.48 (brs, 1H), 3.71 (q, J=6.1 Hz, 2H), 3.30 (t, J=6.0 Hz, 2H).

### Example 4: tert-butyl (2-(5-(2-((4-(trifluoromethyl)phenyl)amino)pyridin-3-yl)-1,3,4-oxadiazol-2-yl)ethyl)carbamate (Compound 4)

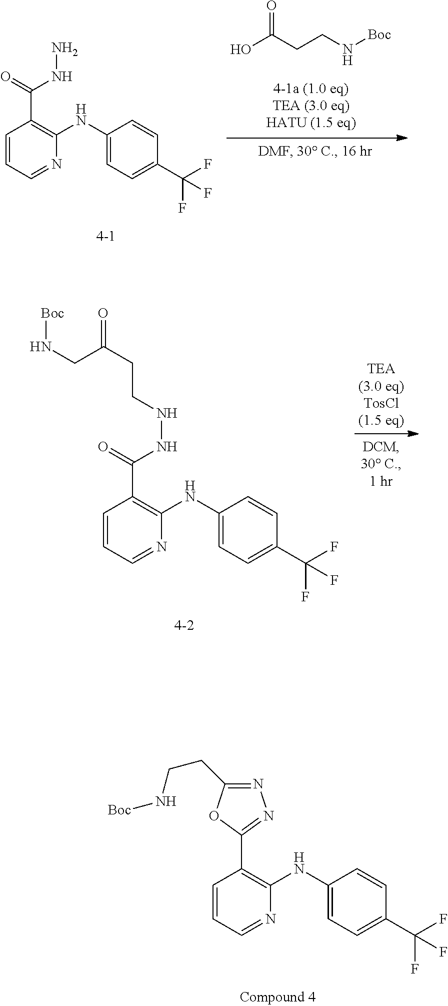 [SMILES: NNC(=O)c1cccnc1Nc1ccc(C(F)(F)F)cc1 ; CC(C)(C)OC(=O)NCCC(=O)O ; CC(C)(C)OC(=O)NCC(=O)CCNNC(=O)c1cccnc1Nc1ccc(C(F)(F)F)cc1 ; CC(C)(C)OC(=O)NCCc1nnc(-c2cccnc2Nc2ccc(C(F)(F)F)cc2)o1]

### tert-butyl (2-oxo-4-(2-(2-((4-(trifluoromethyl)phenyl)amino)nicotinoyl)hydrazinyl)butyl)carbamate

To a solution of 4-1a (300.0 mg, 1.59 mmol, 1.0 eq), HATU (904.3 mg, 2.38 mmol, 1.5 eq) in DMF (2 mL) at 30° C. were added 4-1 (516.7 mg, 1.74 mmol, 1.1 eq) and TEA (481.3 mg, 4.76 mmol, 0.67 mL, 3 eq). The mixture was stirred at 30° C. for 16 h. The residue was diluted with water (20 mL), Na₂CO₃ (20 mL) and extracted with EA (20 mL*3). The combined organic layers were washed with brine (20 mL), dried over Na₂SO₄, filtered and concentrated under reduced pressure to give compound 4-2 (1.04 g, 1.33 mmol, 84.1% yield) as a yellow solid. LCMS (ESI): RT=0.773 min, mass calc. for C₁₉H₁₈F₃N₅O₃ 421.14, m/z found 422.0 [M+H]⁺.

### tert-butyl (2-(5-(2-((4-(trifluoromethyl)phenyl)amino)pyridin-3-yl)-1,3,4-oxadiazol-2-yl)ethyl)carbamate (Compound 4)

To a solution of 4-2 (1.0 g, 1.28 mmol, 1.0 eq) in DCM (10 mL) at 30° C. was added TEA (389.7 mg, 3.85 mmol, 0.54 mL, 3.0 eq) and TosCl (367.1 mg, 1.93 mmol, 1.5 eq). The mixture was stirred at 30° C. for 1 h. The reaction mixture was diluted with water (20 mL) and extracted with EA (20 mL*3). The combined organic layers were washed with brine (30 mL), dried over anhydrous Na₂SO₄, filtered and concentrated under reduced pressure to give a residue, which was purified by flash silica gel chromatography (ISCO®; 20 g SepaFlash® Silica Flash Column, Eluent of 0˜35% Ethyl acetate/Petroleum ether gradient @ 30 mL/min) to give the title compound (270.0 mg, 0.60 mmol, 46.8% yield) as yellow oil. LCMS (ESI): RT=1.042 min, mass calc. for C₂₁H₂₂F₃N₅O₃ 449.17, m/z found 450.3 [M+H]⁺; ¹H NMR (400 MHz, CDCl₃) δ 10.33 (s, 1H), 8.44 (dd, J=1.9, 4.9 Hz, 1H), 8.15 (dd, J=1.9, 7.7 Hz, 1H), 7.95 (d, J=8.5 Hz, 2H), 7.61 (d, J=8.5 Hz, 2H), 6.93 (dd, J=4.9, 7.7 Hz, 1H), 5.12 (brs, 1H), 3.70 (q, J=6.1 Hz, 2H), 3.18 (t, J=6.3 Hz, 2H), 1.44 (s, 9H).

### Example 5: ethyl-[[5-[2-[4-(trifluoromethyl)anilino]-3-pyridyl]-1,3,4-oxadiazol-2-yl]methyl]cyanamide (Compound 5), 3-[5-(ethylaminomethyl)-1,3,4-oxadiazol-2-yl]-N-[4-(trifluoromethyl)phenyl]pyridin-2-amine (Compound 7), and tert-butyl N-ethyl-N-[[5-[2-[4-(trifluoromethyl)anilino]-3-pyridyl]-1,3,4-oxadiazol-2-yl]methyl]carbamate (Compound 8)

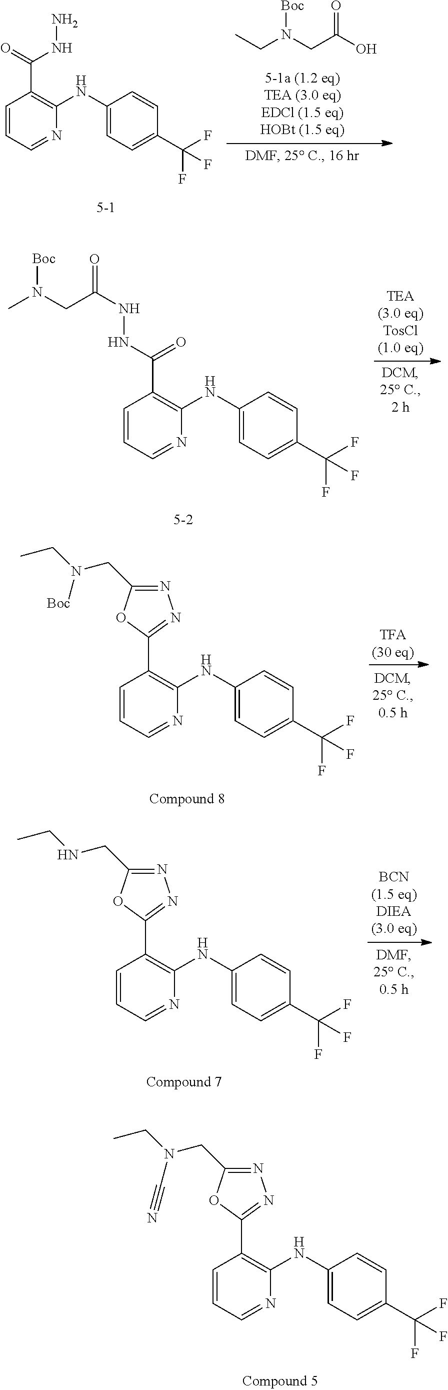 [SMILES: NNC(=O)c1cccnc1Nc1ccc(C(F)(F)F)cc1 ; CCN(CC(=O)O)C(=O)OC(C)(C)C ; CN(CC(=O)NNC(=O)c1cccnc1Nc1ccc(C(F)(F)F)cc1)C(=O)OC(C)(C)C ; CCN(Cc1nnc(-c2cccnc2Nc2ccc(C(F)(F)F)cc2)o1)C(=O)OC(C)(C)C ; CCNCc1nnc(-c2cccnc2Nc2ccc(C(F)(F)F)cc2)o1 ; CCN(C#N)Cc1nnc(-c2cccnc2Nc2ccc(C(F)(F)F)cc2)o1]

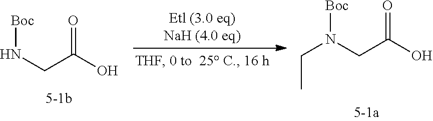

### 2-[tert-butoxycarbonyl(ethyl)amino]acetic acid

To a solution of NaH (913.6 mg, 22.84 mmol, 60%, 4.0 eq) in THF (30 mL) at 0° C. was added a solution of 5-1b (1 g, 5.71 mmol, 1 eq) in THF (10 mL). EtI (2.67 g, 17.13 mmol, 1.4 mL, 3.0 eq) was added to the above solution. The resulting mixture was stirred at 0° C. for 1 hr. The resulting mixture was stirred at 25° C. for 15 hr. The solution was quenched by H₂O at 0° C. and extracted with EA (30 mL). The mixture was acidified at 0° C. with 2N HCl to pH=2-3 and extracted with EA (40 mL*3). Then the combined organic layers were washed with brine (30 mL), dried by Na₂SO₄, filtered and concentrated under reduced pressure to give compound 5-1a (1 g, 4.92 mmol, 86.1% yield) as black brown oil. ¹H NMR (400 MHz, CDCl₃) δ 8.41-7.80 (m, 1H), 4.06-3.82 (m, 2H), 3.43-3.20 (m, 2H), 1.45 (br d, J=16.9 Hz, 9H), 1.31-1.22 (m, 1H), 1.12 (t, J=7.1 Hz, 3H).

### tert-butyl N-ethyl-N-[2-oxo-2-[2-[2-[4-(trifluoromethyl)anilino]pyridine-3-carbonyl]hydrazino]ethyl]carbamate

To a solution of 5-1 (300 mg, 1.01 mmol, 1 eq) in DMF (3 mL) was added 5-1a (246.3 mg, 1.21 mmol, 1.2 eq), HOBt (204.7 mg, 1.52 mmol, 1.5 eq), EDCI (290.4 mg, 1.52 mmol, 1.5 eq) and TEA (306.6 mg, 3.03 mmol, 0.4 mL, 3.0 eq). The mixture was stirred at 25° C. for 16 hr. The reaction mixture was concentrated under reduced pressure to give a residue. The residue was diluted with H₂O (10 mL) and extracted with EA (15 mL*3). The combined organic layers were washed with brine (15 mL), dried over Na₂SO₄, filtered and concentrated under reduced pressure to give a residue. The residue was purified by flash silica gel chromatography to give compound 5-2 (350 mg, 0.63 mmol, 62.6% yield) as a yellow solid. LCMS (ESI): RT=0.894 min, mass calc. for C₂₂H₂₆F₃N₅O₄ 481.19, m/z found 482.1 [M+H]⁺.

### tert-butyl N-ethyl-N-[[5-[2-[4-(trifluoromethyl)anilino]-3-pyridyl]-1,3,4-oxadiazol-2-yl]methyl]carbamate (Compound 8)

To a solution of 5-2 in DCM (2 mL) was added TosCl (138.6 mg, 0.73 mmol, 1.0 eq) and TEA (220.7 mg, 2.18 mmol, 0.3 mL, 3.0 eq). The mixture was stirred at 25° C. for 2 hr. The reaction mixture was concentrated under reduced pressure to give a residue. The residue was diluted with H₂O (10 mL) and extracted with EA (15 mL*3). The combined organic layers were washed with brine (15 mL), dried over Na₂SO₄, filtered and concentrated under reduced pressure to give a residue. The residue was purified by prep-HPLC to give Compound 8 (250 mg, 0.54 mmol, 74.2% yield) as a white solid. LCMS (ESI): RT=1.044 min, mass calc. for C₂₂H₂₄F₃N₅O₃ 463.18, m/z found 464.1 [M+H]⁺; ¹H NMR (400 MHz, CDCl₃) δ 10.32 (s, 1H), 8.44 (br d, J=3.5 Hz, 1H), 8.12 (br s, 1H), 7.95 (d, J=8.5 Hz, 2H), 7.61 (d, J=8.5 Hz, 2H), 6.93 (dd, J=4.9, 7.7 Hz, 1H), 4.92-4.64 (m, 2H), 3.42 (br s, 2H), 1.51 (br s, 9H), 1.19 (br s, 3H).

### 3-[5-(ethylaminomethyl)-1,3,4-oxadiazol-2-yl]-N-[4-(trifluoromethyl)phenyl]pyridin-2-amine (Compound 7)

To a solution of Compound 8 (240 mg, 0.52 mmol, 1 eq) in DCM (2 mL) was added TFA (1.77 g, 15.54 mmol, 1.2 mL, 30 eq). The mixture was stirred at 25° C. for 0.5 hr. The reaction mixture was concentrated under reduced pressure to give a residue. The residue was diluted with H₂O (10 mL) and extracted with EA (15 mL*3). The combined organic layers were washed with brine (15 mL), dried over Na₂SO₄, filtered and concentrated under reduced pressure to give desired compound (170 mg, 0.47 mmol, 90.3% yield) as brown oil. The residue (20 mg, crude) was purified by prep-HPLC (column: Welch Xtimate C18 150*25 mm*5 um; mobile phase: [water (0.05% HCl)-ACN]; B %: 20%-50%, 8.5 min) to give Compound 7 (3.4 mg, 8.7 umol, 1.6% yield, HCl) as a white solid. LCMS (ESI): RT=0.750 min, mass calc. for C₁₇H₁₆F₃N₅O 363.13, m/z found 364.0 [M+H]⁺; ¹H NMR (400 MHz, CD₃OD) δ 8.47 (dd, J=1.8, 5.0 Hz, 1H), 8.36 (dd, J=1.8, 7.8 Hz, 1H), 8.01 (d, J=8.5 Hz, 2H), 7.65 (d, J=8.5 Hz, 2H), 7.09 (dd, J=4.9, 7.9 Hz, 1H), 4.76 (s, 2H), 3.39-3.32 (m, 2H), 1.47-1.36 (m, 3H).

### ethyl-[[5-[2-[4-(trifluoromethyl)anilino]-3-pyridyl]-1,3,4-oxadiazol-2-yl]methyl]cyanamide (Compound 5)

To a solution of Compound 7 (50 mg, 0.14 mmol, 1 eq) in DMF (1 mL) was added DIEA (53.36 mg, 0.41 mmol, 71 uL, 3.0 eq) and BrCN (21.9 mg, 0.21 mmol, 15 uL, 1.5 eq). The mixture was stirred at 25° C. for 2 hr. The solution was quenched by addition of H₂O at 0° C. and extracted with EA (10 mL*3). Then the combined organic layers were washed with brine (15 mL), dried by Na₂SO₄, filtered and concentrated under reduced pressure to give a residue. The residue was purified by prep-HPLC (column: Welch Xtimate C18 150*25 mm*5 um; mobile phase: [water (0.05% HCl)-ACN]; B %: 50%-80%, 8.5 min) to give Compound 5 (40.1 mg, 98.1 umol, 71.3% yield) as a white solid. LCMS (ESI): RT=0.904 min, mass calc. for C₁₈H₁₅F₃N₆O 388.13, m/z found 389.0 [M+H]⁺; ¹H NMR (400 MHz, CDCl₃) δ 10.31-10.11 (m, 1H), 8.48 (dd, J=1.8, 4.8 Hz, 1H), 8.22 (dd, J=2.0, 7.8 Hz, 1H), 7.95 (d, J=8.5 Hz, 2H), 7.62 (d, J=8.8 Hz, 2H), 6.97 (dd, J=4.8, 7.8 Hz, 1H), 4.58 (s, 2H), 3.23 (q, J=7.3 Hz, 2H), 1.37 (t, J=7.3 Hz, 3H).

### Example 6: methyl-[[5-[2-[4-(trifluoromethyl)anilino]-3-pyridyl]-1,3,4-oxadiazol-2-yl]methyl]cyanamide (Compound 6), 3-[5-(methylaminomethyl)-1,3,4-oxadiazol-2-yl]-N-[4-(trifluoromethyl)phenyl]pyridin-2-amine (Compound 9), and tert-butyl N-methyl-N-[[5-[2-[4-(trifluoromethyl)anilino]-3-pyridyl]-1,3,4-oxadiazol-2-yl]methyl]carbamate (Compound 10)

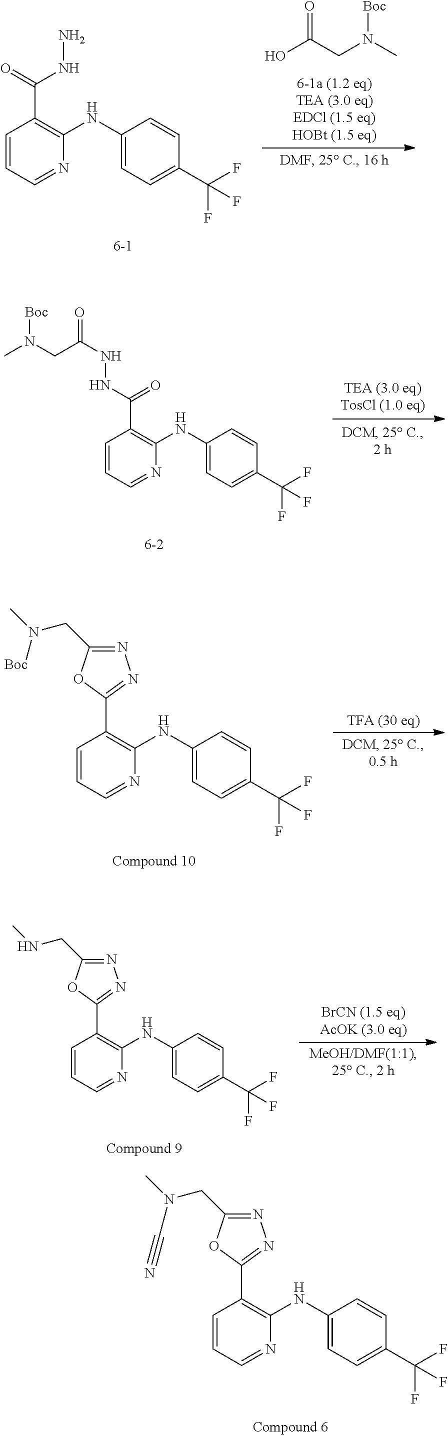 [SMILES: NNC(=O)c1cccnc1Nc1ccc(C(F)(F)F)cc1 ; CN(CC(=O)O)C(=O)OC(C)(C)C ; CN(CC(=O)NNC(=O)c1cccnc1Nc1ccc(C(F)(F)F)cc1)C(=O)OC(C)(C)C ; CN(Cc1nnc(-c2cccnc2Nc2ccc(C(F)(F)F)cc2)o1)C(=O)OC(C)(C)C ; CNCc1nnc(-c2cccnc2Nc2ccc(C(F)(F)F)cc2)o1 ; CN(C#N)Cc1nnc(-c2cccnc2Nc2ccc(C(F)(F)F)cc2)o1]

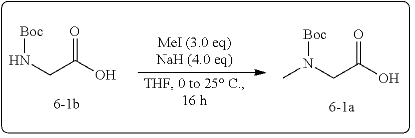

### 2-[tert-butoxycarbonyl(methyl)amino]acetic acid

To a solution of NaH (913.6 mg, 22.84 mmol, 60%, 4.0 eq) in THF (30 mL) at 0° C. was added a solution of 6-1b (1 g, 5.71 mmol, 1 eq) in THF (10 mL). MeI (2.43 g, 17.13 mmol, 1.1 mL, 3.0 eq) was added to the above solution. The mixture was stirred at 0° C. for 1 hr. The resulting mixture was stirred at 25° C. for 15 hr. The solution was quenched by H₂O at 0° C. and extracted with EA (40 mL). The mixture was acidified at 0° C. with 2N HCl to pH=2-3 and extracted with EA (40 mL*3). Then the combined organic layers were washed with brine (40 mL), dried by Na₂SO₄, filtered and concentrated under reduced pressure to give 6-1a (1.0 g, 5.29 mmol, 92.5% yield) as black brown oil. ¹H NMR (400 MHz, CDCl₃) δ 8.68 (br s, 1H), 4.05-3.92 (m, 2H), 2.94 (d, J=3.3 Hz, 3H), 1.45 (br d, J=15.1 Hz, 9H).

### tert-butyl N-methyl-N-[2-oxo-2-[2-[2-[4-(trifluoromethyl)anilino]pyridine-3-carbonyl]hydrazino]ethyl]carbamate

To a solution of 6-1 (300 mg, 1.01 mmol, 1 eq) in DMF (3 mL) was added 6-1a (229.9 mg, 1.22 mmol, 1.2 eq), HOBt (205.2 mg, 1.52 mmol, 1.5 eq), EDCI (291.2 mg, 1.52 mmol, 1.5 eq) and TEA (307.4 mg, 3.04 mmol, 0.4 mL, 3.0 eq). The mixture was stirred at 25° C. for 16 hr. The reaction mixture was concentrated under reduced pressure to give a residue. The residue was diluted with H₂O (20 mL) and extracted with EA (15 mL*3). The combined organic layers were washed with brine (15 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜40% Ethyl acetate/Petroleum ether gradient @ 30 mL/min) to give compound 6-2 (350 mg, 0.62 mmol, 61.3% yield) as a yellow solid. LCMS (ESI): RT=0.875 min, mass calc. for C₂₁H₂₄F₃N₅O₄ 467.18 m/z found 468.0 [M+H]⁺.

### tert-butyl N-methyl-N-[[5-[2-[4-(trifluoromethyl)anilino]-3-pyridyl]-1,3,4-oxadiazol-2-yl]methyl]carbamate (Compound 10)

To a solution of 6-2 (350 mg, 0.75 mmol, 1 eq) in DCM (2 mL) was added TosCl (142.8 mg, 0.75 mmol, 1.0 eq) and TEA (227.3 mg, 2.25 mmol, 0.3 mL, 3.0 eq). The mixture was stirred at 25° C. for 2 hr. The reaction mixture was concentrated under reduced pressure to give a residue. The residue was diluted with H₂O (10 mL) and extracted with EA (15 mL*3). The combined organic layers were washed with brine (15 mL), dried over Na₂SO₄, filtered and concentrated under reduced pressure to give a residue. The residue was purified by prep-HPLC (column: Welch Xtimate C18 150*25 mm*5 um; mobile phase: [water (0.05% HCl)-ACN]; B %: 60%-90%, 8.5 min) to give Compound 10 (250 mg, 0.56 mmol, 74.2% yield) as a white solid. LCMS (ESI): RT=1.017 min, mass calc. for C₂₁H₂₂F₃N₅O₃ 449.17, m/z found 450.1 [M+H]⁺; ¹H NMR (400 MHz, CDCl₃) δ 10.32 (s, 1H), 8.44 (br d, J=3.5 Hz, 1H), 8.13 (br s, 1H), 7.95 (d, J=8.5 Hz, 2H), 7.61 (d, J=8.5 Hz, 2H), 6.93 (dd, J=5.0, 7.3 Hz, 1H), 4.82-4.67 (m, 2H), 3.04 (br s, 3H), 1.59 (br s, 9H).

### 3-[5-(methylaminomethyl)-1,3,4-oxadiazol-2-yl]-N-[4-(trifluoromethyl)phenyl]pyridin-2-amine (Compound 9)

To a solution of Compound 10 (240 mg, 0.53 mmol, 1 eq) in DCM (2 mL) was added TFA (1.83 g, 16.02 mmol, 1.2 mL, 30 eq). The mixture was stirred at 25° C. for 0.5 hr. The reaction mixture was concentrated under reduced pressure to give a residue. The residue was diluted with H₂O (10 mL) and extracted with EA (15 mL*3). The combined organic layers were washed with brine (15 mL), dried over Na₂SO₄, filtered and concentrated under reduced pressure to give a product (200 mg, crude) as brown oil. The residue (20 mg, crude) was purified by prep-HPLC (column: Welch Xtimate C18 150*25 mm*5 um; mobile phase: [water (0.05% HCl)-ACN]; B %: 15%-45%, 8.5 min) to give Compound 9 (3.0 mg, 7.9 umol, 1.4% yield, HCl) as a white solid. LCMS (ESI): RT=0.745 min, mass calc. for C₁₆H₁₄F₃N₅O 349.12 m/z found 349.9 [M+H]⁺; ¹H NMR (400 MHz, CD₃OD) δ 8.48 (dd, J=1.8, 5.0 Hz, 1H), 8.35 (dd, J=1.8, 7.8 Hz, 1H), 8.02 (d, J=8.5 Hz, 2H), 7.65 (d, J=8.5 Hz, 2H), 7.09 (dd, J=4.8, 7.8 Hz, 1H), 4.75 (s, 2H), 2.96 (s, 3H).

### methyl-[[5-[2-[4-(trifluoromethyl)anilino]-3-pyridyl]-1,3,4-oxadiazol-2-yl]methyl]cyanamide (Compound 6)

To a solution of Compound 9 (150 mg, 0.43 mmol, 1 eq) in MeOH (1 mL) and DMF (1 mL) was added AcOK (126.4 mg, 1.29 mmol, 3.0 eq) and BrCN (68.2 mg, 0.64 mmol, 47 uL, 1.5 eq). The mixture was stirred at 25° C. for 2 hr. The solution was quenched by addition of H₂O at 0° C. and extracted with EA (10 mL*3). Then the combined organic layers were washed with brine (15 mL), dried by Na₂SO₄, filtered and concentrated under reduced pressure to give a residue. The residue was purified by prep-HPLC (column: Welch Xtimate C18 150*25 mm*5 um; mobile phase: [water (0.05% HCl)-ACN]; B %: 45%-75%, 8.5 min) to give Compound 6 (33.0 mg, 87.4 umol, 20.3% yield) as a white solid. LCMS (ESI): RT=0.889 min, mass calc. for C₁₇H₁₃F₃N₆O 374.11, m/z found 375.0 [M+H]⁺; ¹H NMR (400 MHz, CDCl₃) δ 10.22 (s, 1H), 8.48 (dd, J=2.0, 4.8 Hz, 1H), 8.21 (dd, J=1.8, 7.8 Hz, 1H), 7.95 (d, J=8.5 Hz, 2H), 7.62 (d, J=8.5 Hz, 2H), 6.97 (dd, J=5.0, 7.8 Hz, 1H), 4.55 (s, 2H), 3.05 (s, 3H).

### Example 7: 3-[5-(aminomethyl)-1,3,4-oxadiazol-2-yl]-N-[4-(trifluoromethyl)phenyl]pyridin-2-amine (Compound 11)

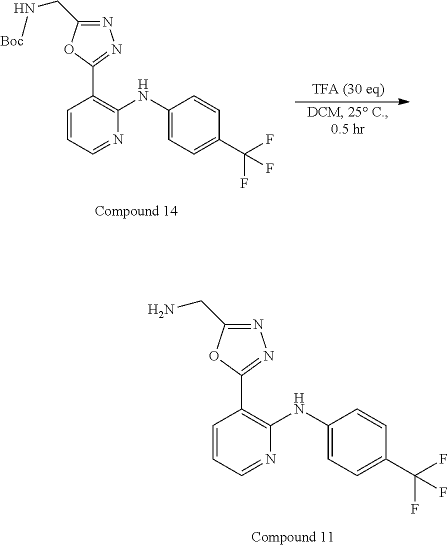 [SMILES: CC(C)(C)OC(=O)NCc1nnc(-c2cccnc2Nc2ccc(C(F)(F)F)cc2)o1 ; NCc1nnc(-c2cccnc2Nc2ccc(C(F)(F)F)cc2)o1]

To a solution of Compound 14 (130 mg, 0.30 mmol, 1 eq) in DCM (1 mL) was added TFA (1.02 g, 8.96 mmol, 0.7 mL, 30 eq). The solution was stirred at 25° C. for 0.5 h. The reaction mixture was concentrated under reduced pressure to give a residue. The residue was diluted with H₂O (10 mL) and extracted with EA (15 mL*3). The combined organic layers were washed with brine (15 mL), dried over Na₂SO₄, filtered and concentrated under reduced pressure to give a product (90 mg, crude) as brown oil. The residue (20 mg, crude) was purified by prep-HPLC (column: Welch Xtimate C18 150*25 mm*5 um; mobile phase: [water (0.05% HCl)-ACN]; B %: 18%-48%, 8.5 min) to give the title compound (2.6 mg, 6.81 umol, 2.8% yield, HCl) as a white solid. LCMS (ESI): RT=0.733 min, mass calc. for C₁₅H₁₂F₃N₅O 335.10, m/z found 335.9 [M+H]⁺; ¹H NMR (400 MHz, CD₃OD) δ 8.47 (dd, J=1.9, 4.9 Hz, 1H), 8.35 (dd, J=1.9, 7.9 Hz, 1H), 8.01 (d, J=8.5 Hz, 2H), 7.65 (d, J=8.8 Hz, 2H), 7.09 (dd, J=5.0, 7.8 Hz, 1H), 4.63 (s, 2H).

### Example 8: (R)-3-methyl-3-(5-(2-((4-(trifluoromethyl)phenyl)amino)pyridin-3-yl)-1,3,4-oxadiazol-2-yl)pyrrolidin-2-one (Compound 12), (S)-3-methyl-3-(5-(2-((4-(trifluoromethyl)phenyl)amino)pyridin-3-yl)-1,3,4-oxadiazol-2-yl)pyrrolidin-2-one (Compound 13), and 3-methyl-3-(5-(2-((4-(trifluoromethyl)phenyl)amino)pyridin-3-yl)-1,3,4-oxadiazol-2-yl)pyrrolidin-2-one (Compound 19)

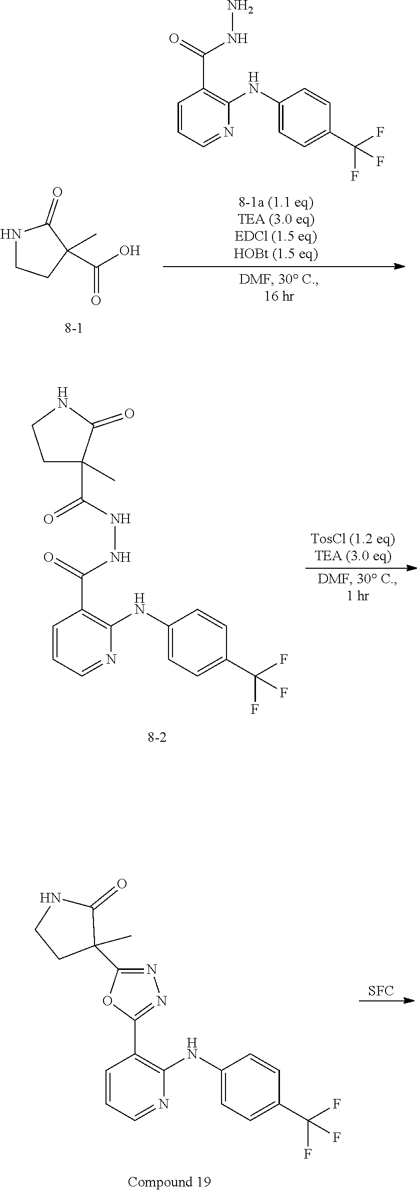

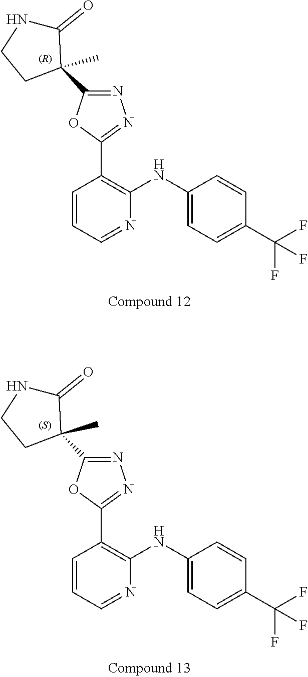

### N′-(3-methyl-2-oxopyrrolidine-3-carbonyl)-2-((4-(trifluoromethyl)phenyl)amino)nicotinohydrazide

To a solution of 8-1 (340.0 mg, 2.38 mmol, 1 eq), EDCI (683.0 mg, 3.56 mmol, 1.5 eq) and HOBt (481.4 mg, 3.56 mmol, 1.5 eq) in DMF (4 mL) at 30° C. were added 8-1a (774.0 mg, 2.6 mmol, 1.1 eq) and TEA (721.1 mg, 7.13 mmol, 1.0 mL, 3 eq). The mixture was stirred at 30° C. for 16 h. L The reaction mixture was concentrated under reduced pressure to give a residue. The residue was diluted with water (20 mL), Na₂CO₃ (20 mL) and extracted with EA (20 mL*3). The combined organic layers were washed with brine (20 mL), dried over Na₂SO₄, filtered and concentrated under reduced pressure to give compound 8-2 (990.0 mg, 1.8 mmol, 76.2% yield) as a yellow solid. LCMS (ESI): RT=0.773 min, mass calc. for C₁₉H₁₈F₃N₅O₃ 421.14, m/z found 422.0 [M+H]⁺

### 3-methyl-3-(5-(2-((4-(trifluoromethyl)phenyl)amino)pyridin-3-yl)-1,3,4-oxadiazol-2-yl)pyrrolidin-2-one (Compound 19)

To a solution of 8-2 (990.0 mg, 2.35 mmol, 1 eq) in DCM (10 mL) at 30° C. was added TEA (713.2 mg, 7.05 mmol, 1 mL, 3 eq) and TosCl (537.5 mg, 2.82 mmol, 1.2 eq). The mixture was stirred at 30° C. for 1 h. The reaction mixture was diluted with water (20 mL) and extracted with EA (20 mL*3). The combined organic layers were washed with brine (30 mL), dried over anhydrous Na2SO4, filtered and concentrated under reduced pressure to give a residue, which was purified by flash silica gel chromatography (ISCO®; 12 g SepaFlash® Silica Flash Column, Eluent of 0˜80% Ethyl acetate/Petroleum ether gradient @ 35 mL/min) to give Compound 19 (574.0 mg, 1.41 mmol, 59.9% yield) as a yellow solid. LCMS (ESI): RT=0.877 min, mass calc. for C₁₉H₁₆F₃N₅O₂ 403.13, m/z found 404.0 [M+H]⁺; ¹H NMR (400 MHz, CDCl₃) δ 10.32 (s, 1H), 8.43 (dd, J=1.9, 4.9 Hz, 1H), 8.20 (dd, J=2.0, 7.8 Hz, 1H), 7.94 (d, J=8.5 Hz, 2H), 7.60 (d, J=8.5 Hz, 2H), 6.92 (dd, J=5.0, 7.8 Hz, 1H), 6.18 (s, 1H), 3.70-3.63 (m, 1H), 3.54 (dt, J=5.4, 8.7 Hz, 1H), 3.05 (ddd, J=5.1, 7.9, 13.1 Hz, 1H), 2.36 (ddd, J=6.1, 7.7, 13.3 Hz, 1H), 1.80 (s, 3H).

### (R)-3-methyl-3-(5-(2-((4-(trifluoromethyl)phenyl)amino)pyridin-3-yl)-1,3,4-oxadiazol-2-yl)pyrrolidin-2-one (Compound 12) and (S)-3-methyl-3-(5-(2-((4-(trifluoromethyl)phenyl)amino)pyridin-3-yl)-1,3,4-oxadiazol-2-yl)pyrrolidin-2-one (Compound 13)

Compound 19 was separated by SFC: (column: DAICEL CHIRALPAK AS (250 mm*30 mm, 10 um); mobile phase: [0.1% NH₃H₂O ETOH]; B %: 25%-25%, min) to give Compound 12 (229.9 mg, 0.57 mmol, 32.8% yield) as a yellow solid and Compound 13 (254.7 mg, 0.62 mmol, 35.9% yield) as a yellow solid. Compound 12 LCMS (ESI): RT=0.919 min, mass calc. for C₁₉H₁₆F₃N₅O₂ 403.13, m/z found 404.2 [M+H]⁺; ¹H NMR (400 MHz, CDCl₃) δ 10.33 (s, 1H), 8.44 (dd, J=1.9, 4.9 Hz, 1H), 8.20 (dd, J=1.9, 7.7 Hz, 1H), 7.94 (d, J=8.5 Hz, 2H), 7.60 (d, J=8.5 Hz, 2H), 6.93 (dd, J=4.8, 7.8 Hz, 1H), 5.88 (brs, 1H), 3.72-3.65 (m, 1H), 3.55 (dt, J=5.1, 8.5 Hz, 1H), 3.06 (ddd, J=4.9, 7.9, 13.1 Hz, 1H), 2.41-2.33 (m, 1H), 1.80 (s, 3H). Compound 13 LCMS (ESI): RT=0.922 min, mass calc. for C₁₉H₁₆F₃N₅O₂ 403.13, m/z found 404.2 [M+H]⁺; ¹H NMR (400 MHz, CDCl₃) δ 10.33 (s, 1H), 8.44 (dd, J=1.8, 4.8 Hz, 1H), 8.20 (dd, J=1.8, 7.8 Hz, 1H), 7.94 (d, J=8.6 Hz, 2H), 7.60 (d, J=8.6 Hz, 2H), 6.93 (dd, J=4.9, 7.8 Hz, 1H), 5.88 (s, 1H), 3.72-3.65 (m, 1H), 3.55 (dt, J=5.1, 8.8 Hz, 1H), 3.06 (ddd, J=4.9, 7.9, 13.0 Hz, 1H), 2.41-2.33 (m, 1H), 1.80 (s, 3H).

### Example 9: tert-butyl N-[[5-[2-[4-(trifluoromethyl)anilino]-3-pyridyl]-1,3,4-oxadiazol-2-yl]methyl]carbamate (Compound 14)

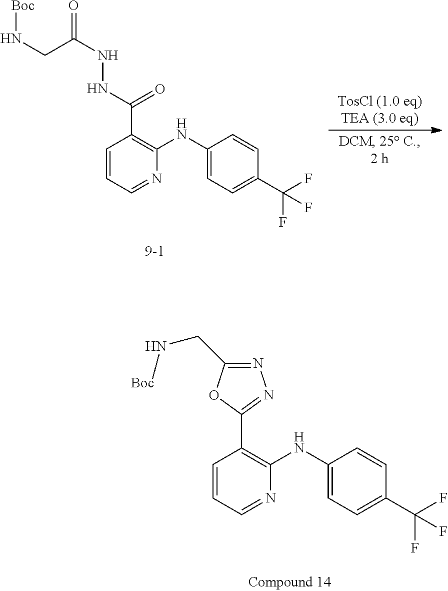 [SMILES: CC(C)(C)OC(=O)NCC(=O)NNC(=O)c1cccnc1Nc1ccc(C(F)(F)F)cc1 ; CC(C)(C)OC(=O)NCc1nnc(-c2cccnc2Nc2ccc(C(F)(F)F)cc2)o1]

To a solution of 9-1 (260.0 mg, 0.57 mmol, 1 eq) in DCM (2 mL) was added TosCl (109.3 mg, 0.57 mmol, 1.0 eq) and TEA (174.1 mg, 1.72 mmol, 0.2 mL, 3.0 eq). The mixture was stirred at 25° C. for 2 hr. The reaction mixture was concentrated under reduced pressure to give a residue. The residue was diluted with H₂O (10 mL) and extracted with EA (15 mL*3). The combined organic layers were washed with brine (15 mL), dried over Na₂SO₄, filtered and concentrated under reduced pressure to give a residue. The residue was purified by flash silica gel chromatography to give the desired compound (130.0 mg, 0.28 mmol, 48.4% yield) as a yellow solid. LCMS (ESI): RT=0.968 min, mass calc. for C₂₀H₂₀F₃N₅O₃ 435.15, m/z found 436.0 [M+H]⁺; ¹H NMR (400 MHz, CDCl₃) δ 10.29 (s, 1H), 8.44 (dd, J=2.0, 4.8 Hz, 1H), 8.15 (dd, J=1.9, 7.7 Hz, 1H), 7.95 (d, J=8.5 Hz, 2H), 7.61 (d, J=8.5 Hz, 2H), 6.93 (dd, J=4.8, 7.8 Hz, 1H), 5.25 (s, 1H), 5.19 (br s, 1H), 4.68 (br d, J=5.8 Hz, 2H), 1.50 (s, 8H).

### Example 10: ethyl-[2-[5-[2-[4-(trifluoromethyl)anilino]-3-pyridyl]-1,3,4-oxadiazol-2-yl]ethyl]cyanamide (Compound 15) and tert-butyl N-ethyl-N-[2-[5-[2-[4-(trifluoromethyl)anilino]-3-pyridyl]-1,3,4-oxadiazol-2-yl]ethyl]carbamate (Compound 17)

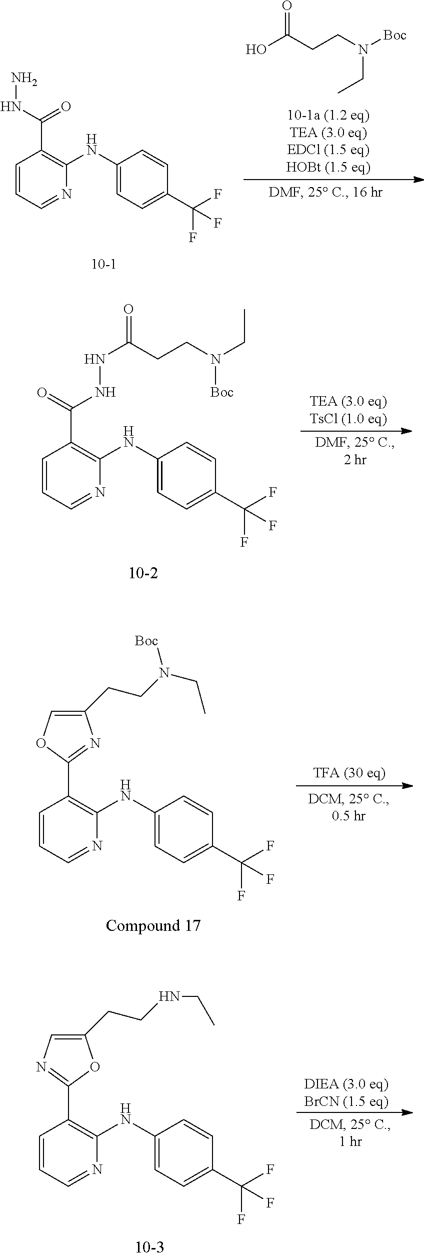 [SMILES: NNC(=O)c1cccnc1Nc1ccc(C(F)(F)F)cc1 ; CCN(CCC(=O)O)C(=O)OC(C)(C)C ; CCN(CCC(=O)NNC(=O)c1cccnc1Nc1ccc(C(F)(F)F)cc1)C(=O)OC(C)(C)C ; CCN(CCc1coc(-c2cccnc2Nc2ccc(C(F)(F)F)cc2)n1)C(=O)OC(C)(C)C ; CCNCCc1cnc(-c2cccnc2Nc2ccc(C(F)(F)F)cc2)o1]

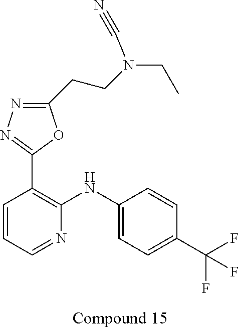 [SMILES: CCN(C#N)CCc1nnc(-c2cccnc2Nc2ccc(C(F)(F)F)cc2)o1]

### tert-butyl N-ethyl-N-[3-oxo-3-[2-[2-[4-(trifluoromethyl)anilino]pyridine-3-carbonyl]hydrazino]propyl]carbamate

To a solution of 10-1a (264.0 mg, 1.22 mmol, 1.2 eq) in DMF (3 mL) was added EDCI (291.2 mg, 1.52 mmol, 1.5 eq), HOBt (205.3 mg, 1.52 mmol, 1.5 eq), TEA (307.4 mg, 3.04 mmol, 0.4 mL, 3.0 eq) and 10-1 (300 mg, 1.01 mmol, 1 eq). The mixture was stirred at 25° C. for 16 hr. The residue was diluted with H₂O (10 mL) and extracted with EA (15 mL*3). The combined organic layers were washed with brine (15 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜80% Ethyl acetate/Petroleum ether gradient @ 30 mL/min) to give 10-2 (400 mg, 0.60 mmol, 58.9% yield) as a white solid. LCMS (ESI): RT=0.912 min, mass calc. for C₂₃H₂₈F₃N₅O₄ 495.21, m/z found 518.1 [M+Na]⁺.

### tert-butyl N-ethyl-N-[2-[5-[2-[4-(trifluoromethyl)anilino]-3-pyridyl]-1,3,4-oxadiazol-2-yl]ethyl]carbamate (Compound 17)

To a solution of 10-2 (100 mg, 0.20 mmol, 1 eq) in DCM (0.5 mL) at 0° C. was added TEA (61.3 mg, 0.61 mmol, 84 uL, 3.0 eq) and TosCl (38.5 mg, 0.20 mmol, 1 eq). The mixture was stirred at 25° C. for 2 h. The reaction mixture was concentrated under reduced pressure to give a residue. The residue was purified by flash silica gel chromatography to give Compound 17 (65 mg, 0.13 mmol, 66.7% yield) as a white solid. LCMS (ESI): RT=1.052 min, mass calc. for C₂₃H₂₆F₃N₅O₃ 477.20, m/z found 478.1 [M+H]⁺; ¹H NMR (400 MHz, CDCl₃) δ 10.34 (s, 1H), 8.43 (br d, J=3.4 Hz, 1H), 8.13 (br s, 1H), 7.95 (d, J=8.5 Hz, 2H), 7.60 (d, J=8.5 Hz, 2H), 6.92 (dd, J=4.8, 7.7 Hz, 1H), 3.75-3.63 (m, 2H), 3.40-3.17 (m, 4H), 1.43 (s, 9H), 1.14 (br t, J=6.3 Hz, 3H).

### 3-[5-[2-(ethylamino)ethyl]-1,3,4-oxadiazol-2-yl]-N-[4-(trifluoromethyl)phenyl]pyridin-2-amine

To a solution of Compound 17 (60 mg, 0.13 mmol, 1 eq) in DCM (0.5 mL) was added TFA (429.8 mg, 3.77 mmol, 0.3 mL, 30 eq). The solution was stirred at 25° C. for 0.5 hr. The reaction mixture was concentrated under reduced pressure to give compound 10-3 (40 mg, 0.11 mmol, 84.3% yield) as a white solid, which was used for the next step without further purification. ¹H NMR (400 MHz, CD₃OD) δ 8.46 (dd, J=1.7, 4.8 Hz, 1H), 8.32 (dd, J=1.8, 7.9 Hz, 1H), 8.01 (d, J=8.6 Hz, 2H), 7.63 (d, J=8.6 Hz, 2H), 7.06 (dd, J=4.9, 7.9 Hz, 1H), 3.66-3.56 (m, 2H), 3.51-3.44 (m, 2H), 2.83 (s, 3H).

### ethyl-[2-[5-[2-[4-(trifluoromethyl)anilino]-3-pyridyl]-1,3,4-oxadiazol-2-yl]ethyl]cyanamide (Compound 15)

To a solution of 10-3 (40 mg, 0.11 mmol, 1 eq) in DCM (0.5 mL) was added DIEA (41.1 mg, 0.32 mmol, 55 uL, 3.0 eq) and BrCN (16.8 mg, 0.16 mmol, 11 uL, 1.5 eq). The mixture was stirred at 25° C. for 1 hr. The solution was quenched by addition of H₂O at 0° C. and extracted with EA (10 mL*3). Then the combined organic layers were washed with brine (15 mL), dried over Na₂SO₄, filtered and concentrated under reduced pressure to give a residue. The residue was purified by prep-HPLC (column: Welch Xtimate C18 150*25 mm*5 um; mobile phase: [water (0.05% HCl)-ACN]; B %: 45%-75%, 8.5 min) to give Compound 15 (14.9 mg, 36.7 umol, 34.6% yield) as a white solid. LCMS (ESI): RT=0.927 min, mass calc. for C₁₉H₁₇F₃N₆O 402.14, m/z found 403.0 [M+H]⁺; ¹H NMR (400 MHz, CDCl₃) δ 10.28 (s, 1H), 8.45 (br d, J=3.8 Hz, 1H), 8.17 (br d, J=7.5 Hz, 1H), 7.95 (br d, J=8.5 Hz, 2H), 7.61 (d, J=8.3 Hz, 2H), 6.94 (dd, J=4.5, 7.5 Hz, 1H), 3.61 (t, J=6.8 Hz, 2H), 3.35 (t, J=6.8 Hz, 2H), 3.19 (q, J=7.1 Hz, 2H), 1.33 (t, J=7.2 Hz, 3H).

### Example 11: methyl-[2-[5-[2-[4-(trifluoromethyl)anilino]-3-pyridyl]-1,3,4-oxadiazol-2-yl]ethyl]cyanamide (Compound 16) and tert-butyl N-methyl-N-[2-[5-[2-[4-(trifluoromethyl)anilino]-3-pyridyl]-1,3,4-oxadiazol-2-yl]ethyl]carbamate (Compound 18)

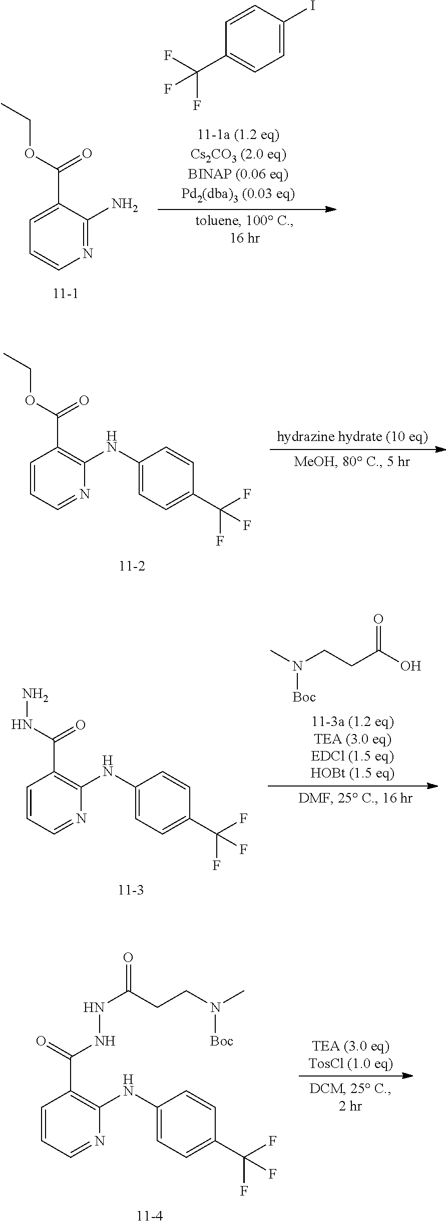 [SMILES: CCOC(=O)c1cccnc1N ; FC(F)(F)c1ccc(I)cc1 ; CCOC(=O)c1cccnc1Nc1ccc(C(F)(F)F)cc1 ; NNC(=O)c1cccnc1Nc1ccc(C(F)(F)F)cc1 ; CN(CCC(=O)O)C(=O)OC(C)(C)C ; CN(CCC(=O)NNC(=O)c1cccnc1Nc1ccc(C(F)(F)F)cc1)C(=O)OC(C)(C)C]

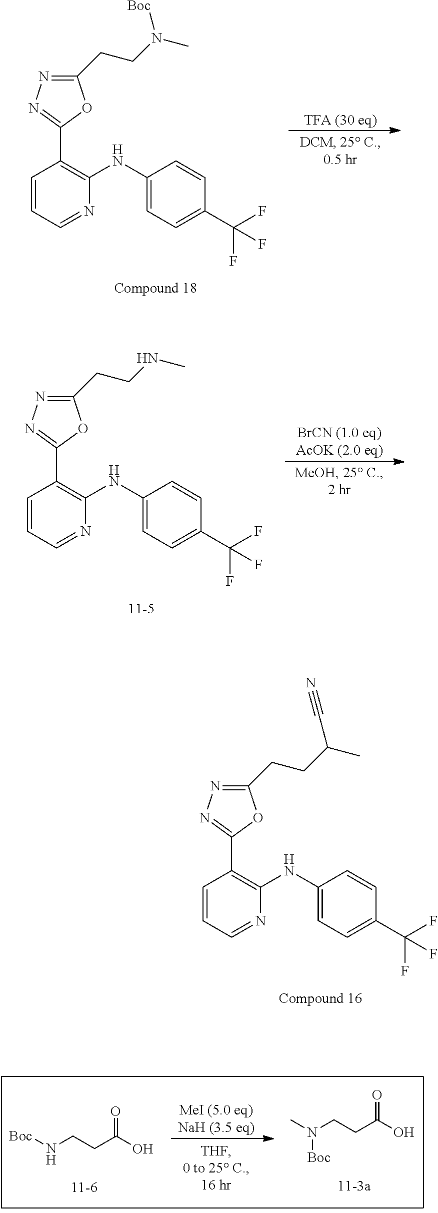 [SMILES: CN(CCc1nnc(-c2cccnc2Nc2ccc(C(F)(F)F)cc2)o1)C(=O)OC(C)(C)C ; CNCCc1nnc(-c2cccnc2Nc2ccc(C(F)(F)F)cc2)o1 ; CC(C#N)CCc1nnc(-c2cccnc2Nc2ccc(C(F)(F)F)cc2)o1 ; CC(C)(C)OC(=O)NCCC(=O)O ; CN(CCC(=O)O)C(=O)OC(C)(C)C]

### 2-[4-(trifluoromethyl)anilino]pyridine-3-carboxylate

To a solution of 11-1a (19.64 g, 72.21 mmol, 10.6 mL, 1.2 eq) in toluene (150 mL) was added 11-1 (10 g, 60.18 mmol, 1 eq), Cs₂CO₃ (39.21 g, 120.35 mmol, 2.0 eq), Pd(dba)₂ (1.04 g, 1.81 mmol, 0.03 eq) and BINAP (2.25 g, 3.61 mmol, 0.06 eq). The solution was stirred at 100° C. for 16 hr. The reaction mixture was concentrated under reduced pressure to give a residue. The residue was diluted with H₂O (30 mL) and extracted with EA (35 mL*3). The combined organic layers were washed with brine (45 mL), dried over Na₂SO₄, filtered and concentrated under reduced pressure to give a residue. The residue was purified by flash silica gel chromatography (ISCO®; 120 g SepaFlash® Silica Flash Column, Eluent of 100% Petroleum ether gradient @ 50 mL/min) to give compound 11-2 (7.4 g, 23.85 mmol, 39.6% yield) as a white solid. ¹H NMR (400 MHz, CDCl₃) δ 10.63-10.36 (m, 1H), 8.42 (dd, J=1.5, 4.6 Hz, 1H), 8.30 (dd, J=1.5, 7.6 Hz, 1H), 7.87 (d, J=8.5 Hz, 2H), 7.58 (d, J=8.5 Hz, 2H), 6.82 (dd, J=4.8, 7.8 Hz, 1H), 4.42 (q, J=7.1 Hz, 2H), 1.44 (t, J=7.1 Hz, 3H).

### 2-[4-(trifluoromethyl)anilino]pyridine-3-carbohydrazide

To a solution of 11-2 (6.4 g, 20.63 mmol, 1 eq) in MeOH (60 mL) was added NH₂—NH₂.H₂O (12.15 g, 206.27 mmol, 11.8 mL, 10 eq). The mixture was stirred at 80° C. for 5 hr. The reaction mixture was concentrated under reduced pressure to give a residue. The residue was diluted with H₂O (30 mL) and extracted with EA (45 mL*3). The combined organic layers were washed with brine (45 mL), dried over Na₂SO₄, filtered and concentrated under reduced pressure to give compound 11-3 (6.7 g, crude) as a yellow solid, which was used directly for next step. ¹H NMR (400 MHz, CDCl₃) δ 10.62-10.23 (m, 1H), 8.39 (dd, J=1.6, 4.8 Hz, 1H), 7.83 (d, J=8.4 Hz, 2H), 7.71 (dd, J=1.7, 7.7 Hz, 1H), 7.57 (d, J=8.5 Hz, 2H), 7.49 (br s, 1H), 6.79 (dd, J=4.9, 7.6 Hz, 1H), 4.38-3.63 (m, 2H).

### 3-[tert-butoxycarbonyl(methyl)amino]propanoic acid

To a solution of 11-6 (5 g, 26.43 mmol, 1 eq) in THF (50 mL) at 0° C. was added MeI (18.75 g, 132.13 mmol, 8.2 mL, 5.0 eq) and NaH (3.70 g, 92.49 mmol, 60%, 3.5 eq). The solution was stirred at 0° C. for 1 hr. The resulting mixture was stirred at 25° C. for 15 hr. The solution was quenched by H₂O at 0° C. and extracted with EA (60 mL*3). Then the combined organic layers were washed with brine (45 mL), dried by Na₂SO₄, filtered and concentrated under reduced pressure to give compound 11-3a (7 g, crude) as black brown oil, which was used for the next step without further purification. ¹H NMR (400 MHz, CDCl₃) δ 3.52 (br t, J=6.7 Hz, 2H), 2.94-2.83 (m, 3H), 2.60 (br s, 2H), 1.45 (s, 9H).

### tert-butyl N-methyl-N-[3-oxo-3-[2-[2-[4-(trifluoromethyl)anilino]pyridine-3-carbonyl]hydrazino]propyl]carbamate

To a solution of 11-3a (246.3 mg, 1.21 mmol, 1.2 eq) in DMF (3 mL) was added EDCI (290.4 mg, 1.52 mmol, 1.5 eq), HOBt (204.7 mg, 1.52 mmol, 1.5 eq), TEA (306.6 mg, 3.03 mmol, 0.4 mL, 3.0 eq) and 11-3 (300 mg, 1.01 mmol, 1 eq). The mixture was stirred at 25° C. for 16 hr. The residue was diluted with H₂O (10 mL) and extracted with EA (15 mL*3). The combined organic layers were washed with brine (15 mL), dried over Na₂SO₄, filtered and concentrated under reduced pressure to give a residue. The residue was purified by flash silica gel chromatography (ISCO®; 4 g SepaFlash® Silica Flash Column, Eluent of 0˜100% Ethyl acetate/Petroleum ether gradient @ 30 mL/min) to give compound 11-4 (400 mg, 0.80 mmol, 78.9% yield) as a white solid. LCMS (ESI): RT=0.881 min, mass calc. for C₂₂H₂₆F₃N₅O₄ 481.19, m/z found 482.1 [M+H]⁺.

### tert-butyl N-methyl-N-[2-[5-[2-[4-(trifluoromethyl)anilino]-3-pyridyl]-1,3,4-oxadiazol-2-yl]ethyl]carbamate (Compound 18)

To a solution of 11-4 (150 mg, 0.31 mmol, 1 eq) in DCM (0.5 mL) at 0° C. was added TEA (94.6 mg, 0.93 mmol, 0.1 mL, 3.0 eq) and TosCl (59.4 mg, 0.31 mmol, 1 eq). The mixture was stirred at 25° C. for 2 h. The reaction mixture was concentrated under reduced pressure to give a residue. The residue was purified by flash silica gel chromatography (ISCO®; 4 g SepaFlash® Silica Flash Column, Eluent of 0˜20% Ethyl acetate/Petroleum ether gradient @30 mL/min) to give Compound 18 (70 mg, 0.14 mmol, 47.5% yield) as a white solid. LCMS (ESI): RT=1.011 min, mass calc. for C₂₂H₂₄F₃N₅O₃ 463.18, m/z found 464.1 [M+H]⁺; ¹H NMR (400 MHz, CDCl₃) δ 10.34 (br s, 1H), 8.43 (br d, J=3.5 Hz, 1H), 8.15 (br s, 1H), 7.95 (d, J=8.5 Hz, 2H), 7.61 (d, J=8.6 Hz, 2H), 6.93 (dd, J=4.9, 7.8 Hz, 1H), 3.76-3.72 (m, 2H), 3.22 (br s, 2H), 2.94 (br s, 3H), 1.40 (br s, 9H).

### 3-[5-[2-(methylamino)ethyl]-1,3,4-oxadiazol-2-yl]-N-[4-(trifluoromethyl)phenyl]pyridin-2-amine

To a solution of Compound 18 (60.0 mg, 0.13 mmol, 1 eq) in DCM (0.5 mL) was added TFA (442.8 mg, 3.88 mmol, 0.3 mL, 30 eq). The solution was stirred at 25° C. for 0.5 hr. The reaction mixture was concentrated under reduced pressure to give compound 11-5 (40.0 mg, 0.11 mol, 85% yield) as a white solid. ¹H NMR (400 MHz, CD₃OD) δ 8.50-8.37 (m, 1H), 8.32 (dd, J=1.8, 7.9 Hz, 1H), 8.01 (d, J=8.6 Hz, 2H), 7.63 (d, J=8.6 Hz, 2H), 7.06 (dd, J=4.9, 7.9 Hz, 1H), 3.66-3.57 (m, 2H), 3.54-3.45 (m, 2H), 2.88-2.81 (m, 3H).

### methyl-[2-[5-[2-[4-(trifluoromethyl)anilino]-3-pyridyl]-1,3,4-oxadiazol-2-yl]ethyl]cyanamide (Compound 16)

To a solution of 11-5 (40.0 mg, 0.11 mmol, 1 eq) in DCM (0.5 mL) was added DIEA (42.7 mg, 0.33 mmol, 57 uL, 3.0 eq) and BrCN (17.5 mg, 0.17 mmol, 12 uL, 1.5 eq). The mixture was stirred at 25° C. for 1 hr. The solution was quenched by addition of H₂O at 0° C. and extracted with EA (10 mL*3). Then the combined organic layers were washed with brine (15 mL), dried by Na₂SO₄, filtered and concentrated under reduced pressure to give a residue. The residue was purified by prep-HPLC (column: Welch Xtimate C18 150*25 mm*5 um; mobile phase: [water (0.05% HCl)-ACN]; B %: 45%-75%, 8.5 min) to give Compound 16 (21.4 mg, 53.5 umol, 48.6% yield) as a white solid. LCMS (ESI): RT=0.909 min, mass calc. for C₁₈H₁₅F₃N₆O 388.13, m/z found 388.9 [M+H]⁺; ¹H NMR (400 MHz, CDCl₃) δ 10.28 (br s, 1H), 8.45 (br d, J=3.3 Hz, 1H), 8.17 (br d, J=7.3 Hz, 1H), 7.95 (br d, J=8.3 Hz, 2H), 7.61 (br d, J=8.3 Hz, 2H), 6.94 (br dd, J=4.8, 7.3 Hz, 1H), 3.60 (t, J=6.8 Hz, 2H), 3.35 (t, J=6.8 Hz, 2H), 3.00 (s, 3H).

### Example 12: tert-butyl N-ethyl-N-[2-[5-[2-[4-(trifluoromethyl)anilino]-3-pyridyl]-1,3,4-oxadiazol-2-yl]ethyl]carbamate (Compound 17)

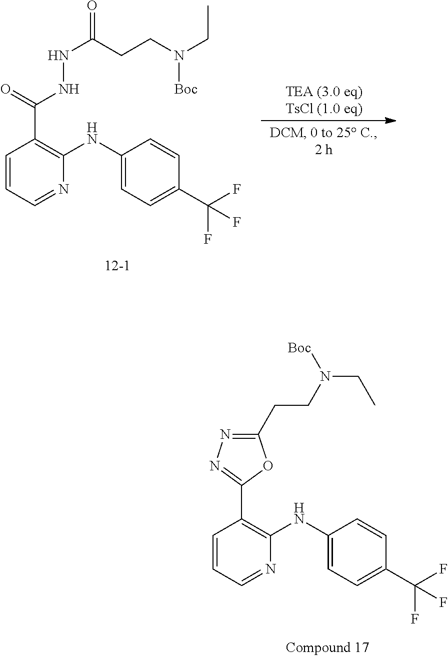 [SMILES: CCN(CCC(=O)NNC(=O)c1cccnc1Nc1ccc(C(F)(F)F)cc1)C(=O)OC(C)(C)C ; CCN(CCc1nnc(-c2cccnc2Nc2ccc(C(F)(F)F)cc2)o1)C(=O)OC(C)(C)C]

To a solution of 12-1 (100 mg, 0.20 mmol, 1 eq) in DCM (0.5 mL) at 0° C. was added TEA (61.3 mg, 0.61 mmol, 84 uL, 3.0 eq) and TosCl (38.5 mg, 0.20 mmol, 1 eq). The mixture was stirred at 25° C. for 2 h. LCMS showed that 60% of desired product was detected. The reaction mixture was concentrated under reduced pressure to give a residue. The residue was purified by flash silica gel chromatography (ISCO®; 4 g SepaFlash® Silica Flash Column, Eluent of 0˜20% Ethyl acetate/Petroleum ether gradient @ 30 mL/min) to give the desired compound (65 mg, 0.13 mmol, 66.7% yield) as a white solid. LCMS (ESI): RT=1.052 min, mass calc. for C₂₃H₂₆F₃N₅O₃ 477.20, m/z found 478.1 [M+H]⁺; ¹H NMR (400 MHz, CDCl₃) δ 10.34 (s, 1H), 8.43 (br d, J=3.4 Hz, 1H), 8.13 (br s, 1H), 7.95 (d, J=8.5 Hz, 2H), 7.60 (d, J=8.5 Hz, 2H), 6.92 (dd, J=4.8, 7.7 Hz, 1H), 3.75-3.63 (m, 2H), 3.40-3.17 (m, 4H), 1.43 (s, 9H), 1.14 (br t, J=6.3 Hz, 3H).

### Example 13: 3-Methyl-3-{5-[3-(4-trifluoromethyl-phenylamino)-pyrazin-2-yl]-[1,3,4]oxadiazol-2-yl}-pyrrolidin-2-one (Compound 20)

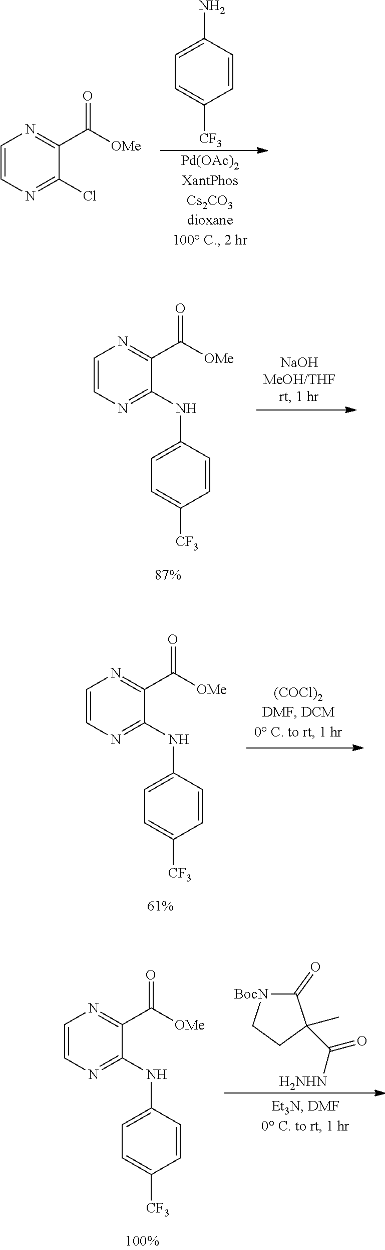

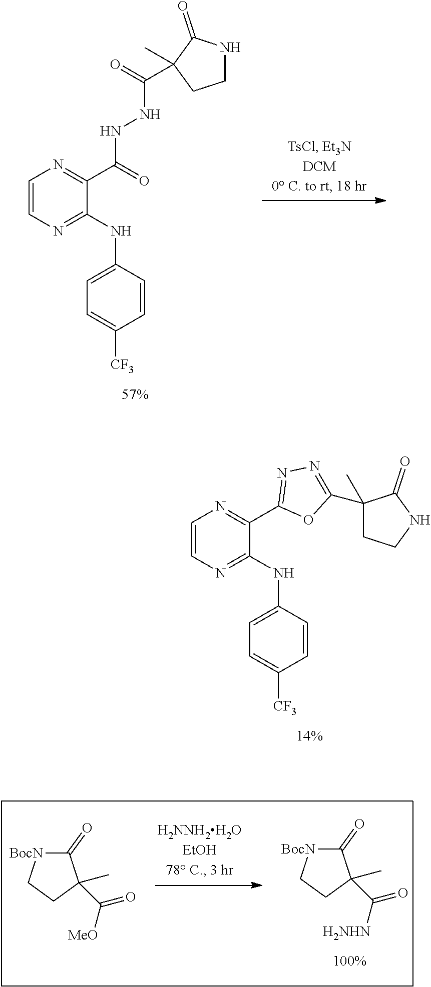

### tert-Butyl 3-(hydrazinecarbonyl)-3-methyl-2-oxopyrrolidine-1-carboxylate

To a suspension of 1-(tert-butyl) 3-methyl 3-methyl-2-oxopyrrolidine-1,3-dicarboxylate (prepared according to WO2016138532, 1 equiv.) in EtOH (0.25M) was added hydrazine monohydrate (5 equiv.). The reaction mixture was stirred at reflux for 3 hr. After cooling to rt, the mixture was concentrated, and the residue was further dried overnight to afford the product, 100% yield.

### 3-(4-Trifluoromethyl-phenylamino)-pyrazine-2-carboxylic acid methyl ester

To a thoroughly N₂ purged solution of Methyl 3-chloro-pyrazine-2-carboxylic acid methyl ester (1 equiv.), 4-(trifluoromethyl)aniline (1.05 equiv.), XantPhos (0.05 equiv.), Cs₂CO₃ (2 equiv.) in dioxane (0.2M) was added Pd(OAc)₂ (0.05 equiv.). The reaction mixture was heated to 100° C. for 2 hr. The mixture was cooled and carefully added to rapidly stirring H₂O. The suspension was allowed to stir an additional 30 min at room temperature, whereupon the solid was filtered and rinsed with H₂O. The solid was dissolved in DCM, and passed over a short silica plug, eluting with DCM, and concentrated to give 3-(4-Trifluoromethyl-phenylamino)-pyrazine-2-carboxylic acid methyl ester, 87% yield. LCMS [M+H]⁺=298.

### 3-(4-Trifluoromethyl-phenylamino)-pyrazine-2-carboxylic acid

3-(4-Trifluoromethyl-phenylamino)-pyrazine-2-carboxylic acid methyl ester was dissolved in MeOH/THF (1:1, 0.1 M). 2 M sodium hydroxide solution (0.05 M) was added dropwise, and the reaction mixture was stirred at rt for 1 hr. After completion (monitored by LCMS), the reaction mixture was neutralized with 1M HCl solution, the precipitates were filtered and washed with small amount of water. The crude compound was used without further purification for the next step, 61% yield. LCMS [M+H]⁺=284.

### 3-(4-Trifluoromethyl-phenylamino)-pyrazine-2-carbonyl chloride

3-(4-Trifluoromethyl-phenylamino)-pyrazine-2-carboxylic acid was dissolved in DCM (0.05 M), and the reaction mixture was cooled to 0° C. under nitrogen protection. Oxalyl chloride (2 equiv.) was added dropwise to the reaction mixture followed by 1 drop of DMF. The reaction was slowly warmed to rt and stirred for 1 hr. After completion of the reaction (monitored by LCMS), solvents were removed under reduced pressure. The crude material was re-dissolved in toluene and evaporated under reduced pressure 3 times to afford the acid chloride, which was used without purification for the next step, 100% yield.

### 3-Methyl-2-oxo-pyrrolidine-3-carboxylic acid N′-[3-(4-trifluoromethyl-phenylamino)-pyrazine-2-carbonyl]-hydrazide

3-(4-Trifluoromethyl-phenylamino)-pyrazine-2-carbonyl chloride was dissolved in DMF (0.05 M) and cooled to 0° C. under nitrogen protection. To the reaction mixture, the hydrazide solution (1.1 equiv., pre-dissolved in DMF) and triethylamine (3 equiv.) were added dropwise at 0° C. The reaction mixture was slowly warmed to rt and stirred for 1 hr. After completion (monitored by LCMS), the reaction mixture was diluted with EtOAc and washed 2× brine and water. The organic layer was separated and dried over Na₂SO₄, filtered, and concentrated under reduced pressure. The crude was purified by silica gel column chromatography (1-10% MeOH/DCM, gradient elution) to afford desired compound, 57% yield (no Boc-protected product was observed in this reaction). LCMS [M+H]⁺=423.

### 3-Methyl-3-{5-[3-(4-trifluoromethyl-phenylamino)-pyrazin-2-yl]-[1,3,4]oxadiazol-2-yl}-pyrrolidin-2-one

To a solution of 3-Methyl-2-oxo-pyrrolidine-3-carboxylic acid N′-[3-(4-trifluoromethyl-phenylamino)-pyrazine-2-carbonyl]-hydrazide in DCM (0.1 M) was added triethylamine (3 equiv.) and TsCl (1 equiv.) at 0° C. The reaction mixture was slowly warmed to rt and stirred overnight. After completion (monitored by LCMS), solvents were removed. The crude material was then re-dissolved in EtOAc and washed with brine. The organic layer was dried over Na₂SO₄, filtered, and concentrated under reduced pressure. The crude was purified by silica gel column chromatography (1-10% MeOH/DCM, gradient elution) to afford the desired compound, 14% yield. LCMS [M+H]⁺=405.

### Example 14: (3S)-3-ethyl-3-[5-[2-[4-(pentafluoro-λ⁶-sulfanyl)anilino]-3-pyridyl]-1,3,4-oxadiazol-2-yl]piperidin-2-one (Compound 21) and (3R)-3-ethyl-3-[5-[2-[4-(pentafluoro-λ⁶-sulfanyl)anilino]-3-pyridyl]-1,3,4-oxadiazol-2-yl]piperidin-2-one (Compound 22)

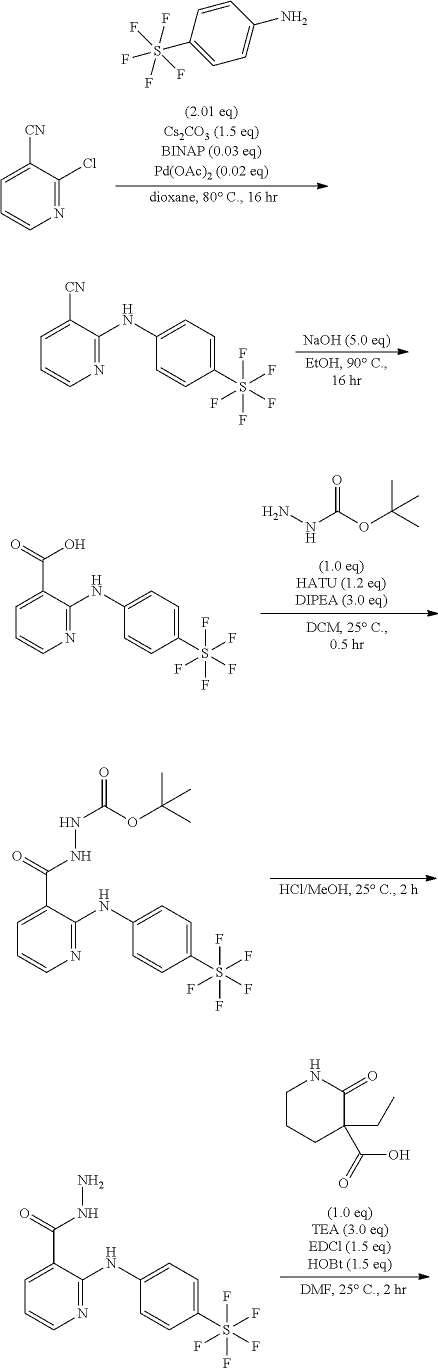

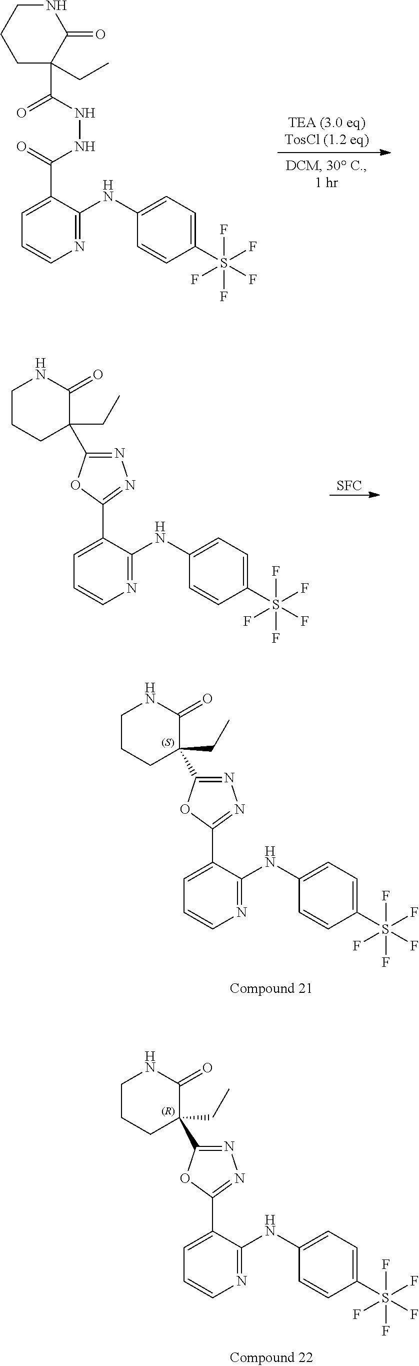

### 2-[4-(Pentafluoro-6-sulfanyl)anilino]pyridine-3-carbonitrile

To a solution of 2-chloropyridine-3-carbonitrile (1.5 g, 10.83 mmol, 1 eq) and 4-(pentafluoro-λ⁶-sulfanyl)aniline (4.7 g, 21.76 mmol, 2.01 eq) in dioxane (15 mL) was added Pd(OAc)₂ (48.6 mg, 0.21 mmol, 0.02 eq) and BINAP (202.2 mg, 0.32 mmol, 0.03 eq) and Cs₂CO₃ (5.2 g, 16.24 mmol, 1.5 eq). The reaction was stirred at 80° C. for 16 hr. The reaction mixture was filtered and the filtrate was concentrated under reduce pressure. The crude product was purified by re-crystallization from PE/EA (6:1) to give 2-[4-(pentafluoro-λ⁶-sulfanyl)anilino]pyridine-3-carbonitrile (2.6 g, 6.65 mmol, 61.4% yield).

### 2-[4-(Pentafluoro-λ6-sulfanyl)anilino]pyridine-3-carboxylic acid

A solution of 2-[4-(pentafluoro-λ⁶-sulfanyl)anilino]pyridine-3-carbonitrile (1 g, 3.11 mmol, 1 eq) in EtOH (20 mL) was added NaOH (4 M, 3.89 mL, 5 eq). The reaction was stirred at 90° C. for 16 hr. The reaction mixture was concentrated under reduce pressure. The reaction mixture was adjusted PH=3-4 with HCl, extracted with EA (25 mL*3). The combined organic layers were dried over Na₂SO₄, concentrated to dryness under reduced pressure. Compound 2-[4-(pentafluoro-λ⁶-sulfanyl)anilino]pyridine-3-carboxylic acid (840 mg, 2.47 mmol, 79.3% yield) was obtained as a yellow solid, which was used into the next step without the further purification.

### Tert-butyl N-[[2-[4-(pentafluoro-λ6-sulfanyl)anilino]pyridine-3-carbonyl]amino]carbamate

To a solution of 2-[4-(pentafluoro-λ⁶-sulfanyl)anilino]pyridine-3-carboxylic acid (750 mg, 2.20 mmol, 1 eq) and tert-butyl N-aminocarbamate (291.3 mg, 2.20 mmol, 1 eq) in DCM (8 mL) was added HATU (1.01 g, 2.64 mmol, 1.2 eq), DIPEA (854.6 mg, 6.61 mmol, 1.15 mL, 3 eq). The reaction was stirred at 25° C. for 0.5 hr. The mixture was diluted with water (15 mL) and the resultant mixture was extracted with DCM (20 mL*3). The combined organic layers were dried over Na₂SO₄, filtered and concentrated to dryness under reduced pressure. The residue was purified by flash silica gel chromatography to give the title compound as a yellow solid. Compound tert-butyl N-[[2-[4-(pentafluoro-λ⁶-sulfanyl)anilino]pyridine-3-carbonyl]amino]carbamate (490 mg, 0.73 mmol, 33.2% yield) was obtained as a yellow solid.

### 2-[4-(Pentafluoro-λ6-sulfanyl)anilino]pyridine-3-carbohydrazide

A solution of tert-butyl N-[[2-[4-(pentafluoro-λ⁶-sulfanyl)anilino]pyridine-3-carbonyl]amino]carbamate (500 mg, 1.10 mmol, 1 eq) in HCl/MeOH (10 mL) was stirred at 25° C. for 2 hr. Compound 2-[4-(pentafluoro-λ⁶-sulfanyl)anilino]pyridine-3-carbohydrazide (350 mg, crude) was obtained as a white solid, which was used into the next step without the further purification.

### N′-(3-ethyl-2-oxo-piperidine-3-carbonyl)-2-[4-(pentafluoro-λ6-sulfanyl)anilino]pyridine-3-carbohydrazide

To a solution of 3-ethyl-2-oxo-piperidine-3-carboxylic acid (80 mg, 0.46 mmol, 1 eq) and 2-[4-(pentafluoro-λ⁶-sulfanyl)anilino]pyridine-3-carbohydrazide (165.5 mg, 0.46 mmol, 1 eq) in DMF (2 mL) was added TEA (141.8 mg, 1.40 mmol, 0.19 mL, 3 eq), HOBt (94.7 mg, 0.70 mmol, 1.5 eq) and EDCI (134.3 mg, 0.70 mmol, 1.5 eq). The reaction was stirred at 25° C. for 2 hr. The residue was purified by flash silica gel chromatography to give the desired compound N′-(3-ethyl-2-oxo-piperidine-3-carbonyl)-2-[4-(pentafluoro-λ⁶-sulfanyl)anilino]pyridine-3-carbohydrazide (205 mg, 0.40 mmol, 86.4% yield) as a yellow oil.

### 3-Ethyl-3-[5-[2-[4-(pentafluoro-λ⁶-sulfanyl)anilino]-3-pyridyl]-1,3,4-oxadiazol-2-yl]piperidin-2-one

To a solution of N′-(3-ethyl-2-oxo-piperidine-3-carbonyl)-2-[4-(pentafluoro-λ⁶-sulfanyl)anilino]pyridine-3-carbohydrazide (430 mg, 0.94 mmol, 1 eq) in DCM (6 mL) was added TEA (257.2 mg, 2.54 mmol, 0.35 mL, 3 eq) and TosCl (193.8 mg, 1.02 mmol, 1.2 eq). The reaction was stirred at 25° C. for 1 hr. The mixture was diluted with water (15 mL) and the resultant mixture was extracted with DCM (20 mL*3). The combined organic layers were dried over Na₂SO₄, filtered and concentrated to dryness under reduced pressure. The residue was purified by flash silica gel chromatography to give the title compound as a colorless oil. Compound 3-ethyl-3-[5-[2-[4-(pentafluoro-λ⁶-sulfanyl)anilino]-3-pyridyl]-1,3,4-oxadiazol-2-yl]piperidin-2-one (125 mg, 0.24 mmol, 28.6% yield) was obtained as a colorless oil.

### (3S)-3-ethyl-3-[5-[2-[4-(pentafluoro-λ6-sulfanyl)anilino]-3-pyridyl]-1,3,4-oxadiazol-2-yl]piperidin-2-one (Compound 21) and (3R)-3-ethyl-3-[5-[2-[4-(pentafluoro-λ⁶-sulfanyl)anilino]-3-pyridyl]-1,3,4-oxadiazol-2-yl]piperidin-2-one (Compound 22)

The 3-ethyl-3-[5-[2-[4-(pentafluoro-λ⁶-sulfanyl)anilino]-3-pyridyl]-1,3,4-oxadiazol-2-yl]piperidin-2-one (85 mg, 0.17 mmol, 1 eq) was separated by SFC (column: DAICEL CHIRALCEL OD-H (250 mm*30 mm, 5 um); mobile phase: [0.1% NH₃H₂O MEOH]; B %: 50%-50%, min) to give (3S)-3-ethyl-3-[5-[2-[4-(pentafluoro-λ⁶-sulfanyl)anilino]-3-pyridyl]-1,3,4-oxadiazol-2-yl]piperidin-2-one (Compound 21) (15.9 mg, 31.7 umol, 18.2% yield) as a white solid and (3R)-3-ethyl-3-[5-[2-[4-(pentafluoro-λ⁶-sulfanyl)anilino]-3-pyridyl]-1,3,4-oxadiazol-2-yl]piperidin-2-one (Compound 22) (38 mg) as a white solid. Compound 22 was further purified by prep-HPLC (column: Welch Xtimate C18 150*25 mm*5 um; mobile phase: [water (0.05% NH₃H₂O)-ACN]; B %: 56%-86%, 7.8 min) to give the desired compound (4.2 mg, 8.6 umol, 5.0% yield) as a white solid. Compound 21: LCMS (ESI): RT=0.996 min, mass calcd for C₂₀H₂₀F₅N₅O₂S 489.46 m/z, found 490.1 [M+H]⁺, ¹H NMR (400 MHz, DMSO-d₆) δ 10.29 (s, 1H), 8.50 (dd, J=1.8, 4.8 Hz, 1H), 8.28 (dd, J=1.9, 7.8 Hz, 1H), 8.03-7.93 (m, 3H), 7.87 (d, J=9.3 Hz, 2H), 7.16 (dd, J=4.9, 7.8 Hz, 1H), 3.28-3.23 (m, 2H), 2.37-2.30 (m, 1H), 2.23-2.09 (m, 2H), 2.09-2.00 (m, 1H), 1.96-1.78 (m, 2H), 0.94 (t, J=7.4 Hz, 3H). Compound 22: LCMS (ESI): RT=1.000 min, mass calcd for C₂₀H₂₀F₅N₅O₂S 490.1 m/z, found 489.46 [M+H]⁺, ¹H NMR (400 MHz, DMSO-d₆) δ 10.30 (s, 1H), 8.50 (dd, J=1.8, 4.8 Hz, 1H), 8.28 (dd, J=1.9, 7.8 Hz, 1H), 8.04-7.91 (m, 3H), 7.87 (d, J=9.3 Hz, 2H), 7.16 (dd, J=4.9, 7.8 Hz, 1H), 3.25 (br s, 2H), 2.35-2.30 (m, 1H), 2.25-2.11 (m, 2H), 2.09-2.02 (m, 1H), 1.96-1.77 (m, 2H), 0.94 (t, J=7.4 Hz, 3H).

### Example 15: (3R)-3-ethyl-3-[5-[2-[4-(pentafluoro-λ⁶-sulfanyl)anilino]-3-pyridyl]-1,3,4-oxadiazol-2-yl]pyrrolidin-2-one (Compound 23) and (3S)-3-ethyl-3-[5-[2-[4-(pentafluoro-λ⁶-sulfanyl)anilino]-3-pyridyl]-1,3,4-oxadiazol-2-yl]pyrrolidin-2-one (Compound 24)

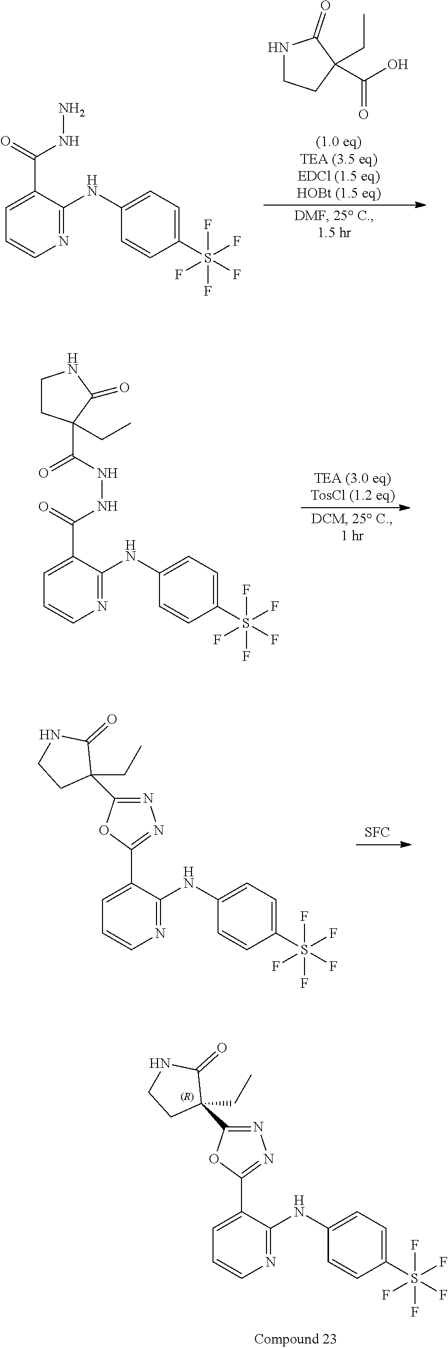

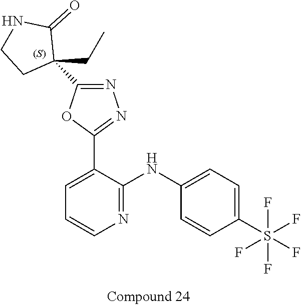

### N′-(3-ethyl-2-oxo-pyrrolidine-3-carbonyl)-2-[4-(pentafluoro-λ⁶-sulfanyl)anilino]pyridine-3-carbohydrazide

To a solution of 3-ethyl-2-oxopyrrolidine-3-carboxylic acid (88.7 mg, 0.56 mmol, 1 eq) and 2-((4-(pentafluoro-λ6-sulfanyl)phenyl)amino)nicotinohydrazide (200 mg, 0.56 mmol, 1.0 eq) in DMF (6 mL) was added HOBt (114.4 mg, 0.84 mmol, 1.5 eq), TEA (199.9 mg, 1.98 mmol, 0.27 mL, 3.5 eq) and EDCI (162.3 mg, 0.84 mol, 1.5 eq). The mixture was stirred at 25° C. for 1.5 hr. The reaction mixture was filtered and concentrated under reduced pressure to give a residue. The residue was purified by column chromatography to give N′-(3-ethyl-2-oxo-pyrrolidine-3-carbonyl)-2-[4-(pentafluoro-λ⁶-sulfanyl)anilino]pyridine-3-carbohydrazide (250 mg, 0.50 mmol, 89% yield) as a white solid.

### 3-Ethyl-3-[5-[2-[4-(pentafluoro-λ⁶-sulfanyl)anilino]-3-pyridyl]-1,3,4-oxadiazol-2-yl]pyrrolidin-2-one

To a solution of N′-(3-ethyl-2-oxo-pyrrolidine-3-carbonyl)-2-[4-(pentafluoro-λ⁶-sulfanyl)anilino]pyridine-3-carbohydrazide (250 mg, 0.50 mmol, 1 eq) in DCM (1 mL) was added TosCl (115.9 mg, 0.60 mmol, 1.2 eq) and TEA (153.8 mg, 1.52 mmol, 0.21 mL, 3.0 eq). The mixture was stirred at 25° C. for 1 hr. The reaction mixture was concentrated under reduced pressure to give a residue. The residue was purified by column chromatography to give 3-Ethyl-3-[5-[2-[4-(pentafluoro-λ⁶-sulfanyl)anilino]-3-pyridyl]-1,3,4-oxadiazol-2-yl]pyrrolidin-2-one (90 mg, 0.18 mmol, 36% yield) as a white solid.

### (3R)-3-ethyl-3-[5-[2-[4-(pentafluoro-λ⁶-sulfanyl)anilino]-3-pyridyl]-1,3,4-oxadiazol-2-yl]pyrrolidin-2-one (Compound 23) and (3S)-3-ethyl-3-[5-[2-[4-(pentafluoro-λ⁶-sulfanyl)anilino]-3-pyridyl]-1,3,4-oxadiazol-2-yl]pyrrolidin-2-one (Compound 24)

3-Ethyl-3-[5-[2-[4-(pentafluoro-λ⁶-sulfanyl)anilino]-3-pyridyl]-1,3,4-oxadiazol-2-yl]pyrrolidin-2-one (90 mg, 0.18 mmol, 1 eq) (90 mg) was separated by prep-SFC (column: DAICEL CHIRALCEL OD (250 mm*30 mm, 10 um); mobile phase: [0.1% NH₃H₂O ETOH]; B %: 50%-50%, min) to give Compound 23 (13.3 mg, 28.1 umol, 14% yield) and Compound 24 (20.7 mg, 42.8 umol, 22% yield). Compound 23: LCMS (ESI): RT=0.981 min, mass calcd for: C₁₉H₁₈F₅N₅O₂S 475.11 m/z found 476.1 [M+H]⁺; ¹H NMR (400 MHz, DMSO-d₆) δ 10.28 (s, 1H), 8.51 (dd, J=1.8, 4.8 Hz, 1H), 8.30 (dd, J=1.8, 7.8 Hz, 1H), 8.22 (s, 1H), 8.00 (br d, J=9.0 Hz, 2H), 7.88 (d, J=9.3 Hz, 2H), 7.17 (dd, J=4.9, 7.8 Hz, 1H), 3.50-3.36 (m, 2H), 2.38-2.31 (m, 1H), 2.25-2.14 (m, 1H), 1.98 (qd, J=7.3, 14.1 Hz, 1H), 0.96 (t, J=7.4 Hz, 3H). Compound 24: LCMS (ESI): RT=0.985 min, mass calcd for: C₁₉H₁₈F₅N₅O₂S 475.11 m/z found 476.1 [M+H]⁺; ¹H NMR (400 MHz, DMSO-d₆) δ 10.28 (s, 1H), 8.51 (dd, J=1.8, 4.8 Hz, 1H), 8.30 (dd, J=1.8, 7.8 Hz, 1H), 8.22 (s, 1H), 8.04-7.95 (m, 2H), 7.88 (d, J=9.3 Hz, 2H), 7.17 (dd, J=4.9, 7.8 Hz, 1H), 3.51-3.37 (m, 2H), 2.78 (ddd, J=4.8, 8.1, 13.1 Hz, 1H), 2.40-2.31 (m, 1H), 2.19 (qd, J=7.2, 14.0 Hz, 1H), 1.98 (qd, J=7.2, 14.1 Hz, 1H), 0.96 (t, J=7.4 Hz, 3H).

### Example 16: 3,4-dimethyl-3-[5-[2-[4-(trifluoromethyl)anilino]-3-pyridyl]-1,3,4-oxadiazol-2-yl]piperazin-2-one (Compound 25)

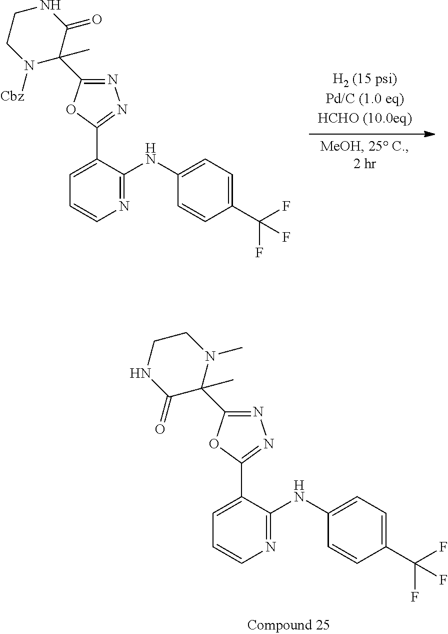 [SMILES: CC1(c2nnc(-c3cccnc3Nc3ccc(C(F)(F)F)cc3)o2)C(=O)NCCN1C(=O)OCc1ccccc1 ; CN1CCNC(=O)C1(C)c1nnc(-c2cccnc2Nc2ccc(C(F)(F)F)cc2)o1]

To a solution of benzyl 2-methyl-3-oxo-2-(5-(2-((4-(trifluoromethyl)phenyl)amino)pyridin-3-yl)-1,3,4-oxadiazol-2-yl)piperazine-1-carboxylate (70.0 mg, 0.13 mmol, 1.0 eq) in MeOH (0.5 mL) was added HCHO (38.0 mg, 1.27 mmol, 34 uL, 10.0 eq) and Pd/C (10.0 mg, 0.13 mmol, 10%, 1.0 eq). The mixture was degassed and purged with H₂ for three times. The mixture was stirred at 25° C. for 2 hr under H₂ (15 psi) atmosphere. The solution was filtered and concentrated under reduced pressure to give a residue. The residue was purified by prep-HPLC (column: 3_Phenomenex Luna C18 75*30 mm*3 um; mobile phase: [water (0.05% HCl)-ACN]; B %: 40%-70%, 6.5 min) to give the title compound (2.5 mg, 5.8 umol, 4.6% yield) as a white solid. LCMS (ESI): RT=0.848 min, mass calc. for C₂₀H₁₉F₃N₆O₂ 432.15, m/z found 433.0 [M+1]+; 1H NMR (400 MHz, CD₃OD) δ 2.16 (s, 3H) 2.76 (s, 3H) 3.46-3.55 (m, 1H) 3.56-3.63 (m, 1H) 3.67 (brd, J=4.27 Hz, 2H) 7.14 (dd, J=7.65, 5.14 Hz, 1H) 7.71 (d, J=8.53 Hz, 2H) 7.96 (d, J=8.53 Hz, 2H) 8.40 (dd, J=5.02, 1.76 Hz, 1H) 8.50 (dd, J=7.78, 2.01 Hz, 1H).

### Example 17: 3-methyl-3-[5-[2-[4-(trifluoromethyl)anilino]-3-pyridyl]-1,3,4-oxadiazol-2-yl]piperazin-2-one (Compound 26)

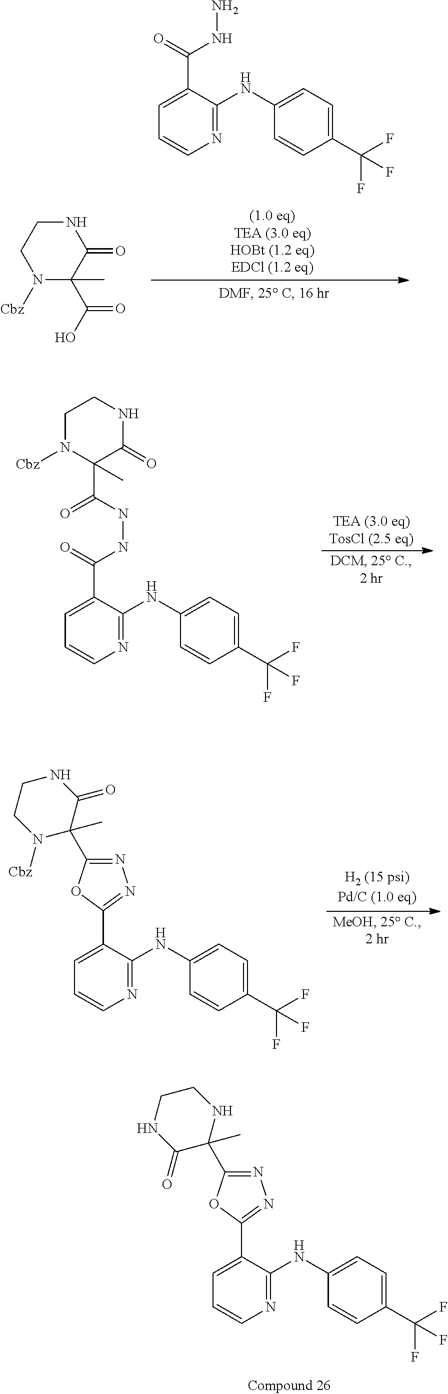 [SMILES: NNC(=O)c1cccnc1Nc1ccc(C(F)(F)F)cc1 ; CC1(C(=O)O)C(=O)NCCN1C(=O)OCc1ccccc1 ; CC1(C(=O)NNC(=O)c2cccnc2Nc2ccc(C(F)(F)F)cc2)C(=O)NCCN1C(=O)OCc1ccccc1 ; CC1(c2nnc(-c3cccnc3Nc3ccc(C(F)(F)F)cc3)o2)C(=O)NCCN1C(=O)OCc1ccccc1 ; CC1(c2nnc(-c3cccnc3Nc3ccc(C(F)(F)F)cc3)o2)NCCNC1=O]

### benzyl 2-methyl-3-oxo-2-[[[2-[4-(trifluoromethyl)anilino]pyridine-3-carbonyl]amino]carbamoyl]piperazine-1-carboxylate

To a solution of 2-((4-(trifluoromethyl)phenyl)amino)nicotinohydrazide (324.3 mg, 1.09 mmol, 1.0 eq) in DMF (3 mL) was added 1-((benzyloxy)carbonyl)-2-methyl-3-oxopiperazine-2-carboxylic acid (320.0 mg, 1.09 mmol, 1.0 eq), EDCI (251.8 mg, 1.31 mmol, 1.2 eq), HOBt (177.5 mg, 1.31 mmol, 1.2 eq) and TEA (332.3 mg, 3.28 mmol, 0.4 mL, 3.0 eq). The mixture was stirred at 25° C. for 16 hr. The residue was diluted with H₂O (10 mL) and extracted with EA (15 mL*3). The combined organic layers were washed with brine (15 mL), dried over Na₂SO₄, filtered and concentrated under reduced pressure to give a residue. The residue was purified by flash silica gel chromatography to give benzyl 2-methyl-3-oxo-2-[[[2-[4-(trifluoromethyl)anilino]pyridine-3-carbonyl]amino]carbamoyl]piperazine-1-carboxylate (130.0 mg, 0.18 mmol, 16.6% yield) as yellow oil. LCMS (ESI): RT=0.830 min, mass calc. for C₂₇H₂₅F₃N₆O₅ 570.18, m/z found 571.1 [M+H]⁺.

### benzyl 2-methyl-3-oxo-2-[5-[2-[4-(trifluoromethyl)anilino]-3-pyridyl]-1,3,4-oxadiazol-2-yl]piperazine-1-carboxylate

To a solution of benzyl 2-methyl-3-oxo-2-[[[2-[4-(trifluoromethyl)anilino]pyridine-3-carbonyl]amino]carbamoyl]piperazine-1-carboxylate (130.0 mg, 0.23 mmol, 1.0 eq) in DCM (2 mL) was added TEA (69.1 mg, 0.68 mmol, 95 uL, 3.0 eq) and TosCl (108.6 mg, 0.57 mmol, 2.5 eq). The mixture was stirred at 25° C. for 2 hr. The reaction mixture was concentrated under reduced pressure to give a residue. The residue was purified by flash silica gel chromatography to give benzyl 2-methyl-3-oxo-2-[5-[2-[4-(trifluoromethyl)anilino]-3-pyridyl]-1,3,4-oxadiazol-2-yl]piperazine-1-carboxylate (210.0 mg, crude) as yellow oil. LCMS (ESI): RT=0.906 min, mass calc. for C₂₇H₂₃F₃N₆O₄ 552.17, m/z found 553.1 [M+H]⁺.

### 3-methyl-3-[5-[2-[4-(trifluoromethyl)anilino]-3-pyridyl]-1,3,4-oxadiazol-2-yl]piperazin-2-one (Compound 26)

To a solution of benzyl 2-methyl-3-oxo-2-[5-[2-[4-(trifluoromethyl)anilino]-3-pyridyl]-1,3,4-oxadiazol-2-yl]piperazine-1-carboxylate (70.0 mg, 0.13 mmol, 1.0 eq) in MeOH (1 mL) was added Pd/C (10.0 mg, 0.13 mmol, 10%, 1.0 eq) and the mixture was degassed and purged with H₂ for three times. The mixture was stirred at 25° C. for 2 hr under H₂ (15 psi) atmosphere. The reaction mixture was concentrated under reduced pressure to give a residue. The residue was purified by prep-HPLC (column: 3_Phenomenex Luna C18 75*30 mm*3 um; mobile phase: [water (0.05% HCl)-ACN]; B %: 35%-65%, 6.5 min) to give the title compound (10.0 mg, 24.0 umol, 19% yield, HCl) as a white solid. LCMS (ESI): RT=0.788 min, mass calc. for C₁₉H₁₇F₃N₆O₂ 418.14, m/z found 419.0 [M+H]⁺; ¹H NMR (400 MHz, CD₃OD) δ 2.24 (s, 3H) 3.71-3.85 (m, 3H) 3.86-3.97 (m, 1H) 7.17 (dd, J=7.78, 5.27 Hz, 1H) 7.73 (d, J=8.53 Hz, 2H) 7.95 (d, J=8.53 Hz, 2H) 8.40 (dd, J=5.27, 1.76 Hz, 1H) 8.56 (dd, J=7.78, 1.76 Hz, 1H).

### Example 18: (3R)-3-methyl-3-[5-[2-[4-(pentafluoro-λ⁶-sulfanyl)anilino]-3-pyridyl]tetrazol-2-yl]pyrrolidin-2-one (Compound 27) and (3R)-3-methyl-3-[5-[2-[4-(pentafluoro-λ⁶-sulfanyl)anilino]-3-pyridyl]tetrazol-2-yl]pyrrolidin-2-one (Compound 28)

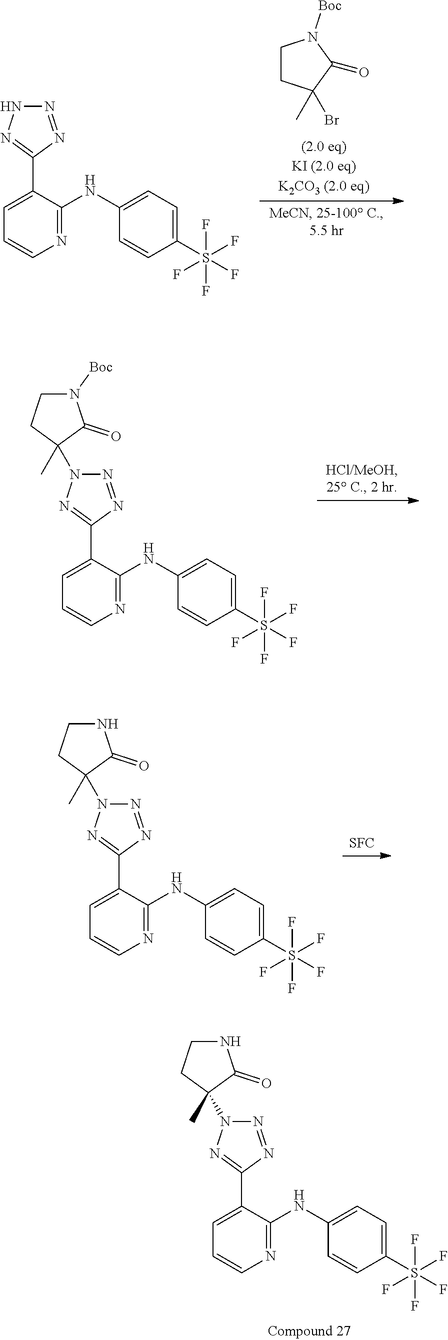 [SMILES: FS(F)(F)(F)(F)c1ccc(Nc2ncccc2-c2nn[nH]n2)cc1 ; CC(C)(C)OC(=O)N1CCC(C)(Br)C1=O ; CC(C)(C)OC(=O)N1CCC(C)(n2nnc(-c3cccnc3Nc3ccc(S(F)(F)(F)(F)F)cc3)n2)C1=O ; CC1(n2nnc(-c3cccnc3Nc3ccc(S(F)(F)(F)(F)F)cc3)n2)CCNC1=O ; "SFC" ; C[C@@]1(n2nnc(-c3cccnc3Nc3ccc(S(F)(F)(F)(F)F)cc3)n2)CCNC1=O]

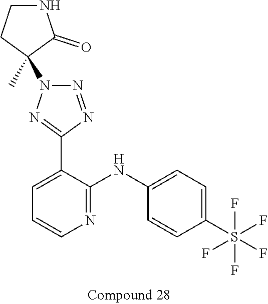 [SMILES: C[C@]1(n2nnc(-c3cccnc3Nc3ccc(S(F)(F)(F)(F)F)cc3)n2)CCNC1=O]

### tert-butyl 3-methyl-2-oxo-3-[5-[2-[4-(pentafluoro-λ⁶-sulfanyl)anilino]-3-pyridyl]tetrazol-2-yl]pyrrolidine-1-carboxylate

To a solution of tert-butyl 3-bromo-3-methyl-2-oxopyrrolidine-1-carboxylate (130.9 mg, 0.36 mmol, 1.0 eq) in MeCN (2 mL) was added K₂CO₃ (99.3 mg, 0.72 mmol, 2.0 eq) and the mixture was stirred at 25° C. for 0.5 hr. N-(4-(pentafluoro-16-sulfanyl)phenyl)-3-(2H-tetrazol-5-yl)pyridin-2-amine (200.0 mg, 0.72 mmol, 2.0 eq) and KI (119.3 mg, 0.72 mmol, 2.0 eq) was added to the above solution and the resulting mixture was stirred at 100° C. for 5 hr. The reaction mixture was concentrated under reduced pressure to give a residue. The residue was purified by flash silica gel chromatography to give tert-butyl 3-methyl-2-oxo-3-[5-[2-[4-(pentafluoro-λ⁶-sulfanyl)anilino]-3-pyridyl]tetrazol-2-yl]pyrrolidine-1-carboxylate (110.0 mg, 78.3 umol, 21.7% yield) as a yellow solid. LCMS (ESI): RT=1.014 min, mass calc. for C₂₂H₂₄F₅N₇O₃S 561.16, m/z found 562.1 [M+H]⁺.

### 3-methyl-3-[5-[2-[4-(pentafluoro-λ⁶-sulfanyl)anilino]-3-pyridyl]tetrazol-2-yl]pyrrolidin-2-one

A solution of tert-butyl 3-methyl-2-oxo-3-[5-[2-[4-(pentafluoro-λ⁶-sulfanyl)anilino]-3-pyridyl]tetrazol-2-yl]pyrrolidine-1-carboxylate (110.0 mg, 78.3 umol, 1.0 eq) in HCl/MeOH (1 mL) was stirred at 25° C. for 2 hr. The reaction mixture was concentrated under reduced pressure to give a residue. The residue was purified by prep-HPLC (column: 3_Phenomenex Luna C18 75*30 mm*3 um; mobile phase: [water (0.05% HCl)-ACN]; B %: 50%-80%, 6.5 min) to give compound 3-methyl-3-[5-[2-[4-(pentafluoro-λ⁶-sulfanyl)anilino]-3-pyridyl]tetrazol-2-yl]pyrrolidin-2-one (12.0 mg, 26.0 umol, 33.1% yield) as a white solid. LCMS (ESI): RT=0.900 min, mass calc. for C₁₇H₁₆F₅N₇OS 461.11, m/z found 462.0 [M+H]⁺.

### (3R)-3-methyl-3-[5-[2-[4-(pentafluoro-λ⁶-sulfanyl)anilino]-3-pyridyl]tetrazol-2-yl]pyrrolidin-2-one (Compound 27) and (3R)-3-methyl-3-[5-[2-[4-(pentafluoro-λ⁶-sulfanyl)anilino]-3-pyridyl]tetrazol-2-yl]pyrrolidin-2-one (Compound 28)

3-Methyl-3-[5-[2-[4-(pentafluoro-λ⁶-sulfanyl)anilino]-3-pyridyl]tetrazol-2-yl]pyrrolidin-2-one (12.0 mg, 26.0 umol, 1.0 eq) was separated by chiral SFC (column: DAICEL CHIRALCEL OJ-H (250 mm*30 mm, 5 um); mobile phase: [0.1% NH₃H₂O ETOH]; B %: 30%-30%, min) to give the title compounds. Compound 27 (1.2 mg, 2.6 umol, 10.1% yield) was obtained as a white solid. LCMS (ESI): RT=0.891 min, mass calc. for C₁₇H₁₆F₅N₇OS 461.11, m/z found 462.0 [M+H]⁺; ¹H NMR (400 MHz, CD₃OD) δ 2.12 (s, 3H) 2.61-2.72 (m, 1H) 3.08 (ddd, J=13.80, 8.16, 5.90 Hz, 1H) 3.54-3.63 (m, 1H) 3.65-3.75 (m, 1H) 7.01-7.15 (m, 1H) 7.75 (d, J=9.29 Hz, 2H) 7.98 (d, J=9.03 Hz, 2H) 8.41 (d, J=3.01 Hz, 1H) 8.55 (d, J=7.78 Hz, 1H). Compound 28 (3.0 mg, 6.5 umol, 25.1% yield) was obtained as a yellow solid. LCMS (ESI): RT=0.892 min, mass calc. for C₁₇H₁₆F₅N₇OS 461.11, m/z found 462.0 [M+H]⁺; ¹H NMR (400 MHz, CD₃OD) δ 2.11 (s, 3H) 2.61-2.72 (m, 1H) 2.98-3.14 (m, 1H) 3.54-3.63 (m, 1H) 3.64-3.73 (m, 1H) 7.05 (dd, J=7.78, 4.77 Hz, 1H) 7.75 (d, J=9.29 Hz, 2H) 7.98 (d, J=9.03 Hz, 2H) 8.40 (dd, J=4.89, 1.88 Hz, 1H) 8.55 (dd, J=7.78, 1.76 Hz, 1H).

### Example 19: (3R)-3-methyl-3-[5-[2-[4-(pentafluoro-λ⁶-sulfanyl)anilino]-3-pyridyl]-1,3,4-oxadiazol-2-yl]piperidin-2-one (Compound 29) and (3S)-3-methyl-3-[5-[2-[4-(pentafluoro-λ⁶-sulfanyl)anilino]-3-pyridyl]-1,3,4-oxadiazol-2-yl]piperidin-2-one (Compound 30)

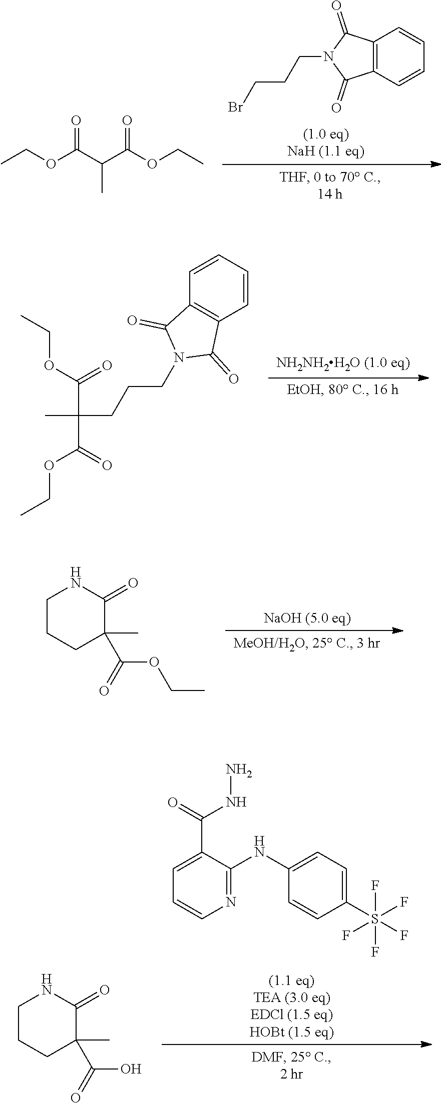 [SMILES: O=C1c2ccccc2C(=O)N1CCCBr ; CCOC(=O)C(C)C(=O)OCC ; CCOC(=O)C(C)(CCCN1C(=O)c2ccccc2C1=O)C(=O)OCC ; CCOC(=O)C1(C)CCCNC1=O ; NNC(=O)c1cccnc1Nc1ccc(S(F)(F)(F)(F)F)cc1 ; CC1(C(=O)O)CCCNC1=O]

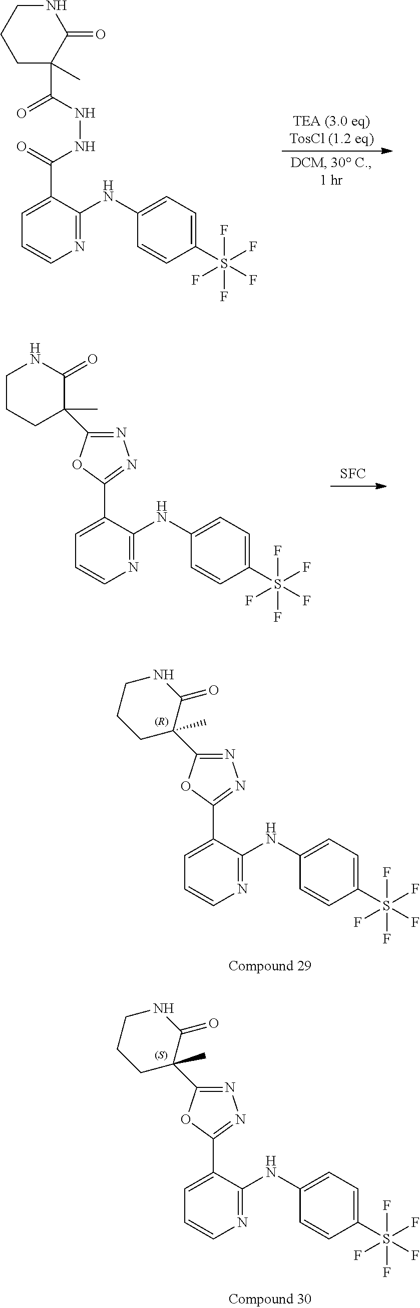

### Diethyl 2-[3-(1,3-dioxoisoindolin-2-yl)propyl]-2-methyl-propanedioate

To a solution of diethyl 2-methylmalonate (2.0 g, 11.48 mmol, 1.96 mL, 1 eq) in THF (20 mL) was added NaH (505.1 mg, 12.63 mmol, 60%, 1.1 eq) at 0° C., and then the reaction mixture was stirred at 0° C. for 0.5 hour. The reaction was cooled to 0° C. After 2-(3-bromopropyl)isoindoline-1,3-dione (3.0 g, 11.48 mmol, 1 eq) was added, the reaction mixture was allowed to warm up to 70° C. and stirred for 16 hours. The mixture was diluted with water (30 mL) and the resultant mixture was extracted with EA (50 mL*3). The combined organic layers were dried over Na₂SO₄, filtered and concentrated to dryness under reduced pressure. The residue was purified by column chromatography over silica gel to afford diethyl 2-[3-(1,3-dioxoisoindolin-2-yl)propyl]-2-methyl-propanedioate (3.05 g, 6.41 mmol, 55.8% yield) as yellow oil.

### Ethyl 3-methyl-2-oxo-piperidine-3-carboxylate

To a solution of diethyl 2-[3-(1,3-dioxoisoindolin-2-yl)propyl]-2-methyl-propanedioate (1 g, 2.77 mmol, 1 eq) in EtOH (25 mL) was added NH₂NH₂.H₂O (162.9 mg, 2.77 mmol, 0.15 mL, 85%, 1 eq), and then the mixture was stirred at 80° C. for 16 hours. The reaction mixture was filtered and the filtrate was concentrated under reduce pressure. The residue was purified by column chromatography over silica gel to afford ethyl 3-methyl-2-oxo-piperidine-3-carboxylate (150 mg, crude) as a white solid.

### 3-Methyl-2-oxo-piperidine-3-carboxylic acid

To a solution of ethyl 3-methyl-2-oxo-piperidine-3-carboxylate (150 mg, 0.80 mmol, 1 eq) in MeOH (3 mL) and H₂O (0.6 mL) was added NaOH (161.9 mg, 4.05 mmol, 5 eq), and then the reaction mixture was stirred at 25° C. for 3 hours. The reaction was adjusted with HCl (2 M) to pH=5, and then the mixture was concentrated under reduced pressure. The residue was washed with DCM:MeOH=10:1 (30 mL) and filtered, the filtrate was concentrated under reduce pressure to give 3-methyl-2-oxo-piperidine-3-carboxylic acid (112 mg, 0.71 mmol, 87.9% yield) was obtained as a white solid.

### N′-(3-Methyl-2-oxo-piperidine-3-carbonyl)-2-[4-(pentafluoro-λ⁶-sulfanyl)anilino]pyridine-3-carbohydrazide

To a solution of 3-methyl-2-oxo-piperidine-3-carboxylic acid (200 mg, 0.51 mmol, 1 eq, HCl) and 2-((4-(pentafluoro-6-sulfanyl)phenyl)amino)nicotinohydrazide (88.4 mg, 0.56 mmol, 1.1 eq) in DMF (3 mL) were added EDCI (147.1 mg, 0.76 mmol, 1.5 eq), TEA (155.3 mg, 1.54 mmol, 0.21 mL, 3 eq) and HOBt (103.7 mg, 0.76 mmol, 1.5 eq), the reaction mixture was stirred at 25° C. for 2 hours. The reaction mixture was concentrated under reduced pressure. The mixture was diluted with water (10 mL) and the resultant mixture was extracted with EA (30 mL*3). The combined organic layers were dried over Na₂SO₄, filtered and concentrated to dryness under reduced pressure. The residue was purified by column chromatography over silica gel (DCM:MeOH=1:0 to 10:1) to afford N′-(3-methyl-2-oxo-piperidine-3-carbonyl)-2-[4-(pentafluoro-λ⁶-sulfanyl)anilino]pyridine-3-carbohydrazide (192 mg, 0.35 mmol, 69.1% yield) as a white solid.

### 3-Methyl-3-[5-[2-[4-(pentafluoro-λ⁶-sulfanyl)anilino]-3-pyridyl]-1,3,4-oxadiazol-2-yl]piperidin-2-one

To a solution of N′-(3-methyl-2-oxo-piperidine-3-carbonyl)-2-[4-(pentafluoro-λ⁶-sulfanyl)anilino]pyridine-3-carbohydrazide (190 mg, 0.38 mmol, 1 eq) and TEA (116.8 mg, 1.16 mmol, 0.16 mL, 3 eq) in DCM (3 mL) was added TosCl (88.0 mg, 0.46 mmol, 1.2 eq), and then the reaction mixture was stirred at 25° C. for 1 hour. The reaction mixture was concentrated under reduced pressure. The mixture was diluted with water (10 mL) and the resultant mixture was extracted with EA (20 mL*3). The combined organic layers were dried over Na₂SO₄, filtered and concentrated to dryness under reduced pressure. The residue was purified by column chromatography over silica gel (petroleum ether:ethyl acetate=1:0 to 0:1) to afford the title compound as a light yellow solid. The crude product was triturated with MeOH (5 mL) and filtered to obtain the title compound 3-methyl-3-[5-[2-[4-(pentafluoro-λ⁶-sulfanyl)anilino]-3-pyridyl]-1,3,4-oxadiazol-2-yl]piperidin-2-one (51 mg, 0.10 mmol, 27.8% yield) as a white solid.

### (3R)-3-methyl-3-[5-[2-[4-(pentafluoro-λ⁶-sulfanyl)anilino]-3-pyridyl]-1,3,4-oxadiazol-2-yl]piperidin-2-one (Compound 29) and (3S)-3-methyl-3-[5-[2-[4-(pentafluoro-λ⁶-sulfanyl)anilino]-3-pyridyl]-1,3,4-oxadiazol-2-yl]piperidin-2-one (Compound 30)

3-Methyl-3-[5-[2-[4-(pentafluoro-λ⁶-sulfanyl)anilino]-3-pyridyl]-1,3,4-oxadiazol-2-yl]piperidin-2-one (51 mg, 0.10 mmol, 1 eq) was separated by SFC (column: Phenomenex-Cellulose-2 (250 mm*30 mm, 5 um); mobile phase: [0.10% NH₃H₂O ETOH]; B %: 45%-45%, min) to give the title compounds. Compound 29 (16.48 mg, 34.1 umol, 31.8% yield): LCMS (ESI): RT=0.967 min, mass calcd for C₁₉H₁₈F₅N₅O₂S 475.11 m/z found 476.1 [M+H]⁺; ¹H NMR (400 MHz, DMSO-d₆) δ 10.31 (s, 1H), 8.51 (dd, J=1.8, 4.8 Hz, 1H), 8.31 (dd, J=1.9, 7.8 Hz, 1H), 8.04-7.97 (m, 2H), 7.95 (br s, 1H), 7.88 (d, J=9.3 Hz, 2H), 7.16 (dd, J=4.9, 7.8 Hz, 1H), 3.29 (br d, J=4.4 Hz, 2H), 2.44-2.36 (m, 1H), 2.03-1.94 (m, 1H), 1.93-1.84 (m, 2H), 1.72 (s, 3H). Compound 30 (14.61 mg, 30.1 umol, 28.1% yield) as a white solid: LCMS (ESI): RT=0.970 min, mass calcd for C₁₉H₁₈F₅N₅O₂S 475.11 m/z found 476.1 [M+H]⁺; ¹H NMR (400 MHz, DMSO-d₆) δ 10.30 (s, 1H), 8.50 (dd, J=1.8, 4.8 Hz, 1H), 8.30 (dd, J=1.8, 7.8 Hz, 1H), 7.99 (br d, J=9.0 Hz, 2H), 7.95 (br s, 1H), 7.88 (br d, J=9.3 Hz, 2H), 7.16 (dd, J=4.9, 7.8 Hz, 1H), 3.30 (br s, 2H), 2.44-2.35 (m, 1H), 2.02-1.94 (m, 1H), 1.93-1.85 (m, 2H), 1.72 (s, 3H).

### Example 20: (3S)-3-ethyl-3-[5-[2-[4-(trifluoromethyl)anilino]-3-pyridyl]-1,3,4-oxadiazol-2-yl]piperidin-2-one (Compound 31) and (3R)-3-ethyl-3-[5-[2-[4-(trifluoromethyl)anilino]-3-pyridyl]-1,3,4-oxadiazol-2-yl]piperidin-2-one (Compound 32)

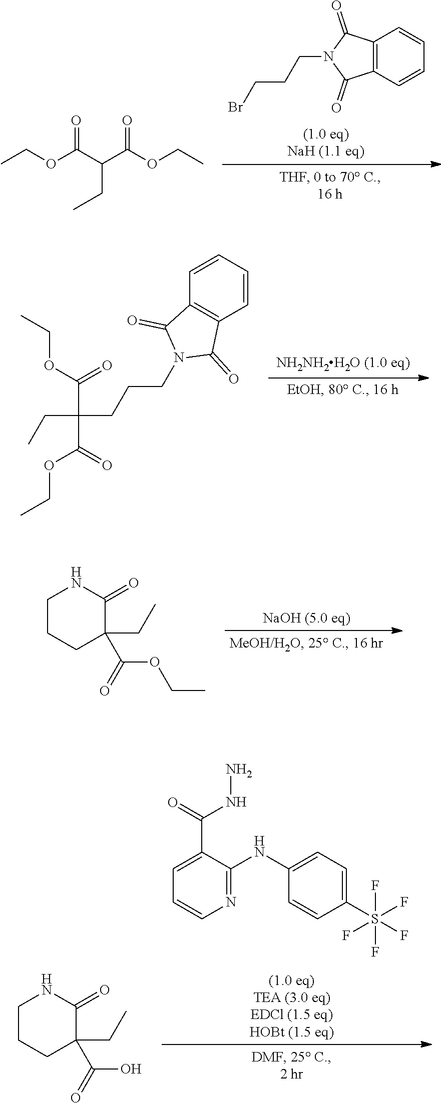 [SMILES: O=C1c2ccccc2C(=O)N1CCCBr ; CCOC(=O)C(CC)C(=O)OCC ; CCOC(=O)C(CC)(CCCN1C(=O)c2ccccc2C1=O)C(=O)OCC ; CCOC(=O)C1(CC)CCCNC1=O ; NNC(=O)c1cccnc1Nc1ccc(S(F)(F)(F)(F)F)cc1 ; CCC1(C(=O)O)CCCNC1=O]

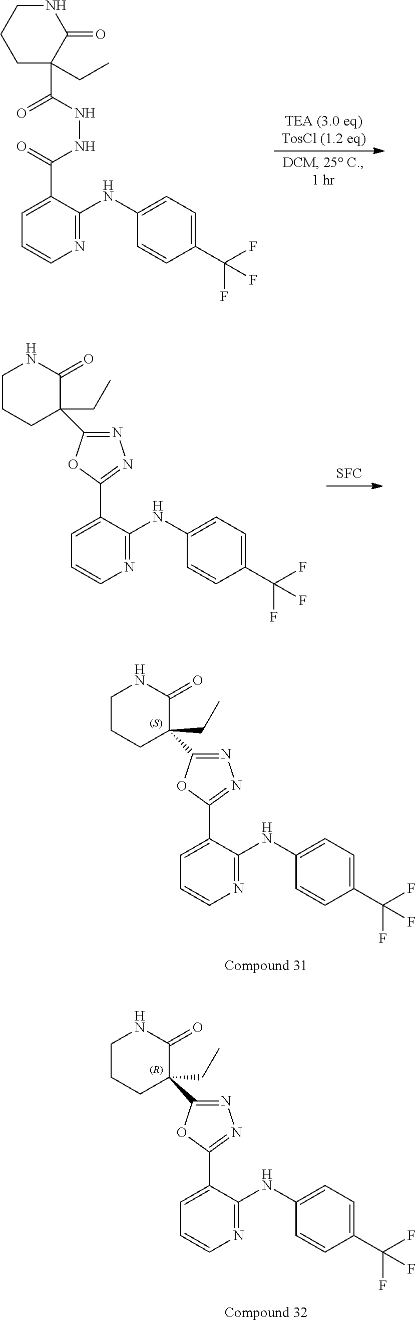

### Diethyl 2-[3-(1,3-dioxoisoindolin-2-yl)propyl]-2-ethyl-propanedioate

To a solution of NaH (328.2 mg, 8.21 mmol, 60%, 1.1 eq) in THF (30 mL) was added diethyl 2-ethylpropanedioate (1.40 g, 7.46 mmol, 1.40 mL, 1 eq)/THF (10 mL) at 0° C. The reaction was stirred at 25° C. for 1 h. 2-(3-bromopropyl)isoindoline-1,3-dione (2 g, 7.46 mmol, 1 eq)/THF (10 mL) was added dropwise. The reaction was stirred at 70° C. for 15 h. The reaction was quenched with H₂O (30 mL) and was concentrated under reduce pressure. The reaction mixture was extracted with EA (50 mL*3). The combined organic layers were dried over Na₂SO₄, filtered and concentrated to dryness under reduced pressure. The residue was purified by flash silica gel chromatography to give the title compound as a colorless oil. Diethyl 2-[3-(1,3-dioxoisoindolin-2-yl)propyl]-2-ethyl-propanedioate (1 g, 2.20 mmol, 29.4% yield) was obtained as a colorless oil.

### Ethyl 3-ethyl-2-oxo-piperidine-3-carboxylate

To a solution of diethyl 2-[3-(1,3-dioxoisoindolin-2-yl)propyl]-2-ethyl-propanedioate (2 g, 5.33 mmol, 1 eq) in EtOH (20 mL) was added NH₂NH₂.H₂O (293.3 mg, 5.86 mmol, 0.28 mL, 1.1 eq). The reaction was stirred at 80° C. for 16 hr. The reaction mixture was filtered and the filtrate was concentrated under reduce pressure. The residue was purified by flash silica gel chromatography to give the desired compound as a colorless oil (400 mg, 2.01 mmol, 37.6% yield).

### 3-Ethyl-2-oxo-piperidine-3-carboxylic acid

To a solution of ethyl 3-ethyl-2-oxo-piperidine-3-carboxylate (400 mg, 2.01 mmol, 1 eq) in MeOH (5 mL) and H₂O (0.5 mL) was added NaOH (401.4 mg, 10.04 mmol, 5 eq). The reaction was stirred at 25° C. for 16 hr. The reaction mixture was adjusted pH=3-5 with 2N HCl. The reaction mixture was concentrated under reduced pressure to give a residue. The residue was dissolved in DCM/MeOH=10/1 (22 mL). The mixture was filtered and the filtrate was concentrated under reduced pressure to give a residue. Compound 3-ethyl-2-oxo-piperidine-3-carboxylic acid (340 mg, crude) was obtained as a yellow solid.

### N′-(3-ethyl-2-oxo-piperidine-3-carbonyl)-2-[4-(trifluoromethyl)anilino]pyridine-3-carbohydrazide

To solution of 3-ethyl-2-oxo-piperidine-3-carboxylic acid (340 mg, 1.99 mmol, 1 eq) and 2-[4-(trifluoromethyl)anilino]pyridine-3-carbohydrazide (588.3 mg, 1.99 mmol, 1 eq) in DMF (5 mL) was added HOBt (402.5 mg, 2.98 mmol, 1.5 eq), TEA (602.9 mg, 5.96 mmol, 0.82 mL, 3 eq) and EDCI (571.1 mg, 2.98 mmol, 1.5 eq). The reaction was stirred at 25° C. for 2 hr. The mixture was diluted with water (20 mL) and the resultant mixture was extracted with EA (25 mL*3). The combined organic layers were dried over Na₂SO₄, filtered and concentrated to dryness under reduced pressure. The residue was purified by flash silica gel chromatography to give the title compound as a yellow oil. N′-(3-ethyl-2-oxo-piperidine-3-carbonyl)-2-[4-(trifluoromethyl)anilino]pyridine-3-carbohydrazide (810 mg, 1.53 mmol, 76.9% yield) was obtained as a yellow oil.

### 3-ethyl-3-[5-[2-[4-(trifluoromethyl)anilino]-3-pyridyl]-1,3,4-oxadiazol-2-yl]piperidin-2-one

To a solution of N′-(3-ethyl-2-oxo-piperidine-3-carbonyl)-2-[4-(trifluoromethyl)anilino]pyridine-3-carbohydrazide (400 mg, 0.89 mmol, 1 eq) in DCM (4 mL) was added TEA (270.1 mg, 2.67 mmol, 0.37 mL, 3 eq) and TosCl (203.6 mg, 1.07 mmol, 1.2 eq). The reaction was stirred at 25° C. for 1 hr. The residue was purified by flash silica gel chromatography to give the title compound as a yellow solid. 3-Ethyl-3-[5-[2-[4-(trifluoromethyl)anilino]-3-pyridyl]-1,3,4-oxadiazol-2-yl]piperidin-2-one (270 mg, 0.61 mmol, 68.9% yield) was obtained as a yellow solid.

### (3S)-3-ethyl-3-[5-[2-[4-(trifluoromethyl)anilino]-3-pyridyl]-1,3,4-oxadiazol-2-yl]piperidin-2-one (Compound 31) and (3R)-3-ethyl-3-[5-[2-[4-(trifluoromethyl)anilino]-3-pyridyl]-1,3,4-oxadiazol-2-yl]piperidin-2-one (Compound 32)

The 3-ethyl-3-[5-[2-[4-(trifluoromethyl)anilino]-3-pyridyl]-1,3,4-oxadiazol-2-yl]piperidin-2-one (95 mg, 0.22 mmol, 1 eq) was separated by SFC (column: Phenomenex-Cellulose-2 (250 mm*30 mm, 10 um); mobile phase: [0.1% NH3H2O ETOH]; B %: 50%-50%, min) to give the title compounds. Compound 31: Compound (3S)-3-ethyl-3-[5-[2-[4-(trifluoromethyl)anilino]-3-pyridyl]-1,3,4-oxadiazol-2-yl]piperidin-2-one (33.3 mg, 77.2 umol, 35.1% yield) was obtained as a white solid. LCMS (ESI): RT=0.961 min, mass calcd for C₂₁H₂₀F₃N₅O₂ 431.41 m/z, found 432.1 [M+H]⁺, ¹H NMR (400 MHz, DMSO-d₆) δ 10.26 (s, 1H), 8.49 (dd, J=1.8, 4.8 Hz, 1H), 8.27 (dd, J=1.9, 7.9 Hz, 1H), 8.06-7.92 (m, 3H), 7.71 (d, J=8.6 Hz, 2H), 7.13 (dd, J=4.9, 7.8 Hz, 1H), 3.28-3.22 (m, 2H), 2.37-2.32 (m, 1H), 2.25-2.01 (m, 3H), 1.98-1.78 (m, 2H), 0.94 (t, J=7.4 Hz, 3H). Compound 32: Compound (3R)-3-ethyl-3-[5-[2-[4-(trifluoromethyl)anilino]-3-pyridyl]-1,3,4-oxadiazol-2-yl]piperidin-2-one (33.3 mg, 77.0 umol, 34.9% yield) was obtained as a white solid. LCMS (ESI): RT=0.963 min, mass calcd for C₂₁H₂₀F₃N₅O₂ 431.41 m/z, found 432.1 [M+H]⁺, ¹H NMR (400 MHz, DMSO-d₆) δ 10.26 (s, 1H), 8.49 (dd, J=1.9, 4.8 Hz, 1H), 8.27 (dd, J=1.9, 7.8 Hz, 1H), 8.01 (d, J=8.5 Hz, 2H), 7.94 (s, 1H), 7.71 (d, J=8.6 Hz, 2H), 7.13 (dd, J=4.9, 7.8 Hz, 1H), 3.28-3.22 (m, 2H), 2.69-2.64 (m, 1H), 2.38-2.32 (m, 1H), 2.23-2.02 (m, 3H), 1.96-1.77 (m, 2H), 0.94 (t, J=7.4 Hz, 3H).

### Example 21: (3R)-3-ethyl-3-[5-[2-[4-(trifluoromethyl)anilino]-3-pyridyl]-1,3,4-oxadiazol-2-yl]pyrrolidin-2-one (Compound 33) and (3S)-3-ethyl-3-[5-[2-[4-(trifluoromethyl)anilino]-3-pyridyl]-1,3,4-oxadiazol-2-yl]pyrrolidin-2-one (Compound 34)

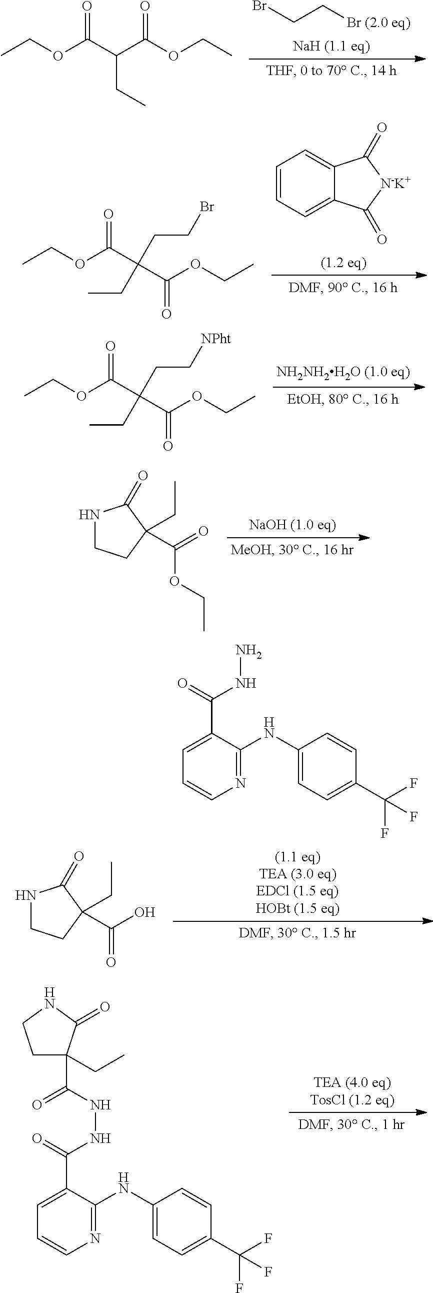

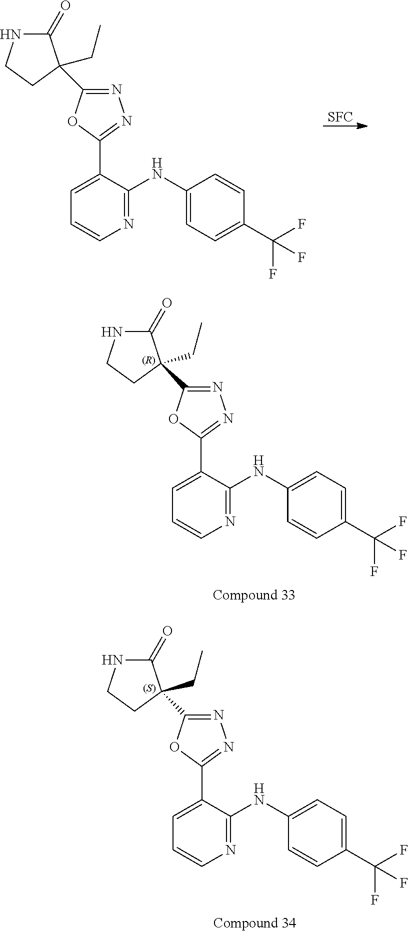

### diethyl 2-(2-bromoethyl)-2-ethyl-propanedioate

To a solution of diethyl 2-ethylmalonate (5 g, 26.56 mmol, 5.0 mL, 1 eq) in THF (50 mL) was added NaH (1.17 g, 29.2 mmol, 60%, 1.1 eq) at 0° C. 1,2-dibromoethane (9.98 g, 53.13 mmol, 4.01 mL, 2.0 eq) was added and then the mixture was stirred at 70° C. for 14 hr. The mixture was quenched with H₂O (25 mL) and diluted with EA (150 mL). The mixture was wished twice with 1M HCl (50 mL), brine (150 mL), dried over Na₂SO₄, filtered and concentrated under reduced pressure to give a residue. The crude product was used into the next step without further purification. Compound diethyl 2-(2-bromoethyl)-2-ethyl-propanedioate (6 g, crude) was obtained as a yellow oil.

### Diethyl 2-[2-(1,3-dioxoisoindolin-2-yl)ethyl]-2-ethyl-propanedioate

To a solution of diethyl 2-(2-bromoethyl)-2-ethyl-propanedioate (3 g, 10.16 mmol, 1 eq) in DMF (30 mL) was added potassium 1,3-dioxoisoindolin-2-ide (2.26 g, 12.2 mmol, 1.2 eq). The mixture was stirred at 90° C. for 16 hr. The mixture was concentrated under reduced pressure to give a residue. The residue was diluted with H₂O (200 mL), extracted with EA (100 mL*3). The combined organic layers were washed with brine (100 mL), dried over Na₂SO₄, filtered and concentrated under reduced pressure to give a residue The residue was purified by column chromatography to give diethyl 2-[2-(1,3-dioxoisoindolin-2-yl)ethyl]-2-ethyl-propanedioate (1.5 g, 3.94 mmol, 38% yield) as a yellow oil.

### Ethyl 3-ethyl-2-oxo-pyrrolidine-3-carboxylate

To a solution of diethyl 2-[2-(1,3-dioxoisoindolin-2-yl)ethyl]-2-ethyl-propanedioate (1.5 g, 4.15 mmol, 1 eq) in EtOH (20 mL) was added NH₂—NH₂.H₂O (244.4 mg, 4.15 mmol, 0.23 mL, 85%, 1 eq). The mixture was stirred at 80° C. for 16 hr. The mixture was filtered. The filtrate was concentrated under reduced pressure to give a residue. The residue was diluted with H₂O (30 mL), extracted with EA (30 mL*3). The combined organic layers were washed with brine (100 mL), dried over Na₂SO₄, filtered and concentrated under reduced pressure to give a residue. The residue was purified by column chromatography to give ethyl 3-ethyl-2-oxo-pyrrolidine-3-carboxylate (0.4 g, 2.16 mmol, 52% yield) as a colorless oil.

### 3-Ethyl-2-oxo-pyrrolidine-3-carboxylic acid

To a solution of ethyl 3-ethyl-2-oxo-pyrrolidine-3-carboxylate (0.4 g, 2.1 mmol, 1 eq) in MeOH (5 mL) and H₂O (1 mL) was added NaOH (86.3 mg, 2.16 mmol, 1 eq). The mixture was stirred at 30° C. for 16 hr. The mixture was concentrated under reduced pressure to give a residue. The residue was diluted with H₂O (10 mL) and adjusted PH=3-4 with 2M HCl. The mixture was concentrated under reduced pressure to give a residue. The residue was dissolved in DCM/MeOH=10/1 (30 mL). The mixture was filtered. The filtrate was concentrated under reduced pressure to give a residue. The residue was dissolved in EtOH (50 mL), then concentrated under reduced pressure to give a residue. The crude product was used into the next step without further purification. Compound 3-ethyl-2-oxo-pyrrolidine-3-carboxylic acid (0.3 g, crude) was obtained as a white solid.

### N′-(3-ethyl-2-oxo-pyrrolidine-3-carbonyl)-2-[4-(trifluoromethyl)anilino]pyridine-3-carbohydrazide

To a solution of 3-ethyl-2-oxo-pyrrolidine-3-carboxylic acid (250 mg, 1.5 mmol, 1 eq) and 2-((4-(trifluoromethyl)phenyl)amino)nicotinohydrazide (518.3 mg, 1.75 mmol, 1.1 eq) in DMF (6 mL) was added HOBt (322.4 mg, 2.39 mmol, 1.5 eq), TEA (482.8 mg, 4.77 mmol, 0.66 mL, 3.0 eq) and EDCI (457.4 mg, 2.39 mmol, 1.5 eq). The mixture was stirred at 25° C. for 1.5 hr. The reaction mixture was filtered and concentrated under reduced pressure to give a residue. The residue was purified by column chromatography to give N′-(3-ethyl-2-oxo-pyrrolidine-3-carbonyl)-2-[4-(trifluoromethyl)anilino]pyridine-3-carbohydrazide (500 mg, 1.06 mmol, 66% yield) as a white solid.

### 3-Ethyl-3-[5-[2-[4-(trifluoromethyl)anilino]-3-pyridyl]-1,3,4-oxadiazol-2-yl]pyrrolidin-2-one

To a solution of N′-(3-ethyl-2-oxo-pyrrolidine-3-carbonyl)-2-[4-(trifluoromethyl)anilino]pyridine-3-carbohydrazide (0.3 g, 0.68 mmol, 1 eq) in DCM (5 mL) was added TosCl (157.6 mg, 0.82 mmol, 1.2 eq) and TEA (278.8 mg, 2.7 mmol, 0.38 mL, 4.0 eq). The mixture was stirred at 30° C. for 1 hr. The reaction mixture was concentrated under reduced pressure to give a residue. The residue was purified by column chromatography to give 3-ethyl-3-[5-[2-[4-(trifluoromethyl)anilino]-3-pyridyl]-1,3,4-oxadiazol-2-yl]pyrrolidin-2-one (250 mg, 0.59 mmol, 86% yield) as a white solid.

### (3R)-3-ethyl-3-[5-[2-[4-(trifluoromethyl)anilino]-3-pyridyl]-1,3,4-oxadiazol-2-yl]pyrrolidin-2-one (Compound 33) and (3S)-3-ethyl-3-[5-[2-[4-(trifluoromethyl)anilino]-3-pyridyl]-1,3,4-oxadiazol-2-yl]pyrrolidin-2-one (Compound 34)

3-ethyl-3-[5-[2-[4-(trifluoromethyl)anilino]-3-pyridyl]-1,3,4-oxadiazol-2-yl]pyrrolidin-2-one (90 mg) was separated by prep-SFC (column: DAICEL CHIRALPAK AD (250 mm*30 mm, 10 um); mobile phase: [0.1% NH₃H₂O ETOH]; B %: 55%-55%, min) to give Compound 33 (36.8 mg, 87 umol, 40% yield) and Compound 34 (41.3 mg, 97.1 umol, 45% yield). Compound 33: LCMS (ESI): RT=0.949 min, mass calcd for: C₂₀H₁₈F₃N₅O₂ 417.14 m/z found 418.1 [M+H]⁺; ¹H NMR (400 MHz, DMSO-d₆) δ 10.24 (s, 1H), 8.50 (dd, J=1.9, 4.9 Hz, 1H), 8.28 (dd, J=2.0, 7.8 Hz, 1H), 8.21 (s, 1H), 8.01 (d, J=8.5 Hz, 2H), 7.71 (d, J=8.8 Hz, 2H), 7.14 (dd, J=4.8, 7.8 Hz, 1H), 3.49-3.41 (m, 1H), 3.40-3.34 (m, 1H), 2.78 (ddd, J=4.8, 8.2, 13.2 Hz, 1H), 2.38-2.30 (m, 1H), 2.19 (qd, J=7.3, 14.1 Hz, 1H), 2.03-1.92 (m, 1H), 0.96 (t, J=7.4 Hz, 3H). Compound 34 LCMS (ESI): RT=0.950 min, mass calcd for: C₂₀H₁₈F₃N₅O₂ 417.14 m/z found 418.1 [M+H]⁺; ¹H NMR (400 MHz, DMSO-d₆) δ 10.23 (s, 1H), 8.49 (dd, J=1.8, 4.8 Hz, 1H), 8.27 (dd, J=1.8, 7.8 Hz, 1H), 8.20 (s, 1H), 8.00 (d, J=8.5 Hz, 2H), 7.70 (d, J=8.8 Hz, 2H), 7.13 (dd, J=4.8, 7.8 Hz, 1H), 3.49-3.41 (m, 1H), 3.40-3.34 (m, 1H), 2.77 (ddd, J=4.8, 8.2, 13.2 Hz, 1H), 2.39-2.28 (m, 1H), 2.18 (sxt, J=7.2 Hz, 1H), 2.03-1.90 (m, 1H), 0.96 (t, J=7.4 Hz, 3H).

### Example 22: (3R)-3-methyl-3-[5-[2-[4-(pentafluoro-λ⁶-sulfanyl)anilino]-3-pyridyl]-1,3,4-oxadiazol-2-yl]pyrrolidin-2-one (Compound 35) and (3S)-3-methyl-3-[5-[2-[4-(pentafluoro-λ⁶-sulfanyl)anilino]-3-pyridyl]-1,3,4-oxadiazol-2-yl]pyrrolidin-2-one (Compound 36)

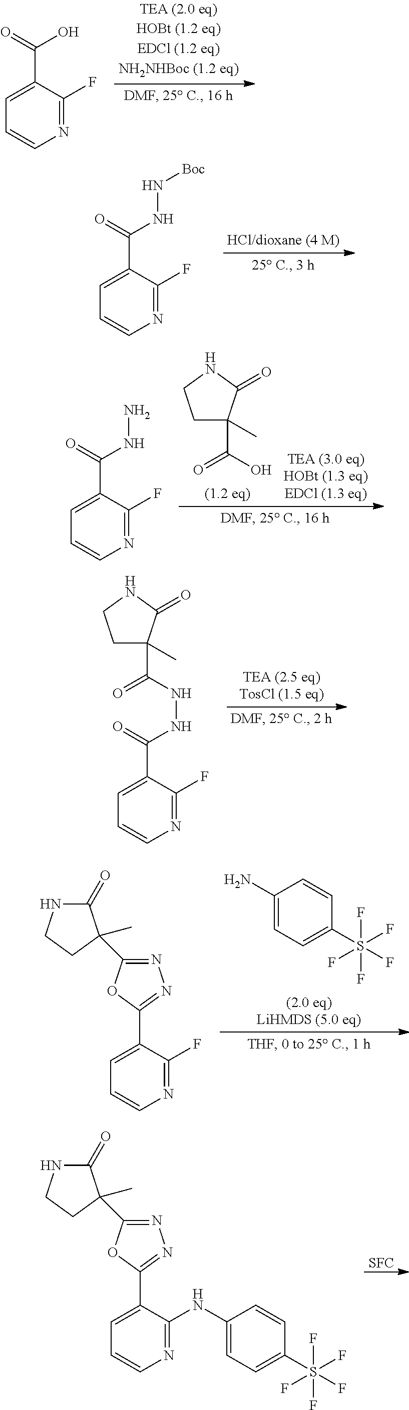

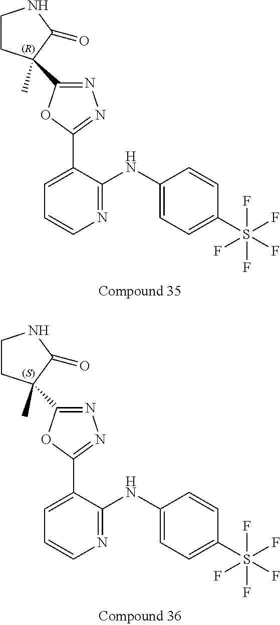

### tert-butyl N-[(2-fluoropyridine-3-carbonyl)amino]carbamate

To a solution of 2-fluoronicotinic acid (500 mg, 3.54 mmol, 1 eq) and tert-butyl N-aminocarbamate (562.0 mg, 4.25 mmol, 1.2 eq) in DMF (5 mL) were added HOBt (574.6 mg, 4.25 mmol, 1.2 eq), EDCI (815.2 mg, 4.25 mmol, 1.2 eq) and TEA (717.1 mg, 7.09 mmol, 0.99 mL, 2 eq). The mixture was stirred at 25° C. for 16 h. The reaction mixture was diluted with H₂O (20 mL) and extracted with EA (30 mL*3). The combined organic layers were washed with brine (20 mL), dried with anhydrous Na₂SO₄, filtered and concentrated under reduced pressure to give a residue. The residue was purified by flash silica gel chromatography to give tert-butyl N-[(2-fluoropyridine-3-carbonyl)amino]carbamate (900 mg, 3.00 mmol, 84.8% yield) as colorless oil.

### 2-fluoropyridine-3-carbohydrazide

A solution of tert-butyl N-[(2-fluoropyridine-3-carbonyl)amino]carbamate (500 mg, 1.96 mmol, 1 eq) in HCl/dioxane (5 mL) was stirred at 25° C. for 3 h. Yellow solid formed. The mixture was filtered. The filter cake was washed with EA (20 mL*2) and concentrated under reduced pressure to give 2-fluoropyridine-3-carbohydrazide (280 mg, crude) as a white solid. ¹H NMR (400 MHz, CD₃OD) 8.52-8.33 (m, 2H), 7.52 (ddd, J=1.8, 5.1, 7.2 Hz, 1H).

### 2-fluoro-N′-(3-methyl-2-oxo-pyrrolidine-3-carbonyl)pyridine-3-carbohydrazide

To a solution of 2-fluoropyridine-3-carbohydrazide (280 mg, 1.80 mmol, 1 eq) and 3-methyl-2-oxopyrrolidine-3-carboxylic acid (310.0 mg, 2.17 mmol, 1.2 eq) in DMF (5 mL) were added HOBt (317.1 mg, 2.35 mmol, 1.3 eq), EDCI (449.8 mg, 2.35 mmol, 1.3 eq) and TEA (547.9 mg, 5.41 mmol, 0.75 mL, 3 eq). The mixture was stirred at 25° C. for 16 h. The mixture was concentrated under reduced pressure to give a residue. The crude product 2-fluoro-N′-(3-methyl-2-oxo-pyrrolidine-3-carbonyl)pyridine-3-carbohydrazide (505 mg, crude, colorless oil) was used into the next step without further purification.

### 3-[5-(2-fluoro-3-pyridyl)-1,3,4-oxadiazol-2-yl]-3-methyl-pyrrolidin-2-one

To a solution of 2-fluoro-N′-(3-methyl-2-oxo-pyrrolidine-3-carbonyl)pyridine-3-carbohydrazide (305 mg, 1.09 mmol, 1 eq) in DCM (3 mL) were added TosCl (311.2 mg, 1.63 mmol, 1.5 eq) and TEA (275.3 mg, 2.72 mmol, 0.38 mL, 2.5 eq). The mixture was stirred at 25° C. for 2 h. The mixture was diluted with H₂O (10 mL), extracted with EA (20 mL*3). The combined organic layer was washed with brine (20 mL), dried over Na₂SO₄, filtered and concentrated under reduced pressure to give a residue. The residue was purified by flash silica gel chromatography to give 3-[5-(2-fluoro-3-pyridyl)-1,3,4-oxadiazol-2-yl]-3-methyl-pyrrolidin-2-one (180 mg, 0.65 mmol, 59.3% yield) as a white solid. LCMS (ESI): RT=0.621 min, mass calc. for C₁₂H₁₁FN₄O₂ 262.09 found 263.1 [M+H]⁺; ¹H NMR (400 MHz, CDCl₃) δ 8.52 (ddd, J=2.0, 7.5, 9.1 Hz, 1H), 8.46-8.36 (m, 1H), 7.39 (ddd, J=1.6, 4.9, 7.5 Hz, 1H), 6.47 (br s, 1H), 3.75-3.61 (m, 1H), 3.59-3.45 (m, 1H), 3.06-2.92 (m, 1H), 2.33 (ddd, J=6.1, 7.7, 13.4 Hz, 1H), 1.79 (s, 3H).

### 3-methyl-3-[5-[2-[4-(pentafluoro-λ⁶-sulfanyl)anilino]-3-pyridyl]-1,3,4-oxadiazol-2-yl]pyrrolidin-2-one

To a solution of 3-[5-(2-fluoro-3-pyridyl)-1,3,4-oxadiazol-2-yl]-3-methyl-pyrrolidin-2-one (120 mg, 0.46 mmol, 1 eq) and 4-(pentafluoro-λ⁶-sulfanyl)aniline (200.6 mg, 0.92 mmol, 2 eq) in THF (3 mL) was added LiHMDS (1 M, 2.29 mL, 5 eq) at 0° C. The mixture was stirred at 25° C. for 1 h. The reaction mixture was diluted with H₂O (10 mL) and extracted with EA (20 mL*3). The combined organic layers were washed with brine (15 mL), dried with anhydrous Na₂SO₄, filtered and concentrated under reduced pressure to give a residue. The residue was purified by flash silica gel chromatography to give 3-methyl-3-[5-[2-[4-(pentafluoro-λ⁶-sulfanyl)anilino]-3-pyridyl]-1,3,4-oxadiazol-2-yl]pyrrolidin-2-one (170 mg, 0.36 mmol, 79.71% yield) as a yellow solid. LCMS (ESI): RT=0.947 min, mass calc. for C₁₈H₁₆F₅N₅O₂S 461.09 found 462.0 [M+H]⁺.

### (3R)-3-methyl-3-[5-[2-[4-(pentafluoro-λ⁶-sulfanyl)anilino]-3-pyridyl]-1,3,4-oxadiazol-2-yl]pyrrolidin-2-one (Compound 35) and (3S)-3-methyl-3-[5-[2-[4-(pentafluoro-λ⁶-sulfanyl)anilino]-3-pyridyl]-1,3,4-oxadiazol-2-yl]pyrrolidin-2-one (Compound 36)

3-methyl-3-[5-[2-[4-(pentafluoro-λ⁶-sulfanyl)anilino]-3-pyridyl]-1,3,4-oxadiazol-2-yl]pyrrolidin-2-one (90 mg, 195.06 umol, 1 eq) was separated by SFC (column: DAICEL CHIRALPAK AD (250 mm*30 mm, 10 um); mobile phase: [0.1% NH₃H₂O IPA]; B %: 35%-35%, min) to give the title compounds. Compound 35 (36.7 mg, 78.5 umol, 40.2% yield). LCMS (ESI): RT=0.946 min, mass calc. for C₁₈H₁₆F₅N₅O₂S 461.09 found 462.0 [M+H]⁺; ¹H NMR (400 MHz, DMSO-d6) δ 10.27 (s, 1H), 8.49 (br d, J=3.5 Hz, 1H), 8.29 (br d, J=7.3 Hz, 1H), 8.22 (br s, 1H), 7.98 (br d, J=8.5 Hz, 2H), 7.87 (br d, J=9.0 Hz, 2H), 7.23-7.07 (m, 1H), 3.43 (br d, J=5.5 Hz, 2H), 2.85-2.71 (m, 1H), 2.32-2.18 (m, 1H), 1.63 (s, 3H). Compound 36 (33.1 mg, 71.0 umol, 36.4% yield). LCMS (ESI): RT=0.946 min, mass calc. for C₁₈H₁₆F₅N₅O₂S 461.09 found 462.0 [M+H]⁺; ¹H NMR (400 MHz, DMSO-d6) δ 10.27 (s, 1H), 8.49 (br d, J=3.3 Hz, 1H), 8.30 (br d, J=7.8 Hz, 1H), 8.22 (s, 1H), 7.99 (br d, J=8.8 Hz, 2H), 7.87 (br d, J=9.3 Hz, 2H), 7.15 (dd, J=4.9, 7.7 Hz, 1H), 3.49-3.40 (m, 3H), 2.86-2.72 (m, 1H), 2.31-2.18 (m, 1H), 1.63 (s, 3H).

### Example 23: 2-methyl-2-(5-(2-((4-(trifluoromethyl)phenyl)amino)pyridin-3-yl)-1,3,4-oxadiazol-2-yl)morpholin-3-one (Compound 37)

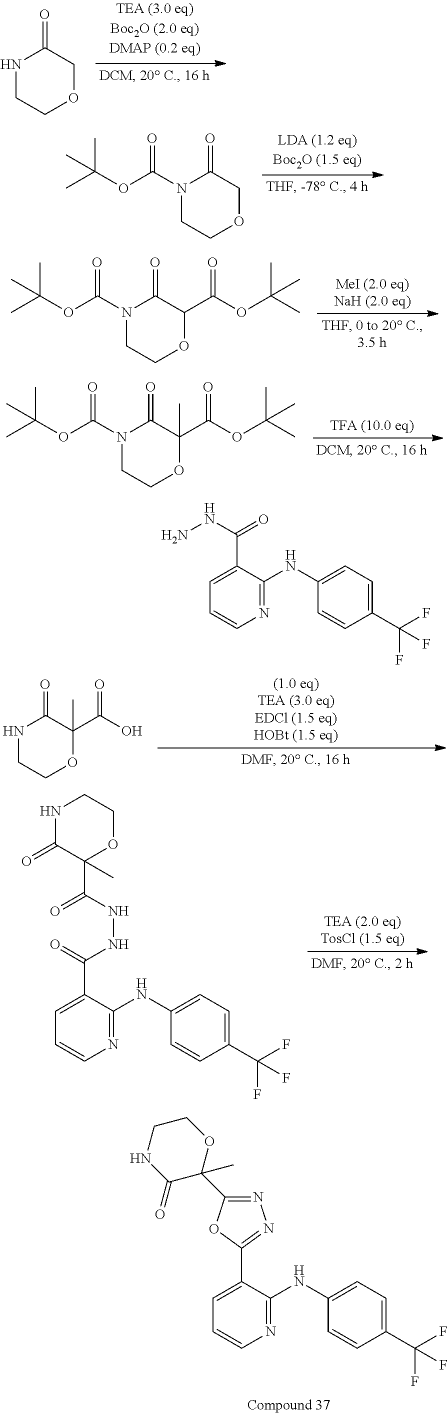

### tert-butyl 3-oxomorpholine-4-carboxylate

To a solution of morpholin-3-one (6.4 g, 63.30 mmol, 1.0 eq) in DCM (100 mL) at 20° C. was added TEA (19.22 g, 189.90 mmol, 26.4 mL, 3.0 eq), DMAP (1.55 g, 12.66 mmol, 0.2 eq) and then Boc₂O (27.63 g, 126.60 mmol, 29.1 mL, 2.0 eq), and the resulting mixture was stirred at 20° C. for 16 h. The reaction mixture was concentrated to remove the solvent and the residue was diluted with water (200 mL) and extracted with EA (100 mL*3). The combined organic layers were washed with water (50 mL*3) and then brine (100 mL), dried over anhydrous Na2SO4, filtered and concentrated under reduced pressure to give a residue. The residue was purified by flash silica gel chromatography to give tert-butyl 3-oxomorpholine-4-carboxylate (12.0 g, 59.64 mmol, 94.21% yield) as yellow oil. ¹H NMR (400 MHz, CDCl₃) δ 4.23 (s, 2H), 3.93-3.87 (m, 2H), 3.79-3.72 (m, 2H), 1.55 (s, 9H).

### di-tert-butyl 3-oxomorpholine-2,4-dicarboxylate

To a solution of tert-butyl 3-oxomorpholine-4-carboxylate (200.0 mg, 1.0 mmol, 1.0 eq) in THF (2 mL) at −78° C. was added LDA (2 M, 0.60 mL, 1.2 eq), and the mixture was stirred at −78° C. for 0.5 h. And then Boc₂O (325.4 mg, 1.49 mmol, 0.7 mL, 1.5 eq) was added into the above mixture at −78° C., and the resulting mixture was stirred at −78° C. for 3.5 h. The reaction mixture was quenched with saturated NH₄Cl solution (10 mL) at −78° C., then diluted with water (20 mL) and extracted with EA (20 mL*3). The combined organic layers were washed with brine (20 mL), dried over anhydrous Na₂SO₄, filtered and concentrated under reduced pressure to give a residue. The reaction mixture was diluted with water (20 mL) and extracted with EA (20 mL*3). The combined organic layers were washed with brine (20 mL), dried over Na₂SO₄, filtered and concentrated under reduced pressure to give a residue, which was purified by flash silica gel chromatography to give di-tert-butyl 3-oxomorpholine-2, 4-dicarboxylate (30 mg, 0.1 mmol, 10.02% yield) as colorless oil. ¹H NMR (400 MHz, CDCl₃) δ 4.66 (d, J=2.5 Hz, 1H), 4.30-4.21 (m, 1H), 3.96-3.88 (m, 1H), 3.86-3.74 (m, 2H), 1.54 (d, J=2.3 Hz, 9H), 1.51 (d, J=2.3 Hz, 9H).

### di-tert-butyl 2-methyl-3-oxomorpholine-2,4-dicarboxylate

To a solution of di-tert-butyl 3-oxomorpholine-2,4-dicarboxylate (550.0 mg, 1.83 mmol, 1.0 eq) in THF (5 mL) at 0° C. was added NaH (146.0 mg, 3.65 mmol, 60%, 2.0 eq) and the mixture was stirred at 0° C. for 0.5 h. And then MeI (518.1 mg, 3.65 mmol, 0.23 mL, 2.0 eq) was added into the above mixture at 0° C. The resulting mixture was stirred at 20° C. for 3 h. The reaction mixture was quenched with water (20 mL) at 0° C., then diluted with water (20 mL) and extracted with EA (20 mL*3). The combined organic layers were washed with brine (20 mL), dried over anhydrous Na₂SO₄, filtered and concentrated under reduced pressure to give a residue, which was purified by flash silica gel chromatography to give di-tert-butyl 2-methyl-3-oxomorpholine-2,4-dicarboxylate (300.0 mg, 0.95 mmol, 52.12% yield) as colorless oil. ¹H NMR (400 MHz, CDCl₃) δ 4.17 (ddd, J=4.0, 8.0, 12.0 Hz, 1H), 3.95 (td, J=4.4, 12.0 Hz, 1H), 3.86-3.74 (m, 2H), 1.62 (s, 3H), 1.55 (s, 9H), 1.50 (s, 9H).

### 2-methyl-3-oxomorpholine-2-carboxylic acid

To a solution of di-tert-butyl 2-methyl-3-oxomorpholine-2,4-dicarboxylate (50.0 mg, 0.16 mmol, 1.0 eq) in DCM (1 mL) at 20° C. was added TFA (180.8 mg, 1.59 mmol, 0.1 mL, 10.0 eq), and the mixture was stirred at 20° C. for 16 h. The reaction mixture was concentrated under reduced pressure to give 2-methyl-3-oxomorpholine-2-carboxylic acid (25.0 mg, 0.16 mmol, 99.08% yield) as a yellow solid. ¹H NMR (400 MHz, DMSO-d6) δ 13.02 (brs, 1H), 8.15 (brs, 1H), 3.94 (ddd, J=3.5, 8.7, 11.9 Hz, 1H), 3.80 (td, J=3.8, 11.7 Hz, 1H), 3.32-3.27 (m, 1H), 3.23-3.17 (m, 1H), 1.41 (s, 3H).

### 2-methyl-3-oxo-N′-(2-((4-(trifluoromethyl)phenyl)amino)nicotinoyl)morpholine-2-carbohydrazide

To a solution of 2-methyl-3-oxomorpholine-2-carboxylic acid (50.0 mg, 0.31 mmol, 1.0 eq), EDCI (90.3 mg, 0.47 mmol, 1.5 eq) and HOBt (63.4 mg, 0.47 mmol, 1.5 eq) in DMF (1 mL) at 20° C. were added 2-((4-(trifluoromethyl)phenyl)amino)nicotinohydrazide (93.1 mg, 0.31 mmol, 1.0 eq) and TEA (95.4 mg, 0.94 mmol, 0.1 mL, 3.0 eq). The mixture was stirred at 20° C. for 16 h. The reaction mixture was diluted with water (20 mL) and extracted with EA (20 mL*3). The combined organic layers were washed with brine (20 mL), dried over Na₂SO₄, filtered and concentrated under reduced pressure to give 2-methyl-3-oxo-N′-(2-((4-(trifluoromethyl)phenyl)amino)nicotinoyl)morpholine-2-carbohydrazide (110.0 mg, 0.25 mmol, 80.0% yield) as a yellow solid. LCMS (ESI): RT=0.809 min, mass calc. for C₁₉H₁₈F₃N₅O₄ 437.13, m/z found 438.1 [M+H]⁺.

### 2-methyl-2-(5-(2-((4-(trifluoromethyl)phenyl)amino)pyridin-3-yl)-1,3,4-oxadiazol-2-yl)morpholin-3-one (Compound 37)

To a solution of 2-methyl-3-oxo-N′-(2-((4-(trifluoromethyl)phenyl)amino)nicotinoyl)morpholine-2-carbohydrazide (100.0 mg, 0.23 mmol, 1.0 eq) in DCM (1 mL) at 20° C. was added TEA (69.4 mg, 0.69 mmol, 95 uL, 3.0 eq) and TosCl (65.4 mg, 0.34 mmol, 1.5 eq). The mixture was stirred at 20° C. for 2 h. The reaction mixture was concentrated under reduced pressure to give a residue, which was purified by prep-HPLC: (column: 3_Phenomenex Luna C18 75*30 mm*3 um; mobile phase: [water (0.05% HCl)-ACN]; B %: 36%-66%, 8.5 min) and prep-TLC (PE/EA=0:1, UV) to give the title compound (10.8 mg, 26 umol, 11.2% yield) as a white solid. LCMS (ESI): RT=0.928 min, mass calc. for C₁₉H₁₆F₃N₅O₃ 419.12, m/z found 420.1 [M+H]⁺; ¹H NMR (400 MHz, DMSO-d₆) δ 10.20 (s, 1H), 8.60 (brd, J=2.5 Hz, 1H), 8.51 (dd, J=1.8, 4.8 Hz, 1H), 8.33 (dd, J=1.8, 7.8 Hz, 1H), 8.00 (d, J=8.5 Hz, 2H), 7.71 (d, J=8.8 Hz, 2H), 7.14 (dd, J=4.8, 7.8 Hz, 1H), 4.03-3.92 (m, 2H), 3.51-3.41 (m, 1H), 3.33-3.27 (m, 1H), 1.88 (s, 3H).

### Example 24: 2-amino-4-methyl-4-[5-[2-[4-(trifluoromethyl)anilino]-3-pyridyl]-1,3,4-oxadiazol-2-yl]-3H-pyrrol-5-one (Compound 38)

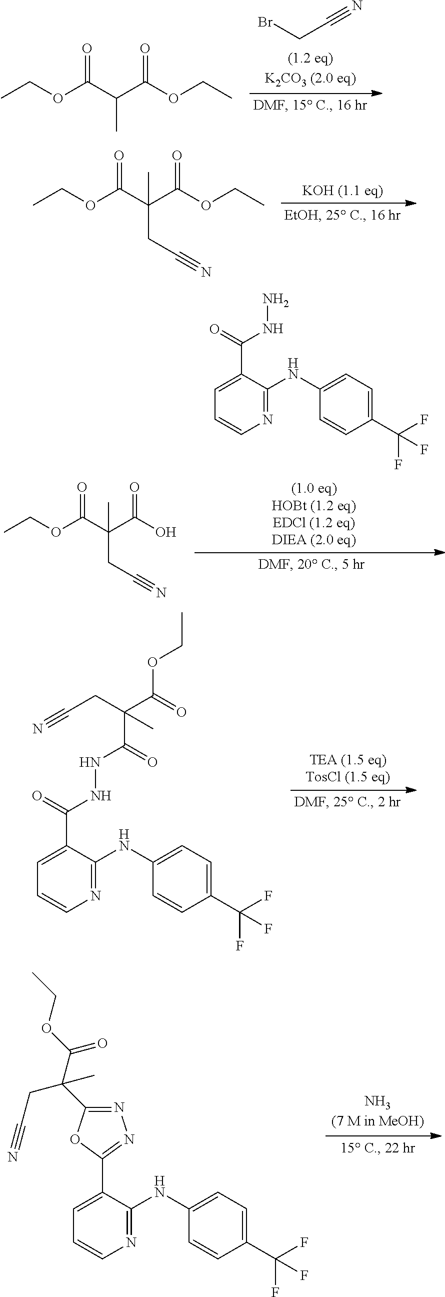

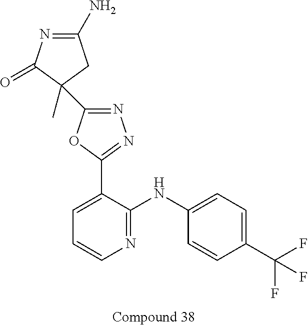 [SMILES: CC1(c2nnc(-c3cccnc3Nc3ccc(C(F)(F)F)cc3)o2)CC(N)=NC1=O]

### diethyl 2-(cyanomethyl)-2-methyl-propanedioate

To a solution of compound diethyl 2-methylmalonate (5 g, 28.70 mmol, 4.9 mL, 1 eq) in DMF (50 mL) were added compound 2-bromoacetonitrile (4.13 g, 34.44 mmol, 2.3 mL, 1.2 eq) and K₂CO₃ (7.93 g, 57.41 mmol, 2 eq). The mixture was stirred at 15° C. for 24 hr. The reaction mixture was filtered and the filtrate was concentrated in vacuum. The residue was diluted with EA (150 mL), washed with H₂O (20 mL), brine (20 mL) and dried over Na₂SO₄, filtered and concentrated in vacuum. The residue was purified by flash silica gel chromatography. HNMR showed that compound diethyl 2-(cyanomethyl)-2-methyl-propanedioate (1.5 g, 7.03 mmol, 24.5% yield) was obtained as colorless oil.

### 2-(cyanomethyl)-3-ethoxy-2-methyl-3-oxo-propanoic acid

To a solution of diethyl 2-(cyanomethyl)-2-methyl-propanedioate (1.5 g, 7.03 mmol, 1 eq) in EtOH (15 mL) was added KOH (434.1 mg, 7.74 mmol, 1.1 eq) in EtOH (2 mL). The mixture was stirred at 25° C. for 16 hr. The reaction mixture was diluted with saturated aq.NaHCO₃ (3 mL), extracted with EA (10 mL). Then the aqueous phase was adjusted pH=3 with 1M.aq.HCl, extracted with EA (15 mL*3). The combined organic phase was washed with H₂O (10 mL), brine (10 mL) and dried over Na₂SO₄, filtered and concentrated in vacuum. The crude product was used for the next step directly. H NMR showed that compound 2-(cyanomethyl)-3-ethoxy-2-methyl-3-oxo-propanoic acid (0.85 g, 4.59 mmol, 65.2% yield) was obtained as red oil.

### ethyl 2-(cyanomethyl)-2-methyl-3-oxo-3-[2-[2-[4-(trifluoromethyl)anilino] pyridine-3-carbonyl]hydrazino]propanoate

To a solution of compound 2-(cyanomethyl)-3-ethoxy-2-methyl-3-oxo-propanoic acid (500 mg, 2.70 mmol, 1 eq) and compound 2-((4-(trifluoromethyl)phenyl)amino)nicotinohydrazide (799.9 mg, 2.70 mmol, 1 eq) in DMF (8 mL) were added EDCI (621.1 mg, 3.24 mmol, 1.2 eq), HOBt (437.8 mg, 3.24 mmol, 1.2 eq) and DIEA (697.9 mg, 5.40 mmol, 0.94 mL, 2 eq). The mixture was stirred at 25° C. for 5 hr. The reaction mixture was quenched with H₂O (10 mL), extracted with EA (15 mL*3). The combined organic phase was washed with H₂O (10 mL*3) and brine (10 mL*2), dried over Na₂SO₄, filtered and concentrated in vacuum. The residue was purified by flash silica gel chromatography. Compound ethyl 2-(cyanomethyl)-2-methyl-3-oxo-3-[2-[2-[4-(trifluoromethyl)anilino] pyridine-3-carbonyl]hydrazino]propanoate (200 mg, 0.43 mmol, 15.9% yield) was obtained as yellow oil.

### Ethyl 3-cyano-2-methyl-2-[5-[2-[4-(trifluoromethyl)anilino]-3-pyridyl]-1,3,4-oxadiazol-2-yl]propanoate

To a solution of compound ethyl 2-(cyanomethyl)-2-methyl-3-oxo-3-[2-[2-[4-(trifluoromethyl)anilino] pyridine-3-carbonyl]hydrazino]propanoate (200 mg, 0.43 mmol, 1 eq) in DCM (3 mL) were added TosCl (123 mg, 0.64 mmol, 1.5 eq) and TEA (65.5 mg, 0.64 mmol, 90 uL, 1.5 eq). The mixture was stirred at 25° C. for 2 hr. The reaction mixture was concentrated in vacuum. The residue was purified by flash silica gel chromatography to give ethyl 3-cyano-2-methyl-2-[5-[2-[4-(trifluoromethyl)anilino]-3-pyridyl]-1,3,4-oxadiazol-2-yl]propanoate (75 mg, 0.12 mmol, 28.4% yield) as yellow oil.

### 2-amino-4-methyl-4-[5-[2-[4-(trifluoromethyl)anilino]-3-pyridyl]-1,3,4-oxadiazol-2-yl]-3H-pyrrol-5-one (Compound 38)

A solution of ethyl 3-cyano-2-methyl-2-[5-[2-[4-(trifluoromethyl)anilino]-3-pyridyl]-1,3,4-oxadiazol-2-yl]propanoate (35 mg, 78.5 umol, 1 eq) in ammonia (7 M, 1.1 mL) was stirred at 15° C. for 22 hr. The reaction mixture was concentrated in vacuum. The residue was purified by prep-HPLC (column: Waters Xbridge 150*25 mm*5 um; mobile phase: [water (0.05% NH₃H₂O+10 mM NH4HCO3)-ACN]; B %: 35%-65%, 9.5 min). to give the title compound (15.6 mg, 36.8 umol, 46.8% yield) as a white solid. LCMS (ESI): RT=0.787 min, mass calcd. For C₁₉H₁₅F₃N₆O₂, 416.12 m/z found 417.0 [M+H]⁺. ¹H NMR (400 MHz, DMSO-d₆) δ 10.22 (s, 1H), 8.94 (br s, 1H), 8.67 (br s, 1H), 8.48 (dd, J=1.8, 4.8 Hz, 1H), 8.25 (dd, J=1.5, 7.8 Hz, 1H), 8.00 (br d, J=8.5 Hz, 2H), 7.71 (br d, J=8.5 Hz, 2H), 7.12 (dd, J=4.9, 7.7 Hz, 1H), 3.53 (d, J=17.6 Hz, 1H), 2.99 (d, J=17.8 Hz, 1H), 1.68 (s, 3H).

### Example 25: (S)-3-methyl-3-(5-(2-((4-(trifluoromethyl)phenyl)amino)pyridin-3-yl)-1,3,4-oxadiazol-2-yl)piperidin-2-one (Compound 39) and (R)-3-methyl-3-(5-(2-((4-(trifluoromethyl)phenyl)amino)pyridin-3-yl)-1,3,4-oxadiazol-2-yl)piperidin-2-one (Compound 40)

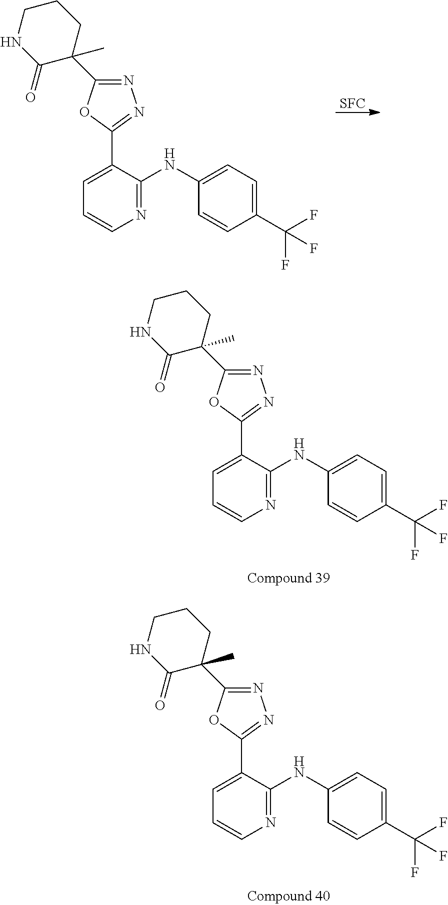 [SMILES: CC1(c2nnc(-c3cccnc3Nc3ccc(C(F)(F)F)cc3)o2)CCCNC1=O ; "SFC" ; C[C@@]1(c2nnc(-c3cccnc3Nc3ccc(C(F)(F)F)cc3)o2)CCCNC1=O ; C[C@]1(c2nnc(-c3cccnc3Nc3ccc(C(F)(F)F)cc3)o2)CCCNC1=O]

3-Methyl-3-(5-(2-((4-(trifluoromethyl)phenyl)amino)pyridin-3-yl)-1,3,4-oxadiazol-2-yl)piperidin-2-one was separated by SFC: (column: DAICEL CHIRALPAK AD (250 mm*30 mm, 10 um); mobile phase: [0.1% NH₃H₂O ETOH]; B %: 50%-50%, min) to give Compound 39 (14.35 mg, 34 umol, 28.47% yield) as a white solid and Compound 40 (19.55 mg, 46 umol, 38.67% yield) as a white solid. Compound 39: LCMS (ESI): RT=0.941 min, mass calc. for C₂₀H₁₈F₃N₅O₂ 417.14, m/z found 418.1 [M+H]⁺; ¹H NMR (400 MHz, CD₃OD) δ 8.44 (brs, 1H), 8.31 (brd, J=6.5 Hz, 1H), 8.02 (brd, J=8.8 Hz, 2H), 7.62 (brd, J=8.8 Hz, 2H), 7.05 (brs, 1H), 3.44 (brs, 1H), 2.52 (brd, J=5.5 Hz, 1H), 2.14-1.94 (m, 4H), 1.84 (s, 3H). Compound 40: LCMS (ESI): RT=0.934 min, mass calc. for C₂₀H₁₈F₃N₅O₂ 417.14, m/z found 418.1 [M+H]⁺; ¹H NMR (400 MHz, DMSO-d₆) δ 10.26 (brs, 1H), 8.49 (brs, 1H), 8.29 (brd, J=7.3 Hz, 1H), 8.00 (brd, J=7.3 Hz, 2H), 7.93 (brs, 1H), 7.71 (brd, J=7.8 Hz, 2H), 7.13 (brs, 1H), 3.23-3.20 (m, 1H), 2.39-2.37 (m, 1H), 2.01-1.81 (m, 4H), 1.71 (brs, 3H).

### Example 26: 3-methyl-3-(5-(2-((4-(trifluoromethyl)phenyl)amino)phenyl)-1,3,4-oxadiazol-2-yl)piperidin-2-one (Compound 41)

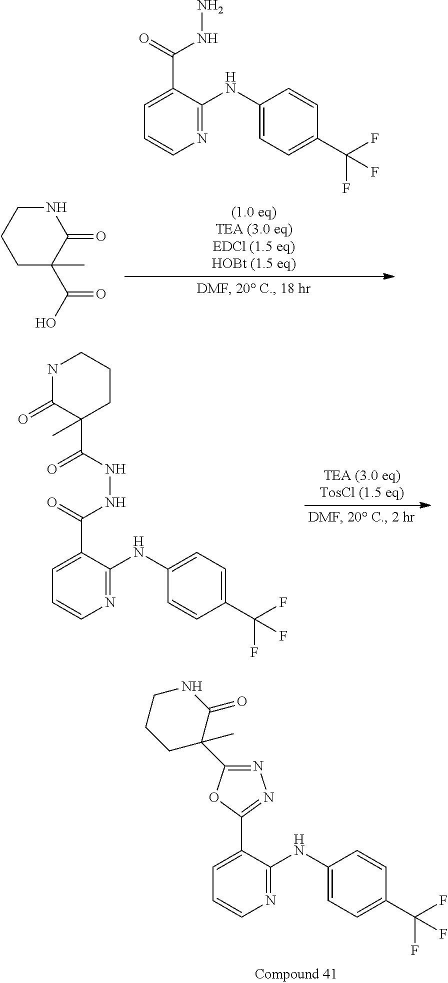

### 3-methyl-2-oxo-N′-(2-((4-(trifluoromethyl)phenyl)amino)benzoyl)piperidine-3-carbohydrazide

To a solution of 3-methyl-2-oxopiperidine-3-carboxylic acid (60.0 mg, 0.38 mmol, 1.0 eq), EDCI (109.8 mg, 0.57 mmol, 1.5 eq) and HOBt (77.4 mg, 0.57 mmol, 1.5 eq) in DMF (2 mL) at 20° C. were added 2-((4-(trifluoromethyl)phenyl)amino)nicotinohydrazide (113.1 mg, 0.38 mmol, 1.0 eq) and TEA (115.9 mg, 1.15 mmol, 0.16 mL, 3.0 eq). The mixture was stirred at 20° C. for 16 h. The reaction mixture was diluted with water (20 mL) and extracted with EA (20 mL*3). The combined organic layers were washed with brine (20 mL), dried over Na₂SO₄, filtered and concentrated under reduced pressure to give compound 3-methyl-2-oxo-N-(2-((4-(trifluoromethyl)phenyl)amino)benzoyl)piperidine-3-carbohydrazide (160.0 mg, 0.24 mmol, 62.6% yield) as a yellow solid. LCMS (ESI): RT=0.867 min, mass calc. for C₂₀H₂₀F₃N₅O₃ 435.15, m/z found 436.2 [M+H]⁺.

### 3-methyl-3-(5-(2-((4-(trifluoromethyl)phenyl)amino)phenyl)-1,3,4-oxadiazol-2-yl)piperidin-2-one (Compound 41)

To a solution of 3-methyl-2-oxo-N′-(2-((4-(trifluoromethyl)phenyl)amino)benzoyl)piperidine-3-carbohydrazide (60.0 mg, 0.14 mmol, 1.0 eq) in DCM (1 mL) at 30° C. were added TEA (41.8 mg, 0.41 mmol, 58 uL, 3.0 eq) and TosCl (39.41 mg, 206.71 umol, 1.5 eq). The mixture was stirred at 20° C. for 2 h. The residue was concentrated under reduced pressure to give a residue, which was purified by prep-HPLC: (column: 3_Phenomenex Luna C18 75*30 mm*3 um; mobile phase: [water (0.05% HCl)-ACN]; B %: 45%-75%, 6.5 min) to give the title compound (10.6 mg, 25 umol, 18.4% yield) as a white solid. LCMS (ESI): RT=0.939 min, mass calc. for C₂₀H₁₈F₃N₅O₂ 417.14, m/z found 418.1 [M+H]⁺; ¹H NMR (400 MHz, DMSO-d₆) δ 10.26 (s, 1H), 8.49 (brd, J=3.5 Hz, 1H), 8.29 (brd, J=6.1 Hz, 1H), 8.00 (brd, J=8.5 Hz, 2H), 7.92 (brs, 1H), 7.71 (brd, J=8.5 Hz, 2H), 7.13 (dd, J=4.8, 7.7 Hz, 1H), 3.30-3.24 (m, 1H), 2.40 (brd, J=6.3 Hz, 1H), 2.01-1.82 (m, 4H), 1.71 (s, 3H).

### Example 27: (3S)-3-methyl-3-[5-[2-[4-(trifluoromethyl)anilino]-3-pyridyl]tetrazol-2-yl]pyrrolidin-2-one (Compound 42) and (3R)-3-methyl-3-[5-[2-[4-(trifluoromethyl)anilino]-3-pyridyl]tetrazol-2-yl]pyrrolidin-2-one (Compound 43)

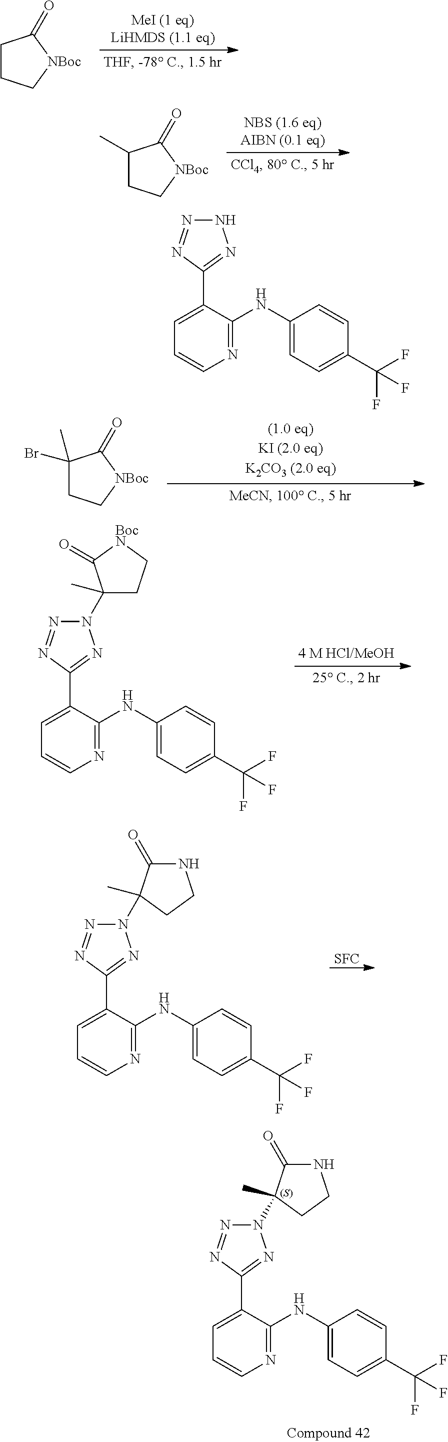

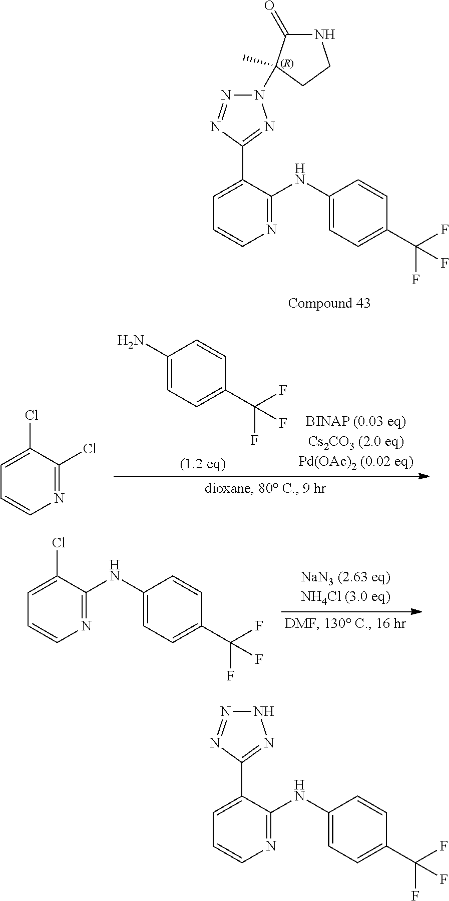

### 2-[4-(trifluoromethyl)anilino]pyridine-3-carbonitrile

To a solution of compound 2-chloronicotinonitrile (5 g, 36.09 mmol, 1 eq) and compound 4-(trifluoromethyl)aniline (6.98 g, 43.30 mmol, 5.37 mL, 1.2 eq) in dioxane (70 mL) were added Pd(OAc)₂ (162.0 mg, 0.72 mmol, 0.02 eq), BINAP (674.1 mg, 1.08 mmol, 0.03 eq) and CS₂CO₃ (23.52 g, 72.17 mmol, 2 eq). The mixture was degassed and purged with N₂ for 3 times and stirred at 80° C. for 3 hr. Then the mixture was stirred at 80° C. for 6 hr. The reaction mixture was filtered and the filtrate was concentrated in vacuum. The residue was triturated with PE/EA=3/1 to give 2-[4-(trifluoromethyl)anilino]pyridine-3-carbonitrile (4.5 g, 15.22 mmol, 42.1% yield) as a yellow solid. LCMS (ESI): RT=0.924 min, mass calcd. For C₁₃H₈F₃N₃, 263.07 m/z found 264.2 [M+H]⁺. ¹H NMR (400 MHz, CDCl₃) δ 8.45 (dd, J=1.8, 5.0 Hz, 1H), 7.85 (dd, J=1.9, 7.7 Hz, 1H), 7.78 (d, J=8.5 Hz, 2H), 7.62 (d, J=8.5 Hz, 2H), 7.19 (br s, 1H), 6.91 (dd, J=4.9, 7.7 Hz, 1H).

### 3-(2H-tetrazol-5-yl)-N-[4-(trifluoromethyl)phenyl]pyridin-2-amine

To a solution of compound 2-[4-(trifluoromethyl)anilino]pyridine-3-carbonitrile (2 g, 7.60 mmol, 1 eq) and NaN₃ (1.3 g, 20.00 mmol, 2.63 eq) in DMF (20 mL) was added NH₄Cl (1.22 g, 22.79 mmol, 3 eq). The mixture was degassed and purged with N₂ for 3 times and stirred at 130° C. for 16 hr. The reaction mixture was poured into 1M.aq.HCl (100 mL), the solid was collected. The crude product was used for the next step directly. 3-(2H-tetrazol-5-yl)-N-[4-(trifluoromethyl)phenyl]pyridin-2-amine (1.6 g, 5.22 mmol, 68.7% yield) was obtained as a yellow solid. ¹H NMR (400 MHz, DMSO-d₆) δ 10.49 (s, 1H), 8.48-8.39 (m, 2H), 8.00 (d, J=8.6 Hz, 2H), 7.69 (d, J=8.6 Hz, 2H), 7.15 (dd, J=4.9, 7.6 Hz, 1H).

### tert-butyl 3-methyl-2-oxo-pyrrolidine-1-carboxylate

To a solution of tert-butyl 2-oxopyrrolidine-1-carboxylate (3 g, 16.20 mmol, 2.75 mL, 1 eq) in THF (30 mL) was added LiHMDS (1 M, 17.82 mL, 1.1 eq) at −78° C. and the mixture was stirred at −78° C. for 0.5 hr. Then MeI (2.30 g, 16.20 mmol, 1.01 mL, 1 eq) was added to the mixture and stirred at −78° C. for 1 hr. The reaction mixture was quenched with NH₄Cl (20 mL), extracted with EA (15 mL*3). The combined organic phase was washed whit H₂O (10 mL), brine (10 mL) and dried over Na₂SO₄, filtered and concentrated in vacuum. The residue was purified by flash silica gel chromatography. Tert-butyl 3-methyl-2-oxo-pyrrolidine-1-carboxylate (750 mg, 3.76 mmol, 23.2% yield) was obtained as yellow oil. ¹H NMR (400 MHz, CDCl₃) δ 3.76 (ddd, J=2.8, 8.6, 11.0 Hz, 1H), 3.57 (ddd, J=7.3, 9.5, 10.8 Hz, 1H), 2.63-2.48 (m, 1H), 2.20 (dddd, J=2.6, 7.2, 8.4, 12.6 Hz, 1H), 1.68-1.55 (m, 1H), 1.52 (s, 9H), 1.22 (d, J=7.0 Hz, 3H).

### tert-butyl 3-bromo-3-methyl-2-oxo-pyrrolidine-1-carboxylate

To a solution of tert-butyl 3-methyl-2-oxo-pyrrolidine-1-carboxylate (750 mg, 3.76 mmol, 1 eq) and NBS (1.07 g, 6.02 mmol, 1.6 eq) in CCl₄ (15 mL) was added AIBN (61.8 mg, 0.37 mmol, 0.1 eq). The mixture was heated to 80° C. and stirred for 5 hr. The reaction mixture was filtered and the filtrate was concentrated in vacuum. The residue was purified by flash silica gel chromatography. Tert-butyl 3-bromo-3-methyl-2-oxo-pyrrolidine-1-carboxylate (550 mg, 1.98 mmol, 52.5% yield) was obtained as a yellow solid. ¹H NMR (400 MHz, CDCl₃) δ 3.84-3.68 (m, 2H), 2.49 (dd, J=5.0, 14.3 Hz, 1H), 2.11 (ddd, J=8.4, 9.7, 14.5 Hz, 1H), 1.92 (s, 3H), 1.55 (s, 9H).

### tert-butyl 3-methyl-2-oxo-3-[5-[2-[4-(trifluoromethyl)anilino]-3-pyridyl]tetrazol-2-yl]pyrrolidine-1-carboxylate

To a solution of compound 3-(2H-tetrazol-5-yl)-N-(4-(trifluoromethyl)phenyl)pyridin-2-amine (247.7 mg, 0.80 mmol, 1 eq) in MeCN (9 mL) were added K₂CO₃ (223.6 mg, 1.62 mmol, 2 eq), KI (268.5 mg, 1.62 mmol, 2 eq) and tert-butyl 3-bromo-3-methyl-2-oxo-pyrrolidine-1-carboxylate (450 mg, 1.62 mmol, 2 eq). The mixture was heated to 100° C. and stirred for 5 hr. The reaction mixture was quenched with H₂O (10 mL), extracted with EA (15 mL*3). The combined organic phase was washed with H₂O (10 ml) and brine (10 mL*2), dried over Na₂SO₄, filtered and concentrated in vacuum. The residue was purified by flash silica gel chromatography to give tert-butyl 3-methyl-2-oxo-3-[5-[2-[4-(trifluoromethyl)anilino]-3-pyridyl]tetrazol-2-yl]pyrrolidine-1-carboxylate (85 mg, 67.5 umol, 8.3% yield) as a white solid. LCMS (ESI): RT=1.024 min, mass calcd. For C₂₃H₂₄F₃N₇O₃, 503.19 m/z found 504.2 [M+H]⁺.

### 3-methyl-3-[5-[2-[4-(trifluoromethyl)anilino]-3-pyridyl]tetrazol-2-yl]pyrrolidin-2-one

A solution of compound tert-butyl 3-methyl-2-oxo-3-[5-[2-[4-(trifluoromethyl)anilino]-3-pyridyl]tetrazol-2-yl]pyrrolidine-1-carboxylate (75 mg, 0.14 mmol, 1 eq) in HCl/MeOH (4 M, 4 mL) was stirred at 25° C. for 2 hr. The reaction mixture was concentrated in vacuum. The crude product was purified by prep-HPLC (column: 3_Phenomenex Luna C18 75*30 mm*3 um; mobile phase: [water (0.05% HCl)-ACN]; B %: 50%-80%, 6.5 min). 3-methyl-3-[5-[2-[4-(trifluoromethyl)anilino]-3-pyridyl]tetrazol-2-yl]pyrrolidin-2-one (20 mg, 49.5 umol, 33.2% yield) was obtained as yellow solid. LCMS (ESI): RT=0.877 min, mass calcd. For C₁₈H₁₆F₃N₇O, 403.14 m/z found 404.0 [M+H]⁺. ¹H NMR (400 MHz, CD₃OD) δ 8.94 (dd, J=1.6, 7.7 Hz, 1H), 8.15 (dd, J=1.6, 5.9 Hz, 1H), 7.90-7.84 (m, 2H), 7.83-7.76 (m, 2H), 7.30 (dd, J=6.0, 7.5 Hz, 1H), 3.73-3.64 (m, 1H), 3.64-3.55 (m, 1H), 3.13 (ddd, J=6.3, 8.0, 13.8 Hz, 1H), 2.68 (ddd, J=4.4, 7.7, 13.6 Hz, 1H), 2.11 (s, 3H).

### (3S)-3-methyl-3-[5-[2-[4-(trifluoromethyl)anilino]-3-pyridyl]tetrazol-2-yl]pyrrolidin-2-one (Compound 42) and (3R)-3-methyl-3-[5-[2-[4-(trifluoromethyl)anilino]-3-pyridyl]tetrazol-2-yl]pyrrolidin-2-one (Compound 43)

The racemate (20 mg, 49.5 umol) was separated by chiral SFC (column: DAICEL CHIRALCEL OJ-H (250 mm*30 mm, 5 um); mobile phase: [0.1% NH₃H₂O ETOH]; B %: 20%-20%, min) to give the title compounds. Compound 42 (7.5 mg, 17.9 umol, 36.2% yield) was obtained as a white solid. LCMS (ESI): RT=0.880 min, mass calcd. For C₁₈H₁₆F₃N₇O, 403.14 m/z found 404.0 [M+H]⁺. ¹H NMR (400 MHz, CD₃OD) δ 8.52 (dd, J=1.9, 7.8 Hz, 1H), 8.37 (dd, J=1.9, 4.8 Hz, 1H), 7.96 (d, J=8.5 Hz, 2H), 7.59 (d, J=8.5 Hz, 2H), 7.01 (dd, J=4.9, 7.8 Hz, 1H), 3.73-3.65 (m, 1H), 3.62-3.55 (m, 1H), 3.07 (ddd, J=5.8, 8.2, 13.8 Hz, 1H), 2.72-2.61 (m, 1H), 2.11 (s, 3H), 1.18 (t, J=7.0 Hz, 1H). Compound 43 (9.3 mg, 22.5 umol, 45.4% yield) was obtained as a white solid. LCMS (ESI): RT=0.880 min, mass calcd. For C₁₈H₁₆F₃N₇O, 403.14 m/z found 404.0 [M+H]⁺. ¹H NMR (400 MHz, CD₃OD) δ 8.60-8.47 (m, 1H), 8.39 (dd, J=1.9, 4.9 Hz, 1H), 7.98 (d, J=8.5 Hz, 2H), 7.60 (d, J=8.5 Hz, 2H), 7.04 (dd, J=4.9, 7.7 Hz, 1H), 3.73-3.65 (m, 1H), 3.63-3.55 (m, 1H), 3.08 (ddd, J=5.9, 8.2, 13.8 Hz, 1H), 2.67 (ddd, J=4.8, 7.8, 13.6 Hz, 1H), 2.12 (s, 3H).

### Example 28: ethyl-[[5-[3-[4-(trifluoromethyl)anilino]pyrazin-2-yl]-1,3,4-oxadiazol-2-yl]methyl]cyanamide (Compound 44) 3-[5-(ethylaminomethyl)-1,3,4-oxadiazol-2-yl]-N-[4-(trifluoromethyl)phenyl]pyrazin-2-amine (Compound 46) and tert-butyl N-ethyl-N-[[5-[3-[4-(trifluoromethyl)anilino]pyrazin-2-yl]-1,3,4-oxadiazol-2-yl]methyl]carbamate (Compound 47)

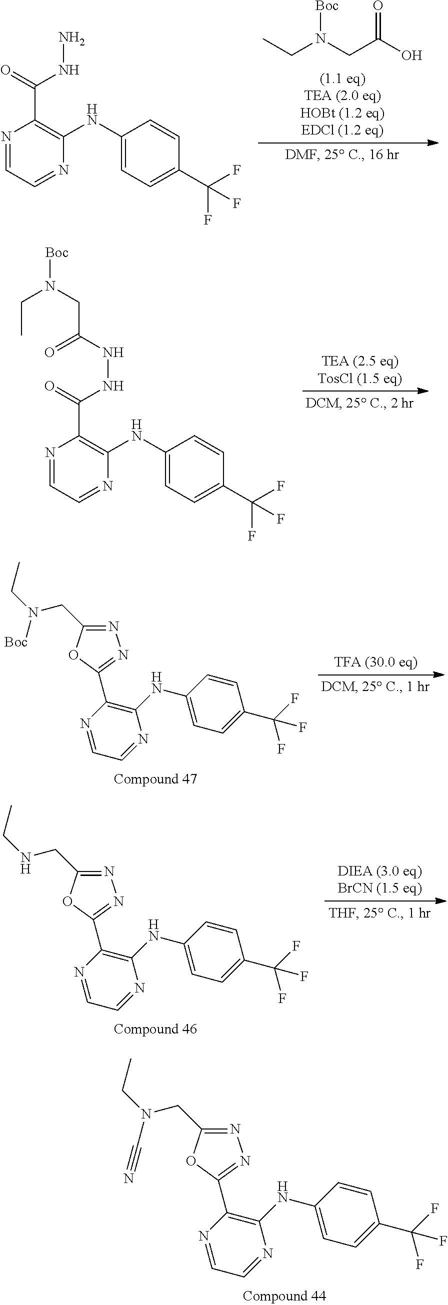 [SMILES: NNC(=O)c1nccnc1Nc1ccc(C(F)(F)F)cc1 ; CCN(CC(=O)O)C(=O)OC(C)(C)C ; CCN(CC(=O)NNC(=O)c1nccnc1Nc1ccc(C(F)(F)F)cc1)C(=O)OC(C)(C)C ; CCN(Cc1nnc(-c2nccnc2Nc2ccc(C(F)(F)F)cc2)o1)C(=O)OC(C)(C)C ; CCNCc1nnc(-c2nccnc2Nc2ccc(C(F)(F)F)cc2)o1 ; CCN(C#N)Cc1nnc(-c2nccnc2Nc2ccc(C(F)(F)F)cc2)o1]

### tert-butyl N-ethyl-N-[2-oxo-2-[2-[3-[4-(trifluoromethyl)anilino]pyrazine-2-carbonyl]hydrazino]ethyl]carbamate

To a solution of 3-((4-(trifluoromethyl)phenyl)amino)pyrazine-2-carbohydrazide (400 mg, 1.35 mmol, 1 eq) and N-(tert-butoxycarbonyl)-N-ethylglycine (301.8 mg, 1.49 mmol, 1.1 eq) DMF (4 mL) were added EDCI (310.6 mg, 1.62 mmol, 1.2 eq), HOBt (218.9 mg, 1.62 mmol, 1.2 eq) and TEA (273.2 mg, 2.70 mmol, 0.36 mL, 2 eq). The mixture was stirred at 25° C. for 16 h. The mixture was diluted with H₂O (15 mL), extracted with EA (30 mL*3). The combined organic layers were washed with brine (30 mL), dried over Na₂SO₄, filtered and concentrated under reduced pressure to give a residue. The residue was purified by flash silica gel chromatography to give tert-butyl N-ethyl-N-[2-oxo-2-[2-[3-[4-(trifluoromethyl)anilino]pyrazine-2-carbonyl]hydrazino]ethyl]carbamate (500 mg, 0.98 mmol, 72.9% yield) as a yellow solid. LCMS (ESI): RT=0.899 min, mass calc. for C₂₁H₂₅F₃N₆O₄ 482.19 found 383.0 [M-Boc+H]⁺.

### tert-butyl N-ethyl-N-[[5-[3-[4-(trifluoromethyl)anilino]pyrazin-2-yl]-1,3,4-oxadiazol-2-yl]methyl]carbamate (Compound 47)

To a solution of tert-butyl N-ethyl-N-[2-oxo-2-[2-[3-[4-(trifluoromethyl)anilino]pyrazine-2-carbonyl]hydrazino]ethyl]carbamate (500 mg, 0.98 mmol, 1 eq) in DCM (5 mL) were added TosCl (281.6 mg, 1.48 mmol, 1.5 eq) and TEA (249.1 mg, 2.46 mmol, 0.34 mL, 2.5 eq). The mixture was stirred at 25° C. for 2 h. The mixture was diluted with H₂O (15 mL), extracted with EA (30 mL*3). The combined organic layers were washed with brine (30 mL), dried over Na₂SO₄, filtered and concentrated under reduced pressure to give a residue. The residue was purified by flash silica gel chromatography to give the desired compound (280 mg, 0.58 mmol, 59.4% yield) as a white solid. LCMS (ESI): RT=0.990 min, mass calc. for C₂₁H₂₃F₃N₆O₃ 464.18 found 465.1 [M+H]⁺; ¹H NMR (400 MHUz, CDCl₃) δ 10.43 (br s, 1H), 8.38 (d, J=2.0 Hz, 1H), 8.22 (d, J=2.0 Hz, 1H), 7.93 (br d, J=8.3 Hz, 2H), 7.64 (d, J=8.5 Hz, 2H), 4.88-4.65 (m, 2H), 3.48 (br s, 2H), 1.49 (br s, 9H), 1.21 (t, J=7.0 Hz, 3H).

### 3-[5-(ethylaminomethyl)-1,3,4-oxadiazol-2-yl]-N-[4-(trifluoromethyl)phenyl]pyrazin-2-amine (Compound 46)

To a solution of tert-butyl N-ethyl-N-[[5-[3-[4-(trifluoromethyl)anilino]pyrazin-2-yl]-1,3,4-oxadiazol-2-yl]methyl]carbamate (250 mg, 0.54 mmol, 1 eq) in DCM (2.5 mL) was added TFA (1.84 g, 16.15 mmol, 1.20 mL, 30 eq). The mixture was stirred at 25° C. for 1 h. The mixture was concentrated under reduced pressure to give the desired compound (220 mg, crude, TFA) as a yellow solid. LCMS (ESI): RT=0.722 min, mass calc. for C₁₆H₁₅F₃N₆O 364.13 found 365.0 [M+H]⁺. (50 mg, 0.10 mmol, 1 eq, TFA) of the product was purified by prep-HPLC (column: Waters Xbridge 150*25 mm*5 um; mobile phase: [water (0.05% NH₃H₂O+10 mM NH₄HCO₃)-ACN]; B %: 46%-76%, 7.8 min) to give the desired compound (15.5 mg, 42 umol, 40.7% yield) as a white solid. LCMS (ESI): RT=0.725 min, mass calc. for C₁₆H₁₅F₃N₆O 364.13 found 365.0 [M+H]⁺; ¹H NMR (400 MHz, CDCl₃) δ 10.45 (s, 1H), 8.39 (d, J=2.3 Hz, 1H), 8.23 (d, J=2.5 Hz, 1H), 7.93 (d, J=8.5 Hz, 2H), 7.65 (d, J=8.8 Hz, 2H), 4.22 (s, 2H), 2.79 (q, J=7.0 Hz, 2H), 1.18 (t, J=7.2 Hz, 3H).

### ethyl-[[5-[3-[4-(trifluoromethyl)anilino]pyrazin-2-yl]-1,3,4-oxadiazol-2-yl]methyl]cyanamide (Compound 44)

To a solution of 3-[5-(ethylaminomethyl)-1,3,4-oxadiazol-2-yl]-N-[4-(trifluoromethyl)phenyl]pyrazin-2-amine (120 mg, 0.25 mmol, 1 eq, TFA) in THF (1.5 mL) were added DIEA (97.3 mg, 0.75 mmol, 0.13 mL, 3 eq) and BrCN (39.9 mg, 0.38 mmol, 28 uL, 1.5 eq). The mixture was stirred at 25° C. for 0.5 h. The mixture was quenched with H₂O (10 mL), extracted with EA (20 mL*3). The combined organic layers were washed with brine (15 mL), dried over Na₂SO₄, filtered and concentrated under reduced pressure to give a residue. The residue was purified by prep-HPLC (column: Waters Xbridge 150*25 mm*5 um; mobile phase: [water (0.05% NH₃H₂O+10 mM NH₄HCO₃)-ACN]; B %: 52%-82%, 7.8 min) to give the desired compound (35.6 mg, 88.6 umol, 35.3% yield) as a yellow solid. LCMS (ESI): RT=0.874 min, mass calc. for C₁₇H₁₄F₃N₇O 389.12 found 390.0 [M+H]⁺; ¹H NMR (400 MHz, CDCl₃) δ 10.29 (s, 1H), 8.40 (d, J=2.3 Hz, 1H), 8.24 (d, J=2.3 Hz, 1H), 7.91 (d, J=8.3 Hz, 2H), 7.64 (d, J=8.5 Hz, 2H), 4.62 (s, 2H), 3.29 (q, J=7.3 Hz, 2H), 1.39 (t, J=7.3 Hz, 3H).

### Example 29: methyl-[[5-[3-[4-(trifluoromethyl)anilino]pyrazin-2-yl]-1,3,4-oxadiazol-2-yl]methyl]cyanamide (Compound 45) 3-[5-(methylaminomethyl)-1,3,4-oxadiazol-2-yl]-N-[4-(trifluoromethyl)phenyl]pyrazin-2-amine (Compound 48) and tert-butyl N-methyl-N-[[5-[3-[4-(trifluoromethyl)anilino]pyrazin-2-yl]-1,3,4-oxadiazol-2-yl]methyl]carbamate (Compound 49)

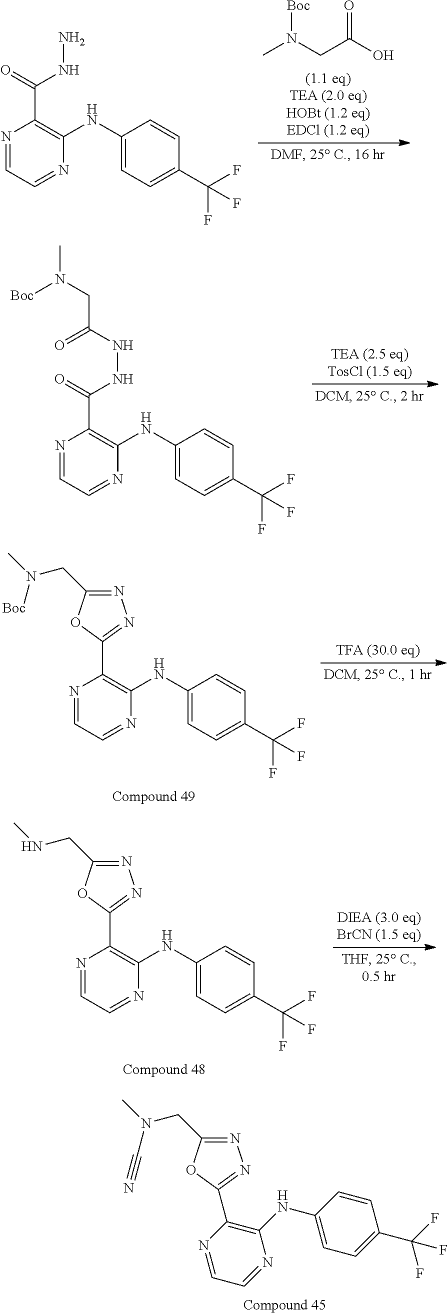 [SMILES: NNC(=O)c1nccnc1Nc1ccc(C(F)(F)F)cc1 ; CN(CC(=O)O)C(=O)OC(C)(C)C ; CN(CC(=O)NNC(=O)c1nccnc1Nc1ccc(C(F)(F)F)cc1)C(=O)OC(C)(C)C ; CN(Cc1nnc(-c2nccnc2Nc2ccc(C(F)(F)F)cc2)o1)C(=O)OC(C)(C)C ; CNCc1nnc(-c2nccnc2Nc2ccc(C(F)(F)F)cc2)o1 ; CN(C#N)Cc1nnc(-c2nccnc2Nc2ccc(C(F)(F)F)cc2)o1]

### tert-butyl N-methyl-N-[2-oxo-2-[2-[3-[4-(trifluoromethyl)anilino]pyrazine-2-carbonyl]hydrazino]ethyl]carbamate

To a solution of 3-((4-(trifluoromethyl)phenyl)amino)pyrazine-2-carbohydrazide (400 mg, 1.35 mmol, 1 eq) and N-(tert-butoxycarbonyl)-N-methyl glycine (280.1 mg, 1.48 mmol, 1.1 eq) DMF (4 mL) were added EDCI (309.6 mg, 1.61 mmol, 1.2 eq), HOBt (218.2 mg, 1.61 mmol, 1.2 eq) and TEA (272.4 mg, 2.69 mmol, 0.37 mL, 2 eq). The mixture was stirred at 25° C. for 16 h. The mixture was concentrated under reduced pressure to give a residue. The residue was diluted with H₂O (20 mL), extracted with EA (40 mL*3). The combined organic layers were washed with brine (30 mL), dried over Na₂SO₄, filtered and concentrated under reduced pressure to give a residue. The residue was purified by flash silica gel chromatography to give tert-butyl N-methyl-N-[2-oxo-2-[2-[3-[4-(trifluoromethyl)anilino]pyrazine-2-carbonyl]hydrazino]ethyl]carbamate (600 mg, 0.73 mol, 54.3% yield) as a yellow solid. LCMS (ESI): RT=0.873 min, mass calc. for C₂₀H₂₃F₃N₆O₄ 468.17 found 369.0 [M-Boc+H]⁺.

### tert-butyl N-methyl-N-[[5-[3-[4-(trifluoromethyl)anilino]pyrazin-2-yl]-1,3,4-oxadiazol-2-yl]methyl]carbamate (Compound 49)

To a solution of tert-butyl N-methyl-N-[2-oxo-2-[2-[3-[4-(trifluoromethyl)anilino]pyrazine-2-carbonyl]hydrazino]ethyl]carbamate (600 mg, 0.73 mmol, 1 eq) in DCM (5 mL) were added TosCl (208.8 mg, 1.10 mmol, 1.5 eq) and TEA (184.7 mg, 1.83 mmol, 0.25 mL, 2.5 eq). The mixture was stirred at 25° C. for 2 h. The mixture was diluted with H₂O (15 mL), extracted with EA (30 mL*3). The combined organic layers were washed with brine (30 mL), dried over Na₂SO₄, filtered and concentrated under reduced pressure to give a residue. The residue was purified by flash silica gel chromatography to give the desired compound (180 mg, 0.38 mmol, 52.4% yield) as a yellow solid. LCMS (ESI): RT=0.973 min, mass calc. for C₂₀H₂₁F₃N₆O₃ 450.16 found 451.1 [M+H]⁺; ¹H NMR (400 MHz, CDCl₃) δ 10.43 (br s, 1H), 8.38 (d, J=2.3 Hz, 1H), 8.22 (d, J=2.4 Hz, 1H), 7.93 (br d, J=8.4 Hz, 2H), 7.64 (d, J=8.6 Hz, 2H), 4.92-4.69 (m, 2H), 3.09 (s, 3H), 1.50 (br d, J=7.9 Hz, 9H).

### 3-[5-(methylaminomethyl)-1,3,4-oxadiazol-2-yl]-N-[4-(trifluoromethyl)phenyl]pyrazin-2-amine (Compound 48)

To a solution of tert-butyl N-methyl-N-[[5-[3-[4-(trifluoromethyl)anilino]pyrazin-2-yl]-1,3,4-oxadiazol-2-yl]methyl]carbamate (160 mg, 0.36 mmol, 1 eq) in DCM (1.5 mL) was added TFA (1.22 g, 10.66 mmol, 0.79 mL, 30 eq). The mixture was stirred at 25° C. for 1 h. The mixture was concentrated under reduced pressure to give a product (170 mg, crude, TFA) as a yellow solid. LCMS (ESI): RT=0.716 min, mass calc. for C₁₅H₁₃F₃N₆O 350.11 found 350.9 [M+H]⁺. The product (40 mg, 86 umol, 1 eq, TFA) was purified by prep-HPLC (column: Waters Xbridge 150*25 mm*5 um; mobile phase: [water (0.05% NH₃H₂O+10 mM NH₄HCO₃)-ACN]; B %: 43%-73%, 7.8 min) to give the desired compound (7.7 mg, 22 umol, 25.7% yield) as a yellow solid. LCMS (ESI): RT=0.709 min, mass calc. for C₁₅H₁₃F₃N₆O 350.11 found 350.9 [M+H]⁺; ¹H NMR (400 MHz, CDCl₃) δ 10.44 (s, 1H), 8.39 (d, J=2.5 Hz, 1H), 8.23 (d, J=2.3 Hz, 1H), 7.93 (d, J=8.5 Hz, 2H), 7.64 (d, J=8.5 Hz, 2H), 4.18 (s, 2H), 2.57 (s, 3H).

### methyl-[[5-[3-[4-(trifluoromethyl)anilino]pyrazin-2-yl]-1,3,4-oxadiazol-2-yl]methyl]cyanamide (Compound 45)

To a solution of 3-[5-(methylaminomethyl)-1,3,4-oxadiazol-2-yl]-N-[4-(trifluoromethyl)phenyl]pyrazin-2-amine (120 mg, 0.26 mmol, 1 eq, TFA) in THF (1.5 mL) were added DIEA (100.2 mg, 0.78 mmol, 0.14 mL, 3 eq) and BrCN (41.1 mg, 0.39 mmol, 29 uL, 1.5 eq). The mixture was stirred at 25° C. for 0.5 h. The mixture was quenched with H₂O (10 mL), extracted with EA (20 mL*3). The combined organic layers were washed with brine (15 mL), dried over Na₂SO₄, filtered and concentrated under reduced pressure to give a residue. The residue was purified by prep-HPLC (column: Waters Xbridge 150*25 mm*5 um; mobile phase: [water (0.05% NH₃H₂O+10 mM NH₄HCO₃)-ACN]; B %: 44%-74%, 9.5 min) to give the desired compound (25.2 mg, 67 umol, 26.0% yield) as a yellow solid. LCMS (ESI): RT=0.859 min, mass calc. for C₁₆H₁₂F₃N₇O 375.11 found 376.0 [M+H]⁺; ¹H NMR (400 MHz, CDCl₃) δ 10.29 (s, 1H), 8.41 (d, J=2.0 Hz, 1H), 8.24 (d, J=2.0 Hz, 1H), 7.92 (d, J=8.5 Hz, 2H), 7.65 (d, J=8.5 Hz, 2H), 4.60 (s, 2H), 3.11 (s, 3H).

### II. Biological Evaluation

### Example A1: YAP Reporter Assay

HEK293T cells stably transfected with 8XTBD luciferase reporter and pRLTK in 384-well plates were treated with the test compounds, starting from 3 μM (final concentration in assay plate), 1:3 dilution, and 10 points in quadruplicates. Post 24-hr incubation with compounds at 37° C. and 5% CO2, cells were lysed and 8XTBD-driven firefly luciferase and control TK-driven renilla luciferase activities were measured using Promega Dual-Luciferase Reporter Assay System.

Reagents: The reagents used for this study are: DMEM: Invitrogen #11960077, Dual-Glo Luciferase Assay System: Promega-E2980, Puromycin Dihydrochloride: Invitrogen-A1113803, 384-well plate: PerkinElmer-6007480, L-GLUTAMINE: Invitrogen-25030164, Hygromycin B: Invitrogen-10687010, and Penicillin-Streptomycin: Merk-TMS-AB2-C

Media: The media used for this assay were: Culture Medium: DMEM+1 ug/mL puromycin+200 ug/mL hygromycin (with 10% FBS+1 mM L-glutamine); and Assay Medium: DMEM (with 10% FBS+1 mM L-glutamine+1×P/S).

Cell Plating: The appropriate media was warmed at 37° C. by water bath: Culture Medium, Assay Medium, 1*D-PBS, 0.05% trypsin-EDTA. The cells were trypsinized after removing all media, then washed with 1*sterile D-PBS and then with 2 ml 0.05% trypsin-EDTA. The cells were then incubated at RT for one minute. Then 10 ml/75 cm2 flask Assay Medium was added to each flask. Using a 10 ml pipette, the cells were then gently resuspended in the media, until the clumps completely disappeared. The cells were then transferred into 50 ml centrifuge tubes and were centrifuged at 800 rpm for 5 mins. The medium was removed and the cells were resuspended with Assay Medium. An aliquot of cells was used to count the cell density (cells/ml). The cell suspension was then diluted with Assay Medium to a concentration of 6×104 cells/ml. 50 ul cells suspension was then plated to 384-well plate (PerkinElmer-6007480), 3×103 cells/well and the cells were incubated in an incubator at 37° C., 5% CO2.

Compound Treatment: In the afternoon (incubation of the plate with 3-4 hrs), the test compounds were added by Echo, starting from 3 uM (final concentration in the assay plate), 1:3 dilution, 10 points, quadruplicates. The plate was placed at 37° C., 5% CO2 incubator for 24 hrs.

Detection: The Dual-Glo Luciferase Reagent was prepared by transferring the contents of one bottle of Dual-Glo Luciferase Buffer to one bottle of Dual-Glo Luciferase Substrate to create the Dual-Glo Luciferase Reagent. Mixing was performed by inversion until the substrate was thoroughly dissolved. After mixing, the reagent was aliquoted into 15 ml tubes. In the afternoon (24 hrs post compound treatment), the DMEM+medium in the 384 well plates were aspirated by Microplate Washer.

Measuring firefly luciferase activity: 20 ul Dual-Glo Luciferase Reagent was added to the 384-well plates. The plates were protected from light to prevent interference with the assay. The plates were shaken for 1 min followed centrifuging plates at 1000 rpm for 30 seconds. After waiting at least 10 minutes, the firefly luminescence was measured by Envision.

Measuring renilla luciferase activity: 20 ul Stop-Glo Reagent was added to the 384-well plates. The plates were shaken for 1 min and then centrifuged at 1000 rpm for 30 seconds. After waiting at least 10 minutes, the renilla luminescence was measured by Envision.

Compound IC₅₀ and maximum inhibition on the firefly luciferase and renilla luciferase activities were reported separately. IC₅₀ for firefly luciferase activity are shown in the Table 2 below.

TABLE 2

| | | Firefly |
|---|---|---|
| Compound | | Luciferase |
| No. | Name | IC₅₀ (μM) |
| 1 | methyl-[2-[5-[3-[4-(trifluoromethyl)anilino]pyrazin-2-yl]- | A |
| | 1,3,4-oxadiazol-2-yl]ethyl]cyanamide | |
| 2 | ethyl-[2-[5-[3-[4-(trifluoromethyl)anilino]pyrazin-2-yl]- | A |
| | 1,3,4-oxadiazol-2-yl]ethyl]cyanamide | |
| 5 | ethyl-[[5-[2-[4-(trifluoromethyl)anilino]-3-pyridyl]-1,3,4- | A |
| | oxadiazol-2-yl]methyl]cyanamide | |
| 6 | methyl-[[5-[2-[4-(trifluoromethyl)anilino]-3-pyridyl]-1,3,4- | A |
| | oxadiazol-2-yl]methyl]cyanamide | |
| 8 | tert-butyl N-ethyl-N-[[5-[2-[4-(trifluoromethyl)anilino]-3- | C |
| | pyridyl]-1,3,4-oxadiazol-2-yl]methyl]carbamate | |
| 9 | 3-[5-(methylaminomethyl)-1,3,4-oxadiazol-2-yl]-N-[4- | B |
| | (trifluoromethyl)phenyl]pyridin-2-amine | |
| 12 | (R)-3-methyl-3-(5-(2-((4- | A |
| | (trifluoromethyl)phenyl)amino)pyridin-3-yl)-1,3,4- | |
| | oxadiazol-2-yl)pyrrolidin-2-one | |
| 13 | (S)-3-methyl-3-(5-(2-((4- | A |
| | (trifluoromethyl)phenyl)amino)pyridin-3-yl)-1,3,4- | |
| | oxadiazol-2-yl)pyrrolidin-2-one | |
| 15 | ethyl-[2-[5-[2-[4-(trifluoromethyl)anilino]-3-pyridyl]-1,3,4- | A |
| | oxadiazol-2-yl]ethyl]cyanamide | |
| 16 | methyl-[2-[5-[2-[4-(trifluoromethyl)anilino]-3-pyridyl]- | A |
| | 1,3,4-oxadiazol-2-yl]ethyl]cyanamide | |
| 19 | 3-methyl-3-(5-(2-((4- | A |
| | (trifluoromethyl)phenyl)amino)pyridin-3-yl)-1,3,4- | |
| | oxadiazol-2-yl)pyrrolidin-2-one | |
| 20 | 3-methyl-3-{5-[3-(4-trifluoromethyl-phenylamino)-pyrazin- | A |
| | 2-yl]-[1,3,4]oxadiazol-2-yl}-pyrrolidin-2-one | |
| 21 | (3S)-3-ethyl-3-[5-[2-[4-(pentafluoro-λ6-sulfanyl)anilino]-3- | A |
| | pyridyl]-1,3,4-oxadiazol-2-yl]piperidin-2-one | |
| 22 | (3R)-3-ethyl-3-[5-[2-[4-(pentafluoro-λ6-sulfanyl)anilino]-3- | B |
| | pyridyl]-1,3,4-oxadiazol-2-yl]piperidin-2-one | |
| 23 | (3R)-3-ethyl-3-[5-[2-[4-(pentafluoro-λ⁶-sulfanyl)anilino]-3- | B |
| | pyridyl]-1,3,4-oxadiazol-2-yl]pyrrolidin-2-one | |
| 24 | (3S)-3-ethyl-3-[5-[2-[4-(pentafluoro-λ⁶-sulfanyl)anilino]-3- | A |
| | pyridyl]-1,3,4-oxadiazol-2-yl]pyrrolidin-2-one | |
| 25 | 3,4-dimethyl-3-[5-[2-[4-(trifluoromethyl)anilino]-3- | B |
| | pyridyl]-1,3,4-oxadiazol-2-yl]piperazin-2-one | |
| 26 | 3-methyl-3-[5-[2-[4-(trifluoromethyl)anilino]-3-pyridyl]- | A |
| | 1,3,4-oxadiazol-2-yl]piperazin-2-one | |
| 27 | (3R)-3-methyl-3-[5-[2-[4-(pentafluoro-λ⁶-sulfanyl)anilino]- | B |
| | 3-pyridyl]tetrazol-2-yl]pyrrolidin-2-one | |
| 28 | (3S)-3-methyl-3-[5-[2-[4-(pentafluoro-λ⁶-sulfanyl)anilino]- | A |
| | 3-pyridyl]tetrazol-2-yl]pyrrolidin-2-one | |
| 29 | (3R)-3-methyl-3-[5-[2-[4-(pentafluoro-λ⁶-sulfanyl)anilino]- | A |
| | 3-pyridyl]-1,3,4-oxadiazol-2-yl]piperidin-2-one | |
| 30 | (3S)-3-methyl-3-[5-[2-[4-(pentafluoro-λ⁶-sulfanyl)anilino]- | B |
| | 3-pyridyl]-1,3,4-oxadiazol-2-yl]piperidin-2-one | |
| 31 | (3S)-3-ethyl-3-[5-[2-[4-(trifluoromethyl)anilino]-3-pyridyl]- | B |
| | 1,3,4-oxadiazol-2-yl]piperidin-2-one | |
| 32 | (3R)-3-ethyl-3-[5-[2-[4-(trifluoromethyl)anilino]-3-pyridyl]- | A |
| | 1,3,4-oxadiazol-2-yl]piperidin-2-one | |
| 33 | (3R)-3-ethyl-3-[5-[2-[4-(trifluoromethyl)anilino]-3-pyridyl]- | A |
| | 1,3,4-oxadiazol-2-yl]pyrrolidin-2-one | |
| 34 | (3S)-3-Ethyl-3-[5-[2-[4-(trifluoromethyl)anilino]-3-pyridyl]- | A |
| | 1,3,4-oxadiazol-2-yl]pyrrolidin-2-one | |
| 35 | (3R)-3-methyl-3-[5-[2-[4-(pentafluoro-λ⁶-sulfanyl)anilino]- | B |
| | 3-pyridyl]-1,3,4-oxadiazol-2-yl]pyrrolidin-2-one | |
| 36 | (3S)-3-methyl-3-[5-[2-[4-(pentafluoro-λ⁶-sulfanyl)anilino]- | A |
| | 3-pyridyl]-1,3,4-oxadiazol-2-yl]pyrrolidin-2-one | |
| 37 | 2-methyl-2-(5-(2-((4- | A |
| | (trifluoromethyl)phenyl)amino)pyridin-3-yl)-1,3,4- | |
| | oxadiazol-2-yl)morpholin-3-one | |
| 38 | 2-amino-4-methyl-4-[5-[2-[4-(trifluoromethyl)anilino]-3- | A |
| | pyridyl]-1,3,4-oxadiazol-2-yl]-3H-pyrrol-5-one | |
| 39 | (S)-3-methyl-3-(5-(2-((4- | A |
| | (trifluoromethyl)phenyl)amino)pyridin-3-yl)-1,3,4- | |
| | oxadiazol-2-yl)piperidin-2-one | |
| 40 | (R)-3-methyl-3-(5-(2-((4- | B |
| | (trifluoromethyl)phenyl)amino)pyridin-3-yl)-1,3,4- | |
| | oxadiazol-2-yl)piperidin-2-one | |
| 41 | 3-methyl-3-(5-(2-((4- | A |
| | (trifluoromethyl)phenyl)amino)phenyl)-1,3,4-oxadiazol-2- | |
| | yl)piperidin-2-one | |
| 42 | (3S)-3-methyl-3-[5-[2-[4-(trifluoromethyl)anilino]-3- | A |
| | pyridyl]tetrazol-2-yl]pyrrolidin-2-one | |
| 43 | (3R)-3-methyl-3-[5-[2-[4-(trifluoromethyl)anilino]-3- | A |
| | pyridyl]tetrazol-2-yl]pyrrolidin-2-one | |
| 44 | ethyl-[[5-[3-[4-(trifluoromethyl)anilino]pyrazin-2-yl]-1,3,4- | A |
| | oxadiazol-2-yl]methyl]cyanamide | |
| 45 | methyl-[[5-[3-[4-(trifluoromethyl)anilino]pyrazin-2-yl]- | A |
| | 1,3,4-oxadiazol-2-yl]methyl]cyanamide | |
| 48 | 3-[5-(methylaminomethyl)-1,3,4-oxadiazol-2-yl]-N-[4- | C |
| | (trifluoromethyl)phenyl]pyrazin-2-amine | |

Note:
Biochemical assay IC₅₀ data are designated within the following ranges:
A: ≤0.1 μM
B: >0.1 μM to ≤1.0 μM
C: >1.0 μM to ≤3.0 μM
D: >3.0 μM

Note: Biochemical assay IC₅₀ data are designated within the following ranges:
A: ≤0.1 μM C: >1.0 μM to ≤3.0 μM
B: >0.1 μM to ≤1.0 μM D: >3.0 μM

### Example A2: Tumor Suppression Assay

The procedures described herein for the tumor suppression assay is as described in PCT/US2013/043752 (WO 2013/188138). Mouse procedures are performed according to the guidelines of approved animal protocol and based on the methods. After the cells are grown to 90%>confluence, these cells are harvested by trypsinization, washed in phosphate-buffered saline (PBS), and resuspended in PBS supplemented with 50% Matrigel (BD Biosciences). An appropriate number of cells is prepared for administration, such as 200 μL per injection site. Immuno-compromised mice are injected on the dorsolateral sites subcutaneously. Any one of the compounds described herein is formulated accordingly and is then administered at a suitable dose. Control mice received vehicle alone. The average tumor diameter (two perpendicular axes of the tumor are measured) are recorded. The data are expressed in tumor volume estimated by ([width]2×length/2). Paired, two-tailed Student's t-test is performed to access the statistical significance.

### Example A3: Cell Proliferation Assay

Cancer cell lines are plated in 384-well plates 24 h before drug treatment. Post incubation for various time periods with the test compounds, starting from 3 μM (final concentration in assay plate), 1:3 dilution, and 10 points in duplicates, the number of viable cells and proliferative cells are determined using CellTiter-Glo® Luminescent Cell Viability Assay Kit (Promega) and Click-iT EdU HCS Assay Kit (Invitrogen) according to the manufacturers' protocols. The IC₅₀ values and maximum % inhibition of the test compounds are calculated using the dose response curves.

### NUMBERED EMBODIMENTS

Embodiment 1 is a compound of Formula (I), or a pharmaceutically acceptable salt or solvate thereof:

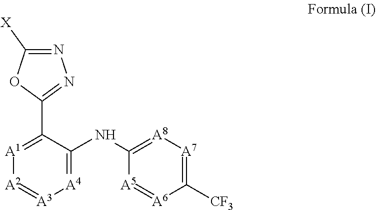

wherein
each A¹, A², A³, and A⁴ is independently N or CR¹;
each A⁵, A⁶, A⁷, and A⁸ is independently N or CR²; wherein at least one of A¹, A², A³, A⁴, A⁵, A⁶, A⁷, and A⁸ is N;
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, or -L⁷-Y³;
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, or

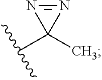 [SMILES: *C1(C)N=N1]

X is H, —CN, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, -L¹-Y¹, -L²-L³-Y², -L²-L³-L⁴-Y², -L⁵-L⁶-L³-Y², or -L⁶-L⁵-L³-Y².
L¹ is substituted or unsubstituted C₁-C₆alkylene;
L² is absent, substituted or unsubstituted C₁-C₆alkylene, substituted or unsubstituted C₃-C₁₀cycloalkylene, or substituted or unsubstituted C₂-C₁₀heterocycloalkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —O—NR³(C═O)—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —(C═O)—NR³S(═O)₂—, —S(═O)₂NR³—(C═O)O—, —O(C═O)—NR³S(═O)₂—, —NR³S(═O)₂NR³—(C═O)—, —(C═O)—NR³S(═O)₂NR³—, —O(C═O)—NR³S(═O)₂—NR³—, —NR³S(═O)₂NR³—(C═O)O—, —OS(═O)₂—, or —S(═O)₂O—;
L⁴ is substituted or unsubstituted C₁-C₆alkylene;
L⁵ is substituted or unsubstituted C₁-C₆alkylene;
L⁶ is substituted or unsubstituted C₃-C₁₀cycloalkylene;
L⁷ is substituted or unsubstituted C₁-C₆alkylene;
Y¹ is —N₃, substituted or unsubstituted C₃-C₁₀cycloalkyl, substituted or unsubstituted C₂-C₁₀ heterocycloalkyl, substituted or unsubstituted aryl, or substituted or unsubstituted heteroaryl;
Y² is H, —CN, —N₃, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b};
Y³ is —Si(R⁷)₃;
each R³ is independently H, —CN, —S(═O)₂(C₁-C₄alkyl), or substituted or unsubstituted C₁-C₆alkyl;
or R³ and Y² on the same N atom are taken together with the N atom to which they are attached to form a substituted or unsubstituted N-containing heterocycle;
each R⁴, R^{4a}, and R^{4b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{4a} and R^{4b} on the same N atom are taken together with the N atom to which they are attached to form a substituted or unsubstituted N-containing heterocycle;
each R⁵, R^{5a}, and R^{5b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{5a} and R^{5b} on the same N atom are taken together with the N atom to which they are attached to form a substituted or unsubstituted N-containing heterocycle; and
each R⁶, R^{6a}, and R^{6b} is independently H or substituted or unsubstituted C₁-C₆alkyl;
or R^{6a} and R^{6b} on the same N atom are taken together with the N atom to which they are attached to form a substituted or unsubstituted N-containing heterocycle; and
each R⁷ is independently substituted or unsubstituted C₁-C₆alkyl.

Embodiment 2 is the compound of embodiment 1, or a pharmaceutically acceptable salt or solvate thereof, wherein the compound of Formula (I) has a structure of Formula (Iaa):

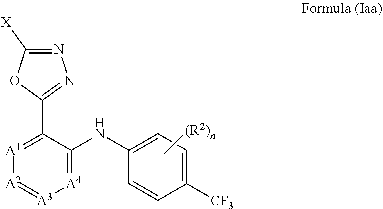

wherein n is 1, 2, 3, or 4.

Embodiment 3 is the compound of embodiment 1, or a pharmaceutically acceptable salt or solvate thereof, wherein the compound of Formula (I) has a structure of Formula (Ibb):

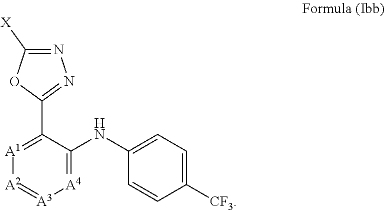

Embodiment 4 is the compound, or pharmaceutically acceptable salt or solvate thereof, of any one of embodiments 1-3, wherein

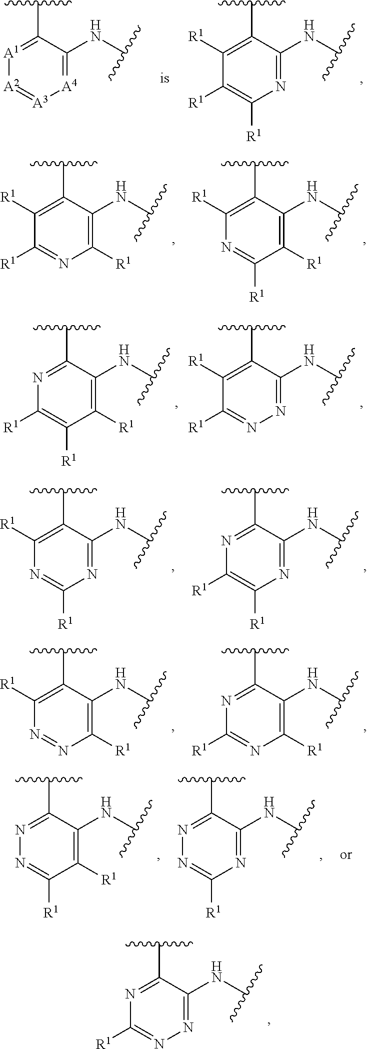

and
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl.

Embodiment 5 is the compound, or pharmaceutically acceptable salt or solvate thereof, of any one of embodiments 1-4, wherein

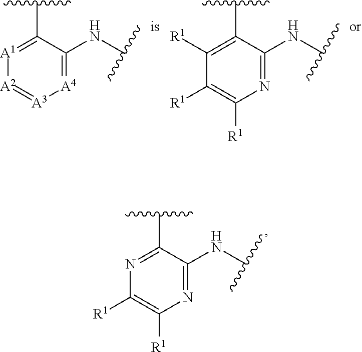

and
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl.

Embodiment 6 is the compound of embodiment 1, or a pharmaceutically acceptable salt or solvate thereof, wherein the compound of Formula (I) has a structure of Formula (Ic):

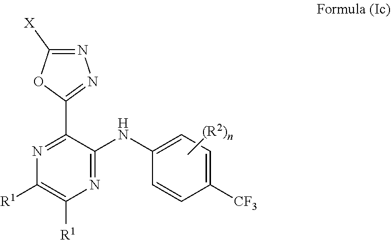

wherein n is 1, 2, 3, or 4.

Embodiment 7 is the compound of embodiment 1, or a pharmaceutically acceptable salt or solvate thereof, wherein the compound of Formula (I) has a structure of Formula (Id):

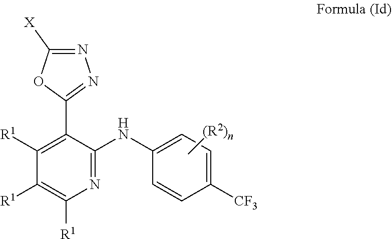

wherein n is 1, 2, 3, or 4.

Embodiment 8 is the compound, or pharmaceutically acceptable salt or solvate thereof, of any one of embodiments 1-7, wherein each R¹ is independently H, halogen, —CN, —OR⁴, —SR⁴, —NR^{4a}R^{4b} substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, or substituted or unsubstituted monocyclic heteroaryl.

Embodiment 9 is the compound, or pharmaceutically acceptable salt or solvate thereof, of embodiment 8, wherein each R¹ is independently H, halogen, substituted or unsubstituted C₁-C₄alkyl, or substituted or unsubstituted C₁-C₄haloalkyl.

Embodiment 10 is the compound, or pharmaceutically acceptable salt or solvate thereof, of embodiment 9, wherein each R¹ is independently H, F, Cl, Br, I, —CH₃, —CH₂CH₃, —CH₂CH₂CH₃, —CH(CH₃)₂, —CH₂CH₂CH₂CH₃, —CH₂CH(CH₃)₂, —C(CH₃)₃, —CH₂F, —CHF₂, —CF₃, or —CH₂CF₃.

Embodiment 11 is the compound, or pharmaceutically acceptable salt or solvate thereof, of embodiment 10, wherein each R¹ is H.

Embodiment 12 is the compound, or pharmaceutically acceptable salt or solvate thereof, of any one of embodiments 2, 6, or 7, wherein

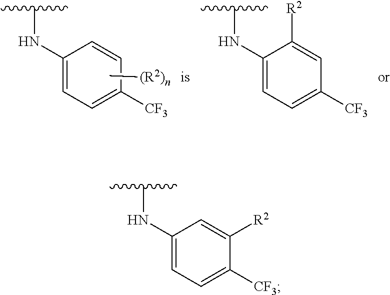

and
R² is H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₁-C₆heteroalkyl, substituted or unsubstituted aralkyl, substituted or unsubstituted aryl, substituted or unsubstituted heteroaryl, or

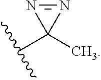 [SMILES: *C1(C)N=N1]

Embodiment 13 is the compound, or pharmaceutically acceptable salt or solvate thereof, of embodiment 12, wherein
R² is H, halogen, —N₃, —CN, —OR⁵, —SR⁵, —S(═O)₂R⁵, —NR^{5a}R^{5b}, —C(═O)OR⁵, substituted or unsubstituted C₁-C₄alkyl, substituted or unsubstituted C₁-C₄haloalkyl, substituted or unsubstituted C₁-C₄heteroalkyl, substituted or unsubstituted benzyl, substituted or unsubstituted phenyl, or substituted or unsubstituted monocyclic heteroaryl; and
each R⁵, R^{5a}, and R^{5b} is independently H, or substituted or unsubstituted C₁-C₄alkyl.

Embodiment 14 is the compound, or pharmaceutically acceptable salt or solvate thereof, of embodiment 12, wherein
R² is H, halogen, —N₃, —OR⁵, —S(═O)₂R⁵, —C(═O)OR⁵, substituted or unsubstituted C₁-C₄alkyl, or substituted or unsubstituted C₁-C₄haloalkyl.

Embodiment 15 is the compound, or pharmaceutically acceptable salt or solvate thereof, of any one of embodiments 2, 6, or 7, wherein

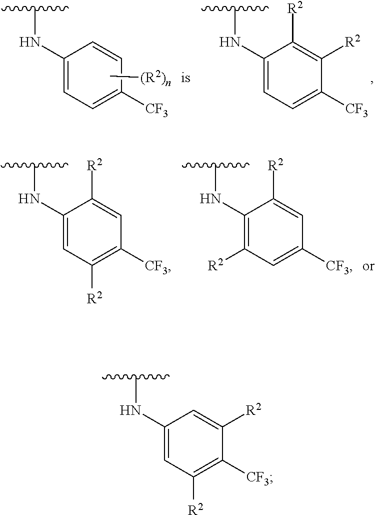

and
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₁-C₆heteroalkyl, substituted or unsubstituted aralkyl, substituted or unsubstituted aryl, substituted or unsubstituted heteroaryl, or

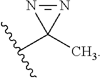 [SMILES: *C1(C)N=N1]

Embodiment 16 is the compound, or pharmaceutically acceptable salt or solvate thereof, of embodiment 15, wherein
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₄alkyl, substituted or unsubstituted C₁-C₄haloalkyl, substituted or unsubstituted C₁-C₄heteroalkyl, substituted or unsubstituted benzyl, substituted or unsubstituted phenyl, or substituted or unsubstituted monocyclic heteroaryl; and
each R⁵, R^{5a}, and R^{5b} is independently H or substituted or unsubstituted C₁-C₄alkyl.

Embodiment 17 is the compound, or pharmaceutically acceptable salt or solvate thereof, of embodiment 15, wherein each R² is independently H, halogen, —N₃, —OR⁵, —S(═O)₂R⁵, —C(═O)OR⁵, substituted or unsubstituted C₁-C₄alkyl, or substituted or unsubstituted C₁-C₄haloalkyl.

Embodiment 18 is the compound, or pharmaceutically acceptable salt or solvate thereof, of embodiment 12 or 15, wherein each R² is H.

Embodiment 19 is the compound, or pharmaceutically acceptable salt or solvate thereof, of any one of embodiments 1-18, wherein X is substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl.

Embodiment 20 is the compound, or pharmaceutically acceptable salt or solvate thereof, of any one of embodiments 1-18, wherein X is substituted or unsubstituted C₁-C₆alkyl, substituted or unsubstituted C₁-C₆haloalkyl, substituted or unsubstituted C₂-C₁₀heterocycloalkyl, substituted or unsubstituted aryl, or substituted or unsubstituted heteroaryl.

Embodiment 21 is the compound, or pharmaceutically acceptable salt or solvate thereof, of any one of embodiments 1-18, wherein X is substituted or unsubstituted C₁-C₆alkyl, substituted or unsubstituted C₁-C₆haloalkyl, substituted or unsubstituted C₃-C₁₀cycloalkyl, substituted or unsubstituted C₂-C₁₀heterocycloalkyl, or substituted or unsubstituted heteroaryl.

Embodiment 22 is the compound, or pharmaceutically acceptable salt or solvate thereof, of embodiment 21, wherein X is substituted or unsubstituted C₁-C₆alkyl.

Embodiment 23 is the compound, or pharmaceutically acceptable salt or solvate thereof, of embodiment 22, wherein X is —CH₃, —CH₂CH₃, —CH₂CH₂CH₃, —CH(CH₃)₂, —CH₂CH₂CH₂CH₃, —CH₂CH(CH₃)₂, or —C(CH₃)₃.

Embodiment 24 is the compound, or pharmaceutically acceptable salt or solvate thereof, of embodiment 22, wherein X is substituted C₁-C₆alkyl.

Embodiment 25 is the compound, or pharmaceutically acceptable salt or solvate thereof, of embodiment 24, wherein
X is C₁-C₆alkyl substituted with 1, 2, or 3 substituents each independently selected from —OR¹¹, —(C═O)R¹¹, NR¹¹(C═O)R¹¹, —(C═O)OR¹¹, —NR¹¹(C═O)OR¹¹, —O(C═O)OR¹¹, —NR^{11a}R^{11b}, —(C═O)NR^{11a}R^{11b}, or —O(C═O)NR^{11a}R^{11b}; wherein
each R¹¹ is independently H or substituted or unsubstituted C₁-C₆alkyl;
each R^{11a} and R^{11b} is independently H, —CN, —OR¹², —SR¹², substituted or unsubstituted C₁-C₆alkyl, or substituted or unsubstituted C₁-C₆haloalkyl; or
R^{11a} and R^{11b} are taken together with the N atom to which they are attached to form a substituted or unsubstituted N-containing heterocycle; and
each R¹² is independently H or substituted or unsubstituted C₁-C₆alkyl.

Embodiment 26 is the compound, or pharmaceutically acceptable salt or solvate thereof, of embodiment 24, wherein
X is C₁-C₆alkyl substituted with 1, 2, or 3 substituents each independently selected from —OR¹¹, NR¹¹(C═O)R¹¹, —NR¹¹(C═O)OR¹¹, —O(C═O)OR¹¹, —NR^{11a}R^{11b}, or —(C═O)NR^{11a}R^{11b}; wherein
each R¹¹ is independently H or substituted or unsubstituted C₁-C₆alkyl;
each R^{11a} and R^{11b} is independently H, —CN, —OR¹², —SR¹², substituted or unsubstituted C₁-C₆alkyl, or substituted or unsubstituted C₁-C₆haloalkyl; or
R^{11a} and R^{11b} are taken together with the N atom to which they are attached to form a substituted or unsubstituted N-containing heterocycle; and
each R¹² is independently H or substituted or unsubstituted C₁-C₆alkyl.

Embodiment 27 is the compound, or pharmaceutically acceptable salt or solvate thereof, of embodiment 24, wherein
X is C₁-C₆alkyl substituted with —NR^{11a}R^{11b}; wherein
each R^{11a} and R^{11b} is independently H, —CN, or substituted or unsubstituted C₁-C₆alkyl; or
R^{11a} and R^{11b} are taken together with the N atom to which they are attached to form a substituted or unsubstituted N-containing heterocycle.

Embodiment 28 is the compound, or pharmaceutically acceptable salt or solvate thereof, of embodiment 24, wherein X is C₁-C₆alkyl substituted with —NH₂, —N(H)CH₃, —N(H)CH₂CH₃, —N(H)CH₂CH₂CH₃, —N(H)CH(CH₃)₂, —N(H)cyclopropyl, —N(CN)CH₃, —N(CN)CH₂CH₃, —N(CN)CH₂CH₂CH₃, —N(CN)CH(CH₃)₂, or —N(CN)cyclopropyl.

Embodiment 29 is the compound, or pharmaceutically acceptable salt or solvate thereof, of embodiment 21, wherein X is substituted or unsubstituted C₁-C₄haloalkyl.

Embodiment 30 is the compound, or pharmaceutically acceptable salt or solvate thereof, of embodiment 29, wherein X is —CH₂F, —CHF₂, —CF₃, —CF₂CH₃, or —CH₂CF₃.

Embodiment 31 is the compound, or pharmaceutically acceptable salt or solvate thereof, of embodiment 21, wherein X is substituted or unsubstituted C₃-C₆cycloalkyl.

Embodiment 32 is the compound, or pharmaceutically acceptable salt or solvate thereof, of embodiment 31, wherein X is substituted or unsubstituted cyclopropyl, substituted or unsubstituted cyclobutyl, substituted or unsubstituted cyclopentyl, or substituted or unsubstituted cyclohexyl.

Embodiment 33 is the compound, or pharmaceutically acceptable salt or solvate thereof, of embodiment 21, wherein X is substituted or unsubstituted C₂-C₆heterocycloalkyl.

Embodiment 34 is the compound, or pharmaceutically acceptable salt or solvate thereof, of embodiment 33, wherein X is substituted or unsubstituted aziridinyl, substituted or unsubstituted azetidinyl, substituted or unsubstituted pyrrolidinyl, substituted or unsubstituted pyrrolidinonyl, substituted or unsubstituted piperidinyl, substituted or unsubstituted oxetanyl, substituted or unsubstituted tetrahydrofuranyl, substituted or unsubstituted tetrahydropyranyl, substituted or unsubstituted thietanyl, substituted or unsubstituted tetrahydrothienyl, substituted or unsubstituted tetrahydrothiopyranyl, substituted or unsubstituted morpholinyl, or substituted or unsubstituted piperazinyl, substituted or unsubstituted 1,3-dioxolanyl, substituted or unsubstituted 1,3-dioxolan-2-onyl, substituted or unsubstituted oxazolidinonyl, substituted or unsubstituted imidazolidin-2-onyl, or substituted or unsubstituted oxadiazolonyl.

Embodiment 35 is the compound, or pharmaceutically acceptable salt or solvate thereof, of embodiment 33, wherein X is substituted or unsubstituted pyrrolidinyl, substituted or unsubstituted pyrrolidinonyl, substituted or unsubstituted piperidinyl, substituted or unsubstituted 1,3-dioxolanyl, substituted or unsubstituted 1,3-dioxolan-2-onyl, substituted or unsubstituted oxazolidinonyl, substituted or unsubstituted oxadiazolidinonyl, substituted or unsubstituted isoxazolidinonyl, substituted or unsubstituted imidazolidin-2-onyl, or substituted or unsubstituted oxadiazolonyl.

Embodiment 36 is the compound, or pharmaceutically acceptable salt or solvate thereof, of embodiment 33, wherein X is pyrrolidinyl, pyrrolidinonyl, oxazolidinonyl, isoxazolidinonyl, or imidazolidin-2-onyl, each substituted with 1, 2, 3, or 4 substituents each independently selected from F, —OH, —CN, substituted or unsubstituted C₁-C₆alkyl, substituted or unsubstituted C₁-C₆alkoxy, substituted or unsubstituted C₁-C₆hydroxyalkyl, substituted or unsubstituted C₁-C₆alkylamino, and substituted or unsubstituted C₁-C₆aminoalkyl.

Embodiment 37 is the compound, or pharmaceutically acceptable salt or solvate thereof, of embodiment 33, wherein X is pyrrolidinyl, pyrrolidinonyl, oxazolidinonyl, isoxazolidinonyl, or imidazolidin-2-onyl, each substituted with 1, 2, 3, or 4 substituents each independently selected from F, —OH, —CN, C₁-C₆alkyl, C₁-C₆alkoxy, C₁-C₆hydroxyalkyl, C₁-C₆alkylamino, and C₁-C₆aminoalkyl.

Embodiment 38 is the compound, or pharmaceutically acceptable salt or solvate thereof, of embodiment 33, wherein X is pyrrolidinyl, pyrrolidinonyl, oxazolidinonyl, isoxazolidinonyl, or imidazolidin-2-onyl, each substituted with 1 or 2 substituents each independently selected from F, —OH, —CN, —CH₃, —CH₂CH₃, —OCH₃, and —OCH₂CH₃.

Embodiment 39 is the compound, or pharmaceutically acceptable salt or solvate thereof, of embodiment 33, wherein X is

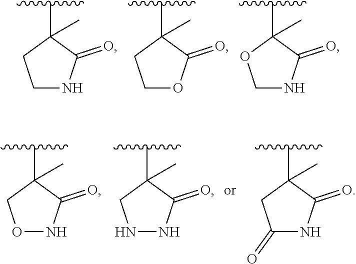

Embodiment 40 is the compound, or pharmaceutically acceptable salt or solvate thereof, of embodiment 33, wherein X is

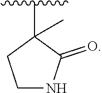 [SMILES: *C1(C)CCNC1=O]

Embodiment 41 is the compound, or pharmaceutically acceptable salt or solvate thereof, of embodiment 21, wherein X is substituted or unsubstituted heteroaryl.

Embodiment 42 is the compound, or pharmaceutically acceptable salt or solvate thereof, of embodiment 41, wherein X is substituted or unsubstituted monocyclic heteroaryl.

Embodiment 43 is the compound, or pharmaceutically acceptable salt or solvate thereof, of embodiment 42, wherein X is substituted or unsubstituted pyridinyl, substituted or unsubstituted imidazolyl, substituted or unsubstituted pyrimidinyl, substituted or unsubstituted pyrazolyl, substituted or unsubstituted triazolyl, substituted or unsubstituted pyrazinyl, substituted or unsubstituted tetrazolyl, substituted or unsubstituted furyl, substituted or unsubstituted thienyl, substituted or unsubstituted isoxazolyl, substituted or unsubstituted thiazolyl, substituted or unsubstituted oxazolyl, substituted or unsubstituted isothiazolyl, substituted or unsubstituted pyrrolyl, substituted or unsubstituted pyridazinyl, substituted or unsubstituted triazinyl, substituted or unsubstituted oxadiazolyl, substituted or unsubstituted thiadiazolyl, or substituted or unsubstituted furazanyl.

Embodiment 44 is the compound, or pharmaceutically acceptable salt or solvate thereof, of any one of embodiments 1-18, wherein X is -L¹-Y¹.

Embodiment 45 is the compound, or pharmaceutically acceptable salt or solvate thereof, of embodiment 44, wherein
L¹ is substituted or unsubstituted C₁-C₄alkylene; and
Y¹ is substituted or unsubstituted C₃-C₆cycloalkyl.

Embodiment 46 is the compound, or pharmaceutically acceptable salt or solvate thereof, of embodiment 45, wherein Y¹ is substituted or unsubstituted cyclopropyl, substituted or unsubstituted cyclobutyl, substituted or unsubstituted cyclopentyl, substituted or unsubstituted cyclohexyl.

Embodiment 47 is the compound, or pharmaceutically acceptable salt or solvate thereof, of embodiment 44, wherein
L¹ is substituted or unsubstituted C₁-C₄alkylene; and
Y¹ is substituted or unsubstituted C₂-C₆ heterocycloalkyl.

Embodiment 48 is the compound, or pharmaceutically acceptable salt or solvate thereof, of embodiment 47, wherein Y¹ is substituted or unsubstituted aziridinyl, substituted or unsubstituted azetidinyl, substituted or unsubstituted pyrrolidinyl, substituted or unsubstituted piperidinyl, substituted or unsubstituted oxetanyl, substituted or unsubstituted tetrahydrofuranyl, substituted or unsubstituted tetrahydropyranyl, substituted or unsubstituted thietanyl, substituted or unsubstituted tetrahydrothienyl, substituted or unsubstituted tetrahydrothiopyranyl, substituted or unsubstituted morpholinyl, or substituted or unsubstituted piperazinyl, substituted or unsubstituted 1,3-dioxolanyl, substituted or unsubstituted 1,3-dioxolan-2-onyl, substituted or unsubstituted oxazolidinonyl, substituted or unsubstituted imidazolidin-2-onyl, or substituted or unsubstituted oxadiazolonyl.

Embodiment 49 is the compound, or pharmaceutically acceptable salt or solvate thereof, of embodiment 44, wherein

L¹ is substituted or unsubstituted C₁-C₄alkylene; and

Y¹ is substituted or unsubstituted phenyl.

Embodiment 50 is the compound, or pharmaceutically acceptable salt or solvate thereof, of embodiment 44, wherein

L¹ is substituted or unsubstituted C₁-C₄alkylene; and

Y¹ is substituted or unsubstituted heteroaryl.

Embodiment 51 is the compound, or pharmaceutically acceptable salt or solvate thereof, of embodiment 50, wherein Y¹ is substituted or unsubstituted monocyclic heteroaryl.

Embodiment 52 is the compound, or pharmaceutically acceptable salt or solvate thereof, of embodiment 51, wherein Y¹ is substituted or unsubstituted pyridinyl, substituted or unsubstituted imidazolyl, substituted or unsubstituted pyrimidinyl, substituted or unsubstituted pyrazolyl, substituted or unsubstituted triazolyl, substituted or unsubstituted pyrazinyl, substituted or unsubstituted tetrazolyl, substituted or unsubstituted furyl, substituted or unsubstituted thienyl, substituted or unsubstituted isoxazolyl, substituted or unsubstituted thiazolyl, substituted or unsubstituted oxazolyl, substituted or unsubstituted isothiazolyl, substituted or unsubstituted pyrrolyl, substituted or unsubstituted pyridazinyl, substituted or unsubstituted triazinyl, substituted or unsubstituted oxadiazolyl, substituted or unsubstituted thiadiazolyl, or substituted or unsubstituted furazanyl.

Embodiment 53 is the compound, or pharmaceutically acceptable salt or solvate thereof, of any one of embodiments 1-18, wherein X is -L²-L³-Y².

Embodiment 54 is the compound, or pharmaceutically acceptable salt or solvate thereof, of embodiment 53, wherein
L² is substituted or unsubstituted C₁-C₄alkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —S(═O)₂NR³—(C═O)O—, —NR³S(═O)₂NR³—(C═O)—, —NR³S(═O)₂NR³—(C═O)O—, or —O—S(═O)₂—;
Y² is independently H, —CN, —N₃, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b}.

Embodiment 55 is the compound, or pharmaceutically acceptable salt or solvate thereof, of embodiment 54, wherein
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —NR³(C═O)O—, —NR³S(═O)₂—, —S(═O)₂NR³—, or —OS(═O)₂—;
Y² is independently H, —CN, —N₃, substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, substituted or unsubstituted monocyclic heteroaryl, —OR⁶, —(C═O)OR⁶, —NR^{6a}R^{6b}, or —(C═O)NR^{6a}R^{6b}; and
each R⁶, R^{6a}, and R^{6b} is independently H or substituted or unsubstituted C₁-C₄alkyl.

Embodiment 56 is the compound, or pharmaceutically acceptable salt or solvate thereof, of embodiment 53, wherein
L² is absent;
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —S(═O)₂NR³—(C═O)O—, —NR³S(═O)₂NR³—(C═O)—, —NR³S(═O)₂NR³—(C═O)O—, or —OS(═O)₂—;
Y² is independently H, —CN, —N₃, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b}.

Embodiment 57 is the compound, or pharmaceutically acceptable salt or solvate thereof, of embodiment 56, wherein
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —NR³(C═O)O—, —NR³S(═O)₂—, —S(═O)₂NR³—, or —OS(═O)₂—;
Y² is independently H, —CN, —N₃, substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, substituted or unsubstituted monocyclic heteroaryl, —OR⁶, —(C═O)OR⁶, —NR^{6a}R^{6b}, or —(C═O)NR^{6a}R^{6b}; and
each R⁶, R^{6a}, and R^{6b} is independently H or substituted or unsubstituted C₁-C₄alkyl.

Embodiment 58 is the compound, or pharmaceutically acceptable salt or solvate thereof, of any one of embodiments 1-18, wherein X is -L²-L³-L⁴-Y².

Embodiment 59 is the compound, or pharmaceutically acceptable salt or solvate thereof, of embodiment 58, wherein
L² is substituted or unsubstituted C₁-C₄alkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —S(═O)₂NR³—(C═O)O—, —NR³S(═O)₂NR³—(C═O)—, —NR³S(═O)₂NR³—(C═O)O—, or —OS(═O)₂—;
L⁴ is substituted or unsubstituted C₁-C₄alkylene;
Y² is independently H, —CN, —N₃, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b}.

Embodiment 60 is the compound, or pharmaceutically acceptable salt or solvate thereof, of embodiment 59, wherein
L³ is —O—, —S—, —(S═O)—, —S(═O)₂—, —NR³—, —(C═O)—, —(C═O)O—, —O(C═O)—, —(C═O)NR³—, —(C═O)NR³—O—, —NR³(C═O)—, —NR³(C═O)NR³—, —NR³(C═O)O—, —NR³S(═O)₂—, —S(═O)₂NR³—, or —OS(═O)₂—;
Y² is independently H, —CN, —N₃, substituted or unsubstituted C₁-C₄alkyl, substituted or unsubstituted C₁-C₄haloalkyl, substituted or unsubstituted C₁-C₄heteroalkyl, substituted or unsubstituted C₃-C₆cycloalkyl, substituted or unsubstituted C₂-C₆heterocycloalkyl, substituted or unsubstituted benzyl, substituted or unsubstituted phenyl, substituted or unsubstituted monocyclic heteroaryl, —OR⁶, —(C═O)OR⁶, —NR^{6a}R^{6b}, or —(C═O)NR^{6a}R^{6b}; and
each R⁶, R^{6a}, and R^{6b} is independently H or substituted or unsubstituted C₁-C₄alkyl.

Embodiment 61 is the compound, or pharmaceutically acceptable salt or solvate thereof, of any one of embodiments 1-60, wherein the compound exhibits an IC₅₀ of no more than about 3.000 μM.

Embodiment 62 is a compound, or pharmaceutically acceptable salt or solvate thereof, wherein the compound is a compound from Table 1, or a pharmaceutically acceptable salt or solvate thereof.

Embodiment 63 is a pharmaceutical composition comprising a pharmaceutically acceptable excipient and a compound of any one of embodiments 1-62, or a pharmaceutically acceptable salt or solvate thereof.

Embodiment 64 is a method for treating a cancer in a subject in need thereof comprising administering a therapeutically effective amount of a compound of any one of embodiments 1-62, or a pharmaceutically acceptable salt or solvate thereof.

The examples and embodiments described herein are for illustrative purposes only and various modifications or changes suggested to persons skilled in the art are to be included within the spirit and purview of this application and scope of the appended claims.

What is claimed is:

1. A compound of Formula (A), or a pharmaceutically acceptable salt or solvate thereof: 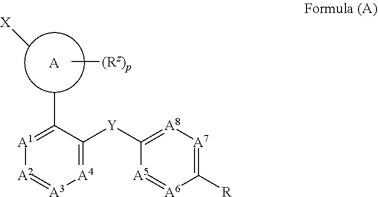
wherein:
at least one of A¹, A², A³, A⁴, A⁵, A⁶, A⁷, and A⁸ is N;
each A¹, A², A³, and A⁴ is independently N or CR¹;
each A⁵, A⁶, A⁷, and A⁸ is independently N or CR²;
ring A is a 5-membered heteroaryl;
R is halogen, nitro, —CN, —O(C₁-C₆fluoroalkyl), —S(C₁-C₆fluoroalkyl), —S(halogen)₅, or substituted or unsubstituted C₁-C₆fluoroalkyl;
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R² is independently H, halogen, —N₃, —CN, —OR⁵, —SR⁵, —S(═O)₂R⁵, —NR^{5a}R^{5b}, —C(═O)OR⁵, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
Y is O, S, or NR³;
X is H, —CN, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted aryl, substituted or unsubstituted heteroaryl, -L²-Y², -L²-L³-Y², or -L²-L³-L⁴-Y²;
each L² and L⁴ is independently absent, substituted or unsubstituted C₁-C₆alkylene, substituted or unsubstituted C₃-C₁₀cycloalkylene, or substituted or unsubstituted C₂-C₁₀heterocycloalkylene;
L³ is —O—, —S—, —(S═O)—, —S(═O)₂—, —NR³—, —(C═O)—, —(C═O)O—, —O(C═O)—, —(C═O)NR³—, —(C═O)NR³—O—, —O—NR³(C═O)—, —NR³(C═O)—, —NR³(C═O)NR³—, —O(C═O)NR³—, —NR³(C═O)O—, —NR³S(═O)₂NR³—, —NR³S(═O)₂—, —S(═O)₂NR³—, —S(═O)₂NR³—(C═O)—, —(C═O)—NR³S(═O)₂—, —S(═O)₂NR³—(C═O)O—, —O(C═O)—NR³S(═O)₂—, —NR³S(═O)₂NR³—(C═O)—, —(C═O)—NR³S(═O)₂NR³—, —O(C═O)—NR³S(═O)₂—NR³—, —NR³S(═O)₂NR³—(C═O)O—, —OS(═O)₂—, or —S(═O)₂O—;
Y² is H, —CN, —N₃, halogen, substituted or unsubstituted C₁-C₆alkyl, substituted or unsubstituted C₁-C₆haloalkyl, substituted or unsubstituted C₂-C₆alkenyl, substituted or unsubstituted C₂-C₆alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, —OR⁶, —(C═O)OR⁶, —NR^{6a}R^{6b}, or —(C═O)NR^{6a}R^{6b};
each R³ is independently H, —CN, —S(═O)₂(C₁-C₄alkyl), or substituted or unsubstituted C₁-C₆alkyl;
or R³ and Y² on the same N atom are taken together with the N atom to which they are attached to form a substituted or unsubstituted N-containing heterocycle;
each R⁴, R^{4a}, and R^{4b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{4a} and R^{4b} on the same N atom are taken together with the N atom to which they are attached to form a substituted or unsubstituted N-containing heterocycle;
each R⁵, R^{5a}, and R^{5b} is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
or R^{5a} and R^{5b} on the same N atom are taken together with the N atom to which they are attached to form a substituted or unsubstituted N-containing heterocycle; and
each R⁶, R^{6a}, and R^{6b} is independently H or substituted or unsubstituted C₁-C₆alkyl; and
or R^{6a} and R^{6b} on the same N atom are taken together with the N atom to which they are attached to form a substituted or unsubstituted N-containing heterocycle;
each R^{z} is independently H, halogen, or substituted or unsubstituted C₁-C₆alkyl; and
p is 1, 2, or 3.

2. The compound of claim 1, or a pharmaceutically acceptable salt or solvate thereof, wherein:
A⁵ is CR²; A⁶ is CR²; A⁷ is CR²; and A⁸ is CR²;
or A⁵ is N; A⁶ is CR²; A⁷ is CR²; and A⁸ is CR²;
or A⁵ is CR²; A⁶ is N; A⁷ is CR²; and A⁸ is CR²;
or A⁵ is CR²; A⁶ is CR²; A⁷ is N; and A⁸ is CR²;
or A⁵ is CR²; A⁶ is CR²; A⁷ is CR²; and A⁸ is N.

3. The compound of claim 1 or claim 2, or a pharmaceutically acceptable salt or solvate thereof, wherein:
A¹ is CR¹; A² is CR¹; A³ is CR¹; and A⁴ is CR¹;
or A¹ is N; A² is CR¹; A³ is CR¹; and A⁴ is CR¹;
or A¹ is CR¹; A² is N; A³ is CR¹; and A⁴ is CR¹;
or A¹ is CR¹; A² is CR¹; A³ is N; and A⁴ is CR¹;
or A¹ is CR¹; A² is CR¹; A³ is CR¹; and A⁴ is N;
or A¹ is CR¹ or N; A² is CR¹; A³ is CR¹; and A⁴ is N.

4. The compound of claim 1 or claim 3, or a pharmaceutically acceptable salt or solvate thereof, wherein the compound has a structure of Formula (A-1): 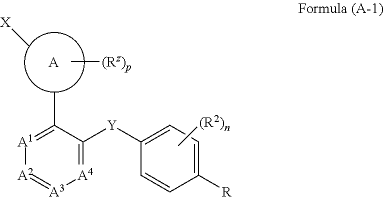
wherein, Y is O, S, or NH; and n is 1, 2, 3, or 4.

5. The compound of any one of claims 1-4, or pharmaceutically acceptable salt or solvate thereof, wherein Y is NH.

6. The compound of claim 5, or pharmaceutically acceptable salt or solvate thereof, wherein 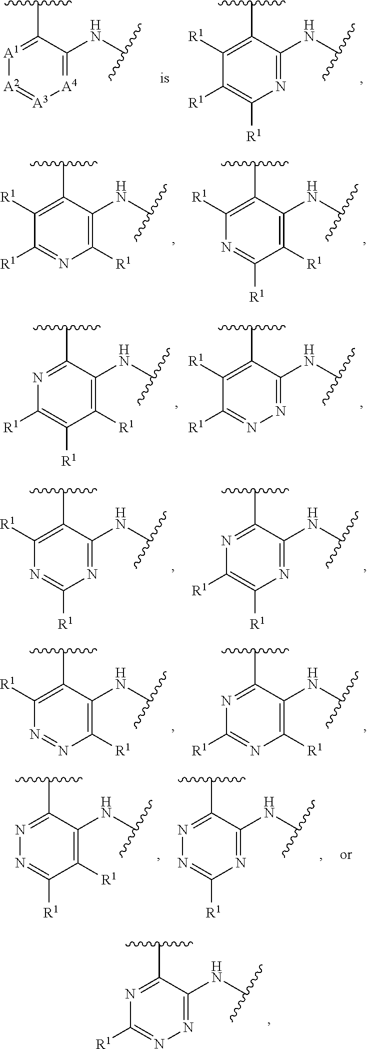
and
each R¹ is independently H, halogen, —CN, —OR⁴, —SR⁴, —NR^{4a}R^{4b}, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl.

7. The compound of claim 5 or claim 6, or pharmaceutically acceptable salt or solvate thereof, wherein 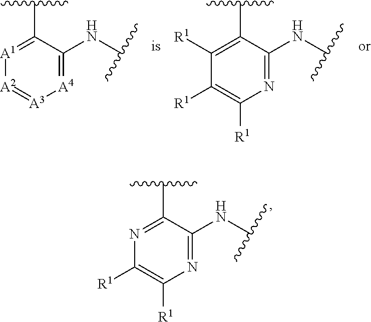
and
each R¹ is independently H, halogen, —CN, —OCH₃, or —CH₃.

8. The compound of any one of claims 1-7, or a pharmaceutically acceptable salt or solvate thereof, wherein the compound has a structure of Formula (A-4): 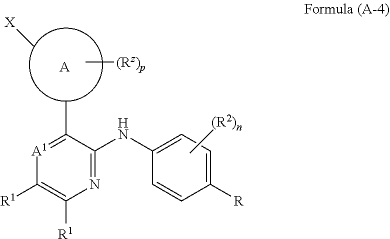
wherein, n is 1, 2, 3, or 4.

9. The compound of any one of claims 1-8, or a pharmaceutically acceptable salt or solvate thereof, wherein the compound has a structure of Formula (A-2): 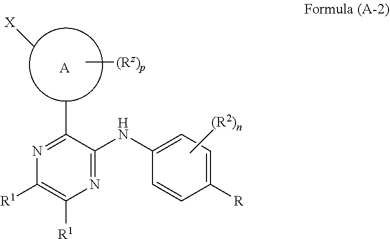
wherein n is 1, 2, 3, or 4.

10. The compound of any one of claims 1-8, or a pharmaceutically acceptable salt or solvate thereof, wherein the compound has a structure of Formula (A-3): 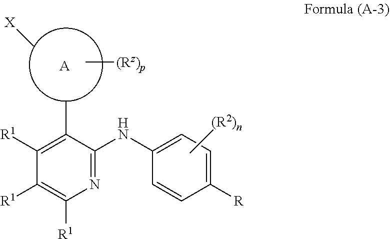
wherein, n is 1, 2, 3, or 4.

11. The compound of any one of claims 1-10, or pharmaceutically acceptable salt or solvate thereof, wherein
each R¹ is H.

12. The compound of any one of claims 1-11, or a pharmaceutically acceptable salt or solvate thereof, wherein ring A is a 5-membered heteroaryl comprising 1-4 N, 0-1 O, and 0-1 S ring atoms.

13. The compound of any one of claims 1-12, or pharmaceutically acceptable salt or solvate thereof, wherein

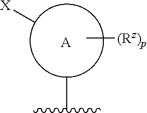

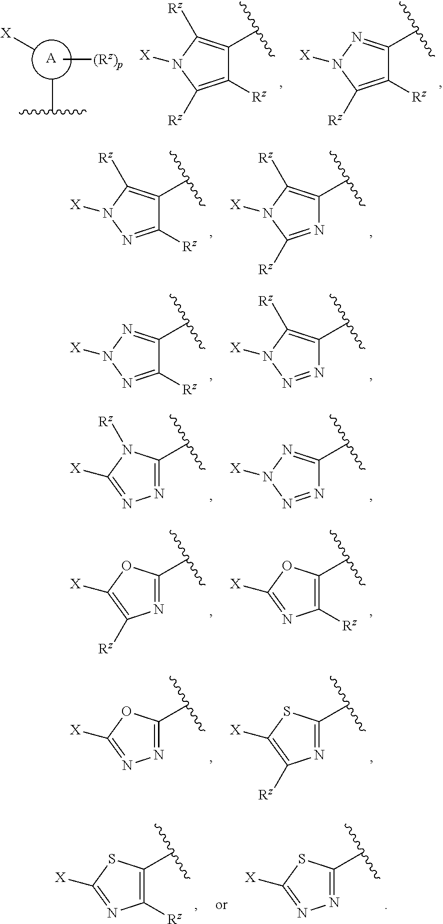

14. The compound of any one of claims 1-13, or pharmaceutically acceptable salt or solvate thereof, wherein

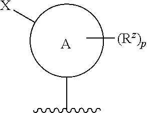

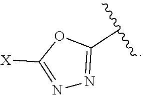

15. The compound of any one of claims 1-13, or pharmaceutically acceptable salt or solvate thereof, wherein

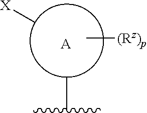

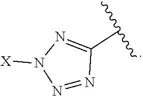

16. The compound of claim 1, or a pharmaceutically acceptable salt or solvate thereof, wherein the compound has the structure of Formula (I):

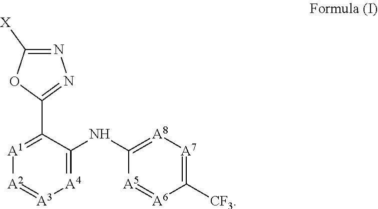

17. The compound of claim 16, or a pharmaceutically acceptable salt or solvate thereof, wherein:
A⁵ is CR²; A⁶ is CR²; A⁷ is CR²; and A⁸ is CR²;
or A⁵ is N; A⁶ is CR²; A⁷ is CR²; and A⁸ is CR²;
or A⁵ is CR²; A⁶ is N; A⁷ is CR²; and A⁸ is CR²;
or A⁵ is CR²; A⁶ is CR²; A⁷ is N; and A⁸ is CR²;
or A⁵ is CR²; A⁶ is CR²; A⁷ is CR²; and A⁸ is N.

18. The compound of claim 16 or claim 17, or a pharmaceutically acceptable salt or solvate thereof, wherein:
A¹ is CR¹; A² is CR¹; A³ is CR¹; and A⁴ is CR¹;
or A¹ is N; A² is CR¹; A³ is CR¹; and A⁴ is CR¹;
or A¹ is CR¹; A² is N; A³ is CR¹; and A⁴ is CR¹;
or A¹ is CR¹; A² is CR¹; A³ is N; and A⁴ is CR¹;
or A¹ is CR¹; A² is CR¹; A³ is CR¹; and A⁴ is N;
or A¹ is CR¹ or N; A² is CR¹; A³ is CR¹; and A⁴ is N.

19. The compound of any one of claims 16-18, or a pharmaceutically acceptable salt or solvate thereof, wherein: 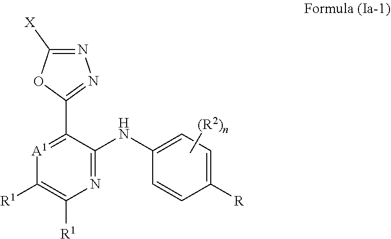
wherein, n is 1, 2, 3, or 4.

20. The compound of any one of claims 1-19, or pharmaceutically acceptable salt or solvate thereof, wherein
each R² is independently H, halogen, —OR⁵, substituted or unsubstituted C₁-C₄alkyl, or substituted or unsubstituted C₁-C₄haloalkyl.

21. The compound of any one of claims 1-20, or pharmaceutically acceptable salt or solvate thereof, wherein each R² is independently H, F, Cl, —OCF₃, or —CF₃.

22. The compound of any one of claims 1-21, or pharmaceutically acceptable salt or solvate thereof, wherein each R² is independently H, F, or Cl.

23. The compound of any one of claims 1-22, or pharmaceutically acceptable salt or solvate thereof, wherein each R² is H.

24. The compound of any one of claims 1-23, or pharmaceutically acceptable salt or solvate thereof, wherein X is substituted or unsubstituted C₁-C₆alkyl, substituted or unsubstituted C₃-C₁₀cycloalkyl, or substituted or unsubstituted C₂-C₁₀heterocycloalkyl.

25. The compound of claim 24, or pharmaceutically acceptable salt or solvate thereof, wherein
X is C₁-C₆alkyl substituted with 1, 2, or 3 substituents each independently selected from —OR¹¹, NR¹¹(C═O)R¹¹, —NR¹¹(C═O)OR¹¹, —O(C═O)OR¹¹, —NR^{11a}R^{11b}, or —(C═O)NR^{11a}R^{11b}; wherein
each R¹¹ is independently H or substituted or unsubstituted C₁-C₆alkyl;
each R^{11a} and R^{11b} is independently H, —CN, —OR¹², —SR¹², substituted or unsubstituted C₁-C₆alkyl, or substituted or unsubstituted C₁-C₆haloalkyl; or
R^{11a} and R^{11b} are taken together with the N atom to which they are attached to form a substituted or unsubstituted N-containing heterocycle; and
each R¹² is independently H or substituted or unsubstituted C₁-C₆alkyl.

26. The compound of claim 24, or pharmaceutically acceptable salt or solvate thereof, wherein
X is C₁-C₆alkyl substituted with —NR^{11a}R^{11b}; wherein
each R^{11a} and R^{11b} is independently H, —CN, or substituted or unsubstituted C₁-C₆alkyl; or
R^{11a} and R^{11b} are taken together with the N atom to which they are attached to form a substituted or unsubstituted N-containing heterocycle.

27. The compound of claim 24, or pharmaceutically acceptable salt or solvate thereof, wherein X is substituted or unsubstituted C₂-C₆heterocycloalkyl.

28. The compound of claim 27, or pharmaceutically acceptable salt or solvate thereof, wherein X is substituted or unsubstituted C₂-C₆heterocycloalkyl comprising 0-2 N, 0-2 O, and 0-2 S ring atoms.

29. The compound of claim 27, or pharmaceutically acceptable salt or solvate thereof, wherein X is substituted or unsubstituted pyrrolidinyl, substituted or unsubstituted pyrrolidinonyl, substituted or unsubstituted piperidinyl, substituted or unsubstituted 1,3-dioxolanyl, substituted or unsubstituted 1,3-dioxolan-2-onyl, substituted or unsubstituted oxazolidinonyl, substituted or unsubstituted oxadiazolidinonyl, substituted or unsubstituted isoxazolidinonyl, substituted or unsubstituted imidazolidin-2-onyl, or substituted or unsubstituted oxadiazolonyl.

30. The compound of claim 27, or pharmaceutically acceptable salt or solvate thereof, wherein X is pyrrolidinyl, pyrrolidinonyl, oxazolidinonyl, isoxazolidinonyl, or imidazolidin-2-onyl, each substituted with 1, 2, 3, or 4 substituents each independently selected from F, —OH, —CN, substituted or unsubstituted C₁-C₆alkyl, substituted or unsubstituted C₁-C₆alkoxy, substituted or unsubstituted C₁-C₆hydroxyalkyl, substituted or unsubstituted C₁-C₆alkylamino, and substituted or unsubstituted C₁-C₆aminoalkyl.

31. The compound of claim 27, or pharmaceutically acceptable salt or solvate thereof, wherein X is pyrrolidinyl, pyrrolidinonyl, oxazolidinonyl, isoxazolidinonyl, or imidazolidin-2-onyl, each substituted with 1 or 2 substituents each independently selected from F, —OH, —CN, —CH₃, —CH₂CH₃, —OCH₃, and —OCH₂CH₃.

32. The compound of claim 27, or pharmaceutically acceptable salt or solvate thereof, wherein X is 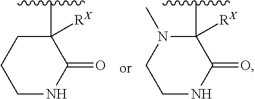
wherein R^{X} is C₁-C₃ alkyl.

33. The compound of claim 27, or pharmaceutically acceptable salt or solvate thereof, wherein X is 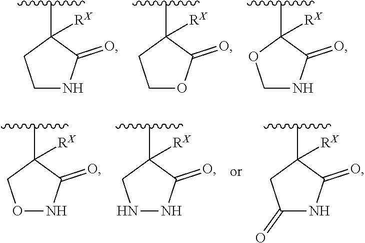
wherein R^{X} is C₁-C₃ alkyl.

34. The compound of claim 27, or pharmaceutically acceptable salt or solvate thereof, wherein X is

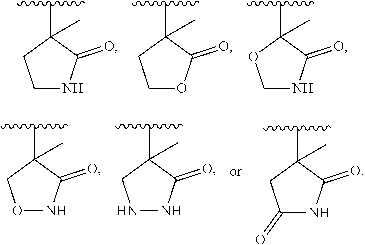

35. The compound of claim 27, or pharmaceutically acceptable salt or solvate thereof, wherein X is

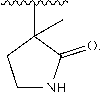 [SMILES: *C1(C)CCNC1=O]

36. The compound of any one of claims 1-35, or a pharmaceutically acceptable salt or solvate thereof, wherein R is F, Cl, —SF₅, —CN, —OCF₃, —CHF₂, or —CF₃.

37. The compound of any one of claims 1-35, or a pharmaceutically acceptable salt or solvate thereof, wherein R is F, Cl, —OCF₃, —CHF₂, or —CF₃.

38. The compound of any one of claims 1-35, or a pharmaceutically acceptable salt or solvate thereof, wherein R is —CF₃ or —SF₅.

39. A compound, or a pharmaceutically acceptable salt or solvate thereof, wherein the compound has one of the following structures: 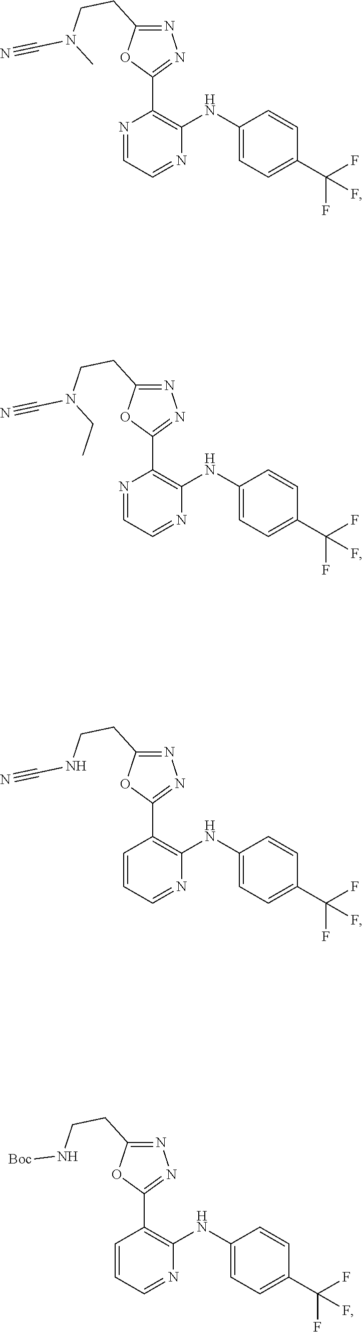 [SMILES: CN(C#N)CCc1nnc(-c2nccnc2Nc2ccc(C(F)(F)F)cc2)o1 ; CCN(C#N)CCc1nnc(-c2nccnc2Nc2ccc(C(F)(F)F)cc2)o1 ; N#CNCCc1nnc(-c2cccnc2Nc2ccc(C(F)(F)F)cc2)o1 ; CC(C)(C)OC(=O)NCCc1nnc(-c2cccnc2Nc2ccc(C(F)(F)F)cc2)o1] 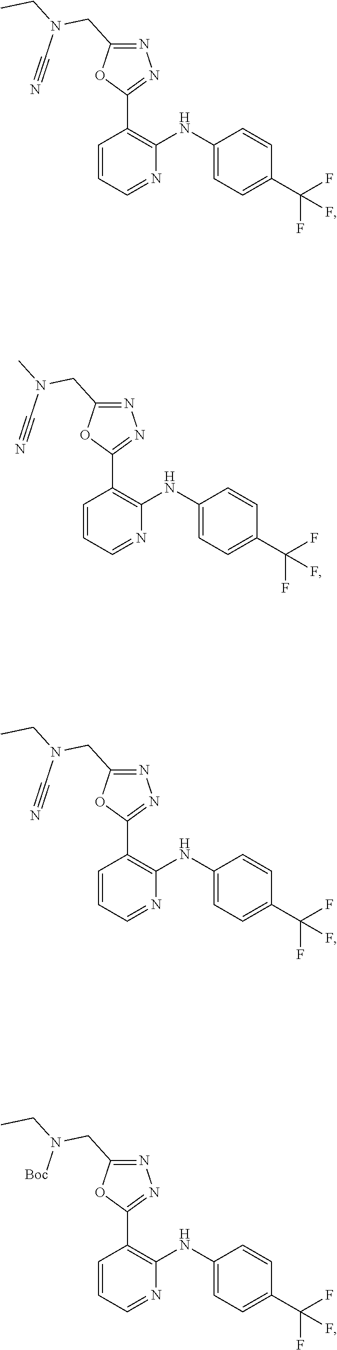 [SMILES: CCN(C#N)Cc1nnc(-c2cccnc2Nc2ccc(C(F)(F)F)cc2)o1 ; CN(C#N)Cc1nnc(-c2cccnc2Nc2ccc(C(F)(F)F)cc2)o1 ; CCN(C#N)Cc1nnc(-c2cccnc2Nc2ccc(C(F)(F)F)cc2)o1 ; CCN(Cc1nnc(-c2cccnc2Nc2ccc(C(F)(F)F)cc2)o1)C(=O)OC(C)(C)C] 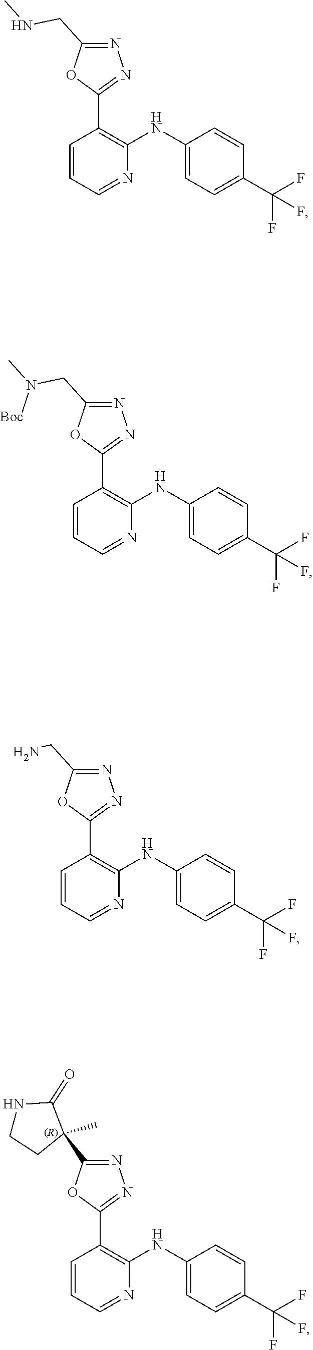 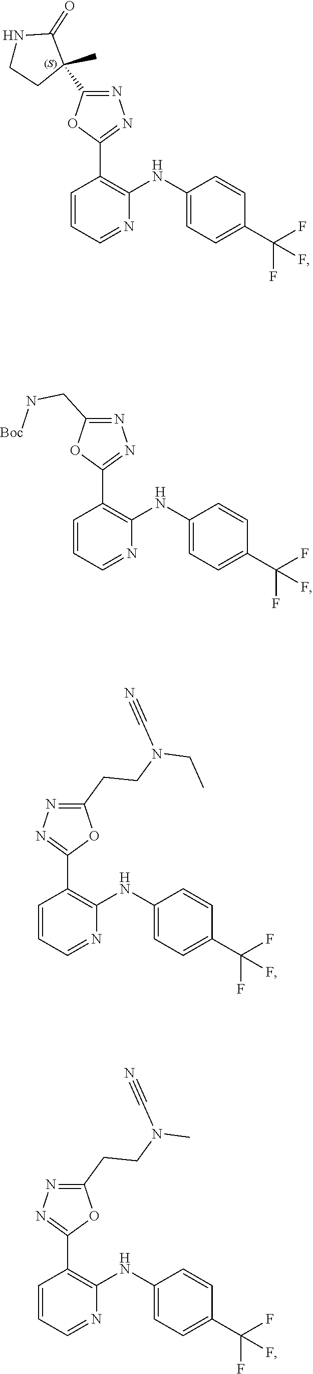 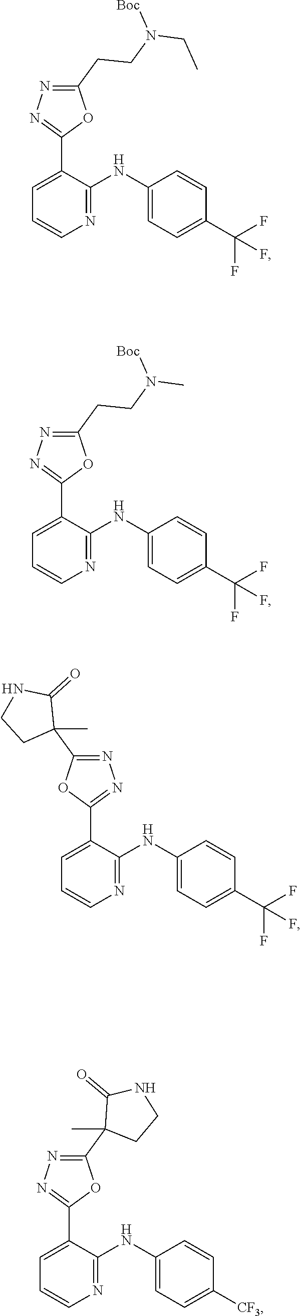 [SMILES: CCN(CCc1nnc(-c2cccnc2Nc2ccc(C(F)(F)F)cc2)o1)C(=O)OC(C)(C)C ; CN(CCc1nnc(-c2cccnc2Nc2ccc(C(F)(F)F)cc2)o1)C(=O)OC(C)(C)C ; CC1(c2nnc(-c3cccnc3Nc3ccc(C(F)(F)F)cc3)o2)CCNC1=O ; CC1(c2nnc(-c3cccnc3Nc3ccc(C(F)(F)F)cc3)o2)CCNC1=O] 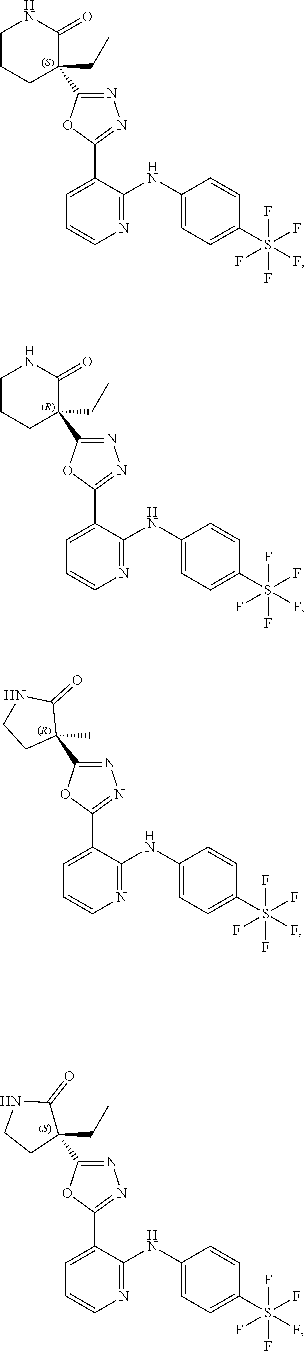 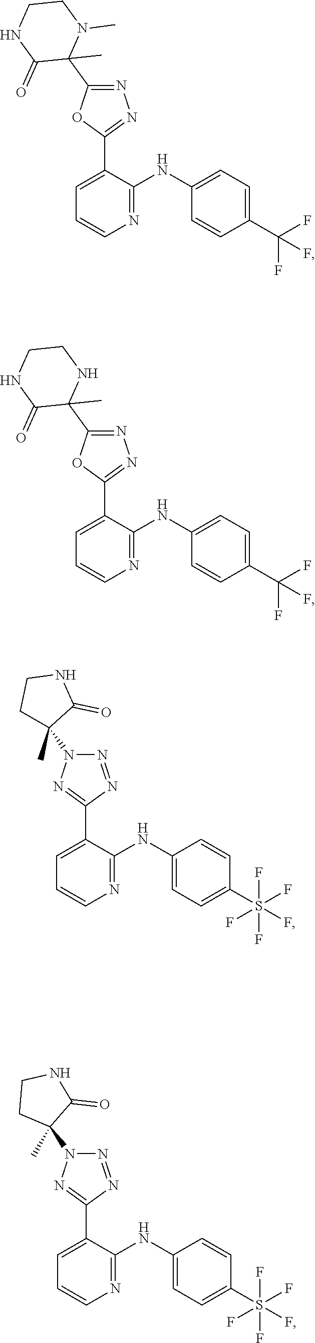 [SMILES: CN1CCNC(=O)C1(C)c1nnc(-c2cccnc2Nc2ccc(C(F)(F)F)cc2)o1 ; CC1(c2nnc(-c3cccnc3Nc3ccc(C(F)(F)F)cc3)o2)NCCNC1=O ; C[C@@]1(n2nnc(-c3cccnc3Nc3ccc(S(F)(F)(F)(F)F)cc3)n2)CCNC1=O ; C[C@]1(n2nnc(-c3cccnc3Nc3ccc(S(F)(F)(F)(F)F)cc3)n2)CCNC1=O] 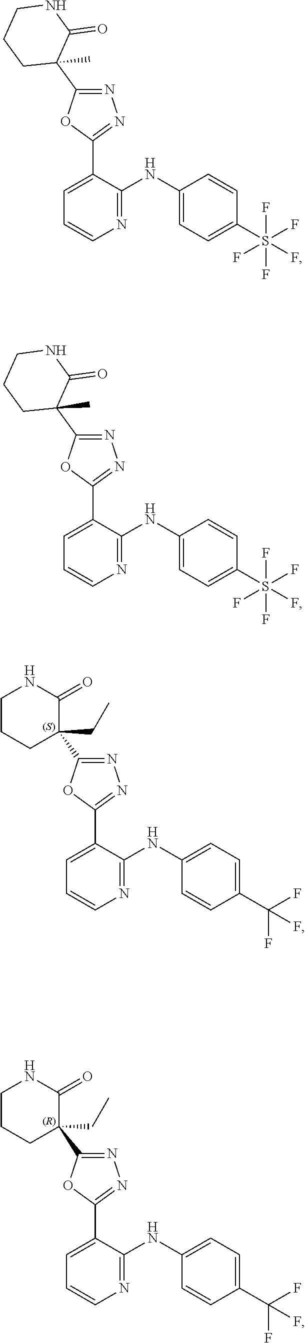 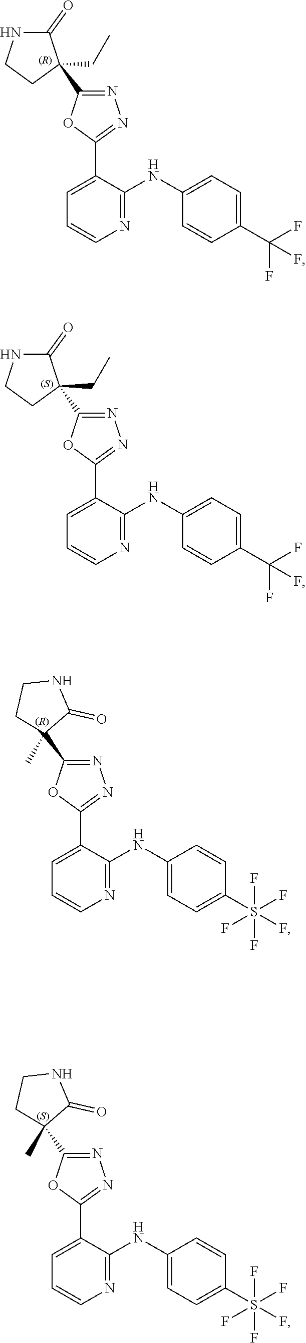 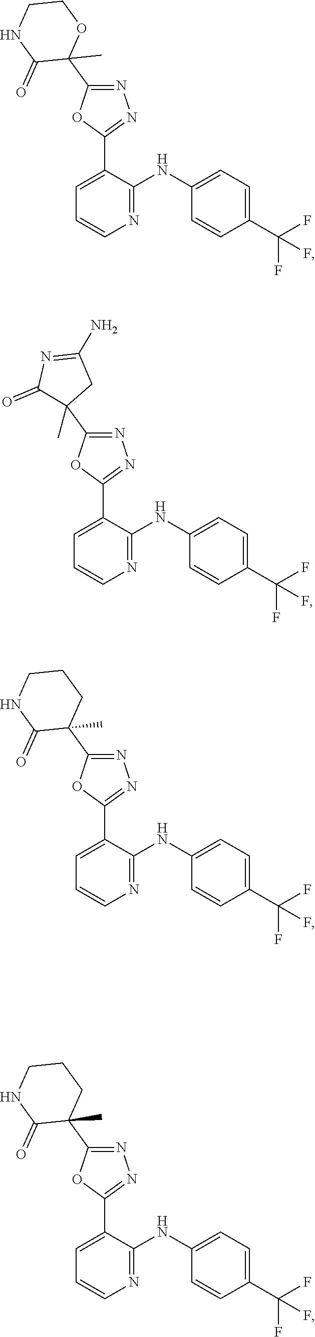 [SMILES: CC1(c2nnc(-c3cccnc3Nc3ccc(C(F)(F)F)cc3)o2)OCCNC1=O ; CC1(c2nnc(-c3cccnc3Nc3ccc(C(F)(F)F)cc3)o2)CC(N)=NC1=O ; C[C@@]1(c2nnc(-c3cccnc3Nc3ccc(C(F)(F)F)cc3)o2)CCCNC1=O ; C[C@]1(c2nnc(-c3cccnc3Nc3ccc(C(F)(F)F)cc3)o2)CCCNC1=O] 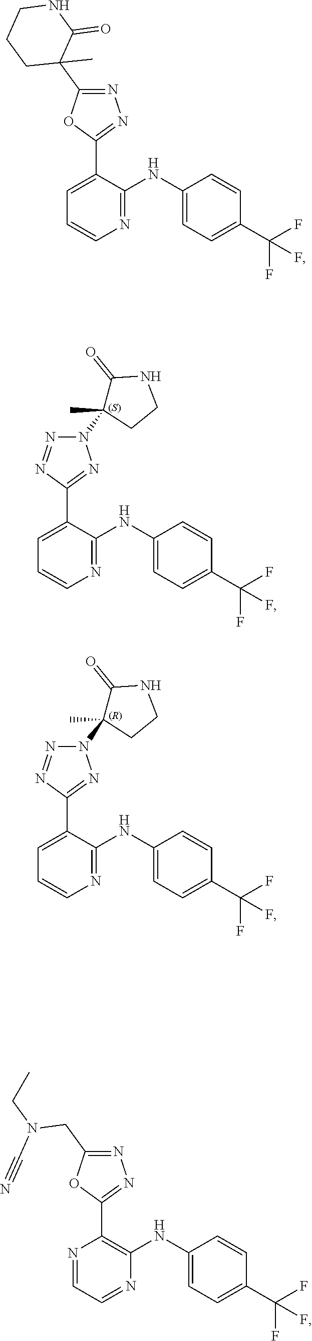 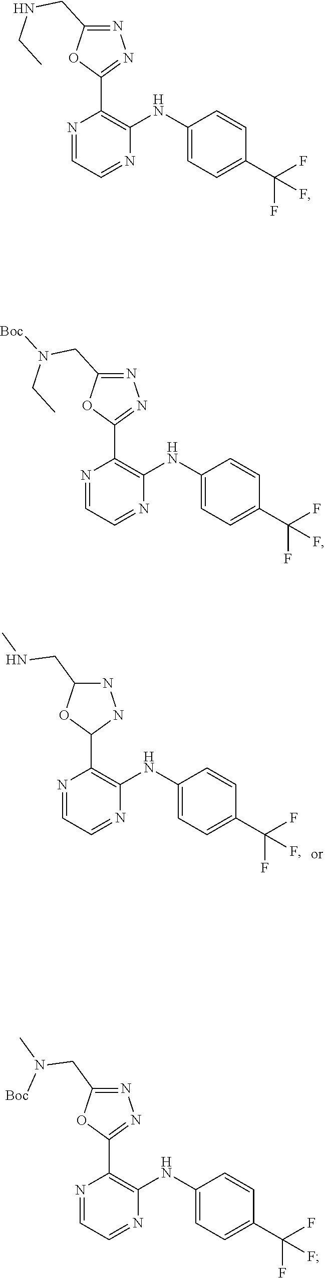 [SMILES: CCNCc1nnc(-c2nccnc2Nc2ccc(C(F)(F)F)cc2)o1 ; CCN(Cc1nnc(-c2nccnc2Nc2ccc(C(F)(F)F)cc2)o1)C(=O)OC(C)(C)C ; CNCC1NNC(c2nccnc2Nc2ccc(C(F)(F)F)cc2)O1 ; CN(Cc1nnc(-c2nccnc2Nc2ccc(C(F)(F)F)cc2)o1)C(=O)OC(C)(C)C]
or a pharmaceutically acceptable salt or solvate thereof.

40. A pharmaceutical composition comprising a pharmaceutically acceptable excipient and a compound of any one of claims 1-39, or a pharmaceutically acceptable salt or solvate thereof.

41. A method of inhibiting one or more of proteins encompassed by, or related to, the Hippo pathway in a subject, comprising administering to a subject a compound of any one of claims 1-39, or a pharmaceutically acceptable salt or solvate thereof.

42. A method of inhibiting transcriptional coactivator with PDZ binding motif/Yes-associated protein transcriptional coactivator (TAZ/YAP) in a subject comprising administering to a subject a compound of any one of claims 1-39, or a pharmaceutically acceptable salt or solvate thereof.

43. The method of claim 41 or claim 42, wherein the subject has cancer, polycystic kidney disease, or liver fibrosis.

44. The method of claim 43, wherein the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

45. A method of treating cancer in a subject in need thereof comprising administering to the subject in need thereof a therapeutically effective amount of a compound of any one of claims 1-39, or a pharmaceutically acceptable salt or solvate thereof.

46. The method of claim 45, wherein the cancer is selected from mesothelioma, hepatocellular carcinoma, meningioma, malignant peripheral nerve sheath tumor, Schwannoma, lung cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer.

47. A method of treating polycystic kidney disease or liver fibrosis in a subject in need thereof comprising administering to the subject in need thereof a therapeutically effective amount of a compound of any one of claims 1-39, or a pharmaceutically acceptable salt or solvate thereof.
